(19) United States
(12) Patent Application Publication
(10) Pub. No.: US 2025/0281514 A1
(43) Pub. Date: Sep. 11, 2025

(54) TREATMENT OF CANCER PATIENTS HAVING KRAS MUTATIONS

(71) Applicant: ASCENTAWITS PHARMACEUTICALS, LTD., Pingshan District, Shenzhen, Guangdong (CN)

(72) Inventors: Jianxin DUAN, Shenzhen, Guangdong (CN); Anrong LI, Foster City, CA (US); Fanying MENG, San Francisco, CA (US); Tianyang QI, Shenzhen, Guangdong (CN)

(21) Appl. No.: 18/693,111

(22) PCT Filed: Sep. 23, 2022
(86) PCT No.: PCT/CN2022/120817

§ 371 Date: Mar. 18, 2024

(30) Foreign Application Priority Data
Sep. 26, 2021 (CN) 202111130589.3

(51) Int. Cl. A61K 31/675; A61P 35/00

(52) U.S. Cl. CPC A61K 31/675; A61P 35/00

(57) ABSTRACT

A method for treating cancer and tumor patients having KRAS mutations, and pharmaceutical use.

### CROSS-REFERENCE TO RELATED APPLICATIONS

This application claims priority to co-pending PCT International Application Serial No. PCT/CN2022/120817 filed Sep. 23, 2022, which claims priority to CN Patent Application No. 202111130589.3 filed on Sep. 26, 2021, the entire content of both of which is incorporated herein by reference.

### TECHNICAL FIELD

The present invention relates to a treatment method of cancer, and in particular to a treatment method of cancer patients having KRAS mutations.

### BACKGROUND

The full name of the KRAS gene is Kirsten ratsarcoma viral oncogene homolog. The protein encoded by the KRAS gene is a small GTPase, which belongs to the RAS protein superfamily. The RAS genes include three types of genes: KRAS, NRAS, and HRAS. The proteins encoded by these genes are GTPases, which act as molecular switches in the pathways that regulate cell proliferation and survival. Based on current COSMIC data, KRAS is the most commonly mutated gene of the three genes (22%), followed by NRAS (8%), and finally HRAS (3%). KRAS gene has a significant impact on human cancers, with KRAS mutations present in approximately 30% of all cancer patients including 90% of pancreatic cancer, 50% of colon cancer, and 25% of lung cancer. In non-small cell lung cancer, KRAS gene mutations account for 20-30% of the cases, mostly present in lung adenocarcinoma, and rarer in lung squamous carcinoma. KRAS gene mutations are mainly concentrated in codon positions 12, 13 and 61, wherein mutations at codon position 12 account for more than 80% of all mutations, including G12A, G12C, G12D, G12R, G12S and G12V. The most common way in which the KRAS gene is activated is point mutation, with 95% of KRAS mutations occurring mainly at codon 12 (>80%) and codon 13 of exon 2. Among the common forms of mutation are KRAS-G12C mutation (accounting for 13% of all KRAS mutations), KRAS-G12V (20%) and KRAS-G12D (29%) mutations (Loong HHF, Du N, Cheng C, Lin H, Guo J, Lin G, Li M, Jiang T, Shi Z, Cui Y, Jin X, Yao J, Xing Y, Yao M, Wang K, Mok TSK, Liu L. KRAS G12C mutations in Asia: A landscape analysis of 11,951 Chinese tumor samples. Transl Lung Cancer Res 2020. doi: 10.21037/tlcr-20-455).

G12C mutation in the KRAS gene, whose mutants have a cysteine residue (glycine at position 12 is changed to cysteine), has been used to design covalent inhibitors with preclinical activity. For example, AMG-510 (the first KRAS-G12C inhibitor in clinical development) and MRTX849 have shown strong anti-tumor activity.

On May 29, 2021, Sotorasib (AMG-510, Lumakras), known as a “revolutionary anti-cancer drug” in the industry and effective against KRAS mutations, was approved by the FDA for the treatment of non-small cell lung cancer patients with KRAS-G12C mutations who have received at least one prior systemic therapy. AMG510 is specifically designed for the mutant subtype KRAS-G12C and has high selectivity. AMG510 can specifically bind to KRAS-G12C in over 6000 proteins, locking in and inactivating it.

On Jun. 24, 2021, the U.S. Food and Drug Administration (FDA) approved breakthrough therapy designation to Adagrasib (MRTX849) for the treatment of patients with non-small cell lung cancer (NSCLC) harboring the KRAS-G12C mutation who have received prior systemic therapy. The drug is an oral inhibitor optimized specifically for the KRAS-G12C mutant. By irreversibly and selectively binding to KRAS-G12C in its inactive state, preventing it from sending cell growth signals and causing cancer cell death, MRTX849 has shown promising safety and anticancer activity in the treatment of non-small cell lung cancer (NSCLC) and colorectal cancer (CRC) harboring the KRAS-G12C mutation, as well as other solid tumors, as demonstrated in preliminary human clinical trials.

However, no drugs have been marketed for the broader G12D subtype of the KRAS mutation.

### SUMMARY OF THE INVENTION

AST-3424 (OBI-3424) is a first-in-class DNA alkylating cancer therapeutic drug targeting overexpression of aldo-keto reductase 1C3 (AKR1C3), which selectively targets cancers overexpressing AKR1C3 and selectively releases a potent DNA alkylating agent in the presence of AKR1C3 enzyme. This selective mode of activation distinguishes AST-3424 from conventional alkylating agents (such as cyclophosphamide and ifosfamide). AKR1C3 overexpression is present in a wide range of treatment-resistant and refractory cancers, including HCC, castration-resistant prostate cancer (CRPC), and T-cell acute lymphoblastic leukemia (T-ALL). AKR1C3 is highly expressed in up to 15 solid and hematologic tumors. Currently, the drug is undergoing phase II clinical trials in China and the United States (US OBI-3424-NCT03592264-phase II, castration prostate cancer and liver cancer; US OBI-3424-NCT04315324-phase II, T-lymphoblastic acute leukemia (T-ALL); China AST-3424-CTR20191399-phase II, solid tumors; ChinaAST-3424-CTR20201915-II phase, T-lymphocytic acute leukemia (T-ALL) and B-lymphocytic acute leukemia (B-ALL)).

AST-3424 and compound

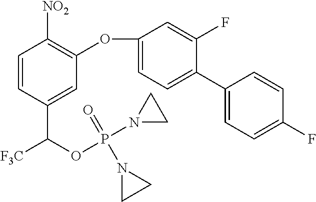 [SMILES: O=[N+]([O-])c1ccc(C(OP(=O)(N2CC2)N2CC2)C(F)(F)F)cc1Oc1ccc(-c2ccc(F)cc2)c(F)c1]

(hereinafter referred to as AST) have been found in an animal in vivo model study of this drug and similar drugs to have good therapeutic efficacy on a KRAS mutant G12D subtype tumor model with high expression of AKR1C3.

Based on the experimental results, the present application provides the following method of treating cancer and pharmaceutical use of the compounds.

A treatment method which uses a drug monotherapy containing an AKR1C3-activated DNA alkylating agent prodrug compound or in combination with other therapeutic drugs for treating cancer and tumor patients having KRAS mutations.

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations.

An AKR1C3-activated DNA alkylating agent prodrug compound means that the compound is a prodrug, and the prodrug molecule reacts with the AKR1C3 enzyme to release a cytotoxic DNA alkylating agent upon reaction.

Specifically, taking AST-3424 as an example, these compounds as aldo-keto reductase AKR1C3 specific substrates, can be quickly and effectively reduced only in cancer cells with high expression of AKR1C3, thereby releasing cytotoxic-DNA alkylating agent AST-2660, and AST-2660 is cross-linked with the DNA, subsequently leads to cancer cells death:

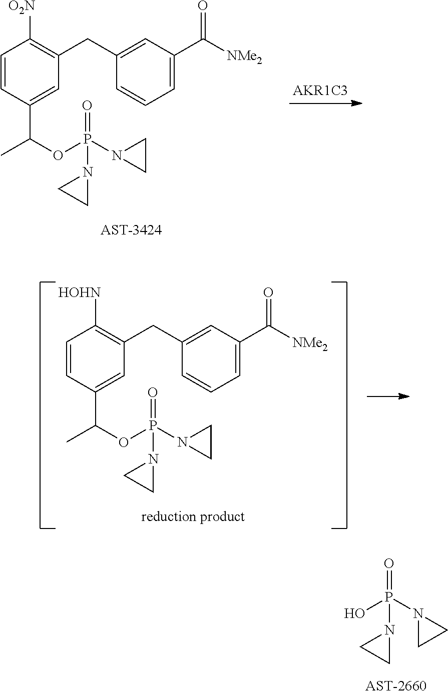

A treatment method which uses a drug monotherapy containing an AKR1C3-activated DNA alkylating agent prodrug compound or in combination with other therapeutic drugs for treating cancer and tumor patients having KRAS mutations, wherein the compound is selected from structural Formula (1) or (2) and salts, esters, solvates, and isotopic isomers thereof:

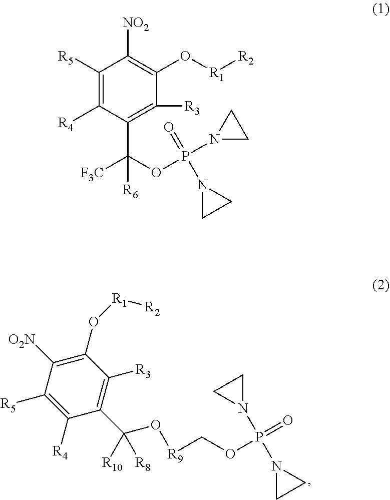

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations, wherein the compound is selected from the structural Formula (1) or (2) and salts, esters, solvates, and isotopic isomers thereof.

Wherein the definitions of R₁, R₂, R₃, R₄, R₅, R₈, R₉, and R₁₀ are described in the claims of Patent Application PCT/CN2020/089692 with Publication No. WO2020228685A1.

Specifically, the groups are defined as follows:
wherein,
R₁ is C₆-C₁₀ aryl or Z-substituted aryl, 4-15 membered heterocycle or Z-substituted heterocycle, 5-15 membered heteroaryl or Z-substituted heteroaryl, a 7-15 membered fused ring or Z-substituted fused ring;
R₂ is hydrogen, a halogen atom, cyano or isocyano, hydroxy, sulfhydryl, amino, OTs, OMS, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15 membered heterocycle or Z-substituted heterocycle, 5-15 membered heteroaryl or Z-substituted heteroaryl, ether having from 1 to 6 carbon atoms or Z-substituted alkoxy having from 1 to 6 carbon atoms, —CONR⁶R⁷, —SO₂NR⁶R⁷, —SO₂R⁶, —OCOO—R⁶, —COOR⁶, —NR⁶COR⁷, —OCOR⁶, —NR⁶SO₂R⁷ or —NR⁶SO₂NR⁶R⁷, or R² together with the atom in the group R¹ to which it is bonded to form a 7-15 membered fused ring or Z-substituted fused ring;
R₃ is hydrogen, halogen, cyano or isocyano, hydroxy, sulfhydryl, amino, OTs, OLCMS, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15 membered heterocycle or Z-substituted heterocycle, 5-15 membered heteroaryl or Z-substituted heteroaryl, C₁-C₆ alkoxy or Z-substituted C₁-C₆ alkoxy, —CONR⁶R⁷, —SO₂NR⁶R⁷, —SO₂R⁶, —OCO—R⁶, —OCOO—R⁶, —COOR⁶, —NR⁶COR⁷, —OCOR⁶, or —NR⁶SO₂R⁷;
R₄ and R₅ are each independently hydrogen, a halogen atom, cyano or isocyano, hydroxy, sulfhydryl, amino, OTs, OLCMS, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15 membered heterocycle or Z-substituted heterocycle, 5-15 membered heteroaryl or Z-substituted heteroaryl, C₁-C₆ alkoxy or Z-substituted C₁-C₆ alkoxy, —CONR⁶R⁷, —SO₂NR⁶R⁷, —SO₂R⁶, —OCOO—R⁶, —COOR⁶, —NR⁶COR⁶, —OCOR⁶ or —NR⁶SO₂R⁷, or R⁴ and R⁵ together with the atom in the benzene ring to which they are bonded to form a 7-15 membered fused ring or Z-substituted fused ring;
R₆ and R₇ are each independently hydrogen, cyano or isocyano, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, C₆-C₁₀ aryl or Z-substituted aryl, 4-15 membered heterocycle or Z-substituted heterocycle, 5-15 membered heteroaryl or Z-substituted heteroaryl, C₁-C₆ alkoxy or Z-substituted C₁-C₆ alkoxy, or R⁶ and R⁷ together with the atom to which they are bonded to form 5-7 membered heterocyclyl or Z-substituted 5-7 membered heterocyclyl;
R₈ and R₁₀ are each independently hydrogen, deuterium, aryl or Z-substituted aryl, C₁-C₆ alkyl or Z-substituted alkyl, C₂-C₆ alkenyl or Z-substituted alkenyl, C₂-C₆ alkynyl or Z-substituted alkynyl, C₃-C₈ cycloalkyl or Z-substituted cycloalkyl, and at least one of R⁸ and R¹⁰ must be hydrogen or deuterium;
R₉ is substituted C₆-C₁₀ aryl which is substituted with at least one fluorine atom or nitro group, substituted 4-15 membered heterocycle which is substituted with at least one fluorine atom or nitro group, or substituted 5-15 membered heteroaryl which is substituted with at least one fluorine atom or nitro group;
the substituent Z is a halogen atom, cyano or isocyano, hydroxy, sulfhydryl, amino, OTs, OMS, C₁-C₃ alkyl or substituted alkyl, C₁-C₃ alkoxy or substituted alkoxy, C₂-C₃ alkenyl or substituted alkenyl, C₂-C₃ alkynyl or substituted alkynyl, C₃-C₈ cycloalkyl or substituted cycloalkyl, an aromatic ring, heterocycle, a heteroaromatic ring and fused ring or a substituted aromatic ring, heterocycle, a heteroaromatic ring and fused ring, the pattern of substitution being mono- or di-substitution;
the substitution in the substituted C₆-C₁₀ aryl, substituted 4-15 membered heterocycle or substituted 5-15 membered heteroaryl in R₉ is a halogen atom, nitro, cyano or isocyano, hydroxy, amino, C₁-C₃ alkyl or alkoxy, alkenyl, alkynyl, cycloalkyl or benzene ring, substituted benzene ring, C₁-C₃ alkoxy or halogen atom-substituted alkoxy.

Specifically, the compounds of Formulae (1) and (2) are selected from the group consisting of:

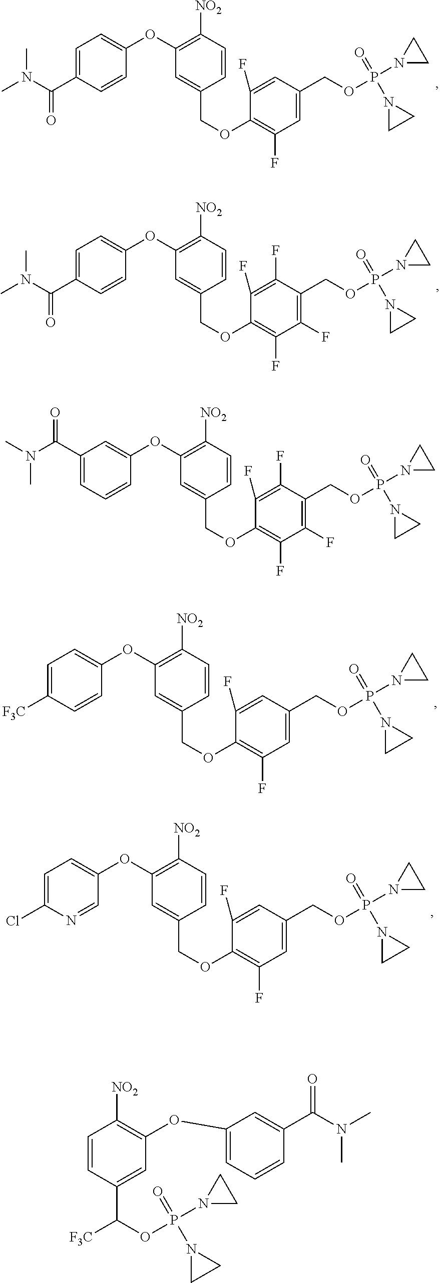 [SMILES: CN(C)C(=O)c1ccc(Oc2cc(COc3c(F)cc(COP(=O)(N4CC4)N4CC4)cc3F)ccc2[N+](=O)[O-])cc1 ; CN(C)C(=O)c1ccc(Oc2cc(COc3c(F)c(F)c(COP(=O)(N4CC4)N4CC4)c(F)c3F)ccc2[N+](=O)[O-])cc1 ; CN(C)C(=O)c1cccc(Oc2cc(COc3c(F)c(F)c(COP(=O)(N4CC4)N4CC4)c(F)c3F)ccc2[N+](=O)[O-])c1 ; O=[N+]([O-])c1ccc(COc2c(F)cc(COP(=O)(N3CC3)N3CC3)cc2F)cc1Oc1ccc(C(F)(F)F)cc1 ; O=[N+]([O-])c1ccc(COc2c(F)cc(COP(=O)(N3CC3)N3CC3)cc2F)cc1Oc1ccc(Cl)nc1 ; CN(C)C(=O)c1cccc(Oc2cc(C(OP(=O)(N3CC3)N3CC3)C(F)(F)F)ccc2[N+](=O)[O-])c1]

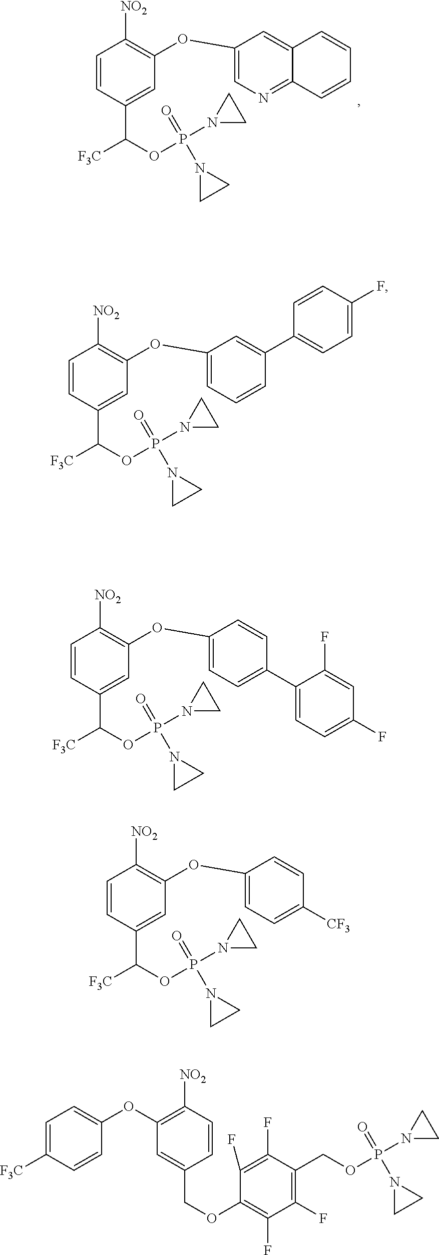 [SMILES: O=[N+]([O-])c1ccc(C(OP(=O)(N2CC2)N2CC2)C(F)(F)F)cc1Oc1cnc2ccccc2c1 ; O=[N+]([O-])c1ccc(C(OP(=O)(N2CC2)N2CC2)C(F)(F)F)cc1Oc1cccc(-c2ccc(F)cc2)c1 ; O=[N+]([O-])c1ccc(C(OP(=O)(N2CC2)N2CC2)C(F)(F)F)cc1Oc1ccc(-c2ccc(F)cc2F)cc1 ; O=[N+]([O-])c1ccc(C(OP(=O)(N2CC2)N2CC2)C(F)(F)F)cc1Oc1ccc(C(F)(F)F)cc1 ; O=[N+]([O-])c1ccc(COc2c(F)c(F)c(COP(=O)(N3CC3)N3CC3)c(F)c2F)cc1Oc1ccc(C(F)(F)F)cc1]

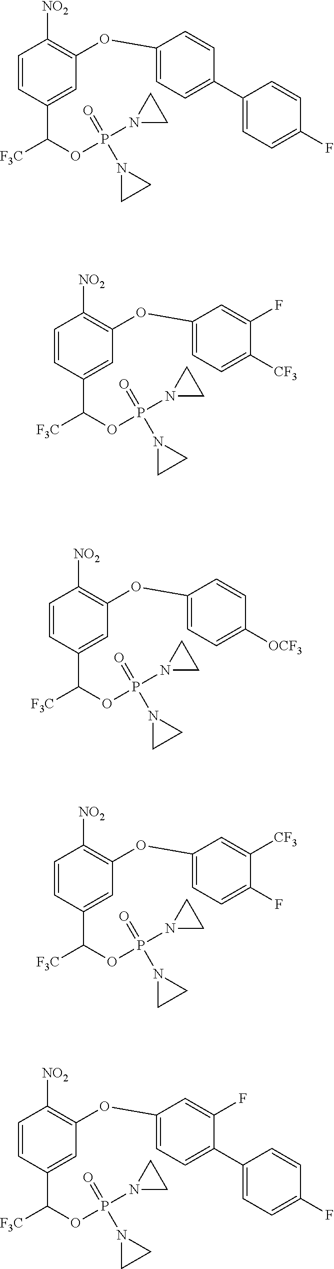 [SMILES: O=[N+]([O-])c1ccc(C(OP(=O)(N2CC2)N2CC2)C(F)(F)F)cc1Oc1ccc(-c2ccc(F)cc2)cc1 ; O=[N+]([O-])c1ccc(C(OP(=O)(N2CC2)N2CC2)C(F)(F)F)cc1Oc1ccc(C(F)(F)F)c(F)c1 ; O=[N+]([O-])c1ccc(C(OP(=O)(N2CC2)N2CC2)C(F)(F)F)cc1Oc1ccc(OC(F)(F)F)cc1 ; O=[N+]([O-])c1ccc(C(OP(=O)(N2CC2)N2CC2)C(F)(F)F)cc1Oc1ccc(F)c(C(F)(F)F)c1 ; O=[N+]([O-])c1ccc(C(OP(=O)(N2CC2)N2CC2)C(F)(F)F)cc1Oc1ccc(-c2ccc(F)cc2)c(F)c1]

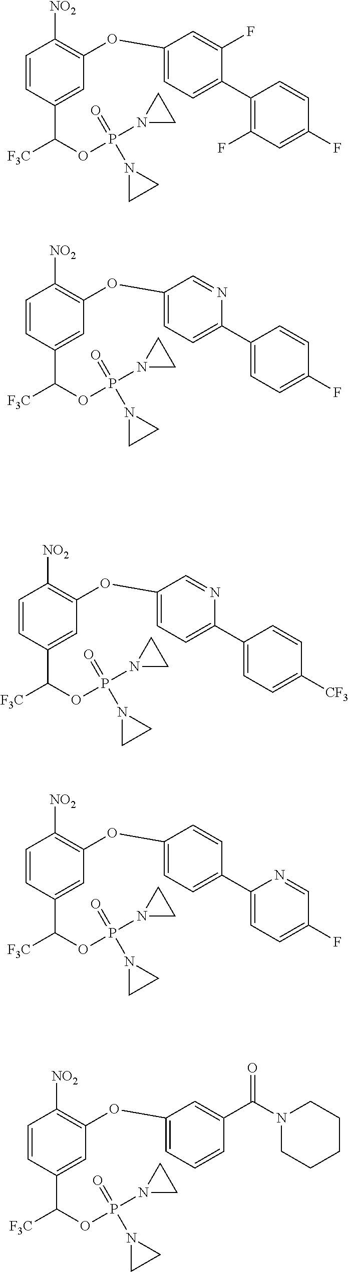 [SMILES: O=[N+]([O-])c1ccc(C(OP(=O)(N2CC2)N2CC2)C(F)(F)F)cc1Oc1ccc(-c2ccc(F)cc2F)c(F)c1 ; O=[N+]([O-])c1ccc(C(OP(=O)(N2CC2)N2CC2)C(F)(F)F)cc1Oc1ccc(-c2ccc(F)cc2)nc1 ; O=[N+]([O-])c1ccc(C(OP(=O)(N2CC2)N2CC2)C(F)(F)F)cc1Oc1ccc(-c2ccc(C(F)(F)F)cc2)nc1 ; O=[N+]([O-])c1ccc(C(OP(=O)(N2CC2)N2CC2)C(F)(F)F)cc1Oc1ccc(-c2ccc(F)cn2)cc1 ; O=C(c1cccc(Oc2cc(C(OP(=O)(N3CC3)N3CC3)C(F)(F)F)ccc2[N+](=O)[O-])c1)N1CCCCC1]

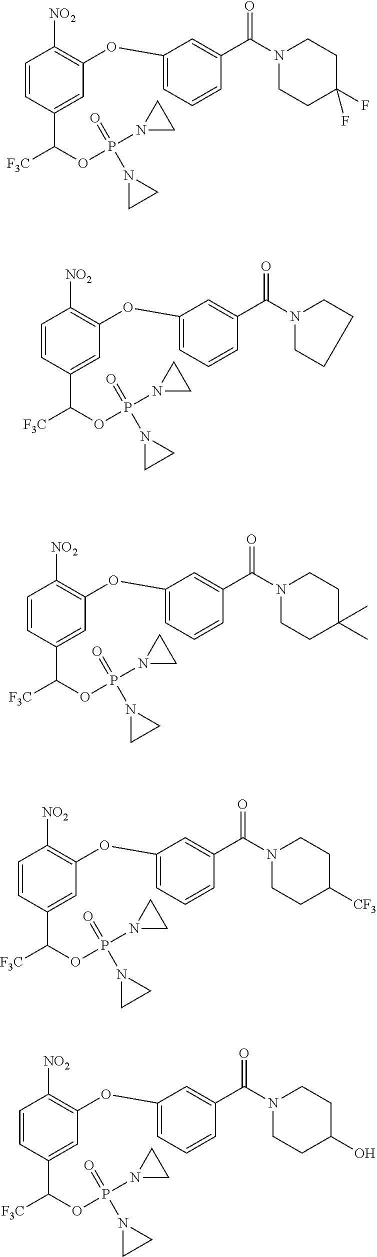 [SMILES: O=C(c1cccc(Oc2cc(C(OP(=O)(N3CC3)N3CC3)C(F)(F)F)ccc2[N+](=O)[O-])c1)N1CCC(F)(F)CC1 ; O=C(c1cccc(Oc2cc(C(OP(=O)(N3CC3)N3CC3)C(F)(F)F)ccc2[N+](=O)[O-])c1)N1CCCC1 ; CC1(C)CCN(C(=O)c2cccc(Oc3cc(C(OP(=O)(N4CC4)N4CC4)C(F)(F)F)ccc3[N+](=O)[O-])c2)CC1 ; O=C(c1cccc(Oc2cc(C(OP(=O)(N3CC3)N3CC3)C(F)(F)F)ccc2[N+](=O)[O-])c1)N1CCC(C(F)(F)F)CC1 ; O=C(c1cccc(Oc2cc(C(OP(=O)(N3CC3)N3CC3)C(F)(F)F)ccc2[N+](=O)[O-])c1)N1CCC(O)CC1]

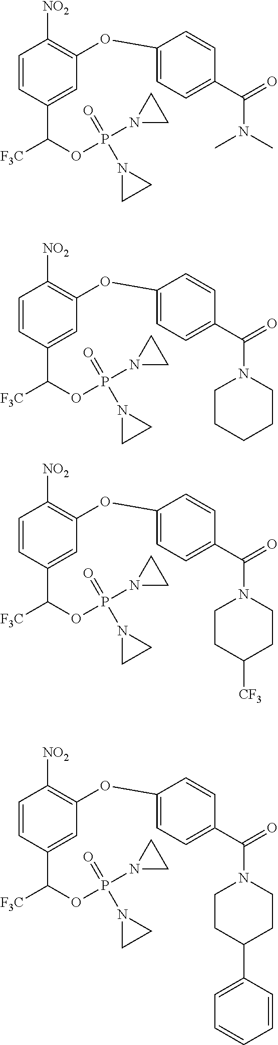 [SMILES: CN(C)C(=O)c1ccc(Oc2cc(C(OP(=O)(N3CC3)N3CC3)C(F)(F)F)ccc2[N+](=O)[O-])cc1 ; O=C(c1ccc(Oc2cc(C(OP(=O)(N3CC3)N3CC3)C(F)(F)F)ccc2[N+](=O)[O-])cc1)N1CCCCC1 ; O=C(c1ccc(Oc2cc(C(OP(=O)(N3CC3)N3CC3)C(F)(F)F)ccc2[N+](=O)[O-])cc1)N1CCC(C(F)(F)F)CC1 ; O=C(c1ccc(Oc2cc(C(OP(=O)(N3CC3)N3CC3)C(F)(F)F)ccc2[N+](=O)[O-])cc1)N1CCC(c2ccccc2)CC1]

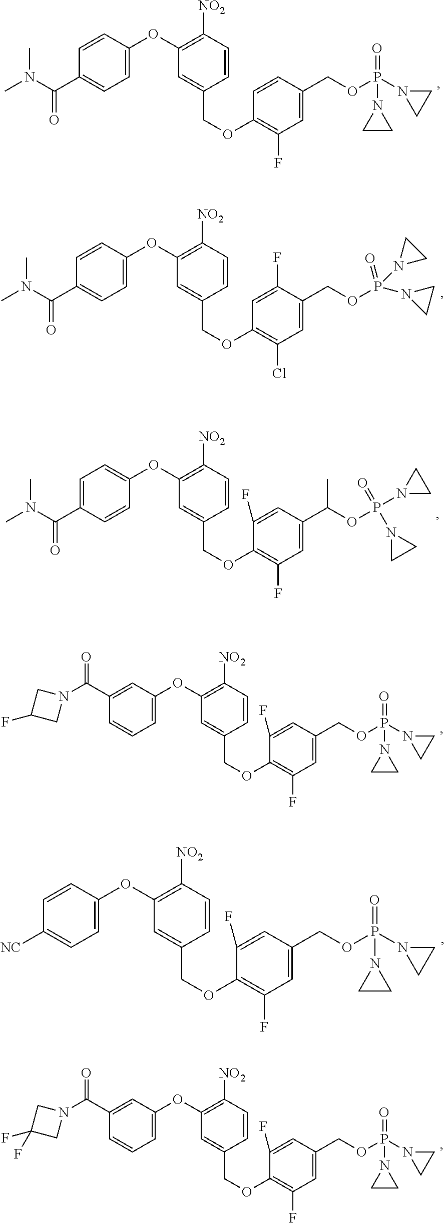 [SMILES: CN(C)C(=O)c1ccc(Oc2cc(COc3ccc(COP(=O)(N4CC4)N4CC4)cc3F)ccc2[N+](=O)[O-])cc1 ; CN(C)C(=O)c1ccc(Oc2cc(COc3cc(F)c(COP(=O)(N4CC4)N4CC4)cc3Cl)ccc2[N+](=O)[O-])cc1 ; CC(OP(=O)(N1CC1)N1CC1)c1cc(F)c(OCc2ccc([N+](=O)[O-])c(Oc3ccc(C(=O)N(C)C)cc3)c2)c(F)c1 ; O=C(c1cccc(Oc2cc(COc3c(F)cc(COP(=O)(N4CC4)N4CC4)cc3F)ccc2[N+](=O)[O-])c1)N1CC(F)C1 ; N#Cc1ccc(Oc2cc(COc3c(F)cc(COP(=O)(N4CC4)N4CC4)cc3F)ccc2[N+](=O)[O-])cc1 ; O=C(c1cccc(Oc2cc(COc3c(F)cc(COP(=O)(N4CC4)N4CC4)cc3F)ccc2[N+](=O)[O-])c1)N1CC(F)(F)C1]

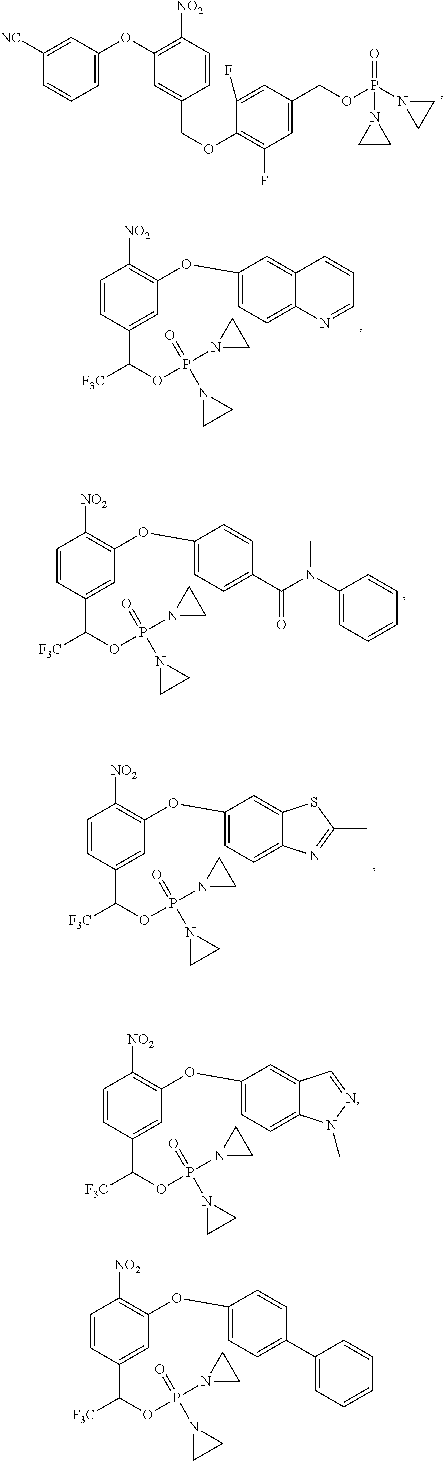 [SMILES: N#Cc1cccc(Oc2cc(COc3c(F)cc(COP(=O)(N4CC4)N4CC4)cc3F)ccc2[N+](=O)[O-])c1 ; O=[N+]([O-])c1ccc(C(OP(=O)(N2CC2)N2CC2)C(F)(F)F)cc1Oc1ccc2ncccc2c1 ; CN(C(=O)c1ccc(Oc2cc(C(OP(=O)(N3CC3)N3CC3)C(F)(F)F)ccc2[N+](=O)[O-])cc1)c1ccccc1 ; Cc1nc2ccc(Oc3cc(C(OP(=O)(N4CC4)N4CC4)C(F)(F)F)ccc3[N+](=O)[O-])cc2s1 ; Cn1ncc2cc(Oc3cc(C(OP(=O)(N4CC4)N4CC4)C(F)(F)F)ccc3[N+](=O)[O-])ccc21 ; O=[N+]([O-])c1ccc(C(OP(=O)(N2CC2)N2CC2)C(F)(F)F)cc1Oc1ccc(-c2ccccc2)cc1]

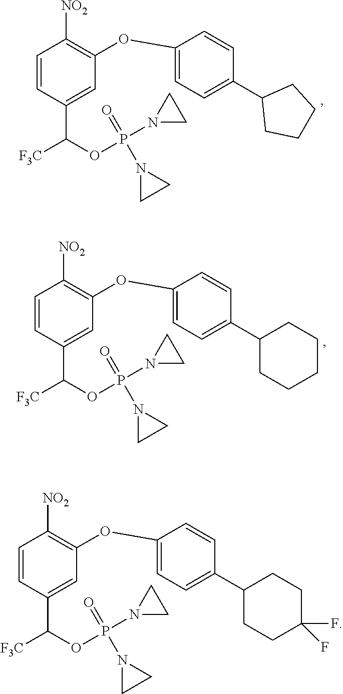 [SMILES: O=[N+]([O-])c1ccc(C(OP(=O)(N2CC2)N2CC2)C(F)(F)F)cc1Oc1ccc(C2CCCC2)cc1 ; O=[N+]([O-])c1ccc(C(OP(=O)(N2CC2)N2CC2)C(F)(F)F)cc1Oc1ccc(C2CCCCC2)cc1 ; O=[N+]([O-])c1ccc(C(OP(=O)(N2CC2)N2CC2)C(F)(F)F)cc1Oc1ccc(C2CCC(F)(F)CC2)cc1]

Specific definitions and meanings of the groups, and the preparation methods and spectral data of the compounds have been described in Patent Application PCT/CN2020/089692 with Publication No. WO2020228685A1, which is incorporated herein by reference in its entirety.

Evidently, the compounds of structural Formula (1) or (2), similar to AST-342 or AST, are prodrugs of AST-2660, and can be activated by the AKR1C3 enzyme to form AST-2660 (an alkylating agent) to exhibit its anti-cancer efficacy:

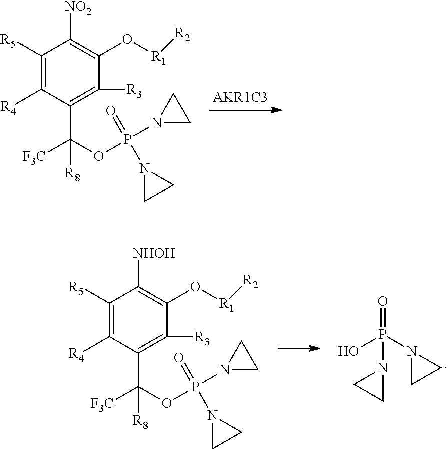

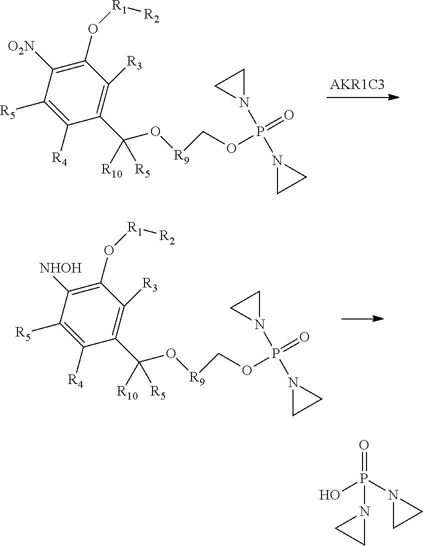

A treatment method which uses a drug monotherapy containing an AKR1C3-activated DNA alkylating agent prodrug compound or in combination with other therapeutic drugs for treating cancer and tumor patients having KRAS mutations, wherein the compound is selected from structural Formula (3) and salts, esters, solvates, and isotopic isomers thereof:

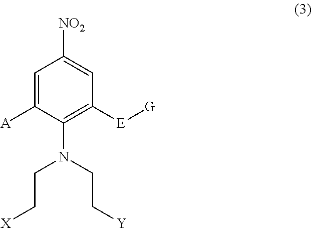

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations, wherein the compound is selected from the structural Formula (3) and salts, esters, solvates, and isotopic isomers thereof:

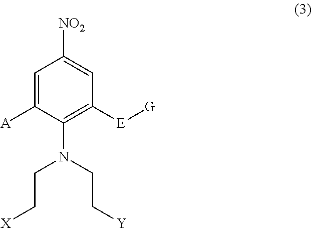

wherein the definitions of A, E, G, X and Y are described in the claims of Patent Application PCT/NZ2019/050030 with Publication No. WO2019190331A1 (corresponding to Chinese Patent Application No. 2019800234236 with Publication No. CN111918864A).

Specifically, the groups are defined as follows:
wherein,
A is H, C₁-C₆ alkyl, C₁-C₆ alkenyl, C₁-C₆ alkynyl, CFH₂, CF₂H, CF₃, F, Cl, Br, I, OCF₃, COR or CON(R)₂;
E is SO or SO₂;
X is C₁, Br, I or OSO₂R;
Y is C₁, Br, I or OSO₂R;
Each R is independently H or C₁-C₆ alkyl;
G is a radical group selected from the group consisting of Formulae (B)-(AA):

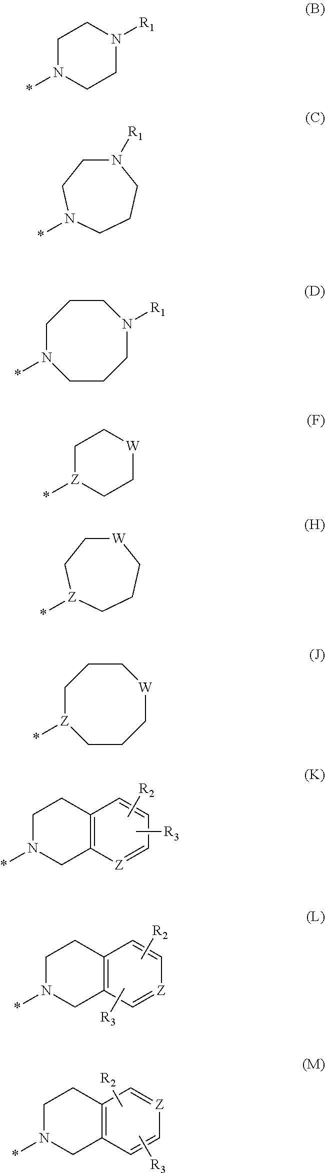

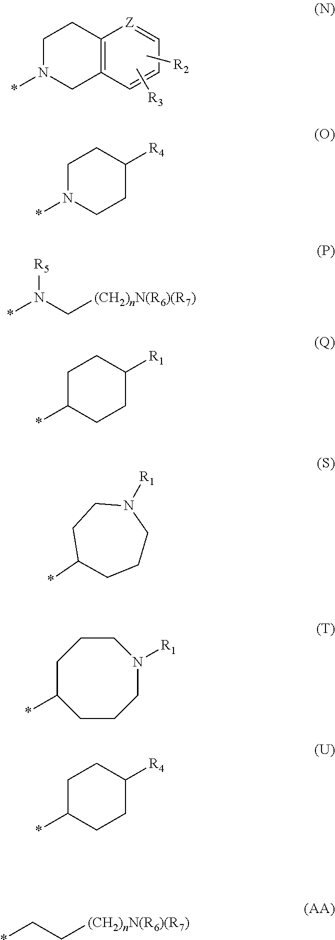

wherein,
R₁ is H, C₁-C₆ alkyl, CH₂(CH₂)ₙOH, CH₂CH(OH)CH₂OH, phenyl, pyridyl, benzyl, or pyridylmethyl, provided that when R₁ is phenyl, pyridyl, benzyl or pyridylmethyl, R₁ is optionally substituted at any available position with C₁-C₆ alkyl, C₁-C₆ alkenyl, C₁-C₆ alkynyl, OR₆, N(R₆)(R₇), CFH₂, CF₂H, CF₃, F, Cl, Br, I, OCF₃, COR₆, CON(R₆)(R₇), SOR₆, SON(R₆)(R₇), SO₂R⁶, SO₂N(R₆)(R₇), CN, or NO₂;
R₂ and R₃ are each independently H, C₁-C₆ alkyl, C₁-C₆ alkenyl, C₁-C₆ alkynyl, OR₆, N(R₆)(R₇), CFH₂, CF₂H, CF₃, F, Cl, Br, I, OCF₃, COR₆, CON(R₆)(R₇), SOR₆, SON(R₆)(R₇), SO₂R⁶, SO₂N(R₆)(R₇), CN or NO₂;
R₄ is N(R₆)(R₇), OH, OCH₂(CH₂)ₙN(R₆)(R₇) or CH₂(CH₂)ₙN(R₆)(R₇);
R₅ is H or a C₁-C₆ alkyl group;
R₆ and R₇ are each independently H or C₁₋₆ alkyl, or R₆ and R₇ together to form substituted or unsubstituted 5-membered or 6-membered heterocycle;
Z is CH or N;
W is CH₂, O, S, SO or SO₂;
n is 0 to 6;
* represents a point of attachment to Formula (I).

Specifically, the compound of Formula (3) is selected from the group consisting of:

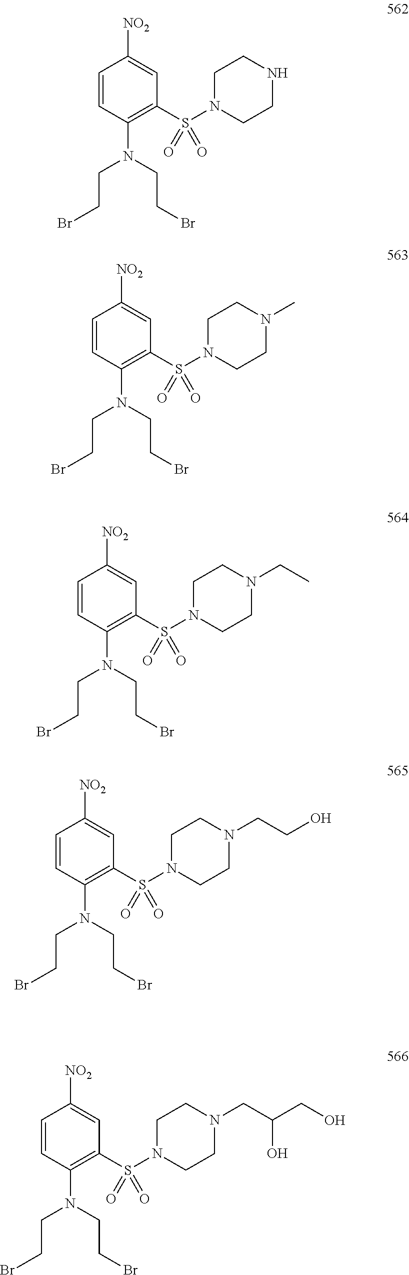 [SMILES: O=[N+]([O-])c1ccc(N(CCBr)CCBr)c(S(=O)(=O)N2CCNCC2)c1 ; CN1CCN(S(=O)(=O)c2cc([N+](=O)[O-])ccc2N(CCBr)CCBr)CC1 ; CCN1CCN(S(=O)(=O)c2cc([N+](=O)[O-])ccc2N(CCBr)CCBr)CC1 ; O=[N+]([O-])c1ccc(N(CCBr)CCBr)c(S(=O)(=O)N2CCN(CCO)CC2)c1 ; O=[N+]([O-])c1ccc(N(CCBr)CCBr)c(S(=O)(=O)N2CCN(CC(O)CO)CC2)c1]

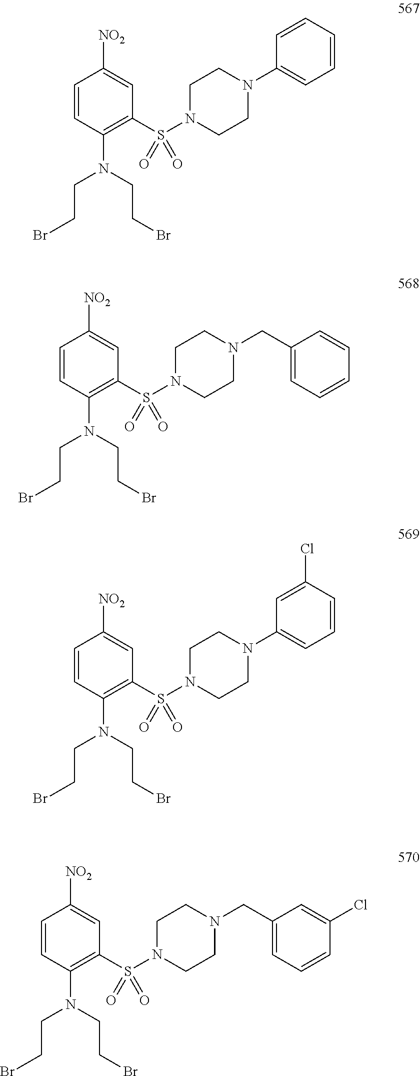 [SMILES: O=[N+]([O-])c1ccc(N(CCBr)CCBr)c(S(=O)(=O)N2CCN(c3ccccc3)CC2)c1 ; O=[N+]([O-])c1ccc(N(CCBr)CCBr)c(S(=O)(=O)N2CCN(Cc3ccccc3)CC2)c1 ; O=[N+]([O-])c1ccc(N(CCBr)CCBr)c(S(=O)(=O)N2CCN(c3cccc(Cl)c3)CC2)c1 ; O=[N+]([O-])c1ccc(N(CCBr)CCBr)c(S(=O)(=O)N2CCN(Cc3cccc(Cl)c3)CC2)c1]

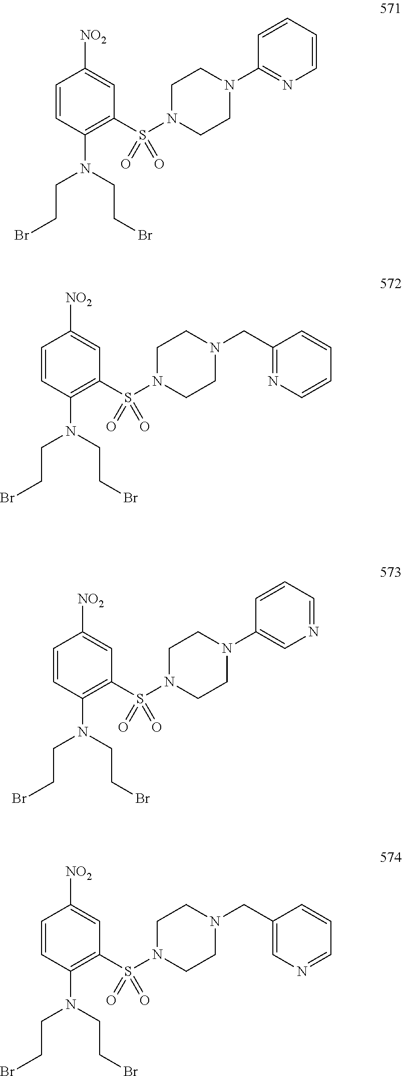 [SMILES: O=[N+]([O-])c1ccc(N(CCBr)CCBr)c(S(=O)(=O)N2CCN(c3ccccn3)CC2)c1 ; O=[N+]([O-])c1ccc(N(CCBr)CCBr)c(S(=O)(=O)N2CCN(Cc3ccccn3)CC2)c1 ; O=[N+]([O-])c1ccc(N(CCBr)CCBr)c(S(=O)(=O)N2CCN(c3cccnc3)CC2)c1 ; O=[N+]([O-])c1ccc(N(CCBr)CCBr)c(S(=O)(=O)N2CCN(Cc3cccnc3)CC2)c1]

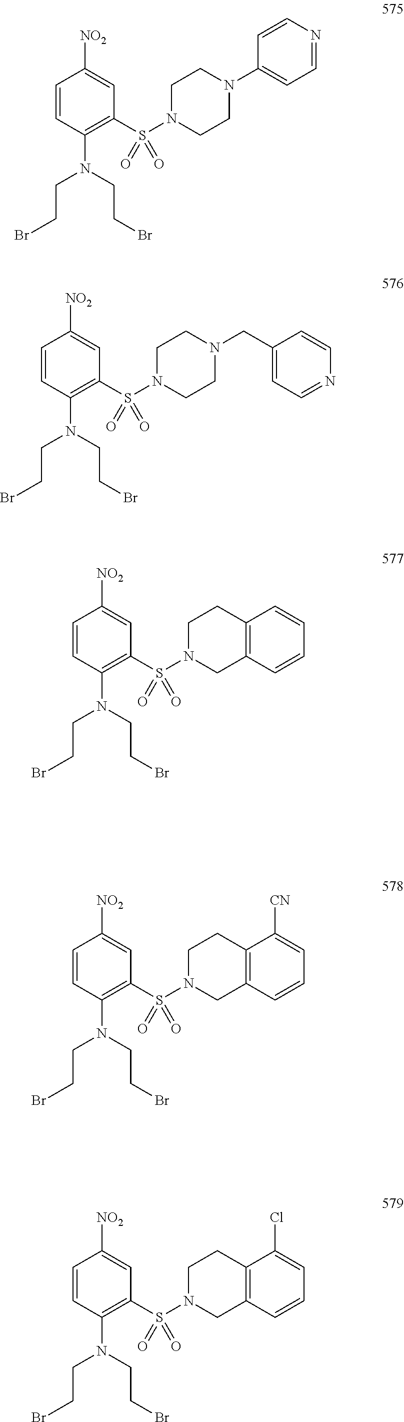 [SMILES: O=[N+]([O-])c1ccc(N(CCBr)CCBr)c(S(=O)(=O)N2CCN(c3ccncc3)CC2)c1 ; O=[N+]([O-])c1ccc(N(CCBr)CCBr)c(S(=O)(=O)N2CCN(Cc3ccncc3)CC2)c1 ; O=[N+]([O-])c1ccc(N(CCBr)CCBr)c(S(=O)(=O)N2CCc3ccccc3C2)c1 ; N#Cc1cccc2c1CCN(S(=O)(=O)c1cc([N+](=O)[O-])ccc1N(CCBr)CCBr)C2 ; O=[N+]([O-])c1ccc(N(CCBr)CCBr)c(S(=O)(=O)N2CCc3c(Cl)cccc3C2)c1]

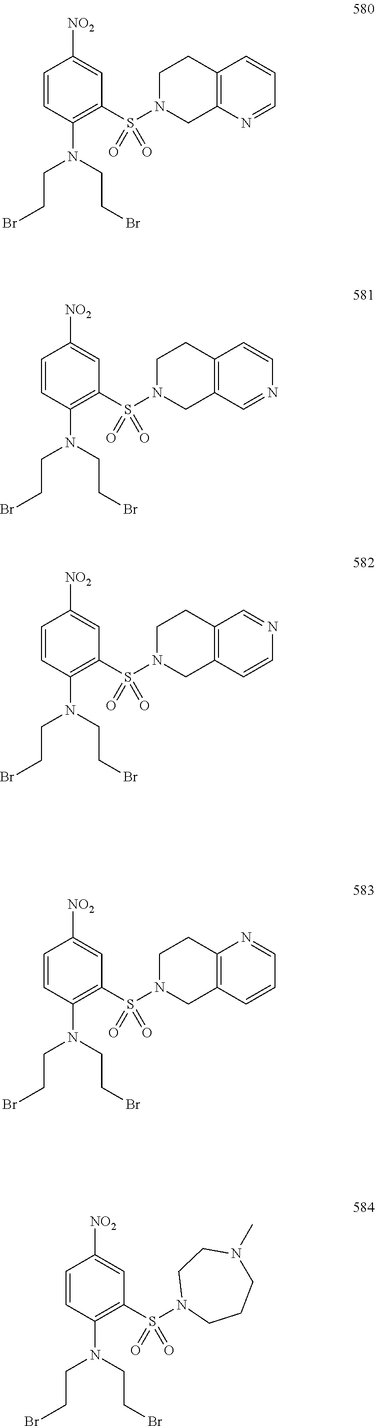 [SMILES: O=[N+]([O-])c1ccc(N(CCBr)CCBr)c(S(=O)(=O)N2CCc3cccnc3C2)c1 ; O=[N+]([O-])c1ccc(N(CCBr)CCBr)c(S(=O)(=O)N2CCc3ccncc3C2)c1 ; O=[N+]([O-])c1ccc(N(CCBr)CCBr)c(S(=O)(=O)N2CCc3cnccc3C2)c1 ; O=[N+]([O-])c1ccc(N(CCBr)CCBr)c(S(=O)(=O)N2CCc3ncccc3C2)c1 ; CN1CCCN(S(=O)(=O)c2cc([N+](=O)[O-])ccc2N(CCBr)CCBr)CC1]

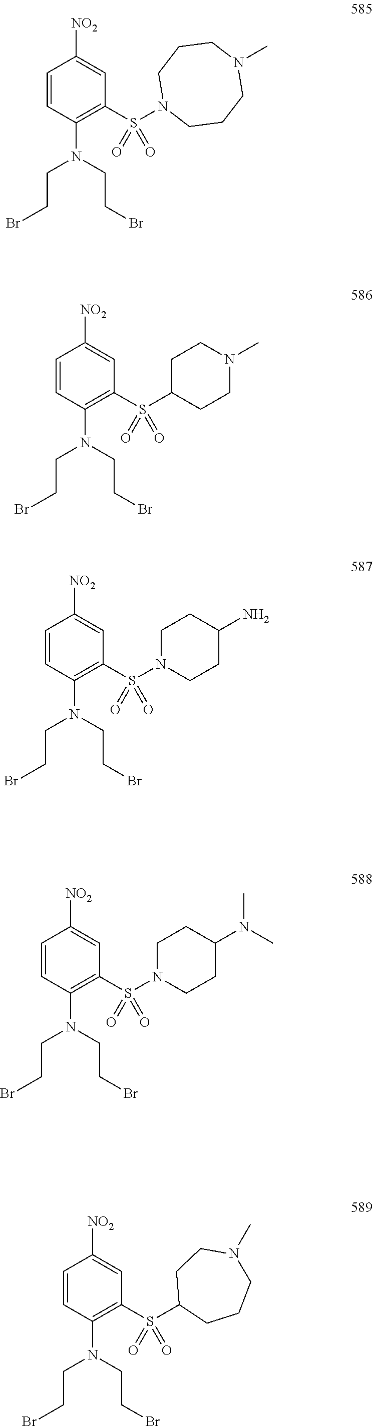 [SMILES: CN1CCCN(S(=O)(=O)c2cc([N+](=O)[O-])ccc2N(CCBr)CCBr)CCC1 ; CN1CCC(S(=O)(=O)c2cc([N+](=O)[O-])ccc2N(CCBr)CCBr)CC1 ; NC1CCN(S(=O)(=O)c2cc([N+](=O)[O-])ccc2N(CCBr)CCBr)CC1 ; CN(C)C1CCN(S(=O)(=O)c2cc([N+](=O)[O-])ccc2N(CCBr)CCBr)CC1 ; CN1CCCC(S(=O)(=O)c2cc([N+](=O)[O-])ccc2N(CCBr)CCBr)CC1]

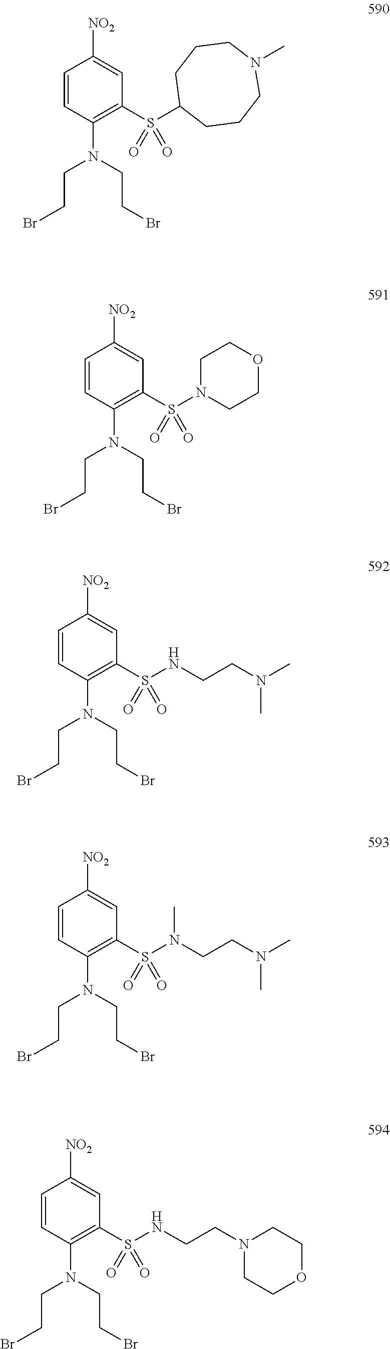 [SMILES: CN1CCCC(S(=O)(=O)c2cc([N+](=O)[O-])ccc2N(CCBr)CCBr)CCC1 ; O=[N+]([O-])c1ccc(N(CCBr)CCBr)c(S(=O)(=O)N2CCOCC2)c1 ; CN(C)CCNS(=O)(=O)c1cc([N+](=O)[O-])ccc1N(CCBr)CCBr ; CN(C)CCN(C)S(=O)(=O)c1cc([N+](=O)[O-])ccc1N(CCBr)CCBr ; O=[N+]([O-])c1ccc(N(CCBr)CCBr)c(S(=O)(=O)NCCN2CCOCC2)c1]

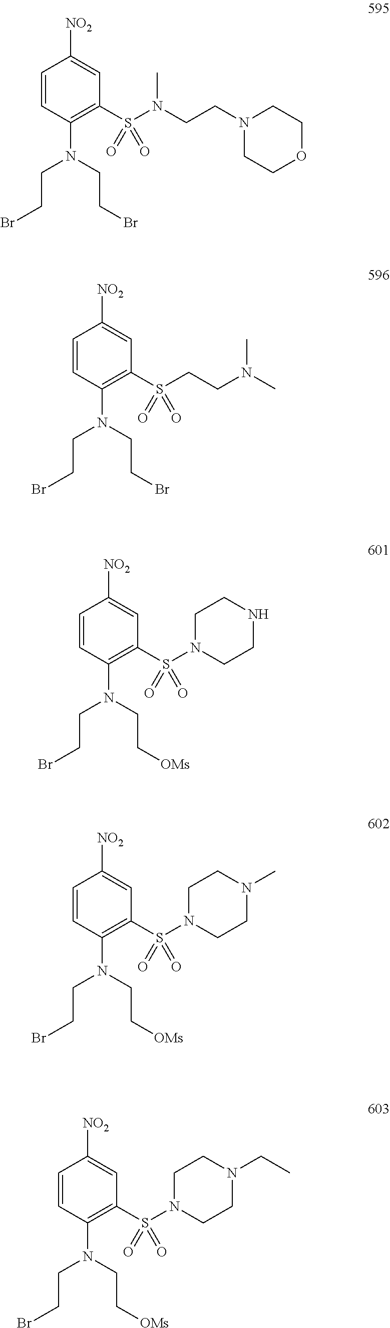 [SMILES: CN(CCN1CCOCC1)S(=O)(=O)c1cc([N+](=O)[O-])ccc1N(CCBr)CCBr ; CN(C)CCS(=O)(=O)c1cc([N+](=O)[O-])ccc1N(CCBr)CCBr ; CS(=O)(=O)OCCN(CCBr)c1ccc([N+](=O)[O-])cc1S(=O)(=O)N1CCNCC1 ; CN1CCN(S(=O)(=O)c2cc([N+](=O)[O-])ccc2N(CCBr)CCOS(C)(=O)=O)CC1 ; CCN1CCN(S(=O)(=O)c2cc([N+](=O)[O-])ccc2N(CCBr)CCOS(C)(=O)=O)CC1]

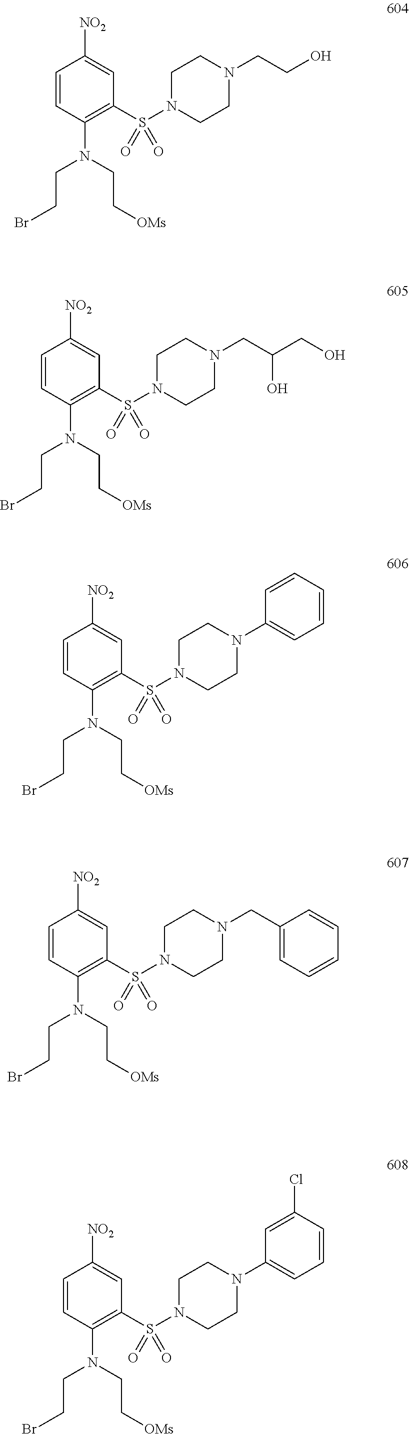 [SMILES: CS(=O)(=O)OCCN(CCBr)c1ccc([N+](=O)[O-])cc1S(=O)(=O)N1CCN(CCO)CC1 ; CS(=O)(=O)OCCN(CCBr)c1ccc([N+](=O)[O-])cc1S(=O)(=O)N1CCN(CC(O)CO)CC1 ; CS(=O)(=O)OCCN(CCBr)c1ccc([N+](=O)[O-])cc1S(=O)(=O)N1CCN(c2ccccc2)CC1 ; CS(=O)(=O)OCCN(CCBr)c1ccc([N+](=O)[O-])cc1S(=O)(=O)N1CCN(Cc2ccccc2)CC1 ; CS(=O)(=O)OCCN(CCBr)c1ccc([N+](=O)[O-])cc1S(=O)(=O)N1CCN(c2cccc(Cl)c2)CC1]

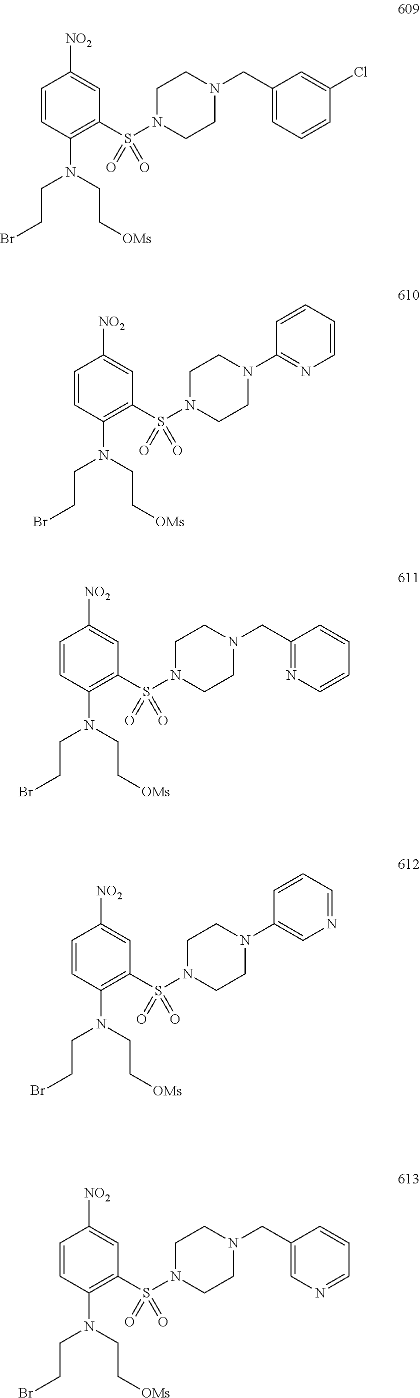 [SMILES: CS(=O)(=O)OCCN(CCBr)c1ccc([N+](=O)[O-])cc1S(=O)(=O)N1CCN(Cc2cccc(Cl)c2)CC1 ; CS(=O)(=O)OCCN(CCBr)c1ccc([N+](=O)[O-])cc1S(=O)(=O)N1CCN(c2ccccn2)CC1 ; CS(=O)(=O)OCCN(CCBr)c1ccc([N+](=O)[O-])cc1S(=O)(=O)N1CCN(Cc2ccccn2)CC1 ; CS(=O)(=O)OCCN(CCBr)c1ccc([N+](=O)[O-])cc1S(=O)(=O)N1CCN(c2cccnc2)CC1 ; CS(=O)(=O)OCCN(CCBr)c1ccc([N+](=O)[O-])cc1S(=O)(=O)N1CCN(Cc2cccnc2)CC1]

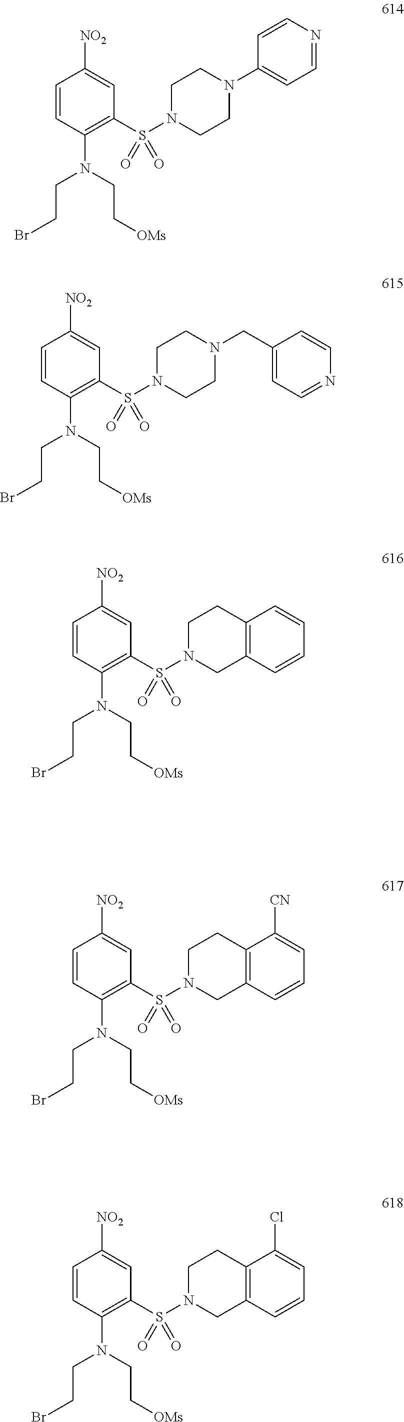 [SMILES: CS(=O)(=O)OCCN(CCBr)c1ccc([N+](=O)[O-])cc1S(=O)(=O)N1CCN(c2ccncc2)CC1 ; CS(=O)(=O)OCCN(CCBr)c1ccc([N+](=O)[O-])cc1S(=O)(=O)N1CCN(Cc2ccncc2)CC1 ; CS(=O)(=O)OCCN(CCBr)c1ccc([N+](=O)[O-])cc1S(=O)(=O)N1CCc2ccccc2C1 ; CS(=O)(=O)OCCN(CCBr)c1ccc([N+](=O)[O-])cc1S(=O)(=O)N1CCc2c(C#N)cccc2C1 ; CS(=O)(=O)OCCN(CCBr)c1ccc([N+](=O)[O-])cc1S(=O)(=O)N1CCc2c(Cl)cccc2C1]

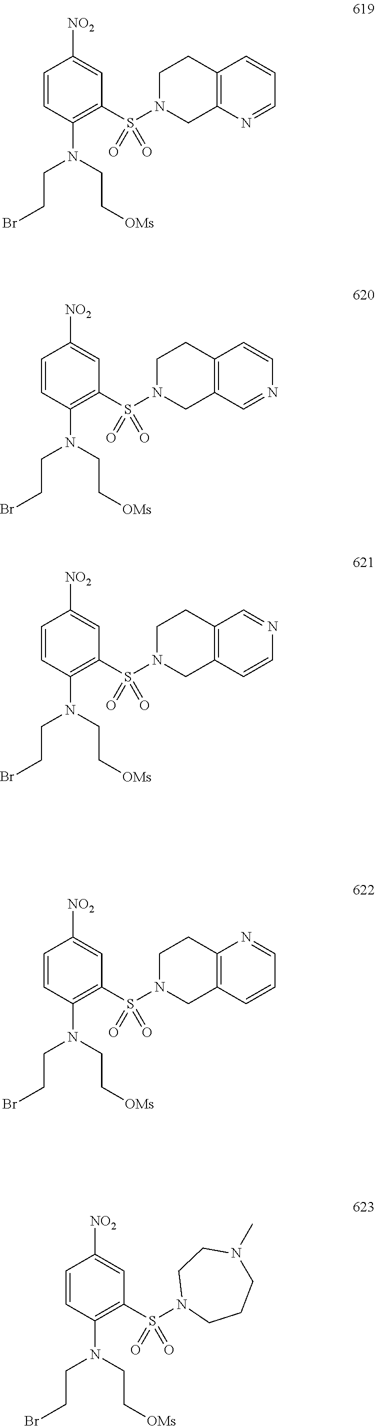 [SMILES: CS(=O)(=O)OCCN(CCBr)c1ccc([N+](=O)[O-])cc1S(=O)(=O)N1CCc2cccnc2C1 ; CS(=O)(=O)OCCN(CCBr)c1ccc([N+](=O)[O-])cc1S(=O)(=O)N1CCc2ccncc2C1 ; CS(=O)(=O)OCCN(CCBr)c1ccc([N+](=O)[O-])cc1S(=O)(=O)N1CCc2cnccc2C1 ; CS(=O)(=O)OCCN(CCBr)c1ccc([N+](=O)[O-])cc1S(=O)(=O)N1CCc2ncccc2C1 ; CN1CCCN(S(=O)(=O)c2cc([N+](=O)[O-])ccc2N(CCBr)CCOS(C)(=O)=O)CC1]

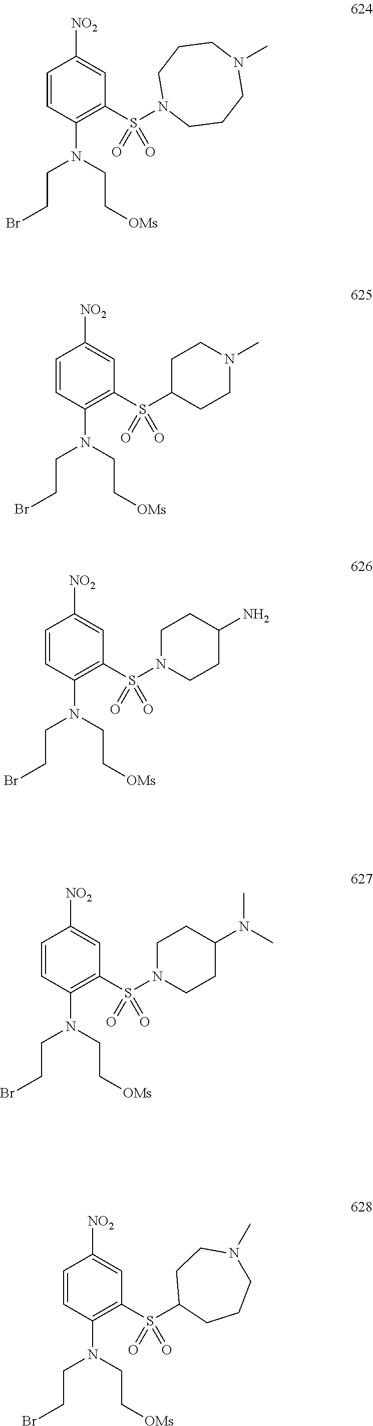 [SMILES: CN1CCCN(S(=O)(=O)c2cc([N+](=O)[O-])ccc2N(CCBr)CCOS(C)(=O)=O)CCC1 ; CN1CCC(S(=O)(=O)c2cc([N+](=O)[O-])ccc2N(CCBr)CCOS(C)(=O)=O)CC1 ; CS(=O)(=O)OCCN(CCBr)c1ccc([N+](=O)[O-])cc1S(=O)(=O)N1CCC(N)CC1 ; CN(C)C1CCN(S(=O)(=O)c2cc([N+](=O)[O-])ccc2N(CCBr)CCOS(C)(=O)=O)CC1 ; CN1CCCC(S(=O)(=O)c2cc([N+](=O)[O-])ccc2N(CCBr)CCOS(C)(=O)=O)CC1]

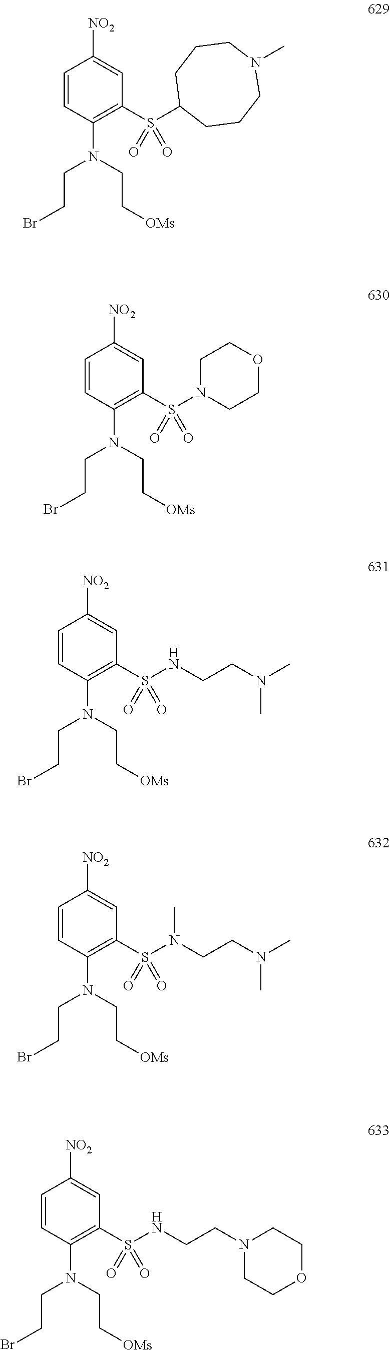 [SMILES: CN1CCCC(S(=O)(=O)c2cc([N+](=O)[O-])ccc2N(CCBr)CCOS(C)(=O)=O)CCC1 ; CS(=O)(=O)OCCN(CCBr)c1ccc([N+](=O)[O-])cc1S(=O)(=O)N1CCOCC1 ; CN(C)CCNS(=O)(=O)c1cc([N+](=O)[O-])ccc1N(CCBr)CCOS(C)(=O)=O ; CN(C)CCN(C)S(=O)(=O)c1cc([N+](=O)[O-])ccc1N(CCBr)CCOS(C)(=O)=O ; CS(=O)(=O)OCCN(CCBr)c1ccc([N+](=O)[O-])cc1S(=O)(=O)NCCN1CCOCC1]

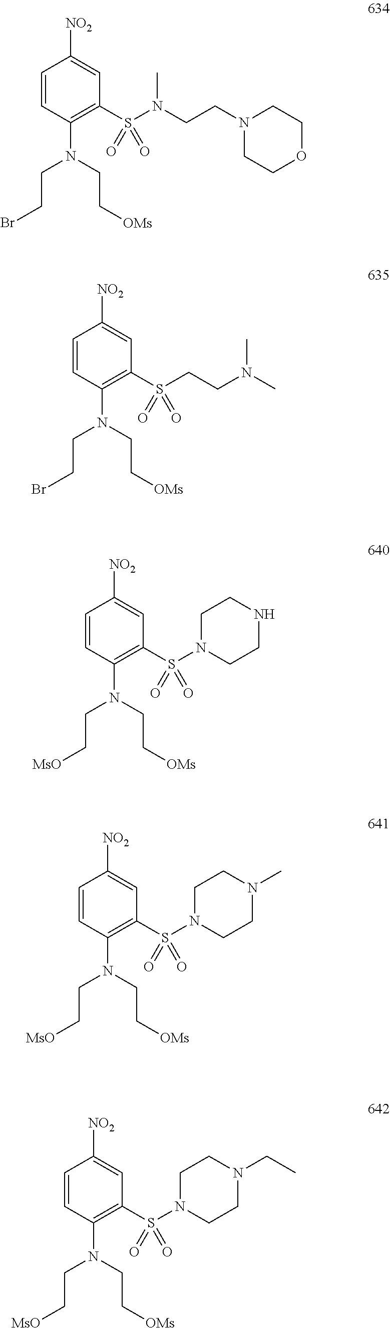 [SMILES: CN(CCN1CCOCC1)S(=O)(=O)c1cc([N+](=O)[O-])ccc1N(CCBr)CCOS(C)(=O)=O ; CN(C)CCS(=O)(=O)c1cc([N+](=O)[O-])ccc1N(CCBr)CCOS(C)(=O)=O ; CS(=O)(=O)OCCN(CCOS(C)(=O)=O)c1ccc([N+](=O)[O-])cc1S(=O)(=O)N1CCNCC1 ; CN1CCN(S(=O)(=O)c2cc([N+](=O)[O-])ccc2N(CCOS(C)(=O)=O)CCOS(C)(=O)=O)CC1 ; CCN1CCN(S(=O)(=O)c2cc([N+](=O)[O-])ccc2N(CCOS(C)(=O)=O)CCOS(C)(=O)=O)CC1]

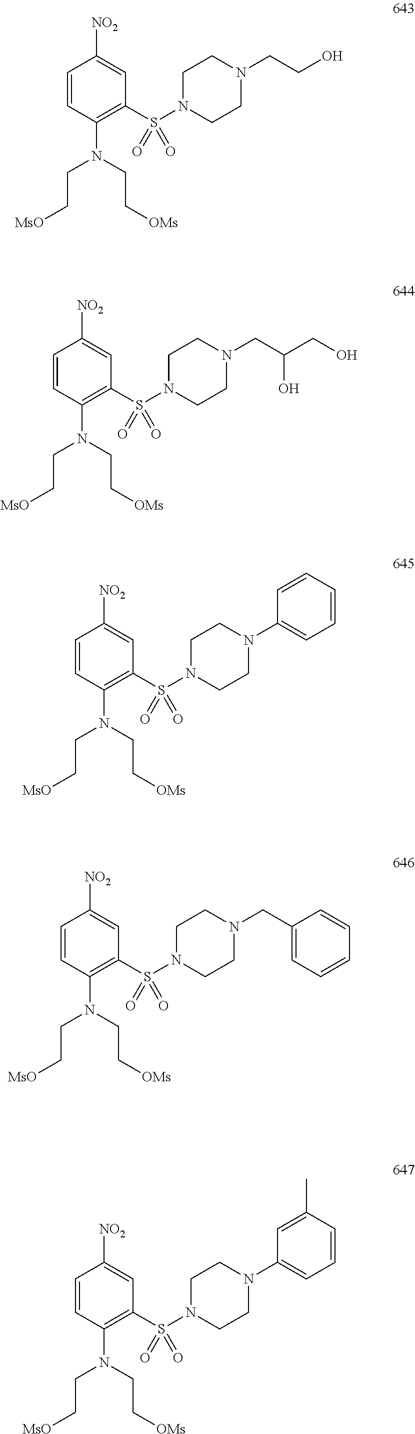 [SMILES: CS(=O)(=O)OCCN(CCOS(C)(=O)=O)c1ccc([N+](=O)[O-])cc1S(=O)(=O)N1CCN(CCO)CC1 ; CS(=O)(=O)OCCN(CCOS(C)(=O)=O)c1ccc([N+](=O)[O-])cc1S(=O)(=O)N1CCN(CC(O)CO)CC1 ; CS(=O)(=O)OCCN(CCOS(C)(=O)=O)c1ccc([N+](=O)[O-])cc1S(=O)(=O)N1CCN(c2ccccc2)CC1 ; CS(=O)(=O)OCCN(CCOS(C)(=O)=O)c1ccc([N+](=O)[O-])cc1S(=O)(=O)N1CCN(Cc2ccccc2)CC1 ; Cc1cccc(N2CCN(S(=O)(=O)c3cc([N+](=O)[O-])ccc3N(CCOS(C)(=O)=O)CCOS(C)(=O)=O)CC2)c1]

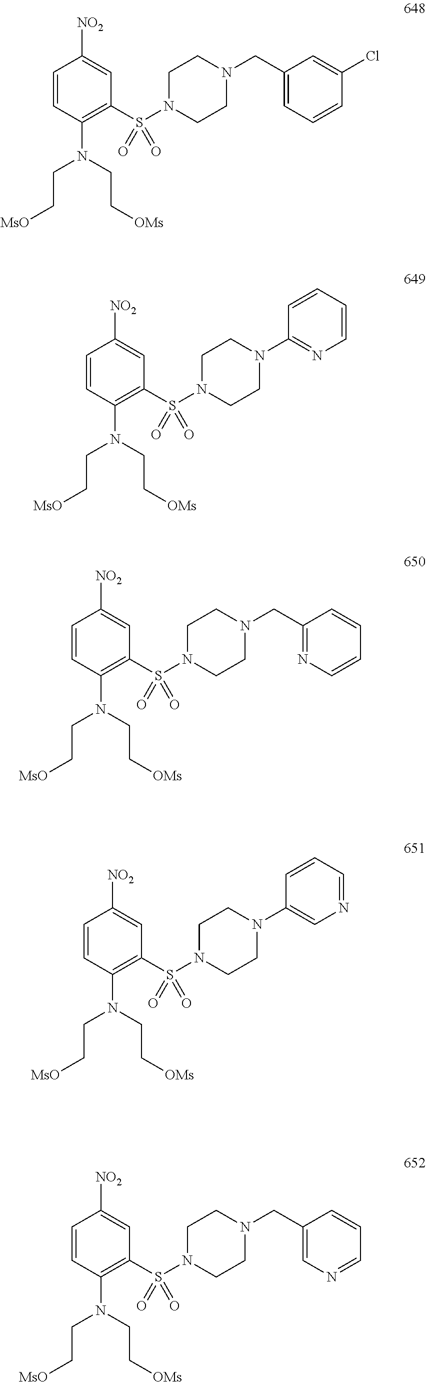 [SMILES: CS(=O)(=O)OCCN(CCOS(C)(=O)=O)c1ccc([N+](=O)[O-])cc1S(=O)(=O)N1CCN(Cc2cccc(Cl)c2)CC1 ; CS(=O)(=O)OCCN(CCOS(C)(=O)=O)c1ccc([N+](=O)[O-])cc1S(=O)(=O)N1CCN(c2ccccn2)CC1 ; CS(=O)(=O)OCCN(CCOS(C)(=O)=O)c1ccc([N+](=O)[O-])cc1S(=O)(=O)N1CCN(Cc2ccccn2)CC1 ; CS(=O)(=O)OCCN(CCOS(C)(=O)=O)c1ccc([N+](=O)[O-])cc1S(=O)(=O)N1CCN(c2cccnc2)CC1 ; CS(=O)(=O)OCCN(CCOS(C)(=O)=O)c1ccc([N+](=O)[O-])cc1S(=O)(=O)N1CCN(Cc2cccnc2)CC1]

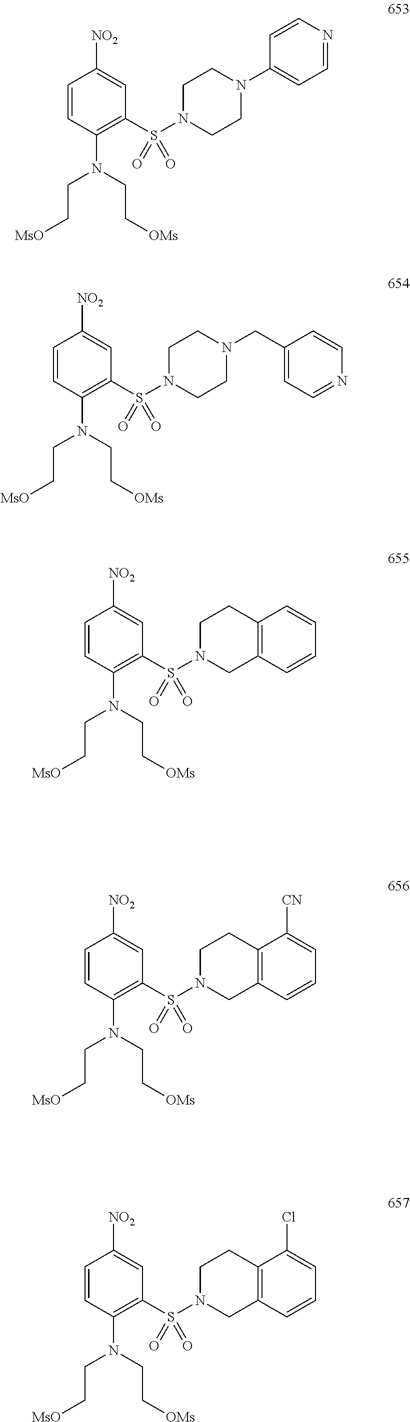 [SMILES: CS(=O)(=O)OCCN(CCOS(C)(=O)=O)c1ccc([N+](=O)[O-])cc1S(=O)(=O)N1CCN(c2ccncc2)CC1 ; CS(=O)(=O)OCCN(CCOS(C)(=O)=O)c1ccc([N+](=O)[O-])cc1S(=O)(=O)N1CCN(Cc2ccncc2)CC1 ; CS(=O)(=O)OCCN(CCOS(C)(=O)=O)c1ccc([N+](=O)[O-])cc1S(=O)(=O)N1CCc2ccccc2C1 ; CS(=O)(=O)OCCN(CCOS(C)(=O)=O)c1ccc([N+](=O)[O-])cc1S(=O)(=O)N1CCc2c(C#N)cccc2C1 ; CS(=O)(=O)OCCN(CCOS(C)(=O)=O)c1ccc([N+](=O)[O-])cc1S(=O)(=O)N1CCc2c(Cl)cccc2C1]

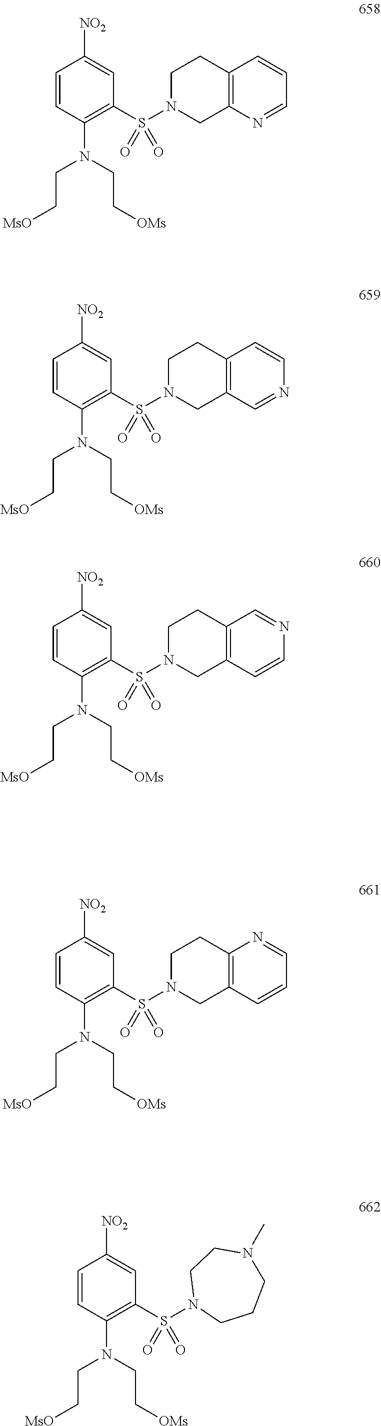 [SMILES: CS(=O)(=O)OCCN(CCOS(C)(=O)=O)c1ccc([N+](=O)[O-])cc1S(=O)(=O)N1CCc2cccnc2C1 ; CS(=O)(=O)OCCN(CCOS(C)(=O)=O)c1ccc([N+](=O)[O-])cc1S(=O)(=O)N1CCc2ccncc2C1 ; CS(=O)(=O)OCCN(CCOS(C)(=O)=O)c1ccc([N+](=O)[O-])cc1S(=O)(=O)N1CCc2cnccc2C1 ; CS(=O)(=O)OCCN(CCOS(C)(=O)=O)c1ccc([N+](=O)[O-])cc1S(=O)(=O)N1CCc2ncccc2C1 ; CN1CCCN(S(=O)(=O)c2cc([N+](=O)[O-])ccc2N(CCOS(C)(=O)=O)CCOS(C)(=O)=O)CC1]

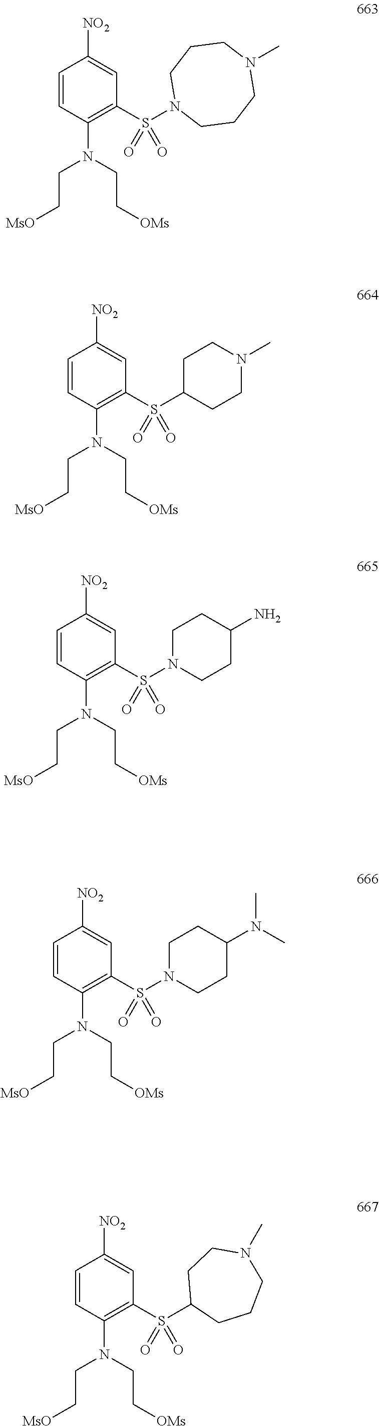 [SMILES: CN1CCCN(S(=O)(=O)c2cc([N+](=O)[O-])ccc2N(CCOS(C)(=O)=O)CCOS(C)(=O)=O)CCC1 ; CN1CCC(S(=O)(=O)c2cc([N+](=O)[O-])ccc2N(CCOS(C)(=O)=O)CCOS(C)(=O)=O)CC1 ; CS(=O)(=O)OCCN(CCOS(C)(=O)=O)c1ccc([N+](=O)[O-])cc1S(=O)(=O)N1CCC(N)CC1 ; CN(C)C1CCN(S(=O)(=O)c2cc([N+](=O)[O-])ccc2N(CCOS(C)(=O)=O)CCOS(C)(=O)=O)CC1 ; CN1CCCC(S(=O)(=O)c2cc([N+](=O)[O-])ccc2N(CCOS(C)(=O)=O)CCOS(C)(=O)=O)CC1]

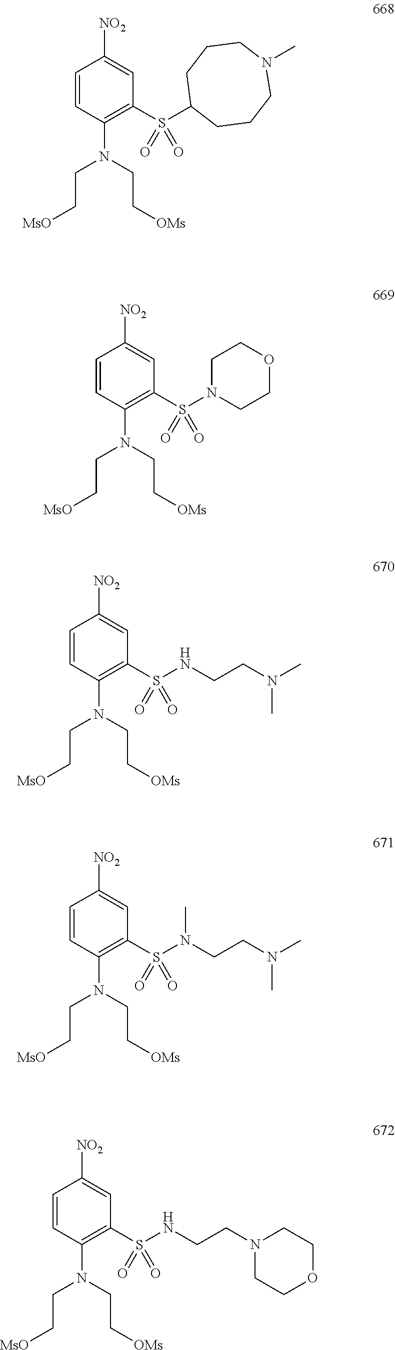 [SMILES: CN1CCCC(S(=O)(=O)c2cc([N+](=O)[O-])ccc2N(CCOS(C)(=O)=O)CCOS(C)(=O)=O)CCC1 ; CS(=O)(=O)OCCN(CCOS(C)(=O)=O)c1ccc([N+](=O)[O-])cc1S(=O)(=O)N1CCOCC1 ; CN(C)CCNS(=O)(=O)c1cc([N+](=O)[O-])ccc1N(CCOS(C)(=O)=O)CCOS(C)(=O)=O ; CN(C)CCN(C)S(=O)(=O)c1cc([N+](=O)[O-])ccc1N(CCOS(C)(=O)=O)CCOS(C)(=O)=O ; CS(=O)(=O)OCCN(CCOS(C)(=O)=O)c1ccc([N+](=O)[O-])cc1S(=O)(=O)NCCN1CCOCC1]

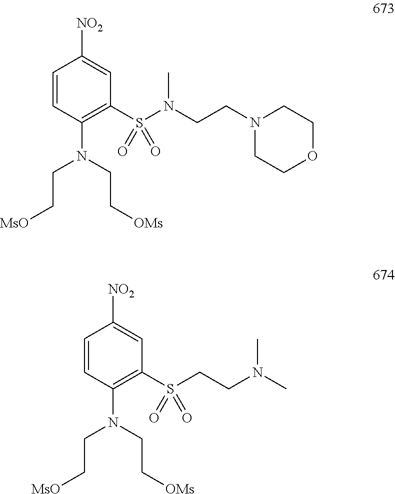 [SMILES: CN(CCN1CCOCC1)S(=O)(=O)c1cc([N+](=O)[O-])ccc1N(CCOS(C)(=O)=O)CCOS(C)(=O)=O ; CN(C)CCS(=O)(=O)c1cc([N+](=O)[O-])ccc1N(CCOS(C)(=O)=O)CCOS(C)(=O)=O]

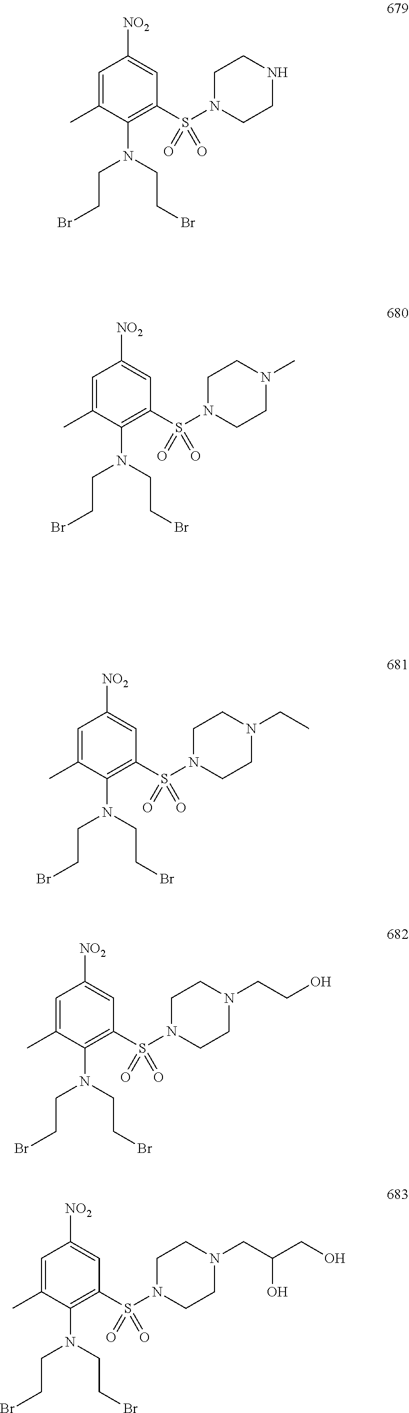 [SMILES: Cc1cc([N+](=O)[O-])cc(S(=O)(=O)N2CCNCC2)c1N(CCBr)CCBr ; Cc1cc([N+](=O)[O-])cc(S(=O)(=O)N2CCN(C)CC2)c1N(CCBr)CCBr ; CCN1CCN(S(=O)(=O)c2cc([N+](=O)[O-])cc(C)c2N(CCBr)CCBr)CC1 ; Cc1cc([N+](=O)[O-])cc(S(=O)(=O)N2CCN(CCO)CC2)c1N(CCBr)CCBr ; Cc1cc([N+](=O)[O-])cc(S(=O)(=O)N2CCN(CC(O)CO)CC2)c1N(CCBr)CCBr]

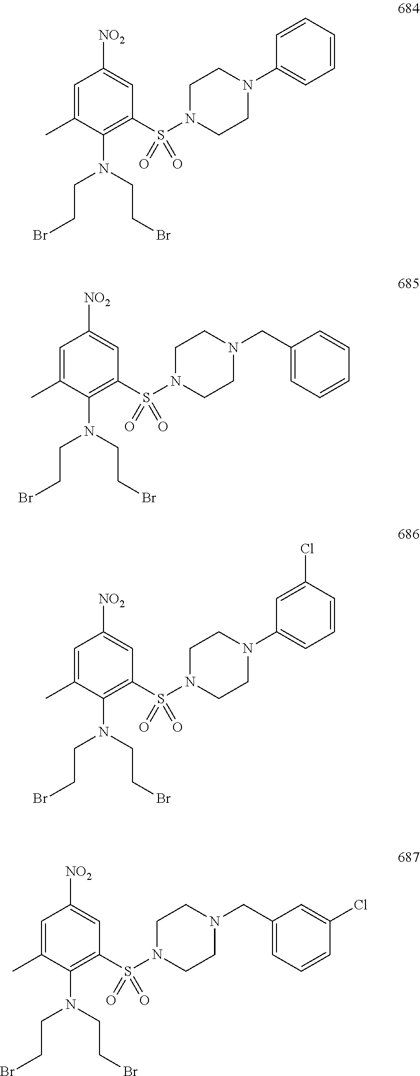 [SMILES: Cc1cc([N+](=O)[O-])cc(S(=O)(=O)N2CCN(c3ccccc3)CC2)c1N(CCBr)CCBr ; Cc1cc([N+](=O)[O-])cc(S(=O)(=O)N2CCN(Cc3ccccc3)CC2)c1N(CCBr)CCBr ; Cc1cc([N+](=O)[O-])cc(S(=O)(=O)N2CCN(c3cccc(Cl)c3)CC2)c1N(CCBr)CCBr ; Cc1cc([N+](=O)[O-])cc(S(=O)(=O)N2CCN(Cc3cccc(Cl)c3)CC2)c1N(CCBr)CCBr]

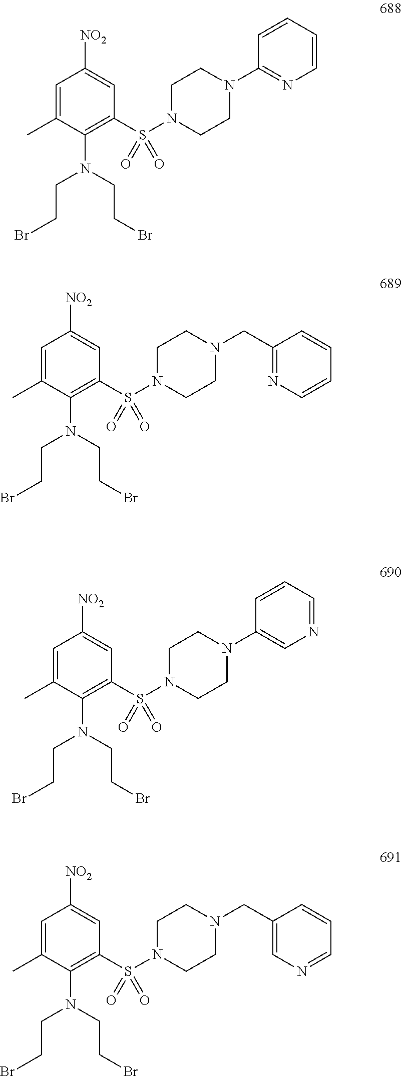 [SMILES: Cc1cc([N+](=O)[O-])cc(S(=O)(=O)N2CCN(c3ccccn3)CC2)c1N(CCBr)CCBr ; Cc1cc([N+](=O)[O-])cc(S(=O)(=O)N2CCN(Cc3ccccn3)CC2)c1N(CCBr)CCBr ; Cc1cc([N+](=O)[O-])cc(S(=O)(=O)N2CCN(c3cccnc3)CC2)c1N(CCBr)CCBr ; Cc1cc([N+](=O)[O-])cc(S(=O)(=O)N2CCN(Cc3cccnc3)CC2)c1N(CCBr)CCBr]

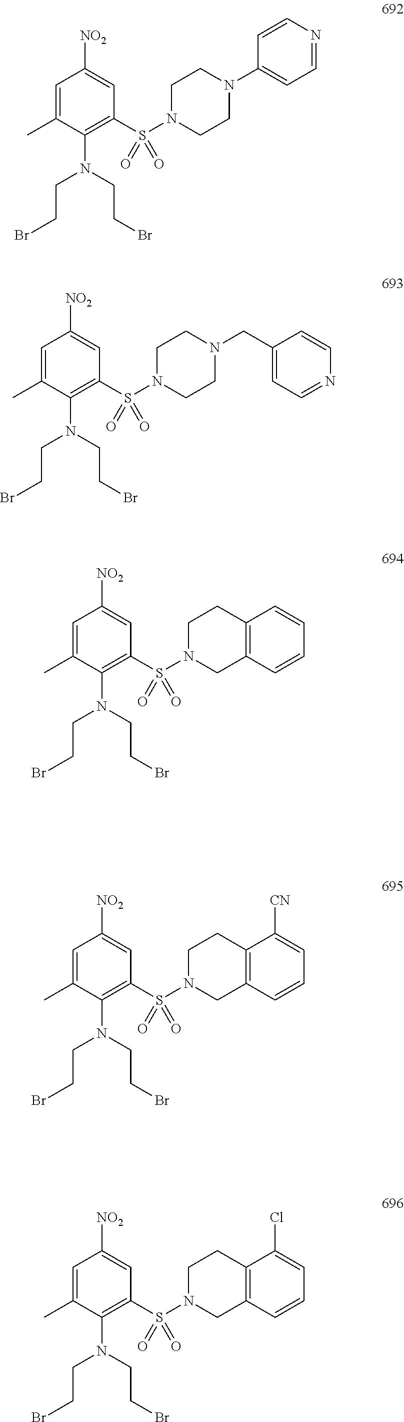 [SMILES: Cc1cc([N+](=O)[O-])cc(S(=O)(=O)N2CCN(c3ccncc3)CC2)c1N(CCBr)CCBr ; Cc1cc([N+](=O)[O-])cc(S(=O)(=O)N2CCN(Cc3ccncc3)CC2)c1N(CCBr)CCBr ; Cc1cc([N+](=O)[O-])cc(S(=O)(=O)N2CCc3ccccc3C2)c1N(CCBr)CCBr ; Cc1cc([N+](=O)[O-])cc(S(=O)(=O)N2CCc3c(C#N)cccc3C2)c1N(CCBr)CCBr ; Cc1cc([N+](=O)[O-])cc(S(=O)(=O)N2CCc3c(Cl)cccc3C2)c1N(CCBr)CCBr]

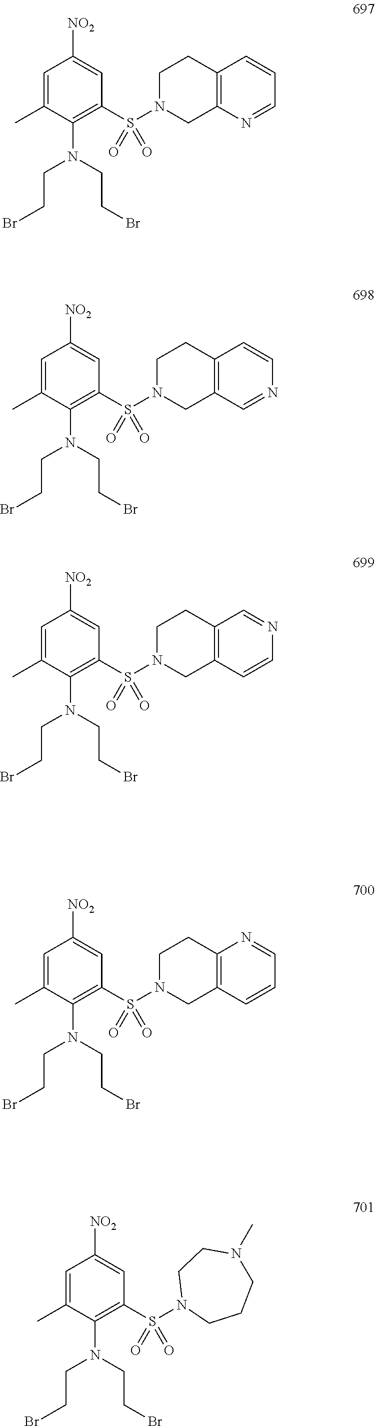 [SMILES: Cc1cc([N+](=O)[O-])cc(S(=O)(=O)N2CCc3cccnc3C2)c1N(CCBr)CCBr ; Cc1cc([N+](=O)[O-])cc(S(=O)(=O)N2CCc3ccncc3C2)c1N(CCBr)CCBr ; Cc1cc([N+](=O)[O-])cc(S(=O)(=O)N2CCc3cnccc3C2)c1N(CCBr)CCBr ; Cc1cc([N+](=O)[O-])cc(S(=O)(=O)N2CCc3ncccc3C2)c1N(CCBr)CCBr ; Cc1cc([N+](=O)[O-])cc(S(=O)(=O)N2CCCN(C)CC2)c1N(CCBr)CCBr]

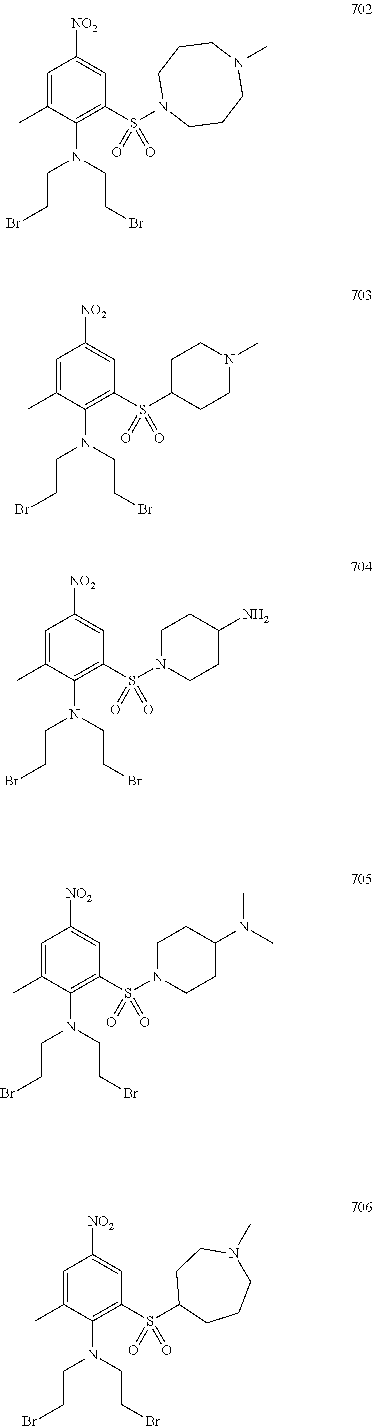 [SMILES: Cc1cc([N+](=O)[O-])cc(S(=O)(=O)N2CCCN(C)CCC2)c1N(CCBr)CCBr ; Cc1cc([N+](=O)[O-])cc(S(=O)(=O)C2CCN(C)CC2)c1N(CCBr)CCBr ; Cc1cc([N+](=O)[O-])cc(S(=O)(=O)N2CCC(N)CC2)c1N(CCBr)CCBr ; Cc1cc([N+](=O)[O-])cc(S(=O)(=O)N2CCC(N(C)C)CC2)c1N(CCBr)CCBr ; Cc1cc([N+](=O)[O-])cc(S(=O)(=O)C2CCCN(C)CC2)c1N(CCBr)CCBr]

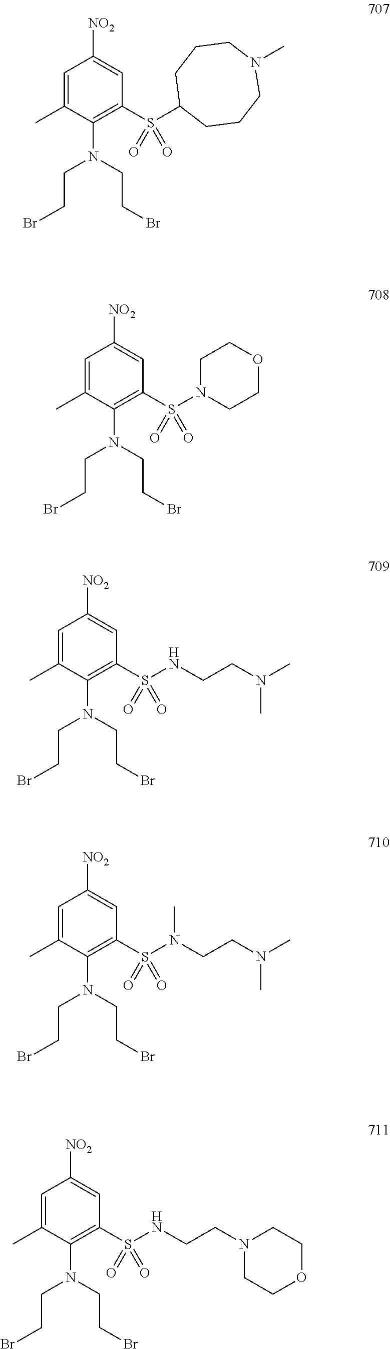 [SMILES: Cc1cc([N+](=O)[O-])cc(S(=O)(=O)C2CCCN(C)CCC2)c1N(CCBr)CCBr ; Cc1cc([N+](=O)[O-])cc(S(=O)(=O)N2CCOCC2)c1N(CCBr)CCBr ; Cc1cc([N+](=O)[O-])cc(S(=O)(=O)NCCN(C)C)c1N(CCBr)CCBr ; Cc1cc([N+](=O)[O-])cc(S(=O)(=O)N(C)CCN(C)C)c1N(CCBr)CCBr ; Cc1cc([N+](=O)[O-])cc(S(=O)(=O)NCCN2CCOCC2)c1N(CCBr)CCBr]

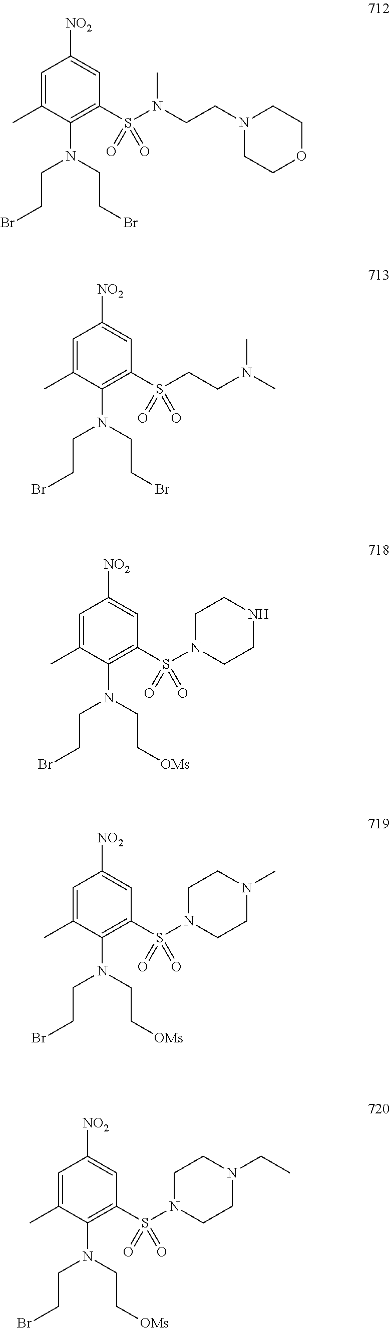 [SMILES: Cc1cc([N+](=O)[O-])cc(S(=O)(=O)N(C)CCN2CCOCC2)c1N(CCBr)CCBr ; Cc1cc([N+](=O)[O-])cc(S(=O)(=O)CCN(C)C)c1N(CCBr)CCBr ; Cc1cc([N+](=O)[O-])cc(S(=O)(=O)N2CCNCC2)c1N(CCBr)CCOS(C)(=O)=O ; Cc1cc([N+](=O)[O-])cc(S(=O)(=O)N2CCN(C)CC2)c1N(CCBr)CCOS(C)(=O)=O ; CCN1CCN(S(=O)(=O)c2cc([N+](=O)[O-])cc(C)c2N(CCBr)CCOS(C)(=O)=O)CC1]

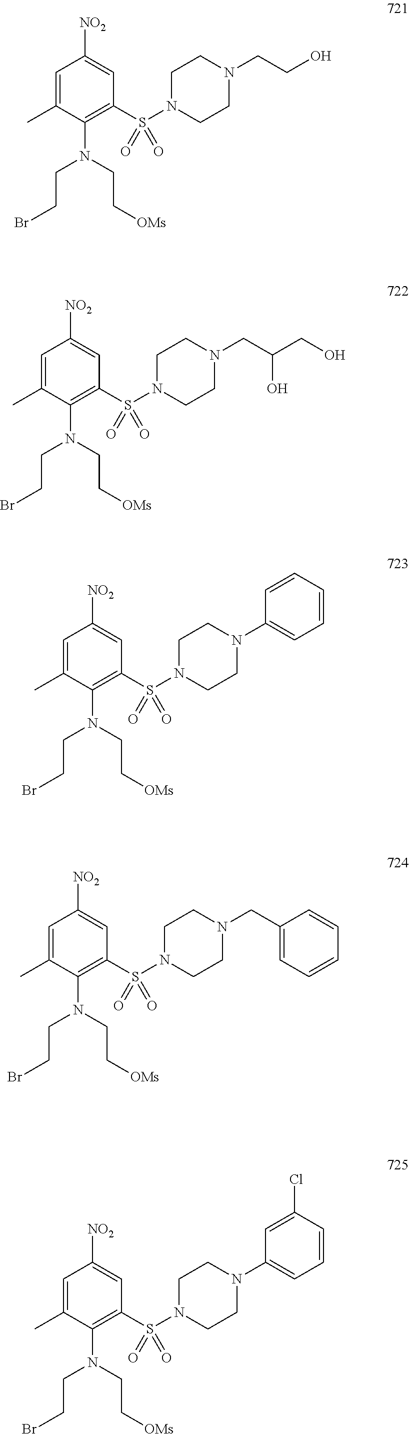 [SMILES: Cc1cc([N+](=O)[O-])cc(S(=O)(=O)N2CCN(CCO)CC2)c1N(CCBr)CCOS(C)(=O)=O ; Cc1cc([N+](=O)[O-])cc(S(=O)(=O)N2CCN(CC(O)CO)CC2)c1N(CCBr)CCOS(C)(=O)=O ; Cc1cc([N+](=O)[O-])cc(S(=O)(=O)N2CCN(c3ccccc3)CC2)c1N(CCBr)CCOS(C)(=O)=O ; Cc1cc([N+](=O)[O-])cc(S(=O)(=O)N2CCN(Cc3ccccc3)CC2)c1N(CCBr)CCOS(C)(=O)=O ; Cc1cc([N+](=O)[O-])cc(S(=O)(=O)N2CCN(c3cccc(Cl)c3)CC2)c1N(CCBr)CCOS(C)(=O)=O]

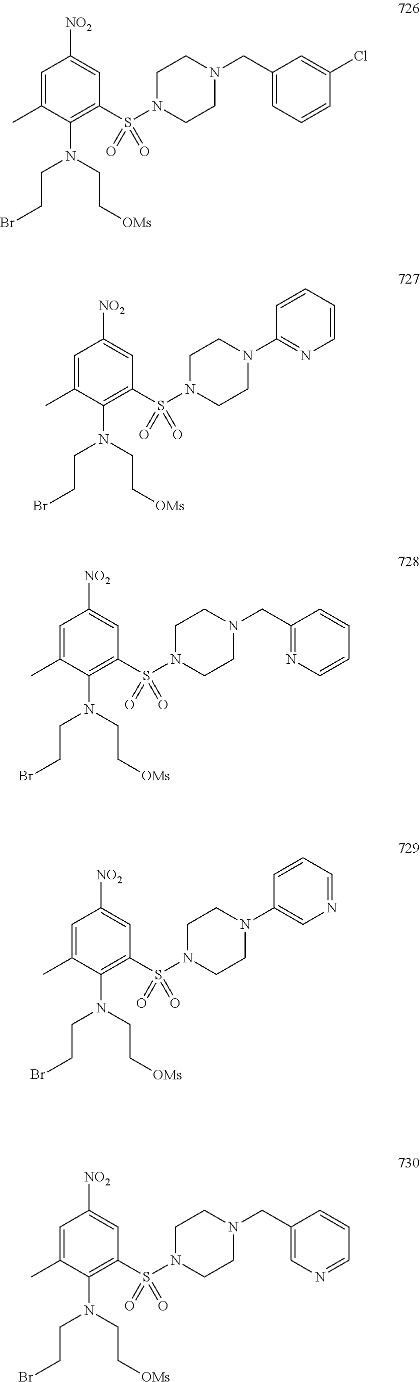 [SMILES: Cc1cc([N+](=O)[O-])cc(S(=O)(=O)N2CCN(Cc3cccc(Cl)c3)CC2)c1N(CCBr)CCOS(C)(=O)=O ; Cc1cc([N+](=O)[O-])cc(S(=O)(=O)N2CCN(c3ccccn3)CC2)c1N(CCBr)CCOS(C)(=O)=O ; Cc1cc([N+](=O)[O-])cc(S(=O)(=O)N2CCN(Cc3ccccn3)CC2)c1N(CCBr)CCOS(C)(=O)=O ; Cc1cc([N+](=O)[O-])cc(S(=O)(=O)N2CCN(c3cccnc3)CC2)c1N(CCBr)CCOS(C)(=O)=O ; Cc1cc([N+](=O)[O-])cc(S(=O)(=O)N2CCN(Cc3cccnc3)CC2)c1N(CCBr)CCOS(C)(=O)=O]

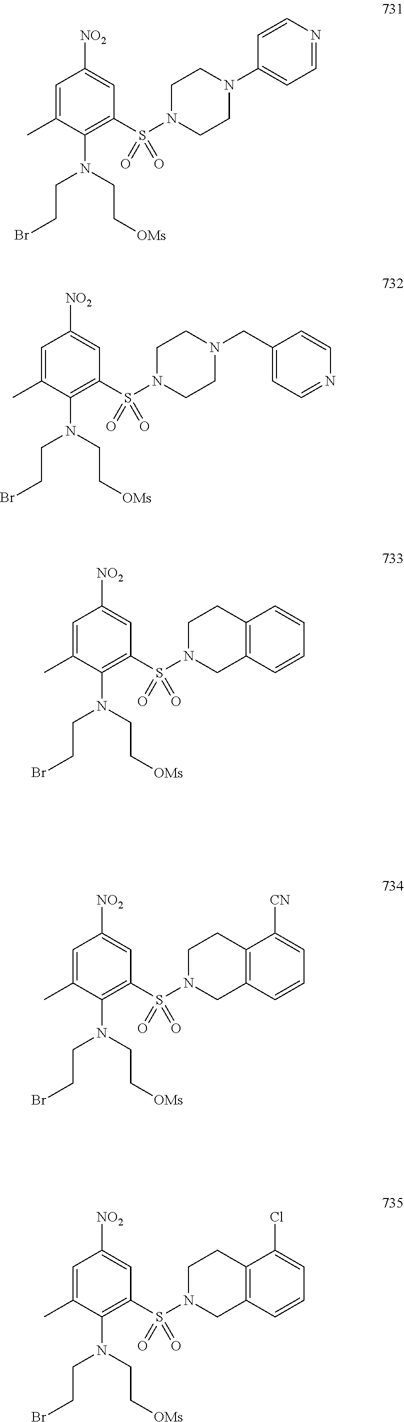 [SMILES: Cc1cc([N+](=O)[O-])cc(S(=O)(=O)N2CCN(c3ccncc3)CC2)c1N(CCBr)CCOS(C)(=O)=O ; Cc1cc([N+](=O)[O-])cc(S(=O)(=O)N2CCN(Cc3ccncc3)CC2)c1N(CCBr)CCOS(C)(=O)=O ; Cc1cc([N+](=O)[O-])cc(S(=O)(=O)N2CCc3ccccc3C2)c1N(CCBr)CCOS(C)(=O)=O ; Cc1cc([N+](=O)[O-])cc(S(=O)(=O)N2CCc3c(C#N)cccc3C2)c1N(CCBr)CCOS(C)(=O)=O ; Cc1cc([N+](=O)[O-])cc(S(=O)(=O)N2CCc3c(Cl)cccc3C2)c1N(CCBr)CCOS(C)(=O)=O]

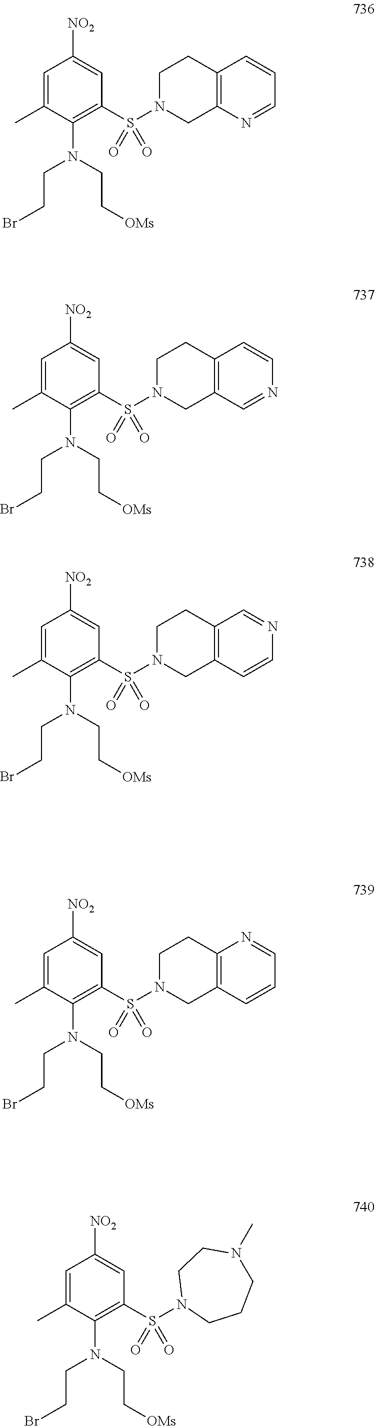 [SMILES: Cc1cc([N+](=O)[O-])cc(S(=O)(=O)N2CCc3cccnc3C2)c1N(CCBr)CCOS(C)(=O)=O ; Cc1cc([N+](=O)[O-])cc(S(=O)(=O)N2CCc3ccncc3C2)c1N(CCBr)CCOS(C)(=O)=O ; Cc1cc([N+](=O)[O-])cc(S(=O)(=O)N2CCc3cnccc3C2)c1N(CCBr)CCOS(C)(=O)=O ; Cc1cc([N+](=O)[O-])cc(S(=O)(=O)N2CCc3ncccc3C2)c1N(CCBr)CCOS(C)(=O)=O ; Cc1cc([N+](=O)[O-])cc(S(=O)(=O)N2CCCN(C)CC2)c1N(CCBr)CCOS(C)(=O)=O]

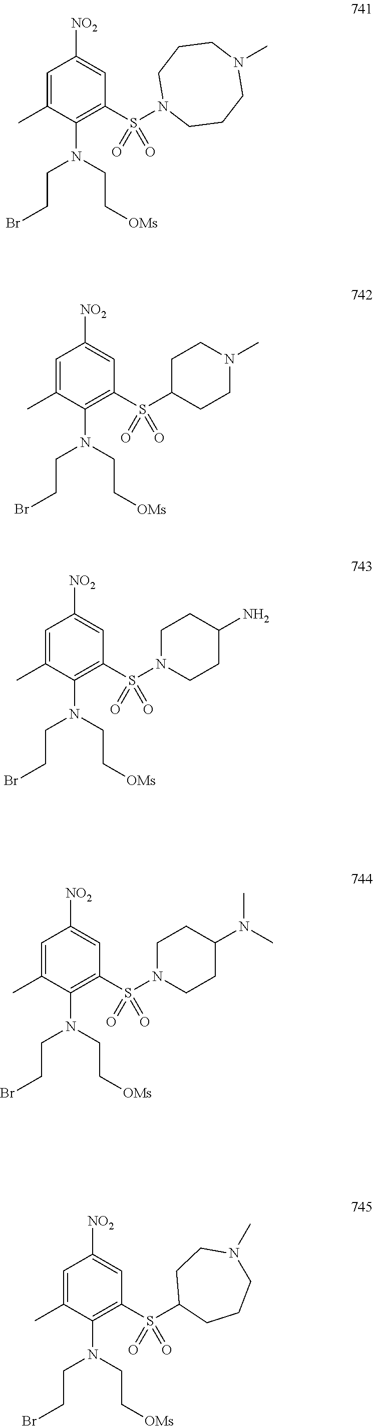 [SMILES: Cc1cc([N+](=O)[O-])cc(S(=O)(=O)N2CCCN(C)CCC2)c1N(CCBr)CCOS(C)(=O)=O ; Cc1cc([N+](=O)[O-])cc(S(=O)(=O)C2CCN(C)CC2)c1N(CCBr)CCOS(C)(=O)=O ; Cc1cc([N+](=O)[O-])cc(S(=O)(=O)N2CCC(N)CC2)c1N(CCBr)CCOS(C)(=O)=O ; Cc1cc([N+](=O)[O-])cc(S(=O)(=O)N2CCC(N(C)C)CC2)c1N(CCBr)CCOS(C)(=O)=O ; Cc1cc([N+](=O)[O-])cc(S(=O)(=O)C2CCCN(C)CC2)c1N(CCBr)CCOS(C)(=O)=O]

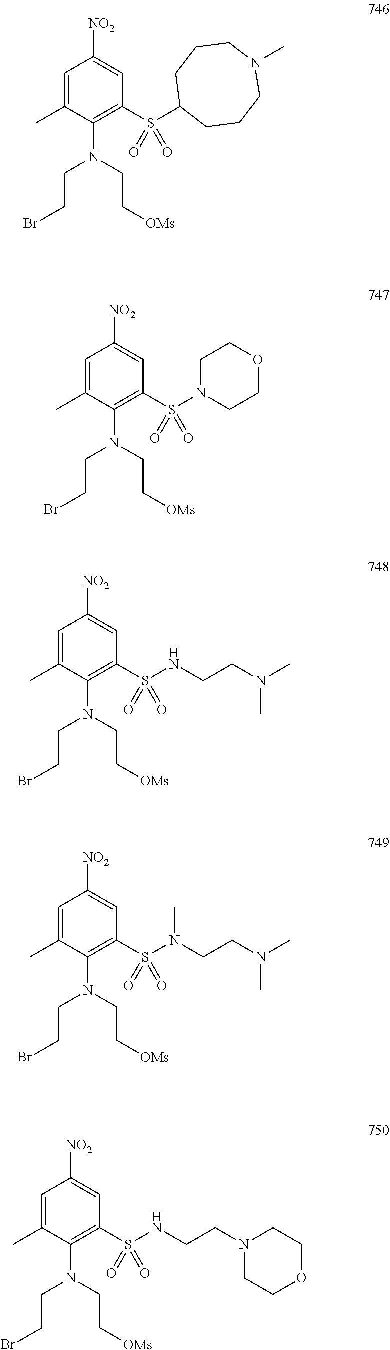 [SMILES: Cc1cc([N+](=O)[O-])cc(S(=O)(=O)C2CCCN(C)CCC2)c1N(CCBr)CCOS(C)(=O)=O ; Cc1cc([N+](=O)[O-])cc(S(=O)(=O)N2CCOCC2)c1N(CCBr)CCOS(C)(=O)=O ; Cc1cc([N+](=O)[O-])cc(S(=O)(=O)NCCN(C)C)c1N(CCBr)CCOS(C)(=O)=O ; Cc1cc([N+](=O)[O-])cc(S(=O)(=O)N(C)CCN(C)C)c1N(CCBr)CCOS(C)(=O)=O ; Cc1cc([N+](=O)[O-])cc(S(=O)(=O)NCCN2CCOCC2)c1N(CCBr)CCOS(C)(=O)=O]

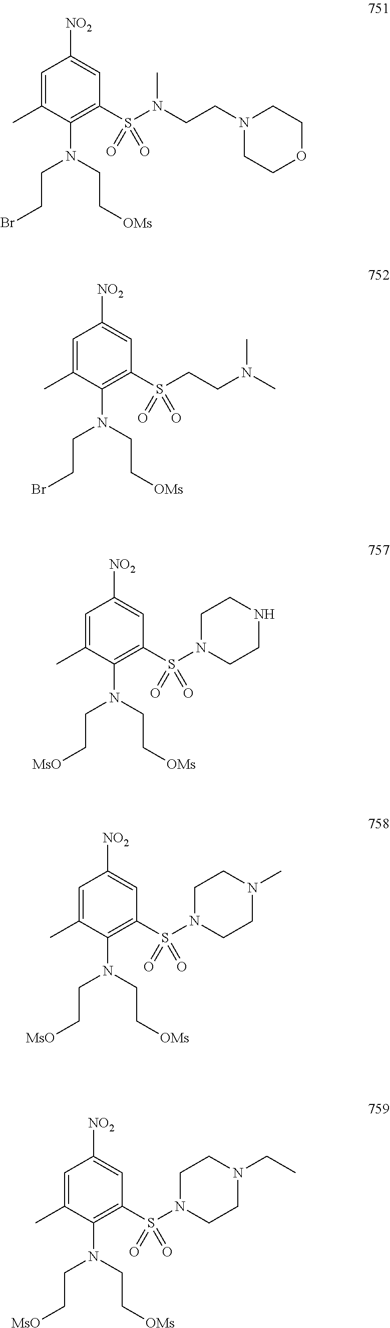 [SMILES: Cc1cc([N+](=O)[O-])cc(S(=O)(=O)N(C)CCN2CCOCC2)c1N(CCBr)CCOS(C)(=O)=O ; Cc1cc([N+](=O)[O-])cc(S(=O)(=O)CCN(C)C)c1N(CCBr)CCOS(C)(=O)=O ; Cc1cc([N+](=O)[O-])cc(S(=O)(=O)N2CCNCC2)c1N(CCOS(C)(=O)=O)CCOS(C)(=O)=O ; Cc1cc([N+](=O)[O-])cc(S(=O)(=O)N2CCN(C)CC2)c1N(CCOS(C)(=O)=O)CCOS(C)(=O)=O ; CCN1CCN(S(=O)(=O)c2cc([N+](=O)[O-])cc(C)c2N(CCOS(C)(=O)=O)CCOS(C)(=O)=O)CC1]

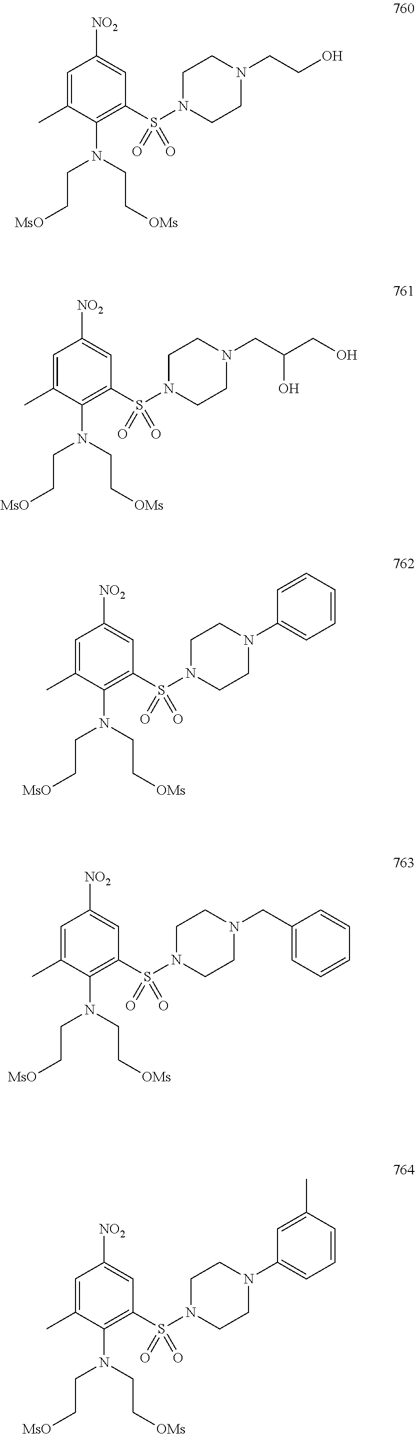 [SMILES: Cc1cc([N+](=O)[O-])cc(S(=O)(=O)N2CCN(CCO)CC2)c1N(CCOS(C)(=O)=O)CCOS(C)(=O)=O ; Cc1cc([N+](=O)[O-])cc(S(=O)(=O)N2CCN(CC(O)CO)CC2)c1N(CCOS(C)(=O)=O)CCOS(C)(=O)=O ; Cc1cc([N+](=O)[O-])cc(S(=O)(=O)N2CCN(c3ccccc3)CC2)c1N(CCOS(C)(=O)=O)CCOS(C)(=O)=O ; Cc1cc([N+](=O)[O-])cc(S(=O)(=O)N2CCN(Cc3ccccc3)CC2)c1N(CCOS(C)(=O)=O)CCOS(C)(=O)=O ; Cc1cccc(N2CCN(S(=O)(=O)c3cc([N+](=O)[O-])cc(C)c3N(CCOS(C)(=O)=O)CCOS(C)(=O)=O)CC2)c1]

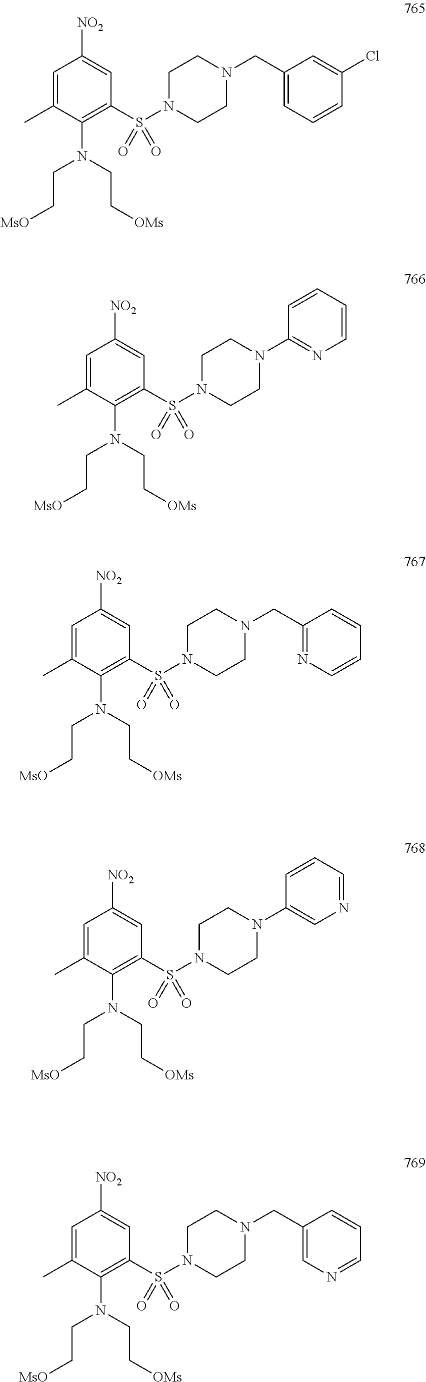 [SMILES: Cc1cc([N+](=O)[O-])cc(S(=O)(=O)N2CCN(Cc3cccc(Cl)c3)CC2)c1N(CCOS(C)(=O)=O)CCOS(C)(=O)=O ; Cc1cc([N+](=O)[O-])cc(S(=O)(=O)N2CCN(c3ccccn3)CC2)c1N(CCOS(C)(=O)=O)CCOS(C)(=O)=O ; Cc1cc([N+](=O)[O-])cc(S(=O)(=O)N2CCN(Cc3ccccn3)CC2)c1N(CCOS(C)(=O)=O)CCOS(C)(=O)=O ; Cc1cc([N+](=O)[O-])cc(S(=O)(=O)N2CCN(c3cccnc3)CC2)c1N(CCOS(C)(=O)=O)CCOS(C)(=O)=O ; Cc1cc([N+](=O)[O-])cc(S(=O)(=O)N2CCN(Cc3cccnc3)CC2)c1N(CCOS(C)(=O)=O)CCOS(C)(=O)=O]

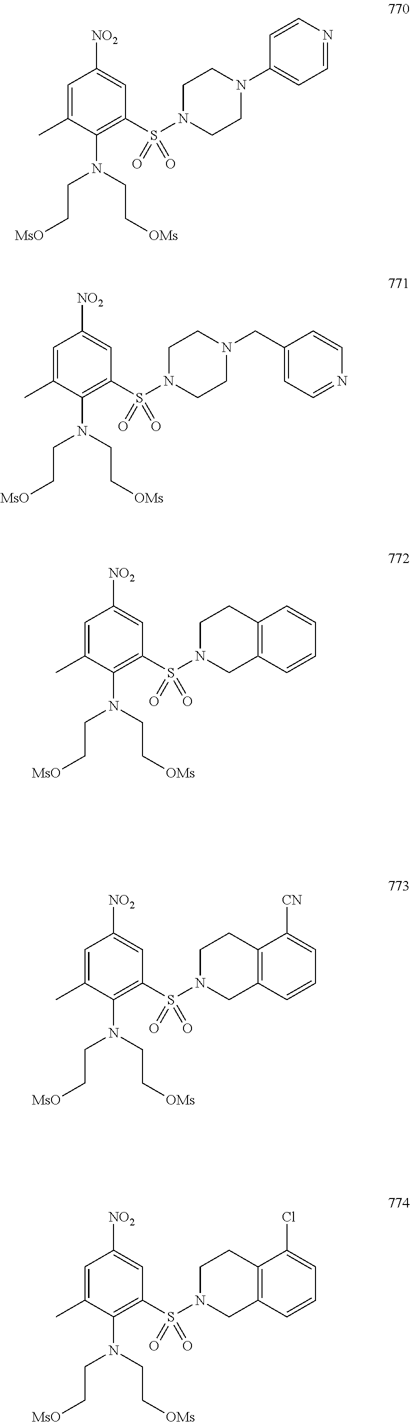 [SMILES: Cc1cc([N+](=O)[O-])cc(S(=O)(=O)N2CCN(c3ccncc3)CC2)c1N(CCOS(C)(=O)=O)CCOS(C)(=O)=O ; Cc1cc([N+](=O)[O-])cc(S(=O)(=O)N2CCN(Cc3ccncc3)CC2)c1N(CCOS(C)(=O)=O)CCOS(C)(=O)=O ; Cc1cc([N+](=O)[O-])cc(S(=O)(=O)N2CCc3ccccc3C2)c1N(CCOS(C)(=O)=O)CCOS(C)(=O)=O ; Cc1cc([N+](=O)[O-])cc(S(=O)(=O)N2CCc3c(C#N)cccc3C2)c1N(CCOS(C)(=O)=O)CCOS(C)(=O)=O ; Cc1cc([N+](=O)[O-])cc(S(=O)(=O)N2CCc3c(Cl)cccc3C2)c1N(CCOS(C)(=O)=O)CCOS(C)(=O)=O]

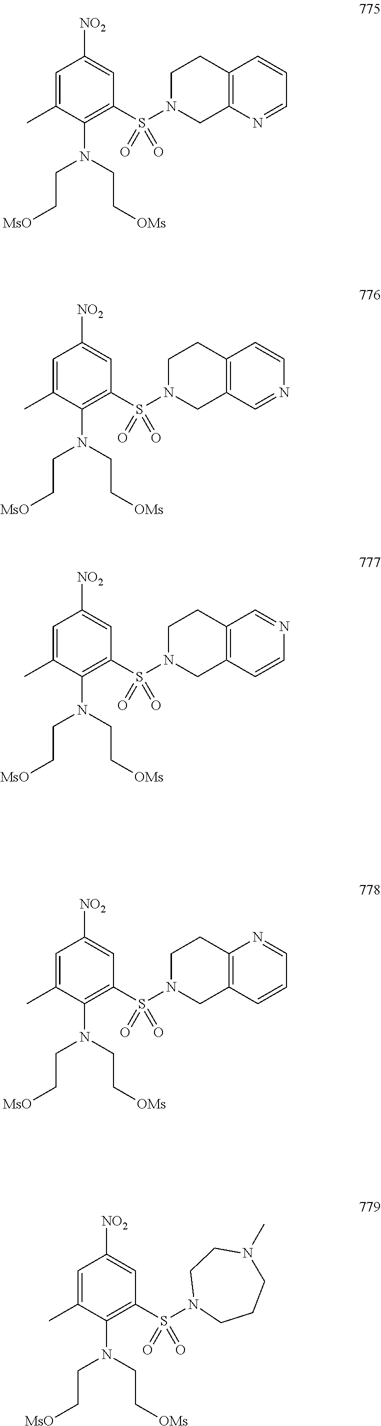 [SMILES: Cc1cc([N+](=O)[O-])cc(S(=O)(=O)N2CCc3cccnc3C2)c1N(CCOS(C)(=O)=O)CCOS(C)(=O)=O ; Cc1cc([N+](=O)[O-])cc(S(=O)(=O)N2CCc3ccncc3C2)c1N(CCOS(C)(=O)=O)CCOS(C)(=O)=O ; Cc1cc([N+](=O)[O-])cc(S(=O)(=O)N2CCc3cnccc3C2)c1N(CCOS(C)(=O)=O)CCOS(C)(=O)=O ; Cc1cc([N+](=O)[O-])cc(S(=O)(=O)N2CCc3ncccc3C2)c1N(CCOS(C)(=O)=O)CCOS(C)(=O)=O ; Cc1cc([N+](=O)[O-])cc(S(=O)(=O)N2CCCN(C)CC2)c1N(CCOS(C)(=O)=O)CCOS(C)(=O)=O]

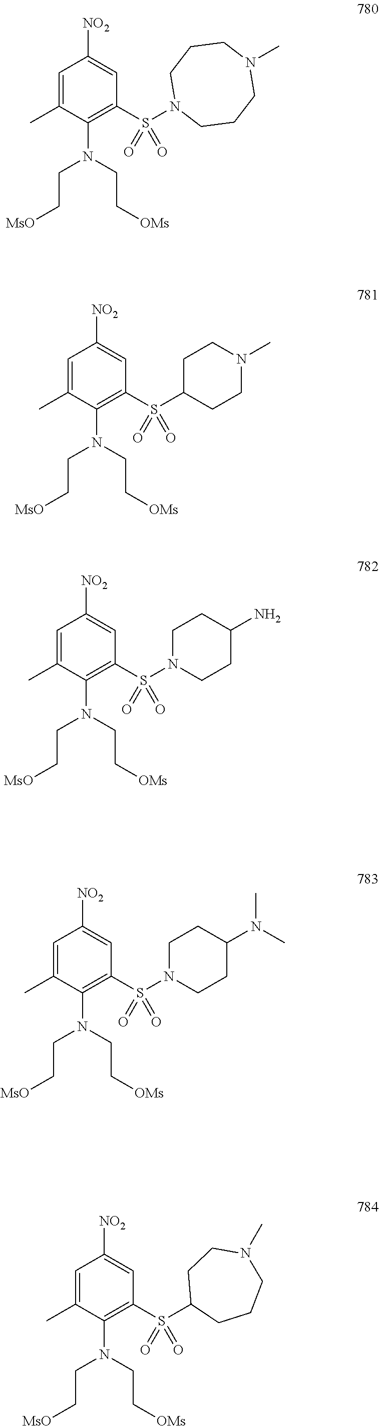 [SMILES: Cc1cc([N+](=O)[O-])cc(S(=O)(=O)N2CCCN(C)CCC2)c1N(CCOS(C)(=O)=O)CCOS(C)(=O)=O ; Cc1cc([N+](=O)[O-])cc(S(=O)(=O)C2CCN(C)CC2)c1N(CCOS(C)(=O)=O)CCOS(C)(=O)=O ; Cc1cc([N+](=O)[O-])cc(S(=O)(=O)N2CCC(N)CC2)c1N(CCOS(C)(=O)=O)CCOS(C)(=O)=O ; Cc1cc([N+](=O)[O-])cc(S(=O)(=O)N2CCC(N(C)C)CC2)c1N(CCOS(C)(=O)=O)CCOS(C)(=O)=O ; Cc1cc([N+](=O)[O-])cc(S(=O)(=O)C2CCCN(C)CC2)c1N(CCOS(C)(=O)=O)CCOS(C)(=O)=O]

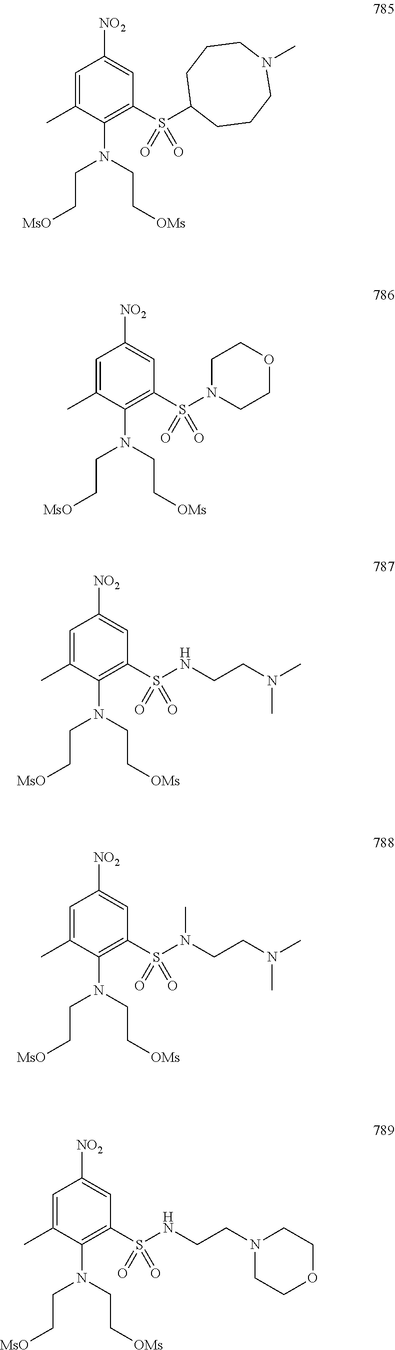 [SMILES: Cc1cc([N+](=O)[O-])cc(S(=O)(=O)C2CCCN(C)CCC2)c1N(CCOS(C)(=O)=O)CCOS(C)(=O)=O ; Cc1cc([N+](=O)[O-])cc(S(=O)(=O)N2CCOCC2)c1N(CCOS(C)(=O)=O)CCOS(C)(=O)=O ; Cc1cc([N+](=O)[O-])cc(S(=O)(=O)NCCN(C)C)c1N(CCOS(C)(=O)=O)CCOS(C)(=O)=O ; Cc1cc([N+](=O)[O-])cc(S(=O)(=O)N(C)CCN(C)C)c1N(CCOS(C)(=O)=O)CCOS(C)(=O)=O ; Cc1cc([N+](=O)[O-])cc(S(=O)(=O)NCCN2CCOCC2)c1N(CCOS(C)(=O)=O)CCOS(C)(=O)=O]

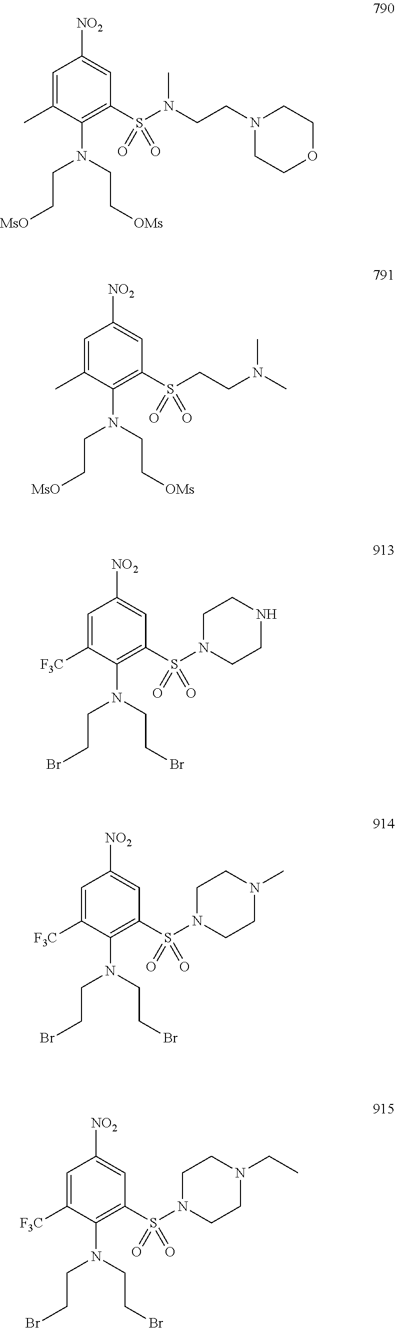 [SMILES: Cc1cc([N+](=O)[O-])cc(S(=O)(=O)N(C)CCN2CCOCC2)c1N(CCOS(C)(=O)=O)CCOS(C)(=O)=O ; Cc1cc([N+](=O)[O-])cc(S(=O)(=O)CCN(C)C)c1N(CCOS(C)(=O)=O)CCOS(C)(=O)=O ; O=[N+]([O-])c1cc(C(F)(F)F)c(N(CCBr)CCBr)c(S(=O)(=O)N2CCNCC2)c1 ; CN1CCN(S(=O)(=O)c2cc([N+](=O)[O-])cc(C(F)(F)F)c2N(CCBr)CCBr)CC1 ; CCN1CCN(S(=O)(=O)c2cc([N+](=O)[O-])cc(C(F)(F)F)c2N(CCBr)CCBr)CC1]

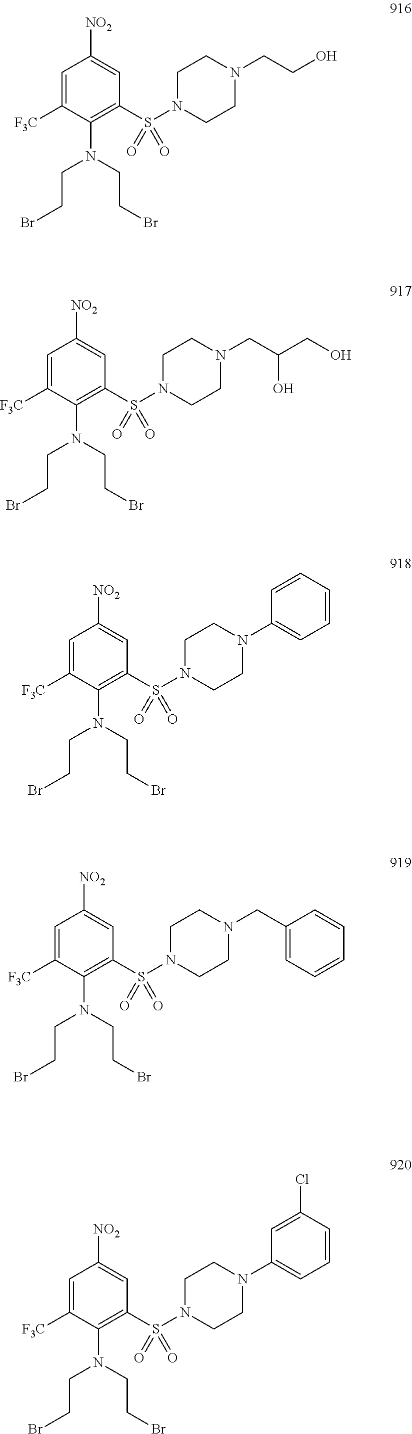 [SMILES: O=[N+]([O-])c1cc(C(F)(F)F)c(N(CCBr)CCBr)c(S(=O)(=O)N2CCN(CCO)CC2)c1 ; O=[N+]([O-])c1cc(C(F)(F)F)c(N(CCBr)CCBr)c(S(=O)(=O)N2CCN(CC(O)CO)CC2)c1 ; O=[N+]([O-])c1cc(C(F)(F)F)c(N(CCBr)CCBr)c(S(=O)(=O)N2CCN(c3ccccc3)CC2)c1 ; O=[N+]([O-])c1cc(C(F)(F)F)c(N(CCBr)CCBr)c(S(=O)(=O)N2CCN(Cc3ccccc3)CC2)c1 ; O=[N+]([O-])c1cc(C(F)(F)F)c(N(CCBr)CCBr)c(S(=O)(=O)N2CCN(c3cccc(Cl)c3)CC2)c1]

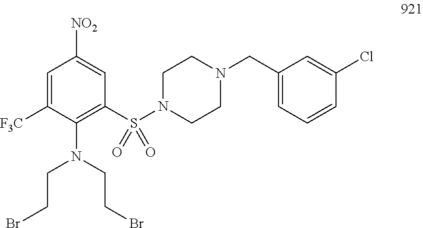 [SMILES: O=[N+]([O-])c1cc(C(F)(F)F)c(N(CCBr)CCBr)c(S(=O)(=O)N2CCN(Cc3cccc(Cl)c3)CC2)c1]

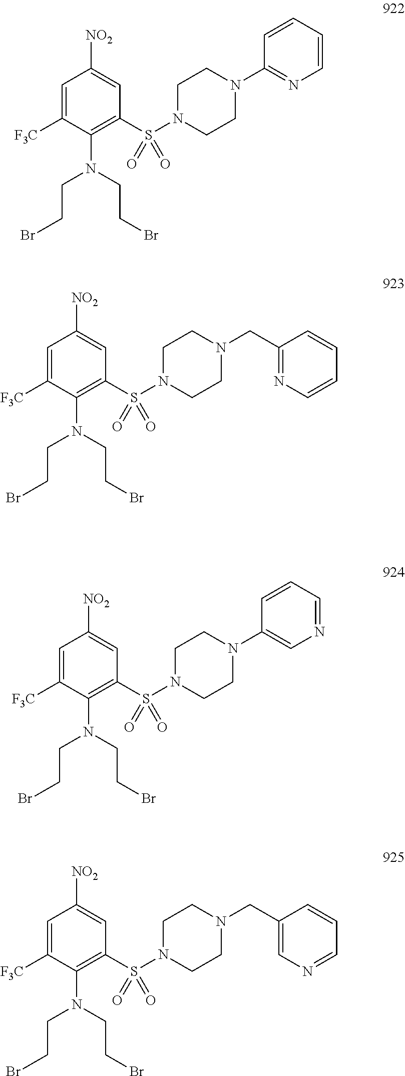 [SMILES: O=[N+]([O-])c1cc(C(F)(F)F)c(N(CCBr)CCBr)c(S(=O)(=O)N2CCN(c3ccccn3)CC2)c1 ; O=[N+]([O-])c1cc(C(F)(F)F)c(N(CCBr)CCBr)c(S(=O)(=O)N2CCN(Cc3ccccn3)CC2)c1 ; O=[N+]([O-])c1cc(C(F)(F)F)c(N(CCBr)CCBr)c(S(=O)(=O)N2CCN(c3cccnc3)CC2)c1 ; O=[N+]([O-])c1cc(C(F)(F)F)c(N(CCBr)CCBr)c(S(=O)(=O)N2CCN(Cc3cccnc3)CC2)c1]

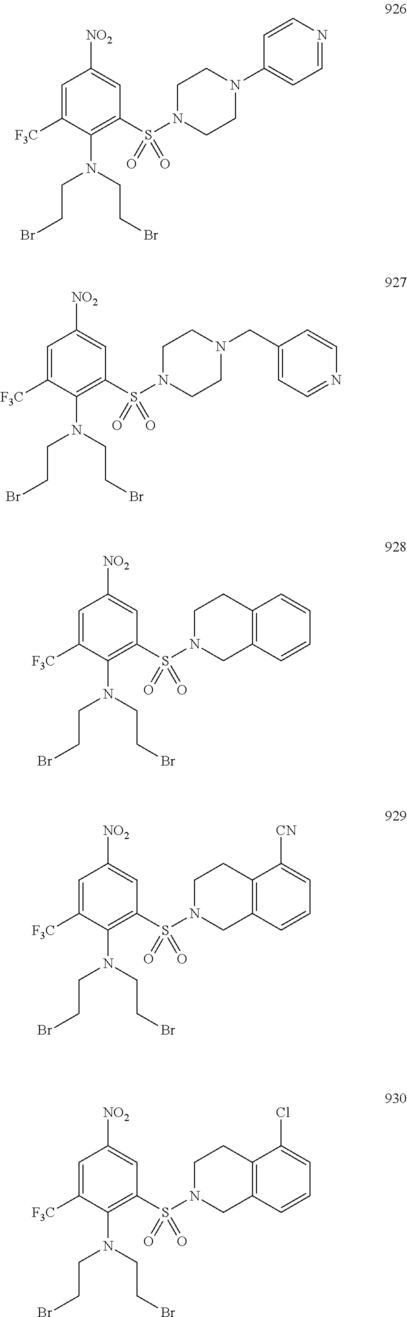 [SMILES: O=[N+]([O-])c1cc(C(F)(F)F)c(N(CCBr)CCBr)c(S(=O)(=O)N2CCN(c3ccncc3)CC2)c1 ; O=[N+]([O-])c1cc(C(F)(F)F)c(N(CCBr)CCBr)c(S(=O)(=O)N2CCN(Cc3ccncc3)CC2)c1 ; O=[N+]([O-])c1cc(C(F)(F)F)c(N(CCBr)CCBr)c(S(=O)(=O)N2CCc3ccccc3C2)c1 ; N#Cc1cccc2c1CCN(S(=O)(=O)c1cc([N+](=O)[O-])cc(C(F)(F)F)c1N(CCBr)CCBr)C2 ; O=[N+]([O-])c1cc(C(F)(F)F)c(N(CCBr)CCBr)c(S(=O)(=O)N2CCc3c(Cl)cccc3C2)c1]

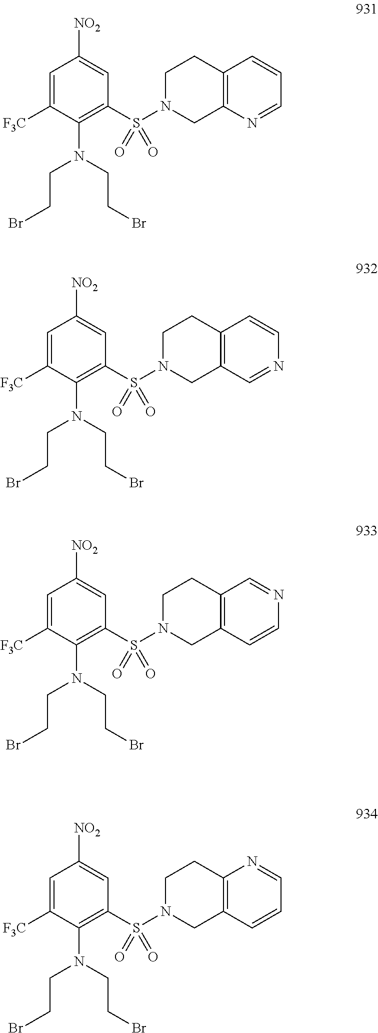 [SMILES: O=[N+]([O-])c1cc(C(F)(F)F)c(N(CCBr)CCBr)c(S(=O)(=O)N2CCc3cccnc3C2)c1 ; O=[N+]([O-])c1cc(C(F)(F)F)c(N(CCBr)CCBr)c(S(=O)(=O)N2CCc3ccncc3C2)c1 ; O=[N+]([O-])c1cc(C(F)(F)F)c(N(CCBr)CCBr)c(S(=O)(=O)N2CCc3cnccc3C2)c1 ; O=[N+]([O-])c1cc(C(F)(F)F)c(N(CCBr)CCBr)c(S(=O)(=O)N2CCc3ncccc3C2)c1]

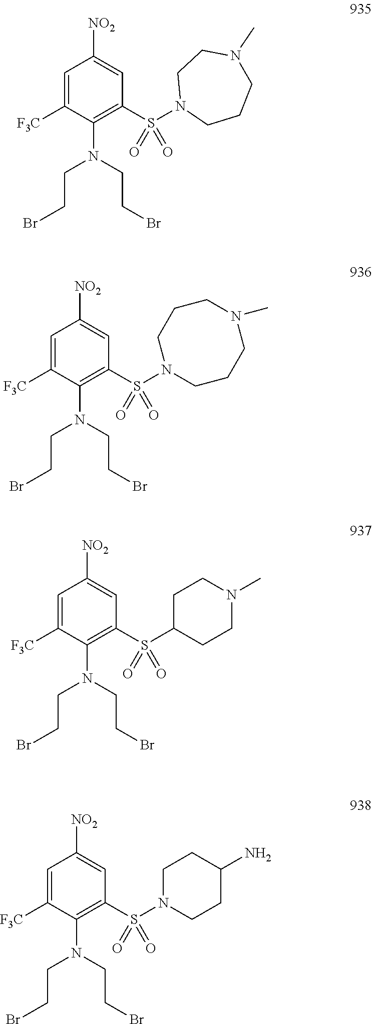 [SMILES: CN1CCCN(S(=O)(=O)c2cc([N+](=O)[O-])cc(C(F)(F)F)c2N(CCBr)CCBr)CC1 ; CN1CCCN(S(=O)(=O)c2cc([N+](=O)[O-])cc(C(F)(F)F)c2N(CCBr)CCBr)CCC1 ; CN1CCC(S(=O)(=O)c2cc([N+](=O)[O-])cc(C(F)(F)F)c2N(CCBr)CCBr)CC1 ; NC1CCN(S(=O)(=O)c2cc([N+](=O)[O-])cc(C(F)(F)F)c2N(CCBr)CCBr)CC1]

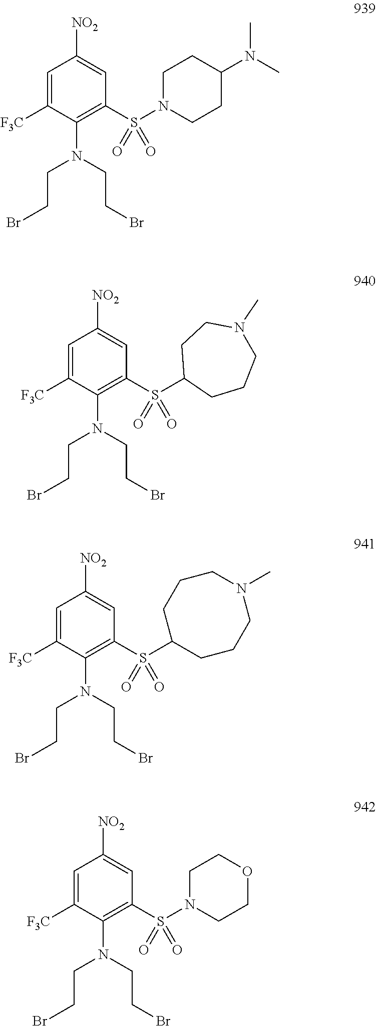 [SMILES: CN(C)C1CCN(S(=O)(=O)c2cc([N+](=O)[O-])cc(C(F)(F)F)c2N(CCBr)CCBr)CC1 ; CN1CCCC(S(=O)(=O)c2cc([N+](=O)[O-])cc(C(F)(F)F)c2N(CCBr)CCBr)CC1 ; CN1CCCC(S(=O)(=O)c2cc([N+](=O)[O-])cc(C(F)(F)F)c2N(CCBr)CCBr)CCC1 ; O=[N+]([O-])c1cc(C(F)(F)F)c(N(CCBr)CCBr)c(S(=O)(=O)N2CCOCC2)c1]

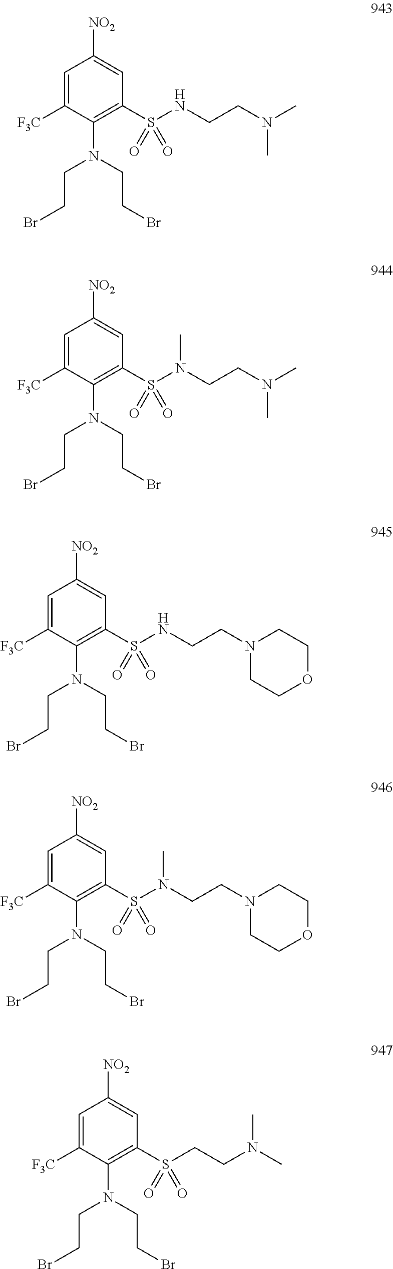 [SMILES: CN(C)CCNS(=O)(=O)c1cc([N+](=O)[O-])cc(C(F)(F)F)c1N(CCBr)CCBr ; CN(C)CCN(C)S(=O)(=O)c1cc([N+](=O)[O-])cc(C(F)(F)F)c1N(CCBr)CCBr ; O=[N+]([O-])c1cc(C(F)(F)F)c(N(CCBr)CCBr)c(S(=O)(=O)NCCN2CCOCC2)c1 ; CN(CCN1CCOCC1)S(=O)(=O)c1cc([N+](=O)[O-])cc(C(F)(F)F)c1N(CCBr)CCBr ; CN(C)CCS(=O)(=O)c1cc([N+](=O)[O-])cc(C(F)(F)F)c1N(CCBr)CCBr]

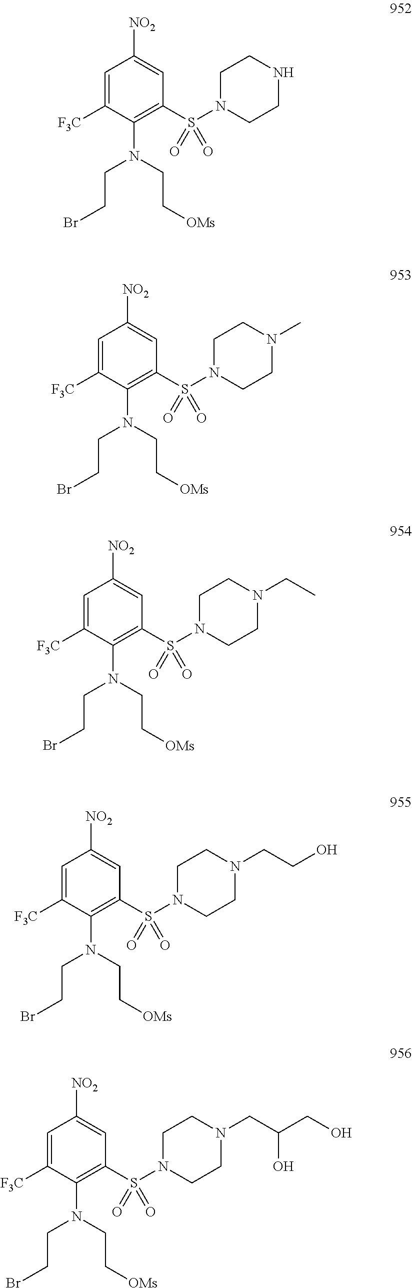 [SMILES: CS(=O)(=O)OCCN(CCBr)c1c(C(F)(F)F)cc([N+](=O)[O-])cc1S(=O)(=O)N1CCNCC1 ; CN1CCN(S(=O)(=O)c2cc([N+](=O)[O-])cc(C(F)(F)F)c2N(CCBr)CCOS(C)(=O)=O)CC1 ; CCN1CCN(S(=O)(=O)c2cc([N+](=O)[O-])cc(C(F)(F)F)c2N(CCBr)CCOS(C)(=O)=O)CC1 ; CS(=O)(=O)OCCN(CCBr)c1c(C(F)(F)F)cc([N+](=O)[O-])cc1S(=O)(=O)N1CCN(CCO)CC1 ; CS(=O)(=O)OCCN(CCBr)c1c(C(F)(F)F)cc([N+](=O)[O-])cc1S(=O)(=O)N1CCN(CC(O)CO)CC1]

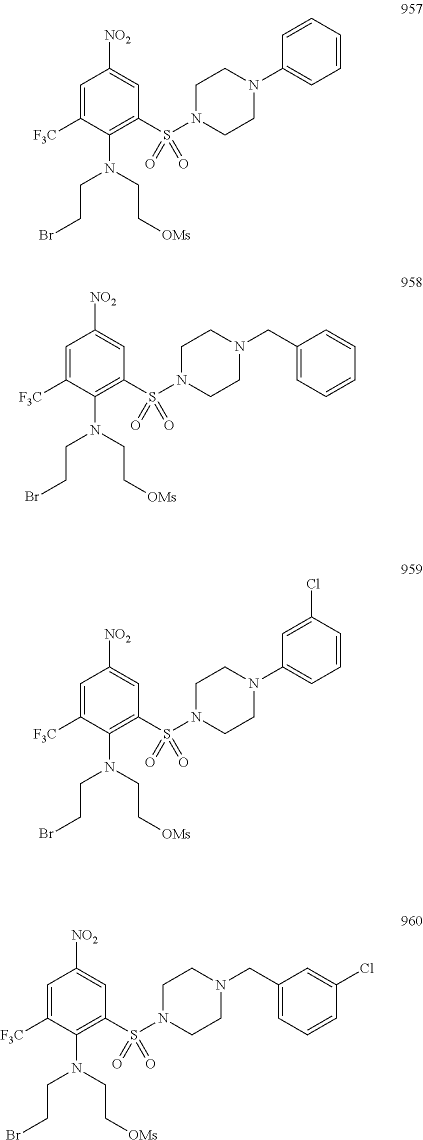 [SMILES: CS(=O)(=O)OCCN(CCBr)c1c(C(F)(F)F)cc([N+](=O)[O-])cc1S(=O)(=O)N1CCN(c2ccccc2)CC1 ; CS(=O)(=O)OCCN(CCBr)c1c(C(F)(F)F)cc([N+](=O)[O-])cc1S(=O)(=O)N1CCN(Cc2ccccc2)CC1 ; CS(=O)(=O)OCCN(CCBr)c1c(C(F)(F)F)cc([N+](=O)[O-])cc1S(=O)(=O)N1CCN(c2cccc(Cl)c2)CC1 ; CS(=O)(=O)OCCN(CCBr)c1c(C(F)(F)F)cc([N+](=O)[O-])cc1S(=O)(=O)N1CCN(Cc2cccc(Cl)c2)CC1]

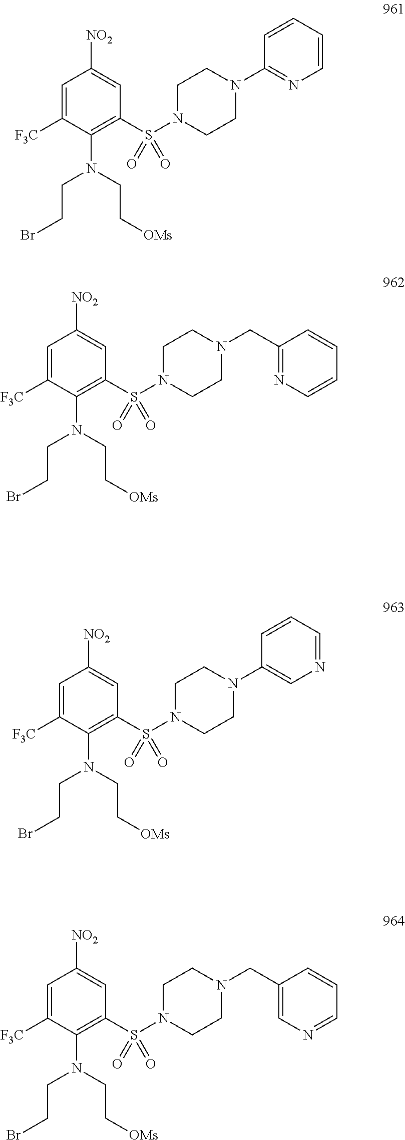 [SMILES: CS(=O)(=O)OCCN(CCBr)c1c(C(F)(F)F)cc([N+](=O)[O-])cc1S(=O)(=O)N1CCN(c2ccccn2)CC1 ; CS(=O)(=O)OCCN(CCBr)c1c(C(F)(F)F)cc([N+](=O)[O-])cc1S(=O)(=O)N1CCN(Cc2ccccn2)CC1 ; CS(=O)(=O)OCCN(CCBr)c1c(C(F)(F)F)cc([N+](=O)[O-])cc1S(=O)(=O)N1CCN(c2cccnc2)CC1 ; CS(=O)(=O)OCCN(CCBr)c1c(C(F)(F)F)cc([N+](=O)[O-])cc1S(=O)(=O)N1CCN(Cc2cccnc2)CC1]

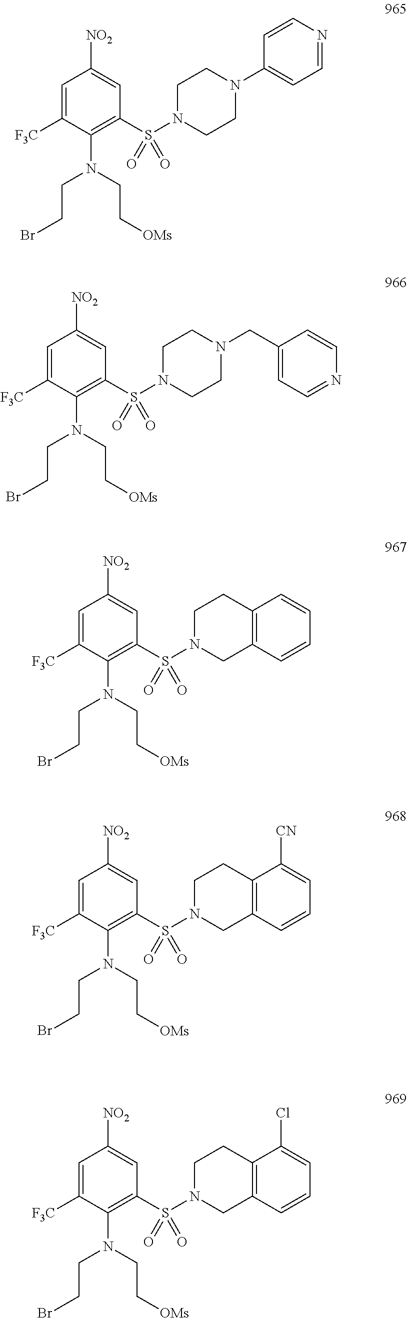 [SMILES: CS(=O)(=O)OCCN(CCBr)c1c(C(F)(F)F)cc([N+](=O)[O-])cc1S(=O)(=O)N1CCN(c2ccncc2)CC1 ; CS(=O)(=O)OCCN(CCBr)c1c(C(F)(F)F)cc([N+](=O)[O-])cc1S(=O)(=O)N1CCN(Cc2ccncc2)CC1 ; CS(=O)(=O)OCCN(CCBr)c1c(C(F)(F)F)cc([N+](=O)[O-])cc1S(=O)(=O)N1CCc2ccccc2C1 ; CS(=O)(=O)OCCN(CCBr)c1c(C(F)(F)F)cc([N+](=O)[O-])cc1S(=O)(=O)N1CCc2c(C#N)cccc2C1 ; CS(=O)(=O)OCCN(CCBr)c1c(C(F)(F)F)cc([N+](=O)[O-])cc1S(=O)(=O)N1CCc2c(Cl)cccc2C1]

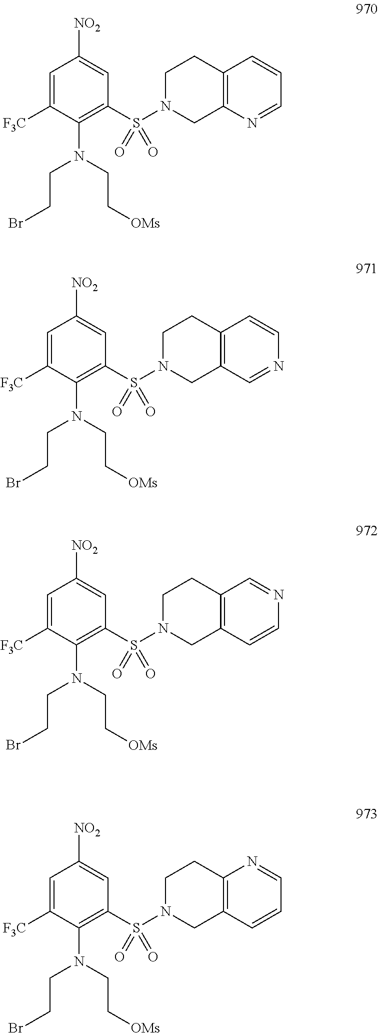 [SMILES: CS(=O)(=O)OCCN(CCBr)c1c(C(F)(F)F)cc([N+](=O)[O-])cc1S(=O)(=O)N1CCc2cccnc2C1 ; CS(=O)(=O)OCCN(CCBr)c1c(C(F)(F)F)cc([N+](=O)[O-])cc1S(=O)(=O)N1CCc2ccncc2C1 ; CS(=O)(=O)OCCN(CCBr)c1c(C(F)(F)F)cc([N+](=O)[O-])cc1S(=O)(=O)N1CCc2cnccc2C1 ; CS(=O)(=O)OCCN(CCBr)c1c(C(F)(F)F)cc([N+](=O)[O-])cc1S(=O)(=O)N1CCc2ncccc2C1]

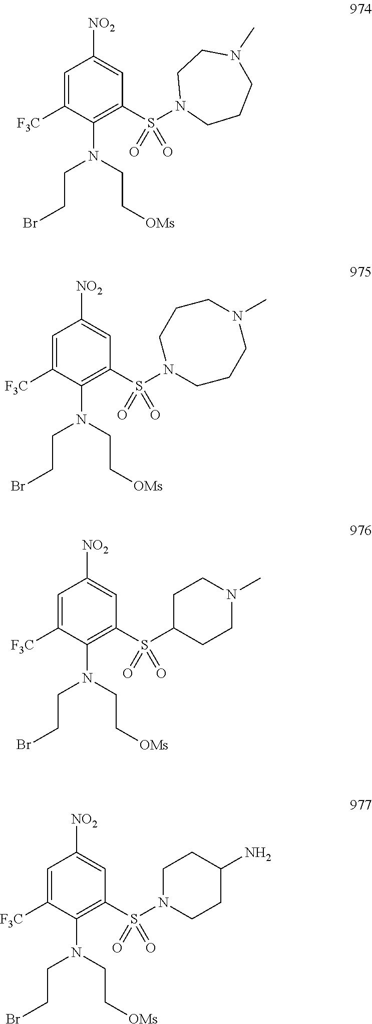 [SMILES: CN1CCCN(S(=O)(=O)c2cc([N+](=O)[O-])cc(C(F)(F)F)c2N(CCBr)CCOS(C)(=O)=O)CC1 ; CN1CCCN(S(=O)(=O)c2cc([N+](=O)[O-])cc(C(F)(F)F)c2N(CCBr)CCOS(C)(=O)=O)CCC1 ; CN1CCC(S(=O)(=O)c2cc([N+](=O)[O-])cc(C(F)(F)F)c2N(CCBr)CCOS(C)(=O)=O)CC1 ; CS(=O)(=O)OCCN(CCBr)c1c(C(F)(F)F)cc([N+](=O)[O-])cc1S(=O)(=O)N1CCC(N)CC1]

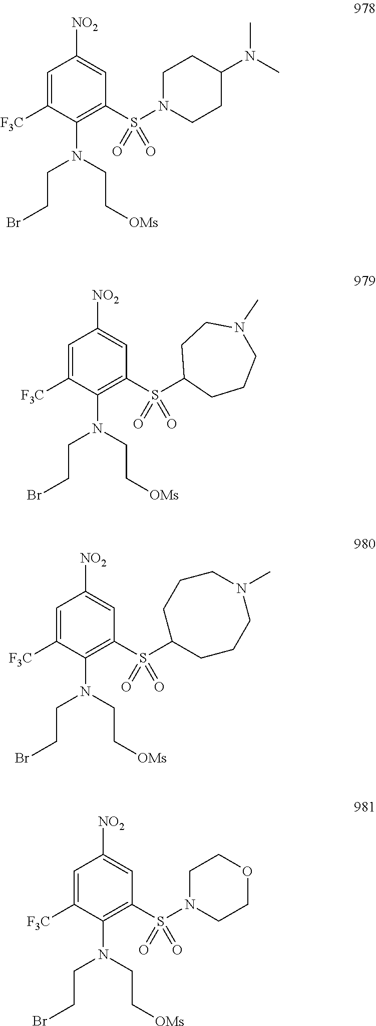 [SMILES: CN(C)C1CCN(S(=O)(=O)c2cc([N+](=O)[O-])cc(C(F)(F)F)c2N(CCBr)CCOS(C)(=O)=O)CC1 ; CN1CCCC(S(=O)(=O)c2cc([N+](=O)[O-])cc(C(F)(F)F)c2N(CCBr)CCOS(C)(=O)=O)CC1 ; CN1CCCC(S(=O)(=O)c2cc([N+](=O)[O-])cc(C(F)(F)F)c2N(CCBr)CCOS(C)(=O)=O)CCC1 ; CS(=O)(=O)OCCN(CCBr)c1c(C(F)(F)F)cc([N+](=O)[O-])cc1S(=O)(=O)N1CCOCC1]

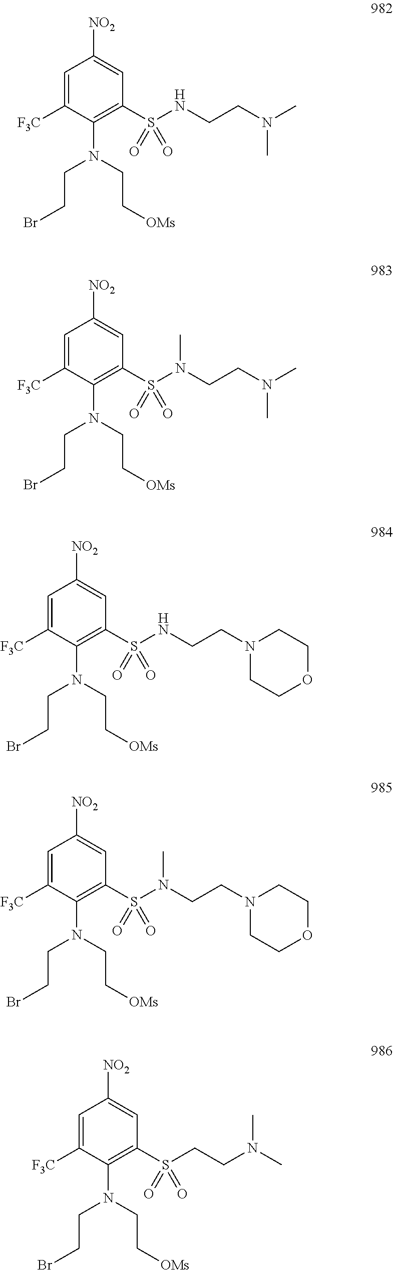 [SMILES: CN(C)CCNS(=O)(=O)c1cc([N+](=O)[O-])cc(C(F)(F)F)c1N(CCBr)CCOS(C)(=O)=O ; CN(C)CCN(C)S(=O)(=O)c1cc([N+](=O)[O-])cc(C(F)(F)F)c1N(CCBr)CCOS(C)(=O)=O ; CS(=O)(=O)OCCN(CCBr)c1c(C(F)(F)F)cc([N+](=O)[O-])cc1S(=O)(=O)NCCN1CCOCC1 ; CN(CCN1CCOCC1)S(=O)(=O)c1cc([N+](=O)[O-])cc(C(F)(F)F)c1N(CCBr)CCOS(C)(=O)=O ; CN(C)CCS(=O)(=O)c1cc([N+](=O)[O-])cc(C(F)(F)F)c1N(CCBr)CCOS(C)(=O)=O]

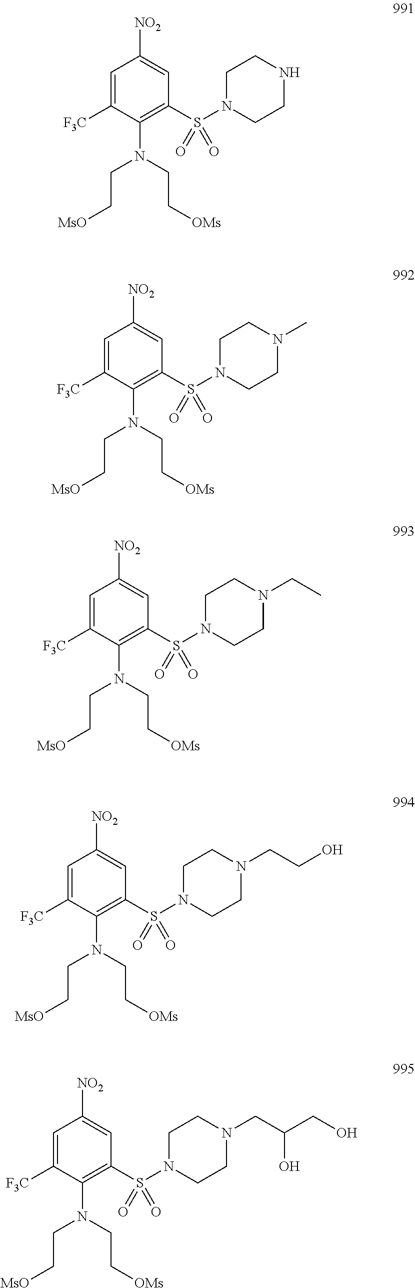 [SMILES: CS(=O)(=O)OCCN(CCOS(C)(=O)=O)c1c(C(F)(F)F)cc([N+](=O)[O-])cc1S(=O)(=O)N1CCNCC1 ; CN1CCN(S(=O)(=O)c2cc([N+](=O)[O-])cc(C(F)(F)F)c2N(CCOS(C)(=O)=O)CCOS(C)(=O)=O)CC1 ; CCN1CCN(S(=O)(=O)c2cc([N+](=O)[O-])cc(C(F)(F)F)c2N(CCOS(C)(=O)=O)CCOS(C)(=O)=O)CC1 ; CS(=O)(=O)OCCN(CCOS(C)(=O)=O)c1c(C(F)(F)F)cc([N+](=O)[O-])cc1S(=O)(=O)N1CCN(CCO)CC1 ; CS(=O)(=O)OCCN(CCOS(C)(=O)=O)c1c(C(F)(F)F)cc([N+](=O)[O-])cc1S(=O)(=O)N1CCN(CC(O)CO)CC1]

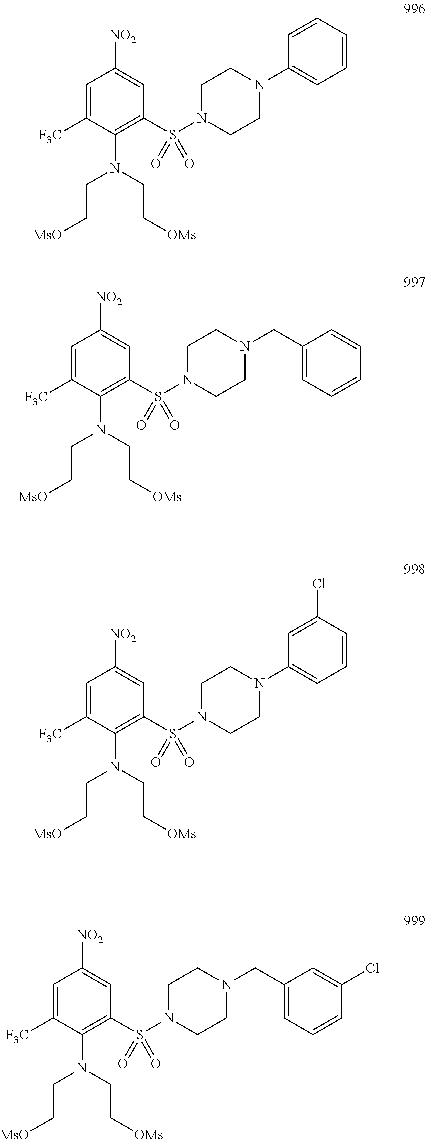 [SMILES: CS(=O)(=O)OCCN(CCOS(C)(=O)=O)c1c(C(F)(F)F)cc([N+](=O)[O-])cc1S(=O)(=O)N1CCN(c2ccccc2)CC1 ; CS(=O)(=O)OCCN(CCOS(C)(=O)=O)c1c(C(F)(F)F)cc([N+](=O)[O-])cc1S(=O)(=O)N1CCN(Cc2ccccc2)CC1 ; CS(=O)(=O)OCCN(CCOS(C)(=O)=O)c1c(C(F)(F)F)cc([N+](=O)[O-])cc1S(=O)(=O)N1CCN(c2cccc(Cl)c2)CC1 ; CS(=O)(=O)OCCN(CCOS(C)(=O)=O)c1c(C(F)(F)F)cc([N+](=O)[O-])cc1S(=O)(=O)N1CCN(Cc2cccc(Cl)c2)CC1]

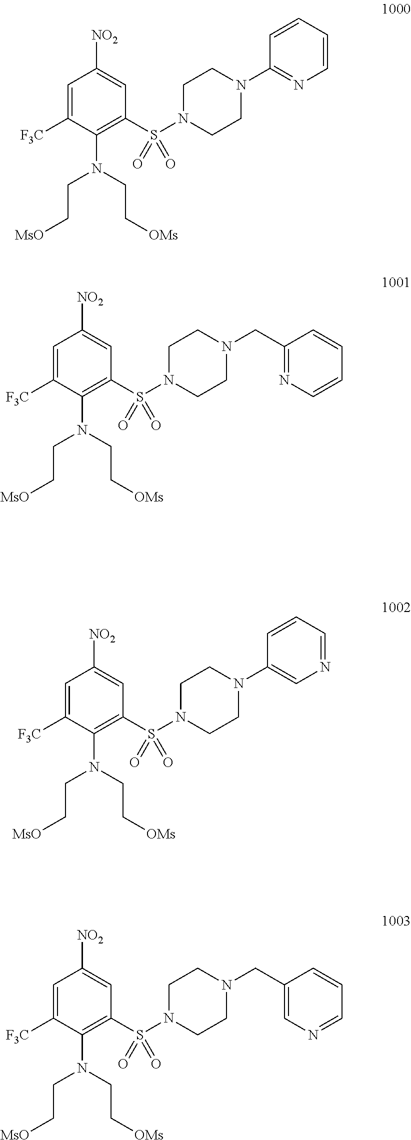 [SMILES: CS(=O)(=O)OCCN(CCOS(C)(=O)=O)c1c(C(F)(F)F)cc([N+](=O)[O-])cc1S(=O)(=O)N1CCN(c2ccccn2)CC1 ; CS(=O)(=O)OCCN(CCOS(C)(=O)=O)c1c(C(F)(F)F)cc([N+](=O)[O-])cc1S(=O)(=O)N1CCN(Cc2ccccn2)CC1 ; CS(=O)(=O)OCCN(CCOS(C)(=O)=O)c1c(C(F)(F)F)cc([N+](=O)[O-])cc1S(=O)(=O)N1CCN(c2cccnc2)CC1 ; CS(=O)(=O)OCCN(CCOS(C)(=O)=O)c1c(C(F)(F)F)cc([N+](=O)[O-])cc1S(=O)(=O)N1CCN(Cc2cccnc2)CC1]

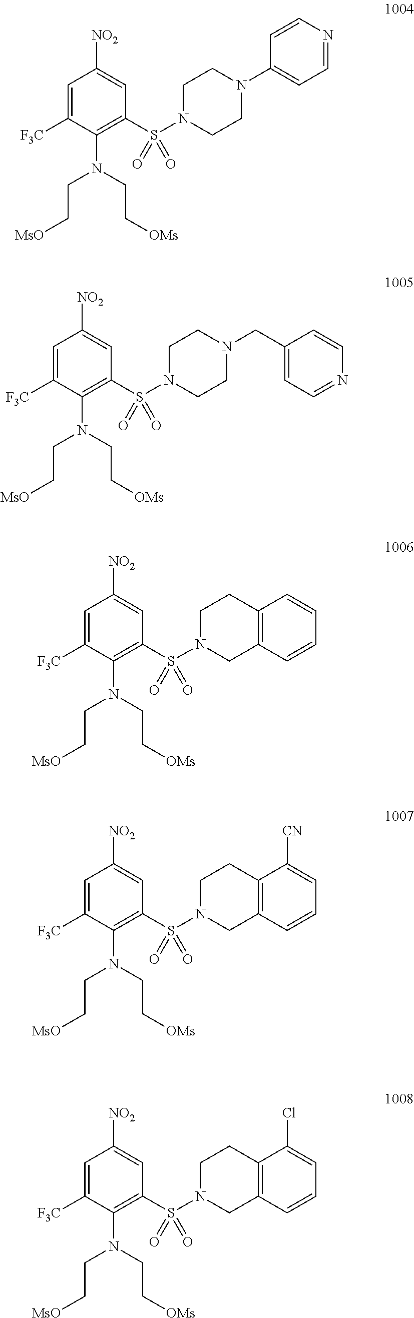 [SMILES: CS(=O)(=O)OCCN(CCOS(C)(=O)=O)c1c(C(F)(F)F)cc([N+](=O)[O-])cc1S(=O)(=O)N1CCN(c2ccncc2)CC1 ; CS(=O)(=O)OCCN(CCOS(C)(=O)=O)c1c(C(F)(F)F)cc([N+](=O)[O-])cc1S(=O)(=O)N1CCN(Cc2ccncc2)CC1 ; CS(=O)(=O)OCCN(CCOS(C)(=O)=O)c1c(C(F)(F)F)cc([N+](=O)[O-])cc1S(=O)(=O)N1CCc2ccccc2C1 ; CS(=O)(=O)OCCN(CCOS(C)(=O)=O)c1c(C(F)(F)F)cc([N+](=O)[O-])cc1S(=O)(=O)N1CCc2c(C#N)cccc2C1 ; CS(=O)(=O)OCCN(CCOS(C)(=O)=O)c1c(C(F)(F)F)cc([N+](=O)[O-])cc1S(=O)(=O)N1CCc2c(Cl)cccc2C1]

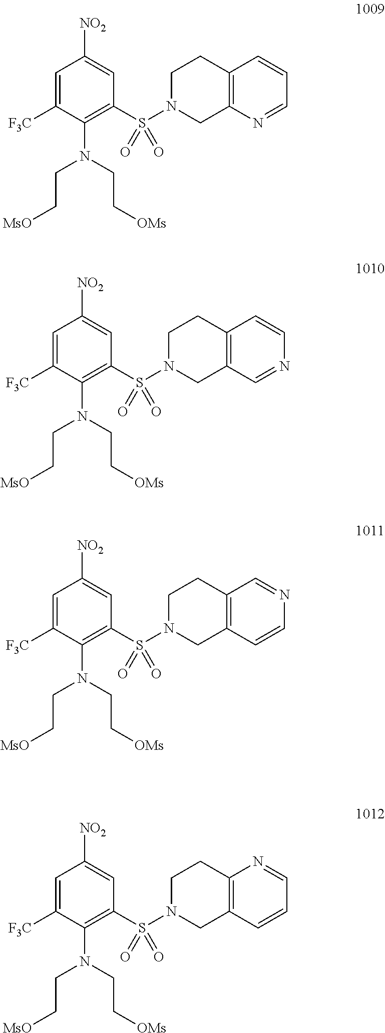 [SMILES: CS(=O)(=O)OCCN(CCOS(C)(=O)=O)c1c(C(F)(F)F)cc([N+](=O)[O-])cc1S(=O)(=O)N1CCc2cccnc2C1 ; CS(=O)(=O)OCCN(CCOS(C)(=O)=O)c1c(C(F)(F)F)cc([N+](=O)[O-])cc1S(=O)(=O)N1CCc2ccncc2C1 ; CS(=O)(=O)OCCN(CCOS(C)(=O)=O)c1c(C(F)(F)F)cc([N+](=O)[O-])cc1S(=O)(=O)N1CCc2cnccc2C1 ; CS(=O)(=O)OCCN(CCOS(C)(=O)=O)c1c(C(F)(F)F)cc([N+](=O)[O-])cc1S(=O)(=O)N1CCc2ncccc2C1]

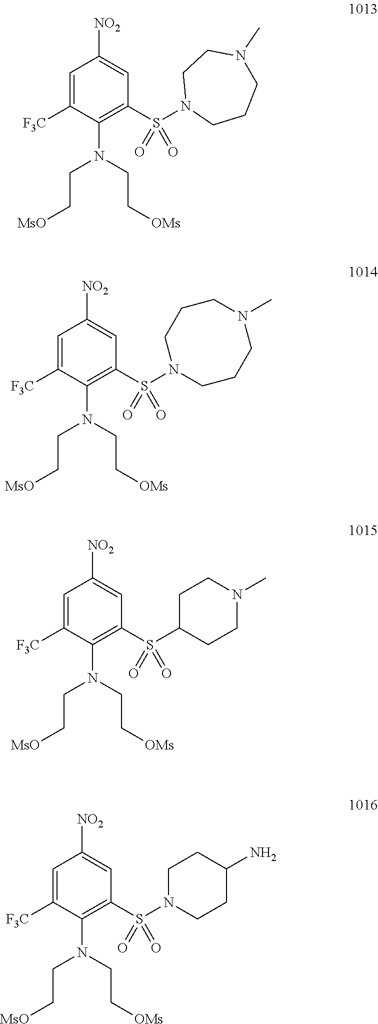 [SMILES: CN1CCCN(S(=O)(=O)c2cc([N+](=O)[O-])cc(C(F)(F)F)c2N(CCOS(C)(=O)=O)CCOS(C)(=O)=O)CC1 ; CN1CCCN(S(=O)(=O)c2cc([N+](=O)[O-])cc(C(F)(F)F)c2N(CCOS(C)(=O)=O)CCOS(C)(=O)=O)CCC1 ; CN1CCC(S(=O)(=O)c2cc([N+](=O)[O-])cc(C(F)(F)F)c2N(CCOS(C)(=O)=O)CCOS(C)(=O)=O)CC1 ; CS(=O)(=O)OCCN(CCOS(C)(=O)=O)c1c(C(F)(F)F)cc([N+](=O)[O-])cc1S(=O)(=O)N1CCC(N)CC1]

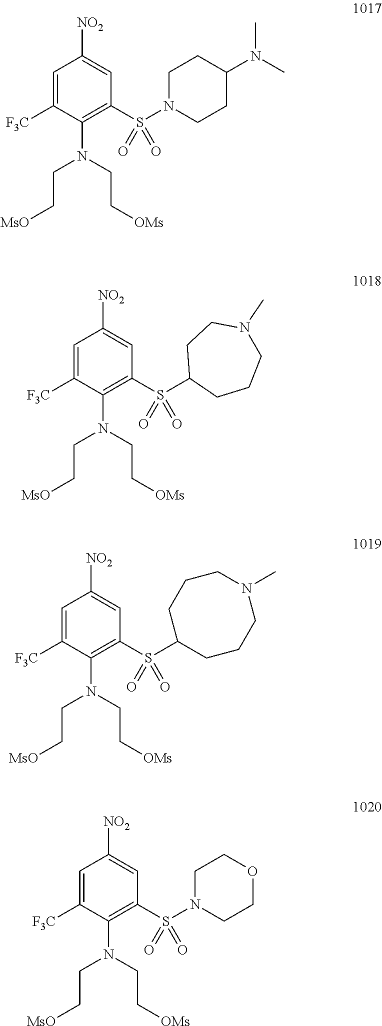 [SMILES: CN(C)C1CCN(S(=O)(=O)c2cc([N+](=O)[O-])cc(C(F)(F)F)c2N(CCOS(C)(=O)=O)CCOS(C)(=O)=O)CC1 ; CN1CCCC(S(=O)(=O)c2cc([N+](=O)[O-])cc(C(F)(F)F)c2N(CCOS(C)(=O)=O)CCOS(C)(=O)=O)CC1 ; CN1CCCC(S(=O)(=O)c2cc([N+](=O)[O-])cc(C(F)(F)F)c2N(CCOS(C)(=O)=O)CCOS(C)(=O)=O)CCC1 ; CS(=O)(=O)OCCN(CCOS(C)(=O)=O)c1c(C(F)(F)F)cc([N+](=O)[O-])cc1S(=O)(=O)N1CCOCC1]

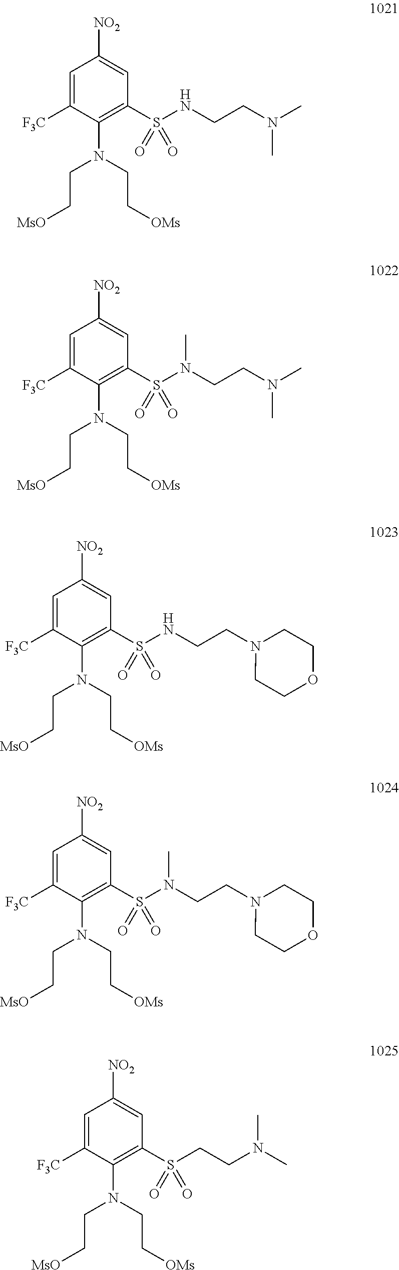 [SMILES: CN(C)CCNS(=O)(=O)c1cc([N+](=O)[O-])cc(C(F)(F)F)c1N(CCOS(C)(=O)=O)CCOS(C)(=O)=O ; CN(C)CCN(C)S(=O)(=O)c1cc([N+](=O)[O-])cc(C(F)(F)F)c1N(CCOS(C)(=O)=O)CCOS(C)(=O)=O ; CS(=O)(=O)OCCN(CCOS(C)(=O)=O)c1c(C(F)(F)F)cc([N+](=O)[O-])cc1S(=O)(=O)NCCN1CCOCC1 ; CN(CCN1CCOCC1)S(=O)(=O)c1cc([N+](=O)[O-])cc(C(F)(F)F)c1N(CCOS(C)(=O)=O)CCOS(C)(=O)=O ; CN(C)CCS(=O)(=O)c1cc([N+](=O)[O-])cc(C(F)(F)F)c1N(CCOS(C)(=O)=O)CCOS(C)(=O)=O]

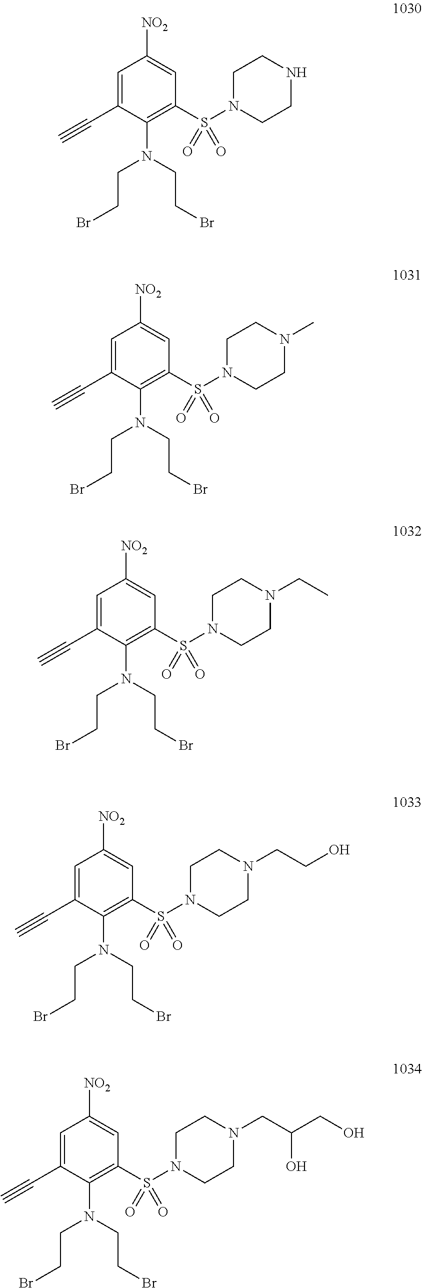

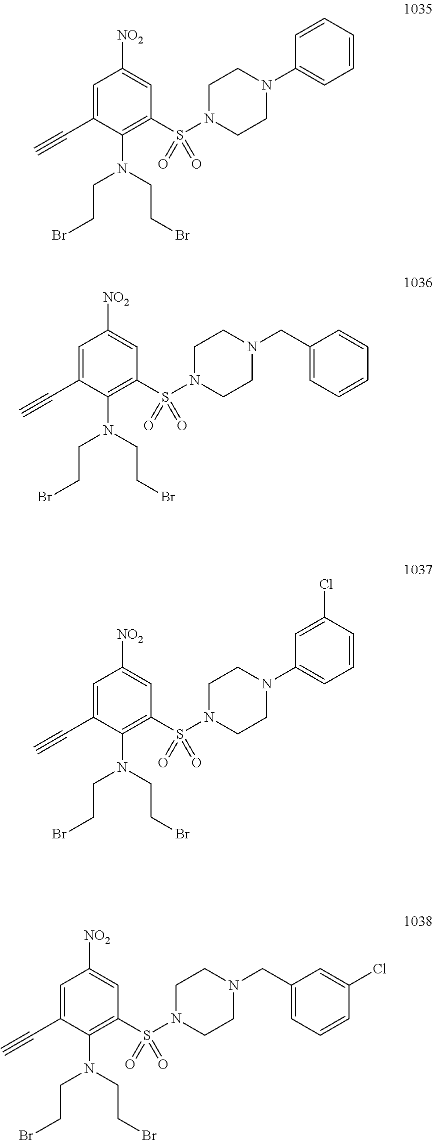 [SMILES: C#Cc1cc([N+](=O)[O-])cc(S(=O)(=O)N2CCN(c3ccccc3)CC2)c1N(CCBr)CCBr ; C#Cc1cc([N+](=O)[O-])cc(S(=O)(=O)N2CCN(Cc3ccccc3)CC2)c1N(CCBr)CCBr ; C#Cc1cc([N+](=O)[O-])cc(S(=O)(=O)N2CCN(c3cccc(Cl)c3)CC2)c1N(CCBr)CCBr ; C#Cc1cc([N+](=O)[O-])cc(S(=O)(=O)N2CCN(Cc3cccc(Cl)c3)CC2)c1N(CCBr)CCBr]

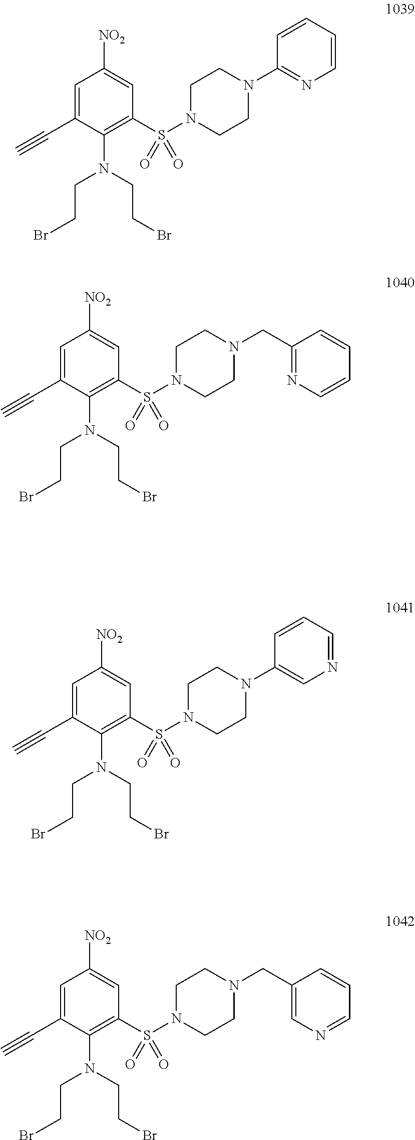 [SMILES: C#Cc1cc([N+](=O)[O-])cc(S(=O)(=O)N2CCN(c3ccccn3)CC2)c1N(CCBr)CCBr ; C#Cc1cc([N+](=O)[O-])cc(S(=O)(=O)N2CCN(Cc3ccccn3)CC2)c1N(CCBr)CCBr ; C#Cc1cc([N+](=O)[O-])cc(S(=O)(=O)N2CCN(c3cccnc3)CC2)c1N(CCBr)CCBr ; C#Cc1cc([N+](=O)[O-])cc(S(=O)(=O)N2CCN(Cc3cccnc3)CC2)c1N(CCBr)CCBr]

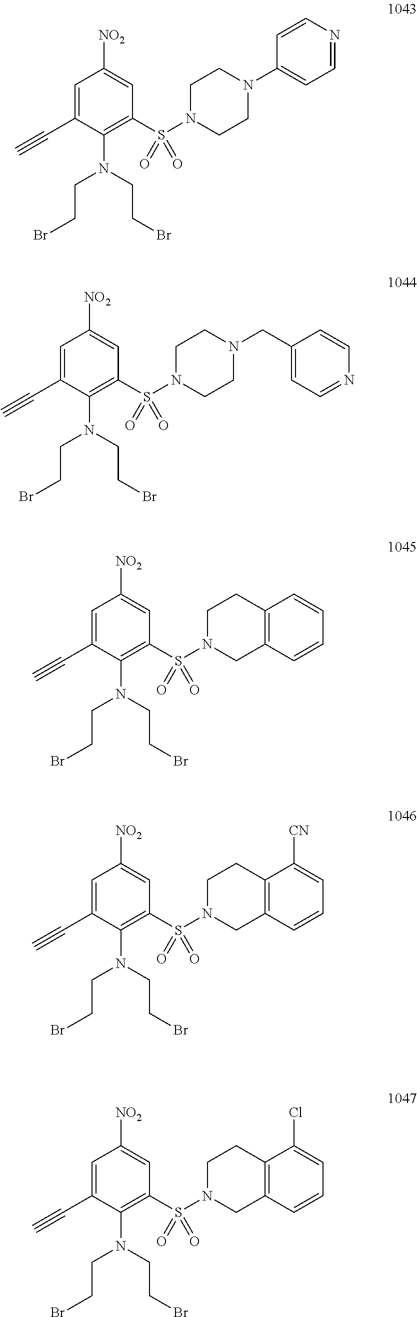 [SMILES: C#Cc1cc([N+](=O)[O-])cc(S(=O)(=O)N2CCN(c3ccncc3)CC2)c1N(CCBr)CCBr ; C#Cc1cc([N+](=O)[O-])cc(S(=O)(=O)N2CCN(Cc3ccncc3)CC2)c1N(CCBr)CCBr ; C#Cc1cc([N+](=O)[O-])cc(S(=O)(=O)N2CCc3ccccc3C2)c1N(CCBr)CCBr ; C#Cc1cc([N+](=O)[O-])cc(S(=O)(=O)N2CCc3c(C#N)cccc3C2)c1N(CCBr)CCBr ; C#Cc1cc([N+](=O)[O-])cc(S(=O)(=O)N2CCc3c(Cl)cccc3C2)c1N(CCBr)CCBr]

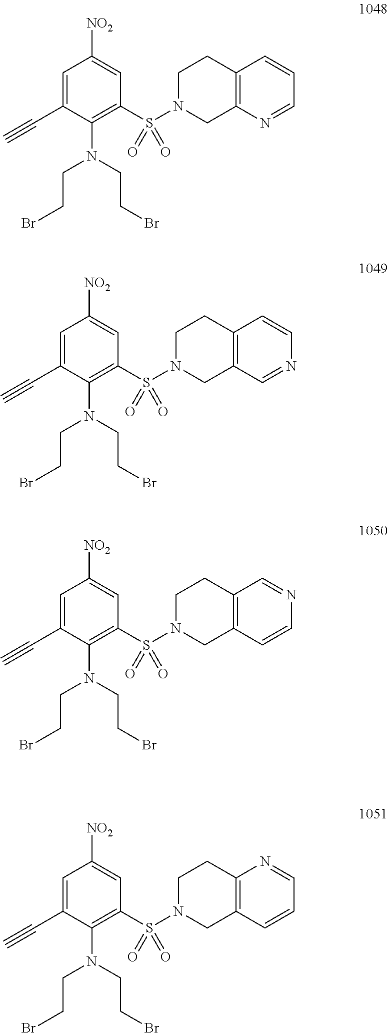 [SMILES: C#Cc1cc([N+](=O)[O-])cc(S(=O)(=O)N2CCc3cccnc3C2)c1N(CCBr)CCBr ; C#Cc1cc([N+](=O)[O-])cc(S(=O)(=O)N2CCc3ccncc3C2)c1N(CCBr)CCBr ; C#Cc1cc([N+](=O)[O-])cc(S(=O)(=O)N2CCc3cnccc3C2)c1N(CCBr)CCBr ; C#Cc1cc([N+](=O)[O-])cc(S(=O)(=O)N2CCc3ncccc3C2)c1N(CCBr)CCBr]

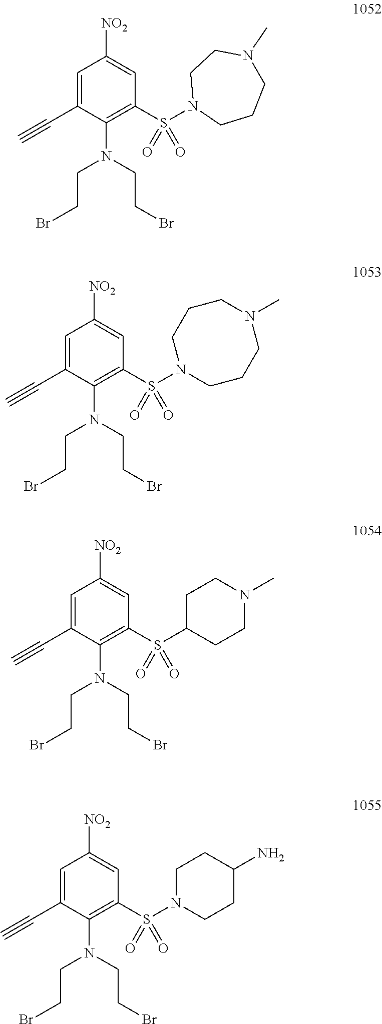

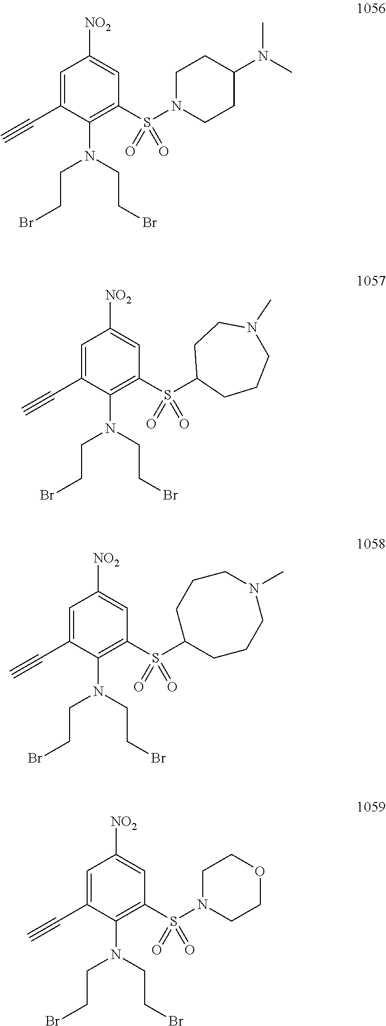

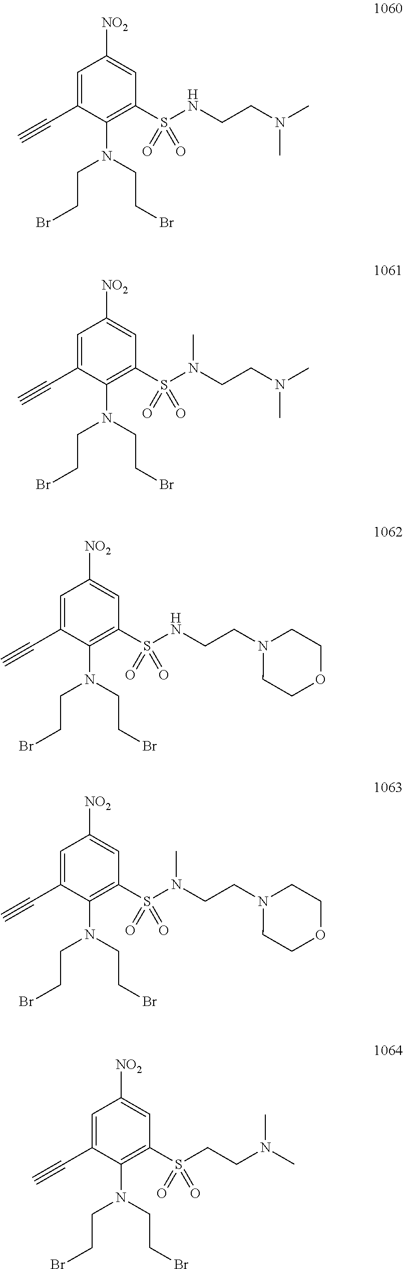

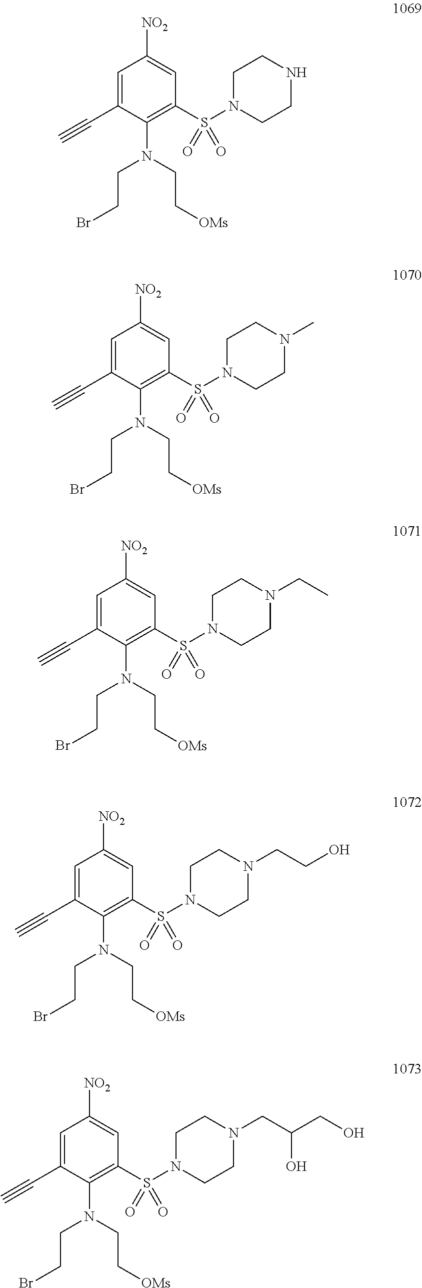

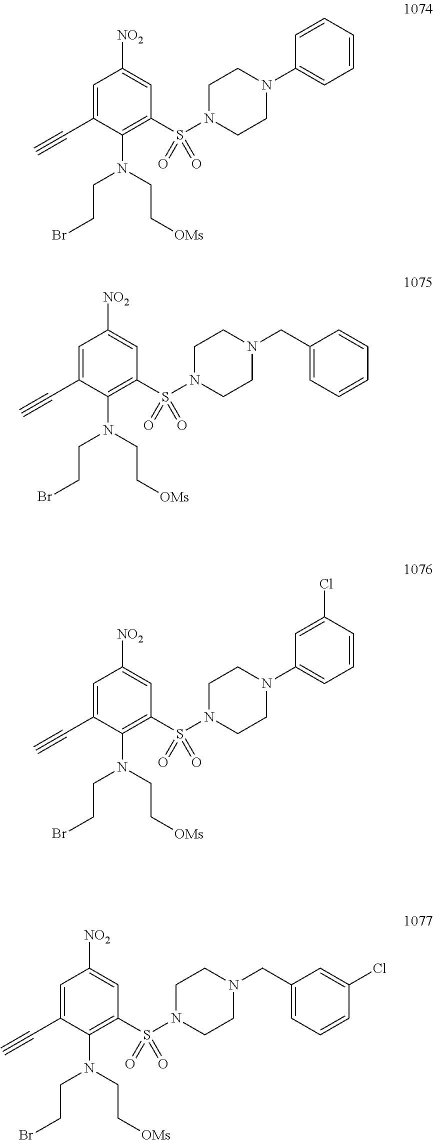 [SMILES: C#Cc1cc([N+](=O)[O-])cc(S(=O)(=O)N2CCN(c3ccccc3)CC2)c1N(CCBr)CCOS(C)(=O)=O ; C#Cc1cc([N+](=O)[O-])cc(S(=O)(=O)N2CCN(Cc3ccccc3)CC2)c1N(CCBr)CCOS(C)(=O)=O ; C#Cc1cc([N+](=O)[O-])cc(S(=O)(=O)N2CCN(c3cccc(Cl)c3)CC2)c1N(CCBr)CCOS(C)(=O)=O ; C#Cc1cc([N+](=O)[O-])cc(S(=O)(=O)N2CCN(Cc3cccc(Cl)c3)CC2)c1N(CCBr)CCOS(C)(=O)=O]

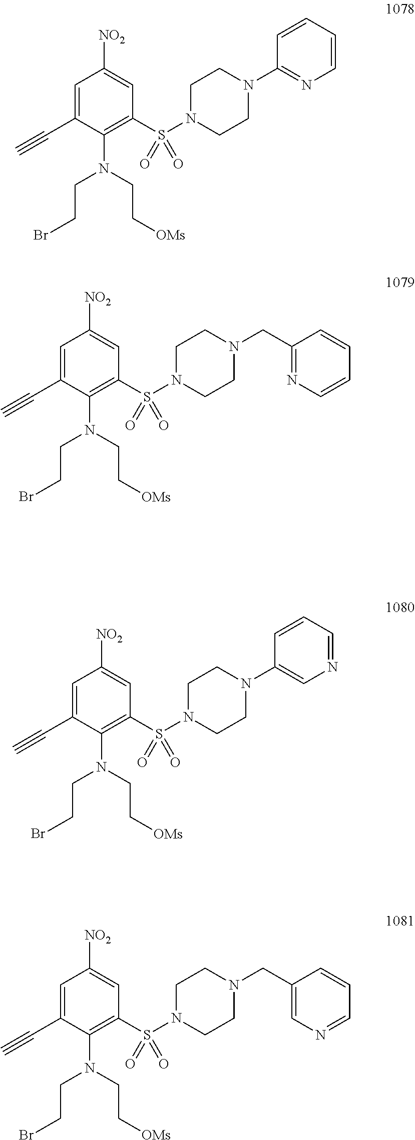 [SMILES: C#Cc1cc([N+](=O)[O-])cc(S(=O)(=O)N2CCN(c3ccccn3)CC2)c1N(CCBr)CCOS(C)(=O)=O ; C#Cc1cc([N+](=O)[O-])cc(S(=O)(=O)N2CCN(Cc3ccccn3)CC2)c1N(CCBr)CCOS(C)(=O)=O ; C#Cc1cc([N+](=O)[O-])cc(S(=O)(=O)N2CCN(c3cccnc3)CC2)c1N(CCBr)CCOS(C)(=O)=O ; C#Cc1cc([N+](=O)[O-])cc(S(=O)(=O)N2CCN(Cc3cccnc3)CC2)c1N(CCBr)CCOS(C)(=O)=O]

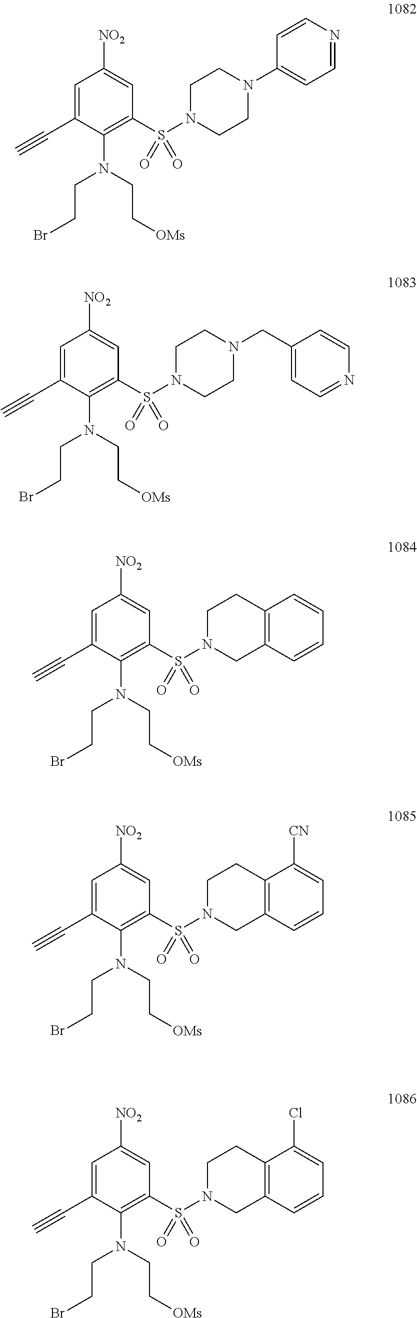 [SMILES: C#Cc1cc([N+](=O)[O-])cc(S(=O)(=O)N2CCN(c3ccncc3)CC2)c1N(CCBr)CCOS(C)(=O)=O ; C#Cc1cc([N+](=O)[O-])cc(S(=O)(=O)N2CCN(Cc3ccncc3)CC2)c1N(CCBr)CCOS(C)(=O)=O ; C#Cc1cc([N+](=O)[O-])cc(S(=O)(=O)N2CCc3ccccc3C2)c1N(CCBr)CCOS(C)(=O)=O ; C#Cc1cc([N+](=O)[O-])cc(S(=O)(=O)N2CCc3c(C#N)cccc3C2)c1N(CCBr)CCOS(C)(=O)=O ; C#Cc1cc([N+](=O)[O-])cc(S(=O)(=O)N2CCc3c(Cl)cccc3C2)c1N(CCBr)CCOS(C)(=O)=O]

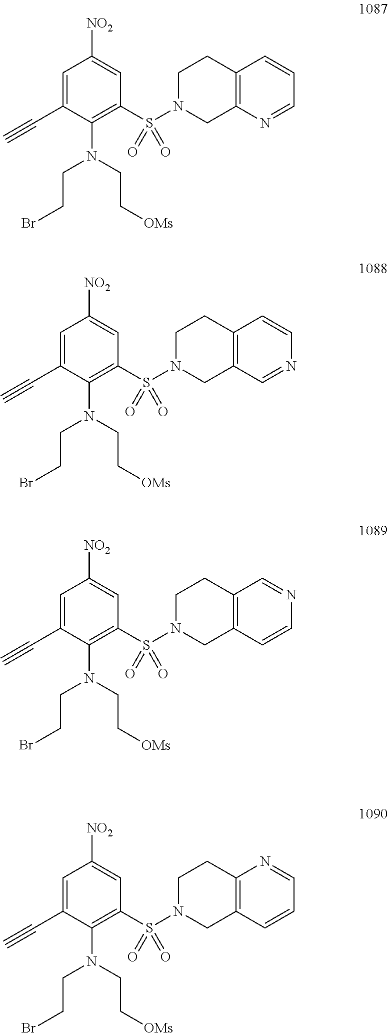 [SMILES: C#Cc1cc([N+](=O)[O-])cc(S(=O)(=O)N2CCc3cccnc3C2)c1N(CCBr)CCOS(C)(=O)=O ; C#Cc1cc([N+](=O)[O-])cc(S(=O)(=O)N2CCc3ccncc3C2)c1N(CCBr)CCOS(C)(=O)=O ; C#Cc1cc([N+](=O)[O-])cc(S(=O)(=O)N2CCc3cnccc3C2)c1N(CCBr)CCOS(C)(=O)=O ; C#Cc1cc([N+](=O)[O-])cc(S(=O)(=O)N2CCc3ncccc3C2)c1N(CCBr)CCOS(C)(=O)=O]

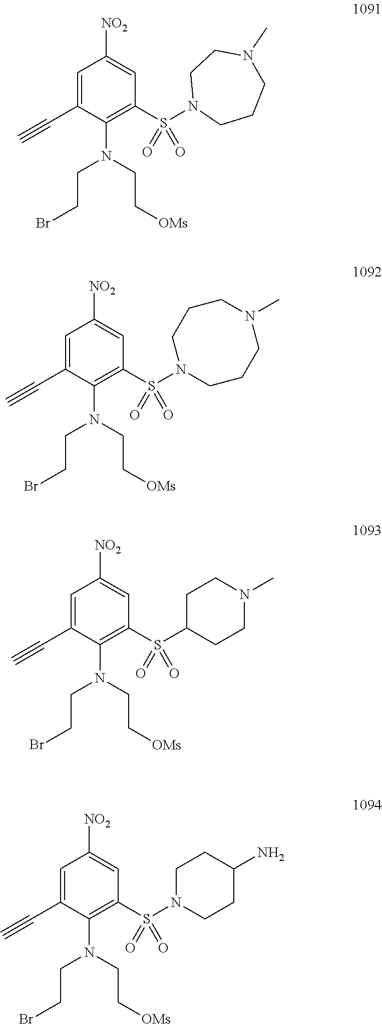

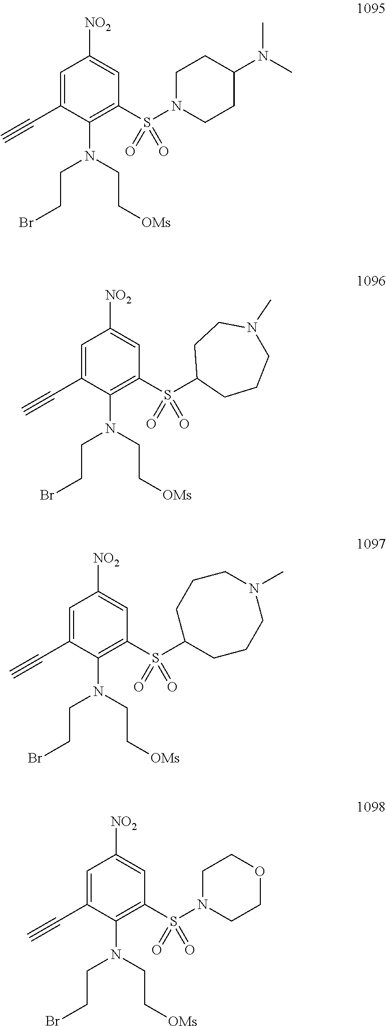

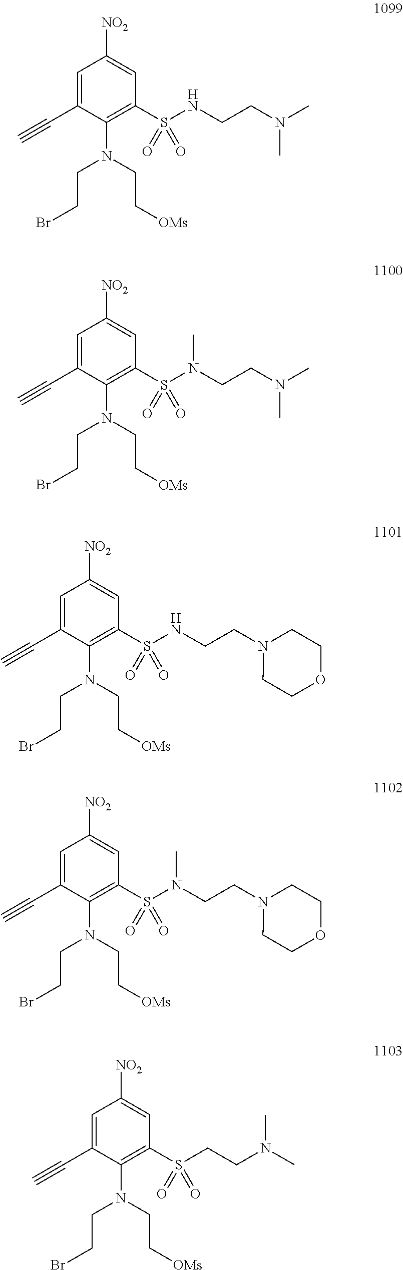

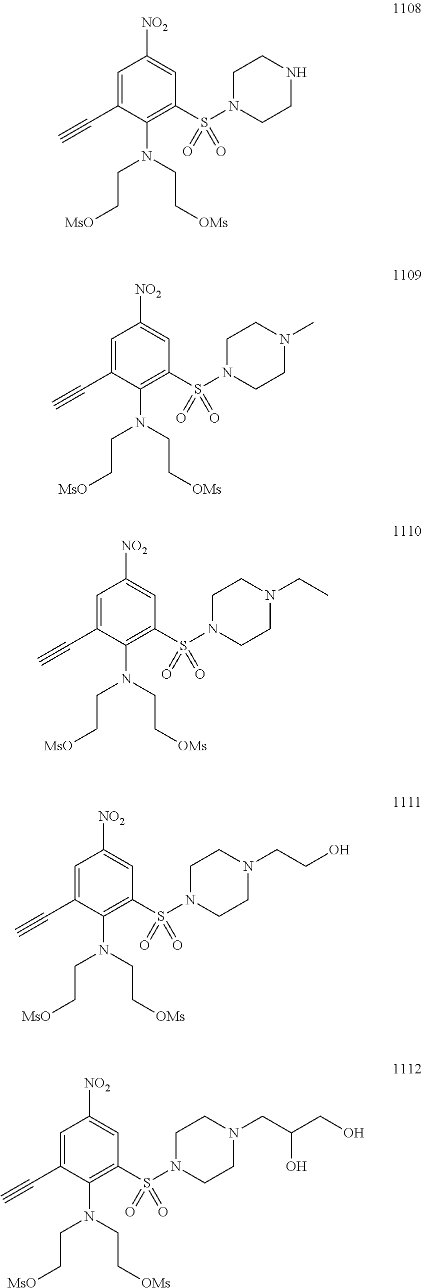

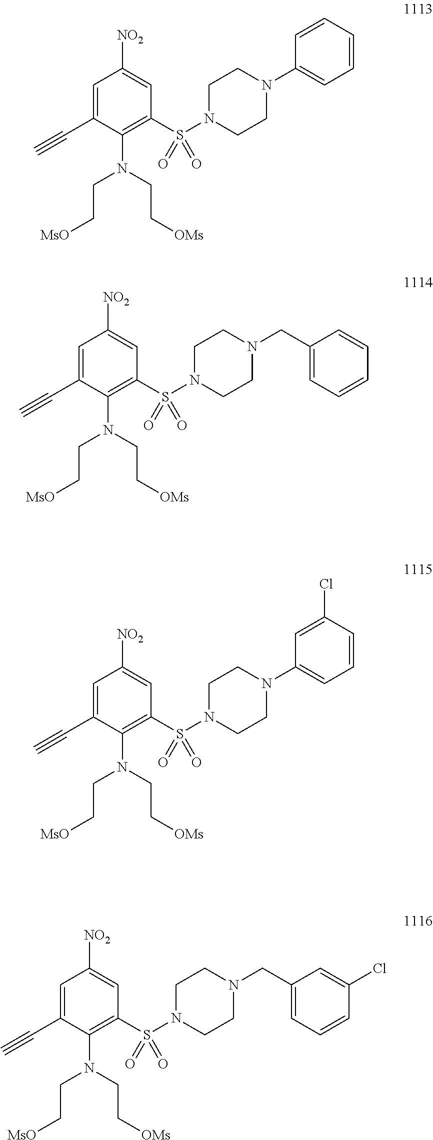 [SMILES: C#Cc1cc([N+](=O)[O-])cc(S(=O)(=O)N2CCN(c3ccccc3)CC2)c1N(CCOS(C)(=O)=O)CCOS(C)(=O)=O ; C#Cc1cc([N+](=O)[O-])cc(S(=O)(=O)N2CCN(Cc3ccccc3)CC2)c1N(CCOS(C)(=O)=O)CCOS(C)(=O)=O ; C#Cc1cc([N+](=O)[O-])cc(S(=O)(=O)N2CCN(c3cccc(Cl)c3)CC2)c1N(CCOS(C)(=O)=O)CCOS(C)(=O)=O ; C#Cc1cc([N+](=O)[O-])cc(S(=O)(=O)N2CCN(Cc3cccc(Cl)c3)CC2)c1N(CCOS(C)(=O)=O)CCOS(C)(=O)=O]

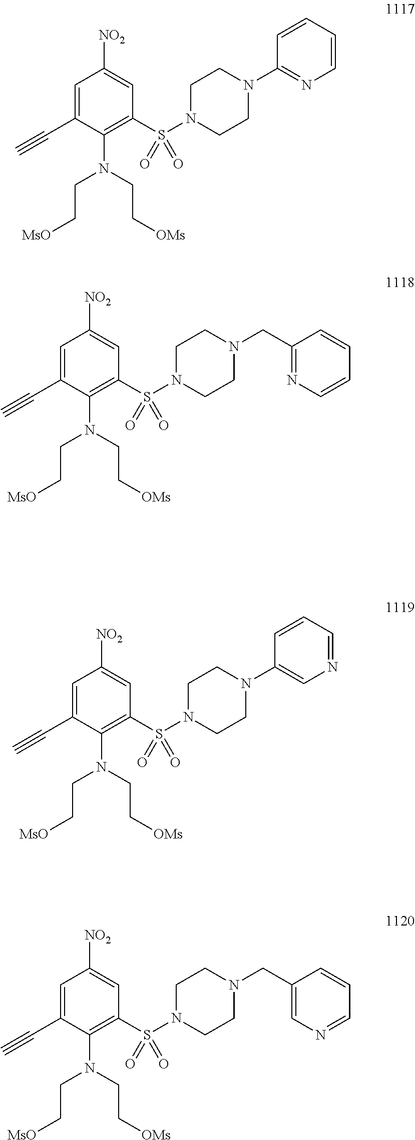 [SMILES: C#Cc1cc([N+](=O)[O-])cc(S(=O)(=O)N2CCN(c3ccccn3)CC2)c1N(CCOS(C)(=O)=O)CCOS(C)(=O)=O ; C#Cc1cc([N+](=O)[O-])cc(S(=O)(=O)N2CCN(Cc3ccccn3)CC2)c1N(CCOS(C)(=O)=O)CCOS(C)(=O)=O ; C#Cc1cc([N+](=O)[O-])cc(S(=O)(=O)N2CCN(c3cccnc3)CC2)c1N(CCOS(C)(=O)=O)CCOS(C)(=O)=O ; C#Cc1cc([N+](=O)[O-])cc(S(=O)(=O)N2CCN(Cc3cccnc3)CC2)c1N(CCOS(C)(=O)=O)CCOS(C)(=O)=O]

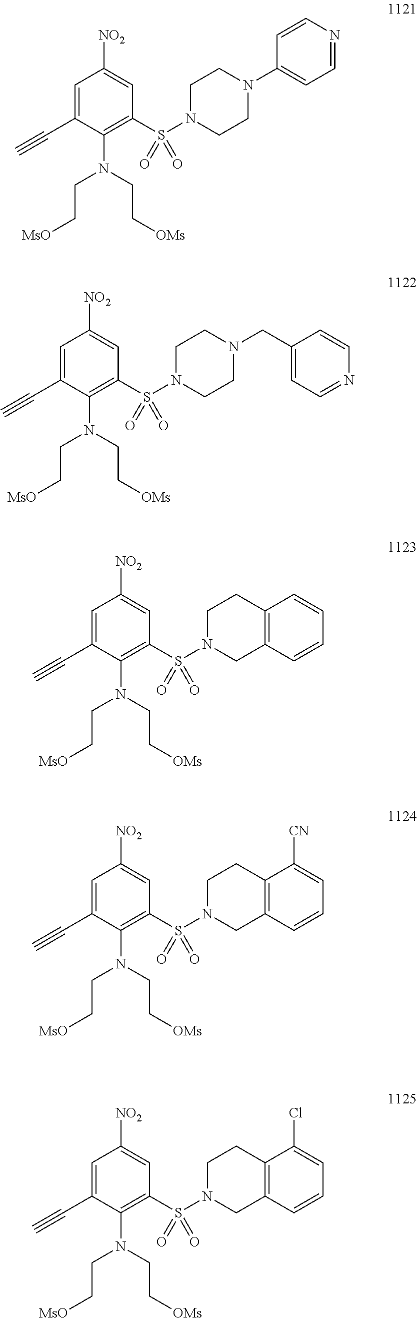 [SMILES: C#Cc1cc([N+](=O)[O-])cc(S(=O)(=O)N2CCN(c3ccncc3)CC2)c1N(CCOS(C)(=O)=O)CCOS(C)(=O)=O ; C#Cc1cc([N+](=O)[O-])cc(S(=O)(=O)N2CCN(Cc3ccncc3)CC2)c1N(CCOS(C)(=O)=O)CCOS(C)(=O)=O ; C#Cc1cc([N+](=O)[O-])cc(S(=O)(=O)N2CCc3ccccc3C2)c1N(CCOS(C)(=O)=O)CCOS(C)(=O)=O ; C#Cc1cc([N+](=O)[O-])cc(S(=O)(=O)N2CCc3c(C#N)cccc3C2)c1N(CCOS(C)(=O)=O)CCOS(C)(=O)=O ; C#Cc1cc([N+](=O)[O-])cc(S(=O)(=O)N2CCc3c(Cl)cccc3C2)c1N(CCOS(C)(=O)=O)CCOS(C)(=O)=O]

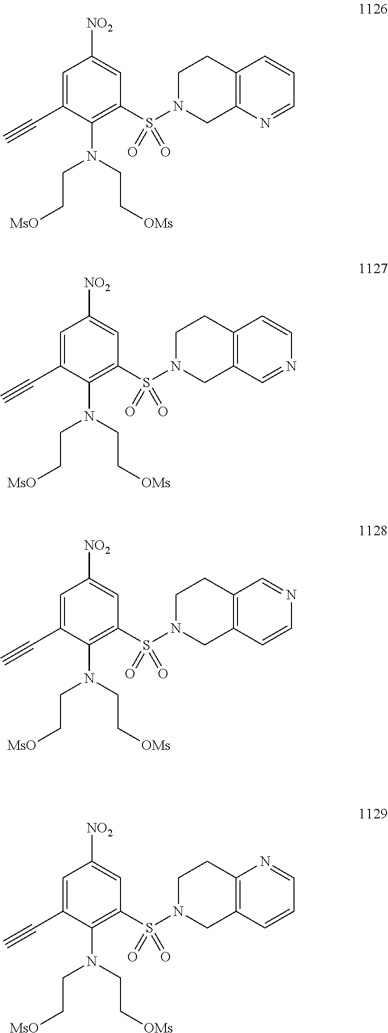 [SMILES: C#Cc1cc([N+](=O)[O-])cc(S(=O)(=O)N2CCc3cccnc3C2)c1N(CCOS(C)(=O)=O)CCOS(C)(=O)=O ; C#Cc1cc([N+](=O)[O-])cc(S(=O)(=O)N2CCc3ccncc3C2)c1N(CCOS(C)(=O)=O)CCOS(C)(=O)=O ; C#Cc1cc([N+](=O)[O-])cc(S(=O)(=O)N2CCc3cnccc3C2)c1N(CCOS(C)(=O)=O)CCOS(C)(=O)=O ; C#Cc1cc([N+](=O)[O-])cc(S(=O)(=O)N2CCc3ncccc3C2)c1N(CCOS(C)(=O)=O)CCOS(C)(=O)=O]

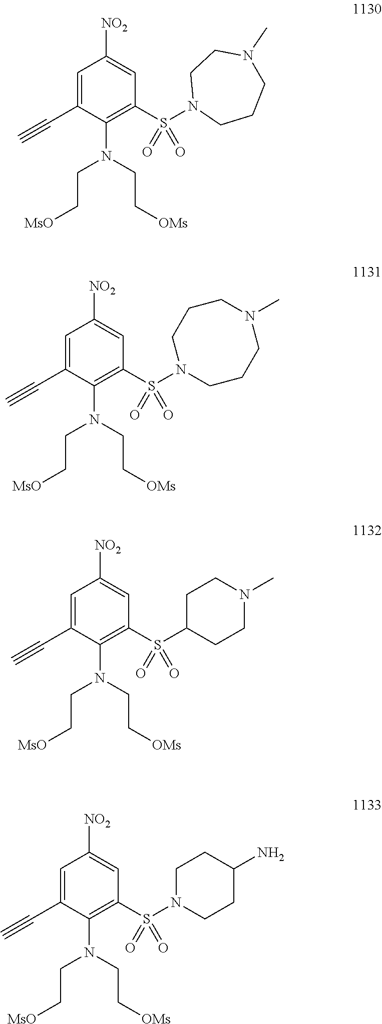

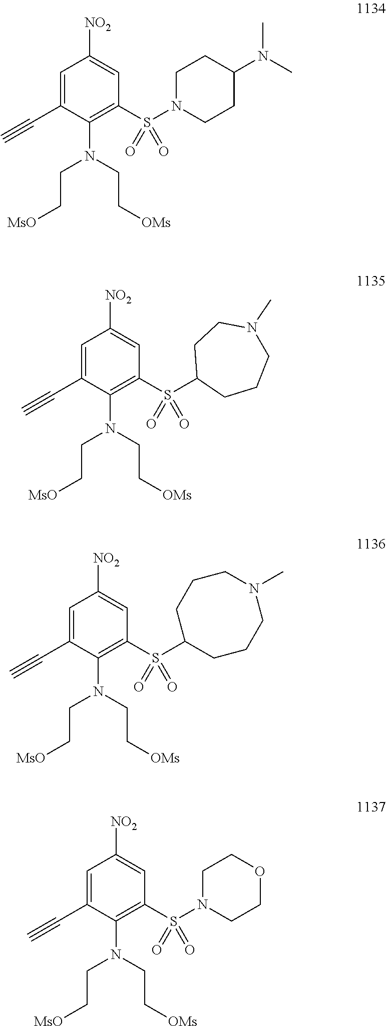

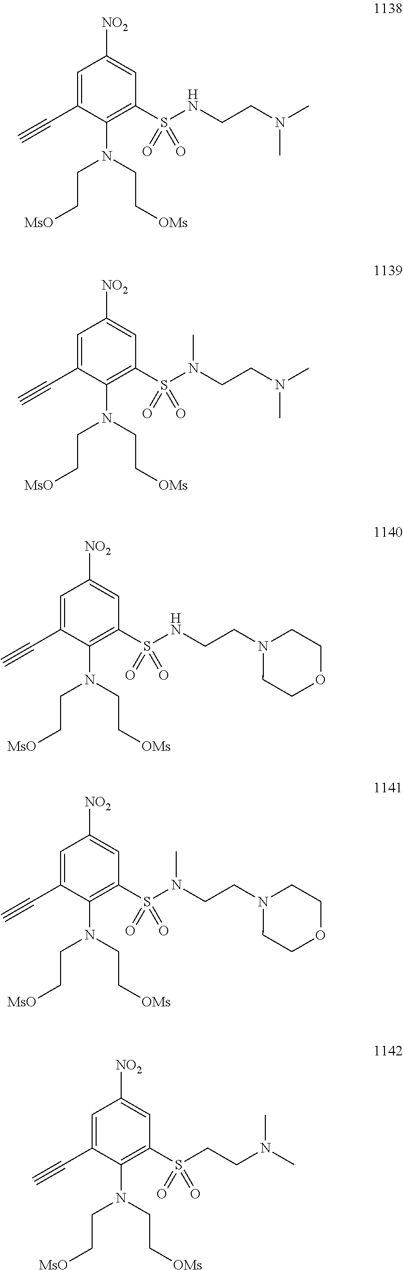

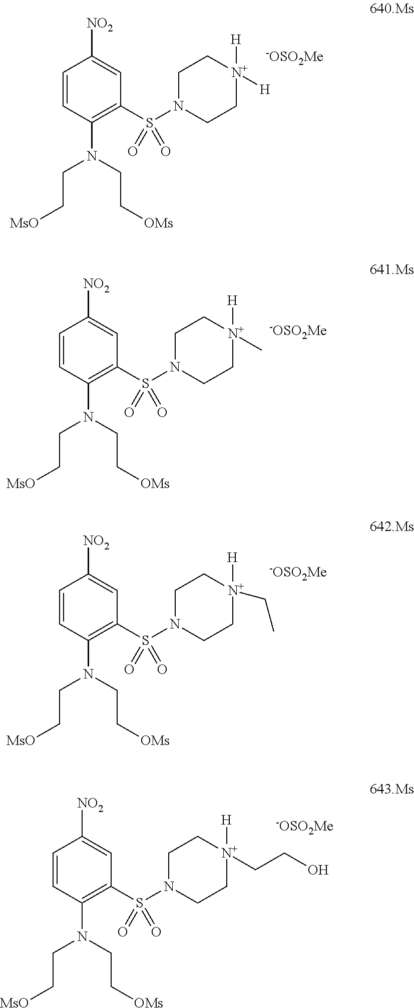

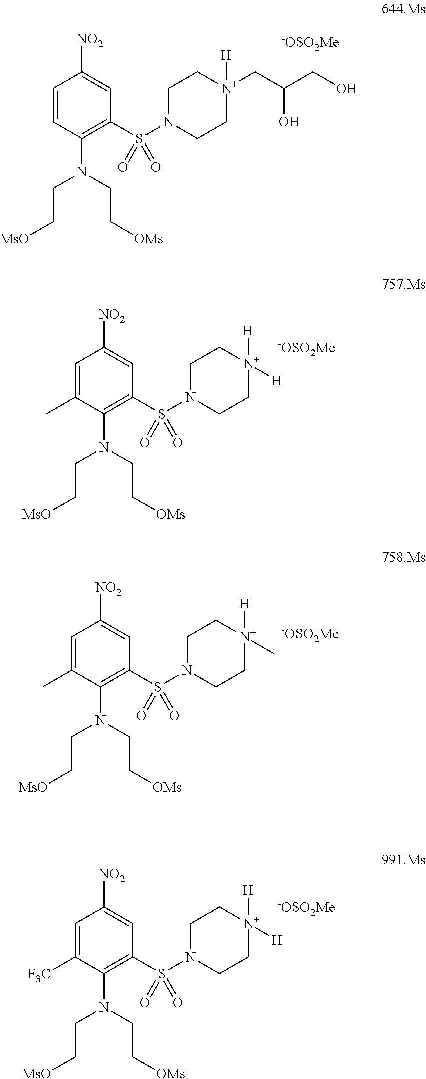

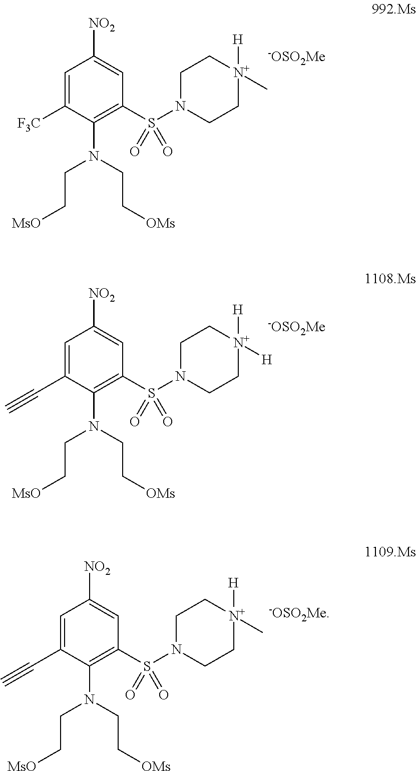

Specific definitions and meanings of the groups, and the preparation methods and spectral data of the compounds have been described in Patent Application PCT/NZ2019/050030 with Publication No. WO2019190331A1 (corresponding to Chinese Patent Application No. 2019800234236 with Publication No. CN111918864A), which is incorporated herein by reference in its entirety.

The compounds of structural Formula (3), similar to AST-3424 or AST, are prodrugs of nitrogen mustard analogues

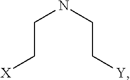

and can be activated by the AKR1C3 enzyme to form nitrogen mustard analogues

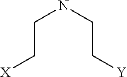

(a DNA alkylating agent) to exhibit its anti-cancer efficacy:

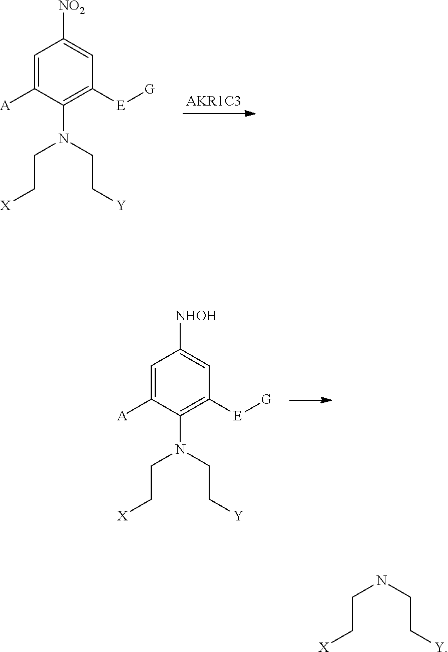

A treatment method which uses a drug monotherapy containing an AKR1C3-activated DNA alkylating agent prodrug compound or in combination with other therapeutic drugs for treating cancer and tumor patients having KRAS mutations, wherein the compound is selected from structural Formula (4) and salts, esters, solvates, and isotopic isomers thereof:

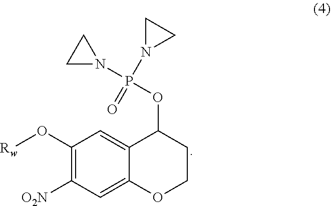

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations, wherein the compound is selected from the structural Formula (4) and salts, esters, solvates, and isotopic isomers thereof:

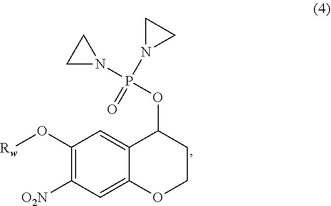

wherein the definition of Rw has been described in the claims of Patent Application PCT/CN2020/120281 with Publication No. WO2021068952A1. Specifically, the groups are defined as follows:
Rw is

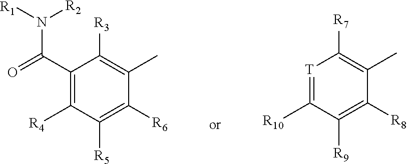

R₁ is H, C₁₋₆ alkyl, C₃₋₆ cycloalkyl, 4-6 membered heterocycloalkyl, 5-6 membered heteroaryl or phenyl, wherein the C₁₋₆ alkyl, C₃₋₆ cycloalkyl, 4-6 membered heterocycloalkyl, 5-6 membered heteroaryl and phenyl are optionally substituted with 1, 2 or 3 R^{a};
each R^{a} is independently H, F, Cl, Br, I, —CN, —OH, C₁₋₃ alkoxy or C₁₋₃ alkyl;
R₂ is H or C₁₋₆ alkyl;
or R₁ and R₂, together with the N atom to which they are attached, to form a 4-6 membered heterocycloalkyl, wherein the 4-6 membered heterocycloalkyl is optionally substituted with 1, 2 or 3 Re;
each Re is independently H, F, Cl, Br, I, —CN, —OH, —NH₂, —OCH₃, —OCH₂CH₃, —CH₃ or —CH₂CH₃;
R₃ is H, F, Cl, Br, I, —OH, —NH₂, C₁₋₃ alkoxy or C₁₋₃ alkyl;
or R₂ and R₃ are attached together to make the structural unit

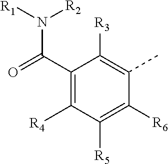

to be

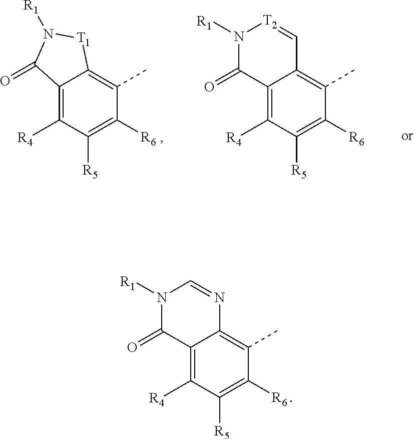

T₁ is —(CR^{c}R^{d})ₘ— or —(CR^{c}R^{d})ₙ—O—;
m is 1, 2 or 3;
n is 1 or 2;
T₂ is Nor CH;
R^{c} and R^{d} are each independently H, F, C₁₋₃ alkyl or C₁₋₃ alkoxy;
R₄, R₅ and R₆ are each independently H, F, Cl, Br, I, C₁₋₃ alkyl or C₁₋₃ alkoxy;
T is N or CH;
R₇ and R₈ are each independently H, F, Cl, Br or I;
R₉ and R₁₀ are each independently H, F, Cl, Br, I, —CN or
the 4-6 membered heterocycloalkyl and 5-6 membered heteroaryl each contain 1, 2, 3 or 4 heteroatoms independently selected from N, —O— and —S—.

Specifically, the compound of Formula (4) is selected from the group consisting of:

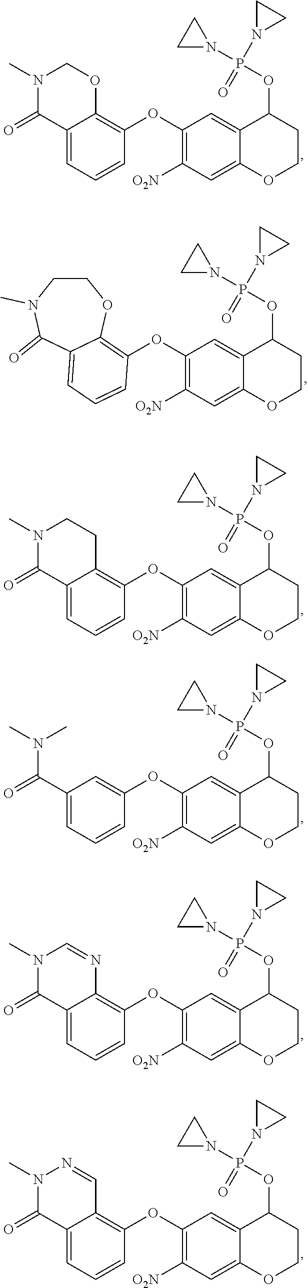

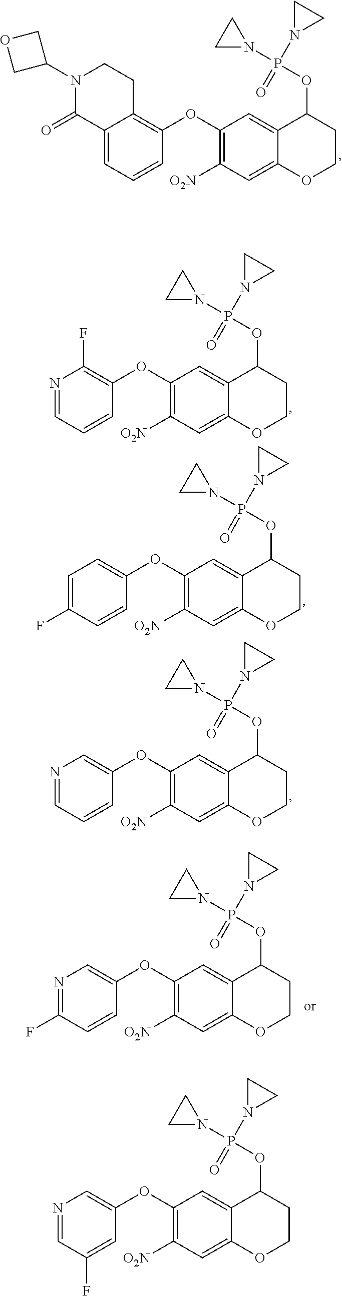

The definitions and meanings of the groups, and the preparation methods and spectral data of the compounds have been described in Patent Application PCT/CN2020/120281 with Publication No. WO2021068952A1, which is incorporated herein by reference in its entirety.

Evidently, the compounds of structural Formula (3), similar to AST-3424 or AST, are prodrugs of AST-2660, and can be activated by the AKR1C3 enzyme to form AST-2660 (a DNA alkylating agent) to exhibit its anti-cancer efficacy:

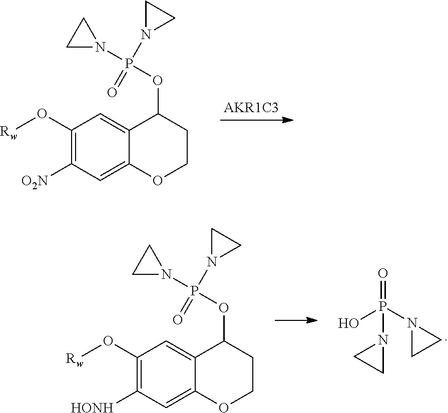

A treatment method which uses a drug monotherapy containing an AKR1C3-activated DNA alkylating agent prodrug compound or in combination with other therapeutic drugs for treating cancer and tumor patients having KRAS mutations, wherein the compound is selected from structural Formula (5) and salts, esters, solvates, and isotopic isomers thereof:

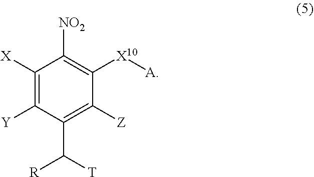

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations, wherein the compound is selected from the structural Formula (5) and salts, esters, solvates, and isotopic isomers thereof:

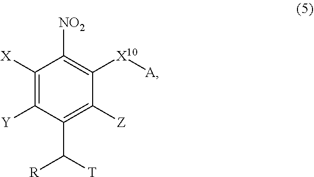

wherein the definitions of X, Y, Z, R, T, A and X¹⁰ have been described in the claims of Patent Application PCT/US2016/021581 with Publication No. WO2016145092A1 (corresponding to Chinese Patent Application No. 2016800150788 with Publication No. CN107530556A).

Specifically, the groups are defined as follows:
X¹⁰ is O, S, SO or SO₂;
A is C₆-C₁₀ aryl, 5-15 membered heteroaryl, or —N═CR¹R²;
each R¹ and R² independently is hydrogen, C₁-C₆ alkyl, C₃-C₅ cycloalkyl, C₆-C₁₀ aryl, 4-15 membered heterocycle, ether, —CONR¹³R¹⁴, or —NR¹³COR¹⁴;
each X, Y, and Z independently is hydrogen, CN, halogeno, C₁-C₆ alkyl, C₂-C₆ alkenyl, C₂-C₆ alkynyl, C₃-C₈ cycloalkyl, C₆-C₁₀ aryl, 4-15 membered heterocycle, ether, —CONR¹³R¹⁴, or —NR¹³COR¹⁴;
R is hydrogen, C₁-C₆ alkyl, C₂-C₆ alkenyl, C₂-C₆ alkynyl, C₃-C₈ cycloalkyl, C₆-C₁₀ aryl, 4-15 membered heterocycle, ether, —CONR¹³R¹⁴, or —NR¹³COR¹⁴;
each R¹³ and R¹⁴ independently is hydrogen, C₁-C₆ alkyl, C₃-C₈ cycloalkyl, C₆-C₁₀ aryl, 4-15 membered heterocycle, or ether;
T comprises a phosphoramidate alkylating agent comprising one or more Z⁵—X⁵—Y⁵ moieties bonded to an —O—P(Z¹) moiety, where Z⁵ is a heteroatom such as nitrogen, sulfur or oxygen, X⁵ is substituted or unsubstituted ethylene, Y⁵ is halogeno or another leaving group, or Z⁵—X⁵—Y⁵ together form an aziridinyl (NCH₂CH₂) moiety, and Z¹ is 0 or S; and
wherein the alkyl, alkenyl, alkynyl, cycloalkyl, aryl, heterocycle, heteroaryl, and ether groups are substituted or unsubstituted.

Specifically, the compound of Formula (5) is selected from the group consisting of:

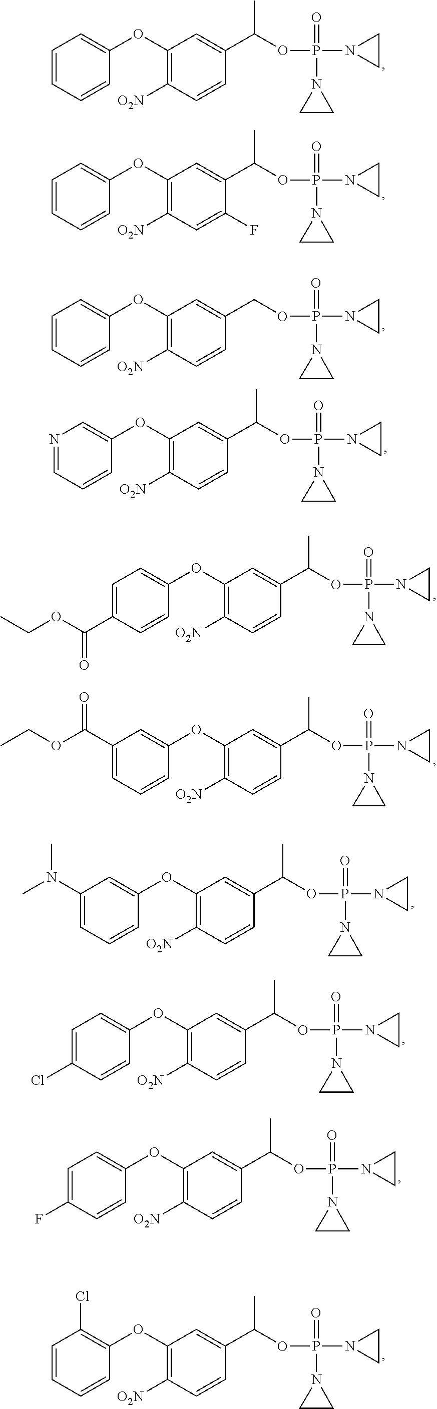

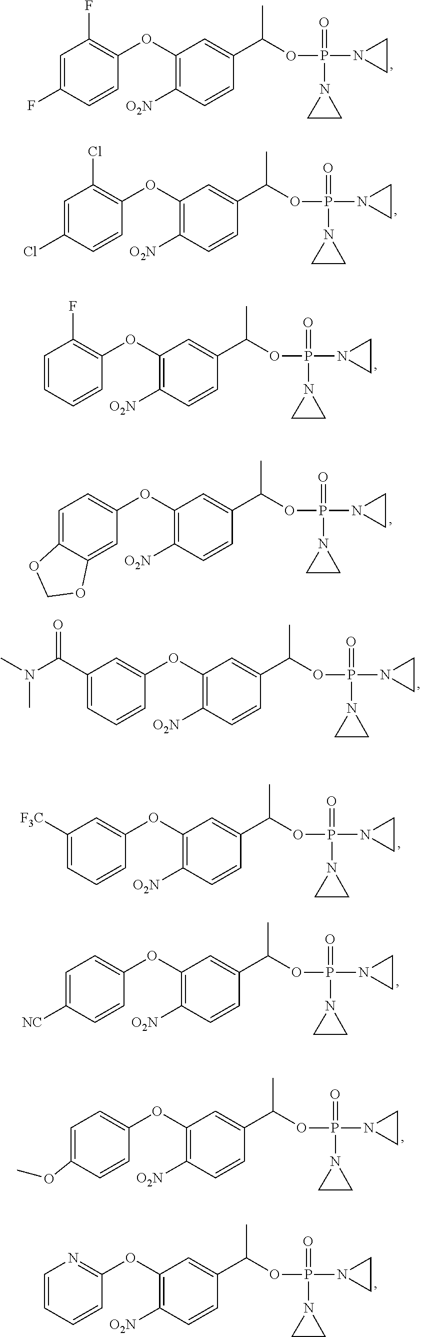

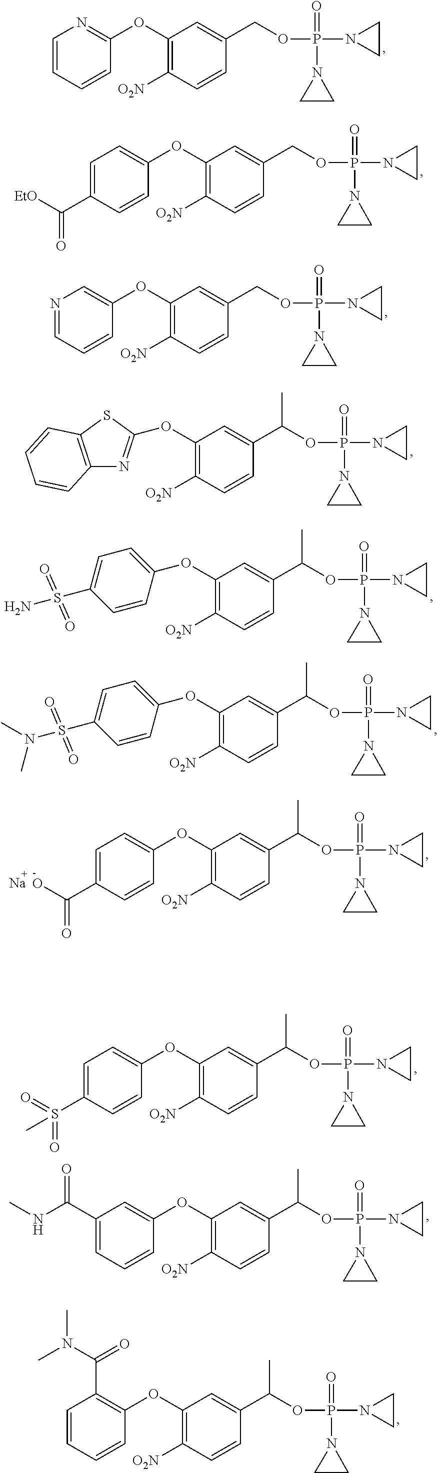

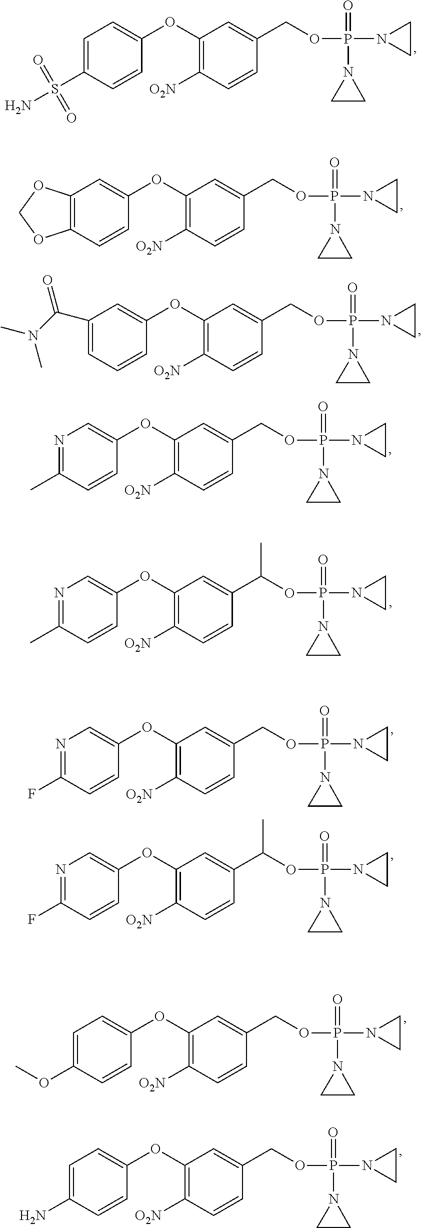

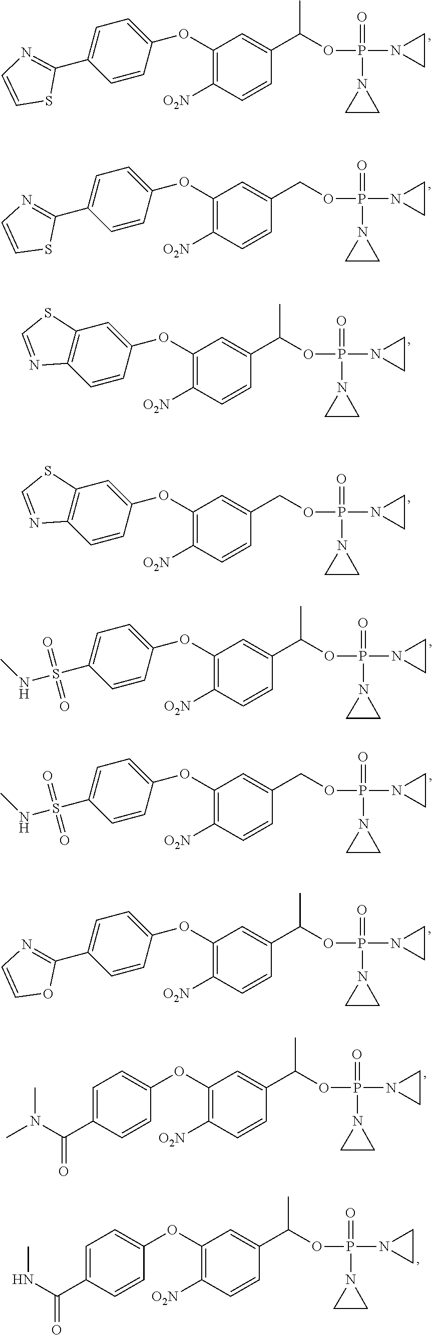

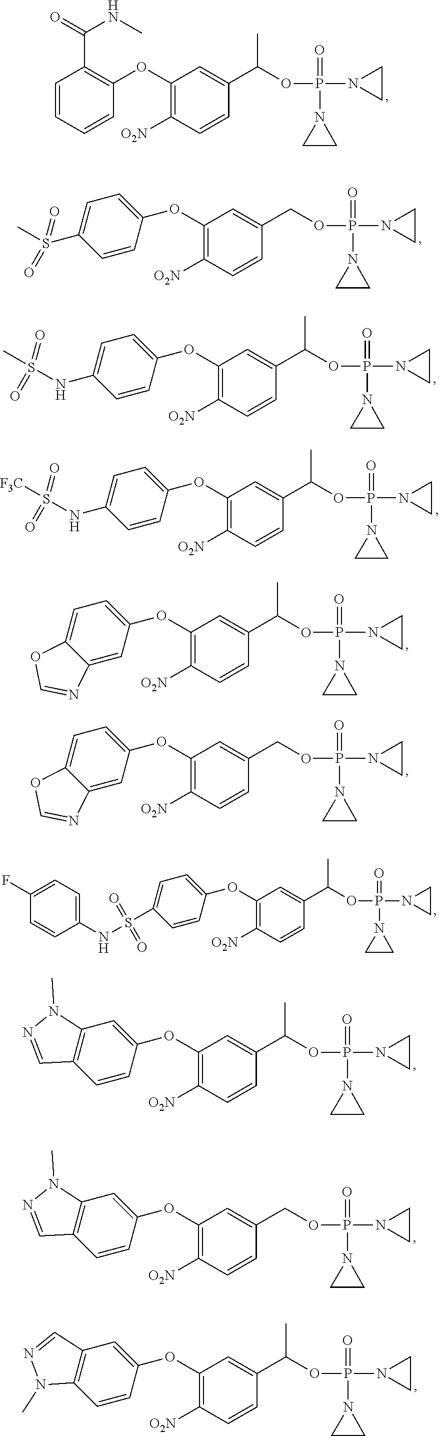

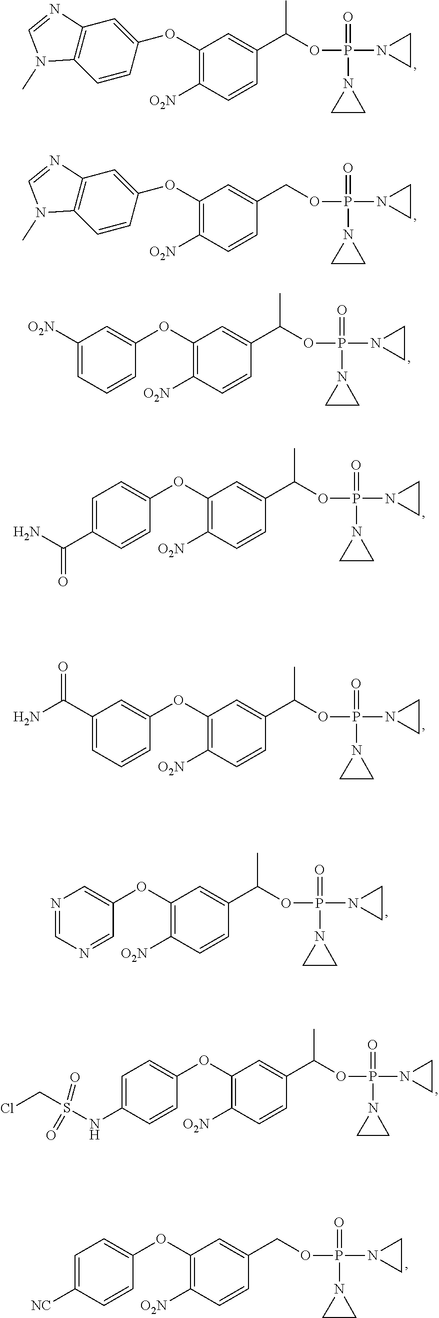

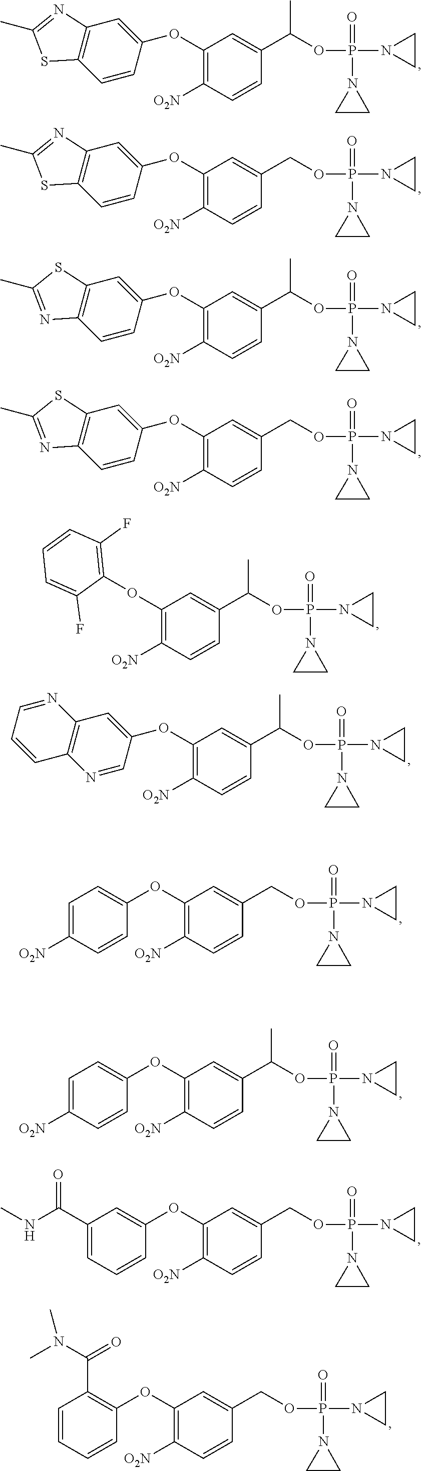

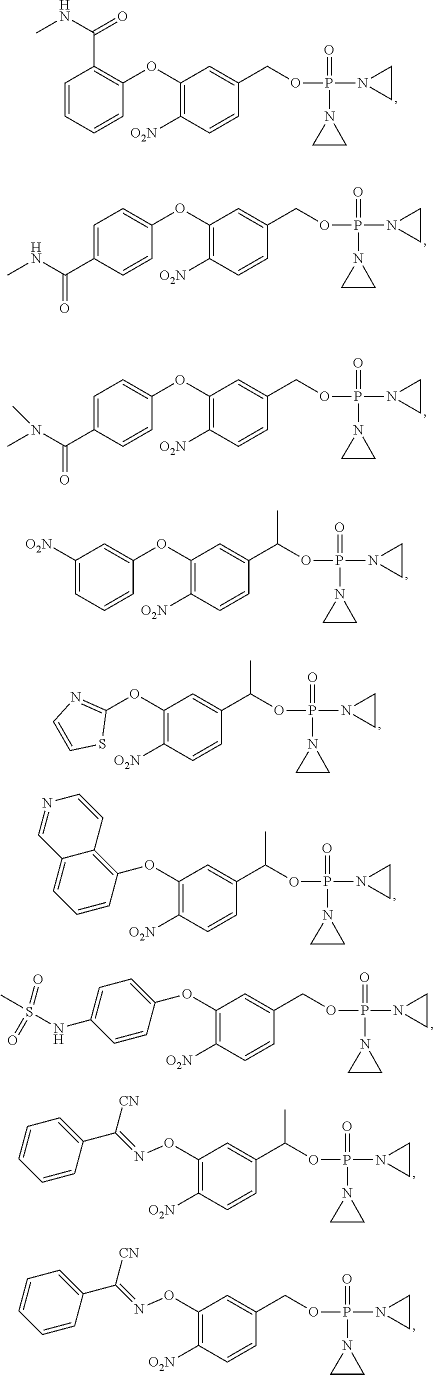

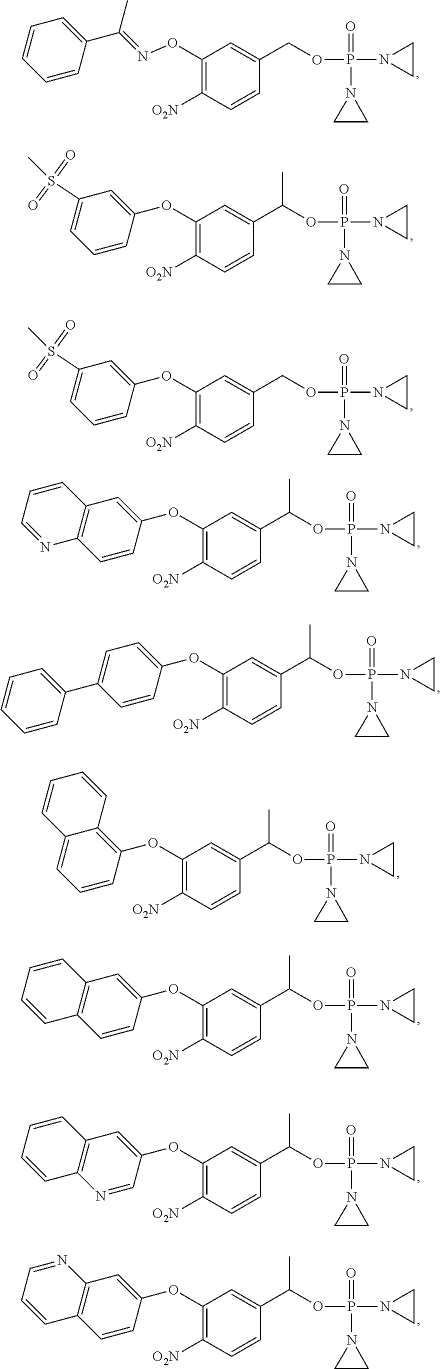

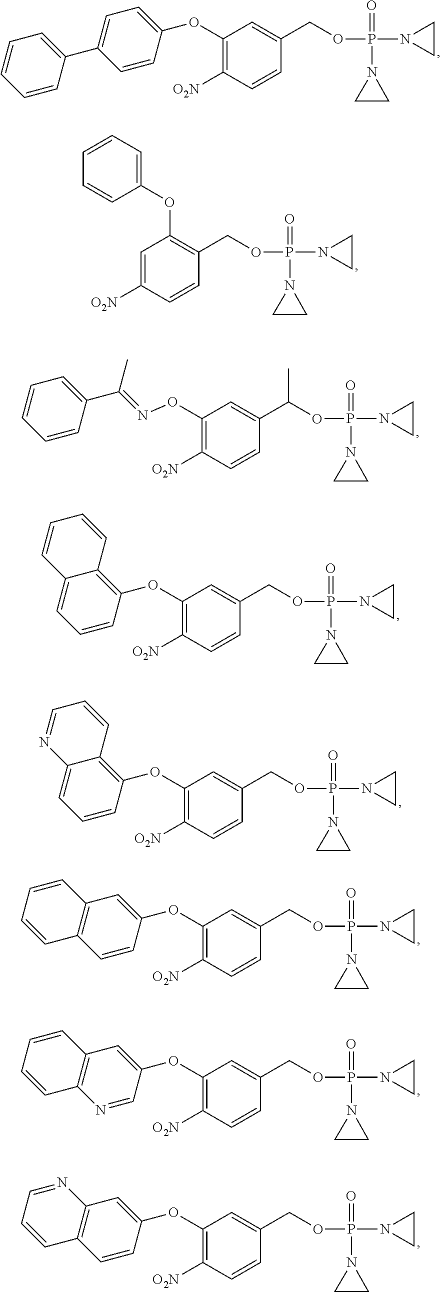

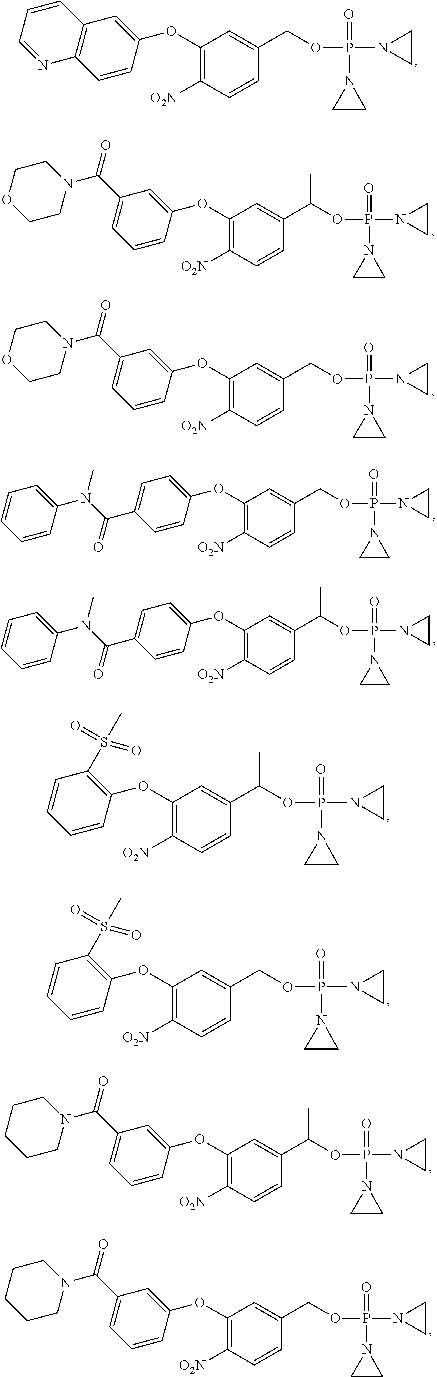

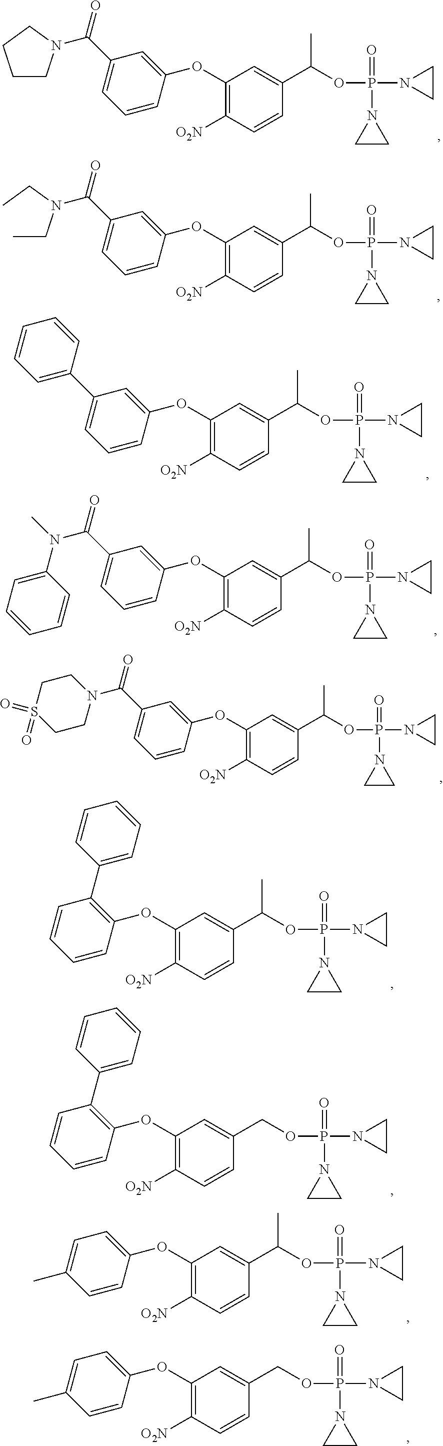

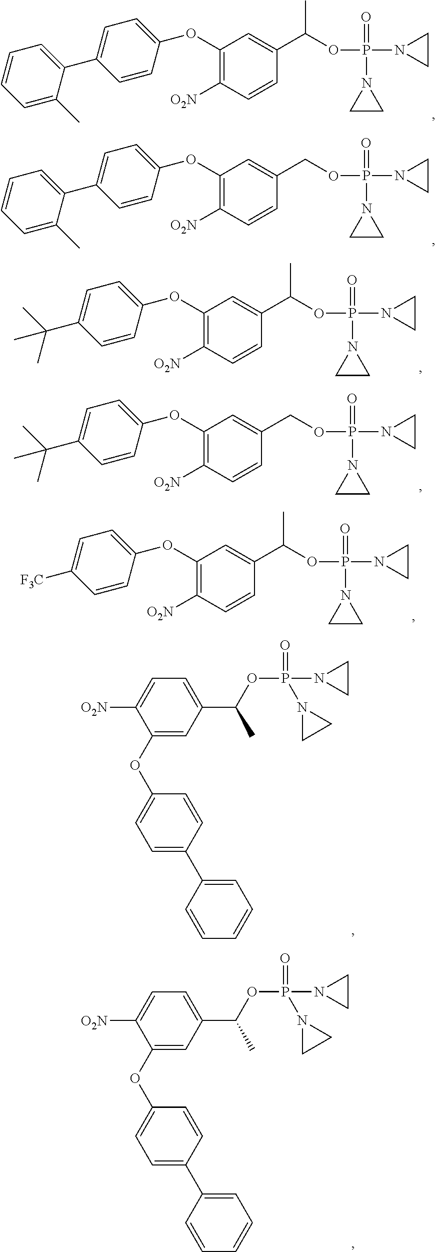

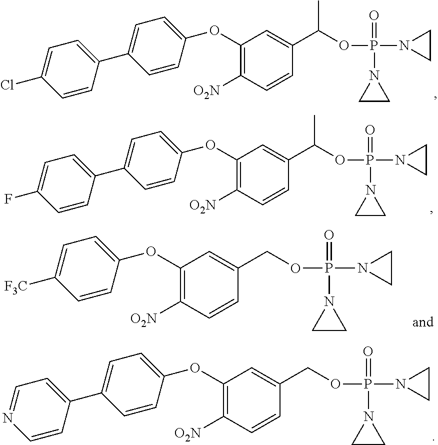

Specific definitions and meanings of the groups, and the preparation methods and spectral data of the compounds have been described in Patent Application PCT/US2016/021581 with Publication No. WO2016145092A1 (corresponding to Chinese Patent Application No. 2016800150788 with Publication No. CN107530556A), which is incorporated herein by reference in its entirety.

Evidently, the compounds of structural Formula (5), similar to AST-3424 or AST, are prodrugs of a phosphoramidate alkylating agent, and can be activated by the AKR1C3 enzyme to form T (a phosphoramidate alkylating agent, AST-2660 is a phosphoramidate alkylating agent) to exert its anti-cancer efficacy:

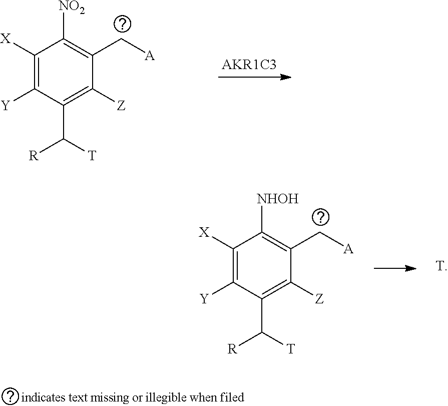

A treatment method which uses a drug monotherapy containing an AKR1C3-activated DNA alkylating agent prodrug compound or in combination with other therapeutic drugs for treating cancer and tumor patients having KRAS mutations, wherein the compound is selected from structural Formula (6) and salts, esters, solvates, and isotopic isomers thereof:

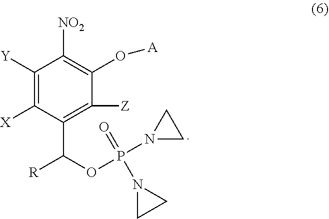

Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations, wherein the compound is selected from the structural Formula (6) and salts, esters, solvates, and isotopic isomers thereof:

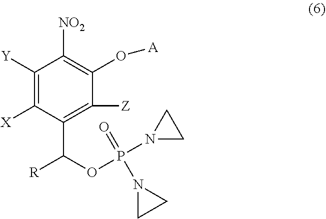

wherein,
A is substituted or unsubstituted C₆-C₁₀ aryl, biaryl or substituted biaryl, 5-15 membered heteroaryl, or —N═CR¹R²; wherein the substituents are selected from the group consisting of halogeno, —CN, —NO₂, —O—(CH₂)—O—, —CO₂H and salt thereof, —OR¹⁰⁰, —CO₂R¹⁰⁰, —CONR¹⁰¹R¹⁰², —NR¹⁰¹R₁₀², —NR¹⁰⁰SO₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₈ alkyl, or C₆-C₁₂ aryl; or R¹⁰¹ and R¹⁰² together with the nitrogen atom to which they are attached to form a 5-7 membered heterocycle;
wherein the alkyl group and the aryl group are each substituted by 1-3 halogeno groups or 1-3 C₁-C₆ alkyl groups;
R¹ and R² are each independently phenyl or methyl;
X, Y and Z are each independently hydrogen or halogeno; and
R is hydrogen or C₁-C₆ alkyl or halogen-substituted alkyl.

Evidently, the “compound” also includes the compound itself as well as solvate, salt, ester or isotopic isomer thereof.

“Cx-Cy” or “Cx-y” before a group refers to a range of the number of carbon atoms that are present in that group. For example, C₁-C₆ alkyl refers to an alkyl group having at least 1 and up to 6 carbon atoms.

“Alkyl” refers to monovalent saturated aliphatic hydrocarbyl groups having from 1 to 10 carbon atoms and, in some embodiments, from 1 to 6 carbon atoms. “Cx-y alkyl” refers to alkyl groups having from x to y carbon atoms. This term includes (by way of example) linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Aryl” refers to an aromatic group having from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8 tetrahydronaphthalene-2-yl is an aryl group as its point of attachment is at the 2-position of the aromatic phenyl ring). “Arylene” refers to a divalent aryl radical having the appropriate hydrogen content.

“Cycloalkyl” refers to a saturated or partially saturated cyclic group having from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cycloalkylene” refers to a divalent cycloalkyl radical having the appropriate hydrogen content.

“Halogeno” refers to one or more of fluoro, chloro, bromo, and iodo.

“Heteroaryl” refers to an aromatic group having from 1 to 14 carbon atoms and 1 to 6 heteroatoms selected from the group consisting of oxygen, nitrogen, and sulfur and includes single ring (e.g. imidazolyl-2-yl and imidazole-5-yl) and multiple ring systems (e.g. imidazopyridyl, benzotriazolyl, benzimidazol-2-yl and benzimidazol-6-yl). For multiple ring systems, including fused, bridged, and spiro ring systems having aromatic and non-aromatic rings, the term “heteroaryl” applies if there is at least one ring heteroatom, and the point of attachment is at an atom of an aromatic ring (e.g., 1,2,3,4-tetrahydroquinolin-6-yl and 5,6,7,8-tetrahydroquinolin-3-yl). In some embodiments, the nitrogen and/or the sulfur ring atom(s) of the heteroaryl group are optionally oxidized to provide N-oxide (N→O), sulfinyl, or sulfonyl moieties. The term heteroaryl includes, but is not limited to, acridinyl, azocinyl, benzimidazolyl, benzofuranyl, benzothiofuranyl, benzothiophenyl, benzoxazolyl, benzothiazolyl, benzotriazolyl, benzotetrazolyl, benzisoxazolyl, benzisothiazolyl, benzothienyl, benzimidazolinyl, carbazolyl, NH-carbazolyl, carbolinyl, chromanyl, chromenyl, cinnolinyl, dithiazinyl, furanyl, furazanyl, imidazolidinyl, imidazolinyl, imidazopyridyl, imidazolyl, indazolyl, indolenyl, indolinyl, indolizinyl, indolyl, isobenzofuranyl, isochromanyl, isoindazolyl, isoindolinyl, isoindolyl, isoquinolinyl, isoquinolyl, isothiazolyl, isoxazolyl, naphthyridinyl, octahydroisoquinolinyl, oxadiazolyl, oxazolidinyl, oxazolyl, pyrimidinyl, phenanthridinyl, phenanthrolinyl, phenazinyl, phenothiazinyl, phenoxathiinyl, phenoxazinyl, phthalazinyl, piperazinyl, pteridinyl, purinyl, pyranyl, pyrazinyl, pyrazolidinyl, pyrazolinyl, pyrazolyl, pyridazinyl, pyridooxazolyl, pyridoimidazolyl, pyridothiazole, pyridinyl, pyridyl, pyrimidinyl, pyrrolyl, quinazolinyl, quinolinyl, quinoxalinyl, quinuclidinyl, tetrahydroisoquinolinyl, tetrahydroquinolinyl, tetrazolyl, thiadiazinyl, thiadiazolyl, thianthrenyl, thiazolyl, thienyl, thienothiazolyl, thienooxazolyl, thienoimidazolyl, thiophenyl, triazinyl and xanthenyl. “Heteroarylene” refers to a divalent heteroaryl radical having the appropriate hydrogen content.

“Heterocyclic” or “heterocycle” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the terms “heterocycle”, “heterocycle”, “heterocycloalkyl” or “heterocyclyl” apply when there is at least one ring heteroatom, and the point of attachment is at an atom of a non-aromatic ring (e.g. 1,2,3,4-tetrahydroquinoline-3-yl, 5,6,7,8-tetrahydroquinoline-6-yl, and decahydroquinolin-6-yl). In some embodiment, the heterocyclic groups herein are 3-15 membered, 4-14 membered, 5-13 membered, 7-12, or 5-7 membered heterocycles. In some other embodiment, the heterocycles contain 4 heteroatoms. In some other embodiment, the heterocycles contain 3 heteroatoms. In another embodiment, the heterocycles contain up to 2 heteroatoms. In some embodiments, the nitrogen and/or sulfur atom(s) of the heterocyclic group are optionally oxidized to provide the N-oxide, sulfmyl, sulfonyl moieties. Heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃₋₁₀) refers to the total number of carbon atoms in the portion of the heterocyclyl group exclusive of the number of heteroatoms. A divalent heterocyclic radical will have the appropriately adjusted hydrogen content.

“Biaryl” refers to a structure in which two aromatic rings are linked by a C—C single bond, such as biphenyl, bipyridine, and the like.

The term “optionally substituted” refers to a substituted or unsubstituted group. The group may be substituted with one or more substituents, such as e.g., 1, 2, 3, 4 or 5 substituents. Preferably, the substituents are selected from the group consisting of oxo, halogeno, —CN, NO₂, —N₂+, —CO₂R¹⁰⁰, —OR¹⁰⁰, —SR¹⁰⁰, —SOR¹⁰⁰, —SO₂R¹⁰⁰, —NR¹⁰⁰SO₂R¹⁰⁰, —NR¹⁰¹R¹⁰², —CONR¹⁰¹R¹⁰², —SO₂NR¹⁰¹R¹⁰², C₁-C₆ alkyl, C₁-C₆ alkoxy, —CR¹⁰⁰=C(R¹⁰⁰)₂, —CCR¹⁰⁰, C₃-C₁₀ cycloalkyl, C₃-C₁₀ heterocyclyl, C₆-C₁₂ aryl and C₂-C₁₂ heteroaryl, or a divalent substituent such as —O—(CH₂)—O—, —O—(CH₂)₂—O—, and, 1-4 methyl substituted version thereof, wherein each R¹⁰⁰, R¹⁰¹, and R¹⁰² independently is hydrogen or C₁-C₅ alkyl; C₃-C₁₂ cycloalkyl; C₃-C₁₀ heterocyclyl; C₆-C₁₂ aryl; or C₂-C₁₂ heteroaryl; or R¹⁰⁰ and R¹⁰² together with the nitrogen atom to which they are attached to form a 5-7 membered heterocycle; wherein each alkyl, cycloalkyl, heterocyclyl, aryl, or heteroaryl is optionally substituted with 1-3 halogeno, 1-3 C₁-C₆ alkyl, 1-3 C₁-C₆ haloalkyl or 1-3 C₁-C₆ alkoxy groups. Preferably, the substituents are selected from the group consisting of chloro, fluoro, —OCH₃, methyl, ethyl, iso-propyl, cyclopropyl, —CO₂H and salts and C₁-C₆ alkyl esters thereof, CONMe₂, CONHMe, CONH₂, —SO₂Me, —SO₂NH₂, —SO₂NMe₂, —SO₂NHMe, —NHSO₂Me, —NHSO₂CF₃, —NHSO₂CH₂C₁, —NH₂, —OCF₃, —CF₃ and —OCHF₂.

Specifically, the compound of Formula (6) is selected from the group consisting of:

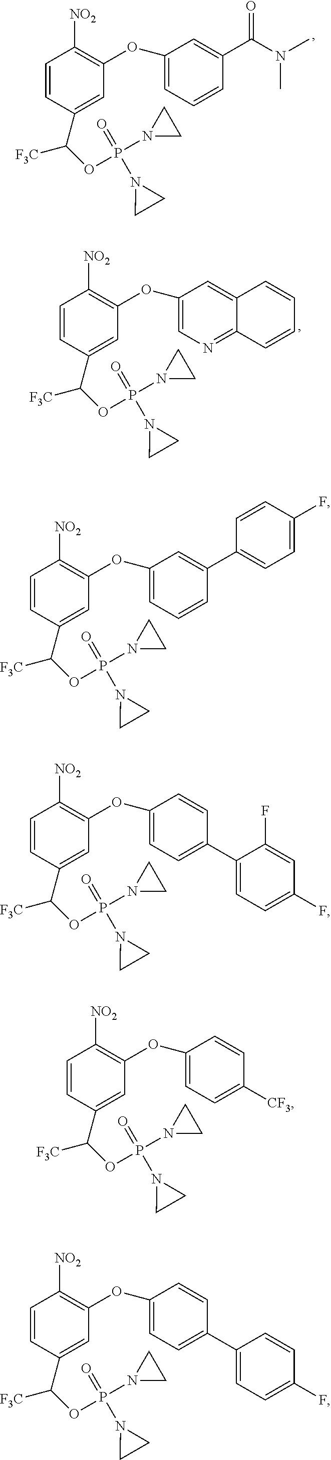 [SMILES: CN(C)C(=O)c1cccc(Oc2cc(C(OP(=O)(N3CC3)N3CC3)C(F)(F)F)ccc2[N+](=O)[O-])c1 ; O=[N+]([O-])c1ccc(C(OP(=O)(N2CC2)N2CC2)C(F)(F)F)cc1Oc1cnc2ccccc2c1 ; O=[N+]([O-])c1ccc(C(OP(=O)(N2CC2)N2CC2)C(F)(F)F)cc1Oc1cccc(-c2ccc(F)cc2)c1 ; O=[N+]([O-])c1ccc(C(OP(=O)(N2CC2)N2CC2)C(F)(F)F)cc1Oc1ccc(-c2ccc(F)cc2F)cc1 ; O=[N+]([O-])c1ccc(C(OP(=O)(N2CC2)N2CC2)C(F)(F)F)cc1Oc1ccc(C(F)(F)F)cc1 ; O=[N+]([O-])c1ccc(C(OP(=O)(N2CC2)N2CC2)C(F)(F)F)cc1Oc1ccc(-c2ccc(F)cc2)cc1]

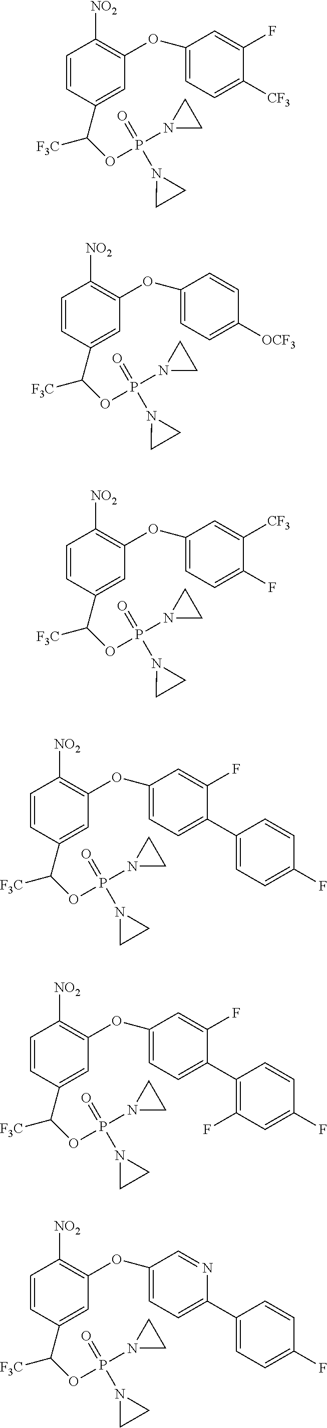 [SMILES: O=[N+]([O-])c1ccc(C(OP(=O)(N2CC2)N2CC2)C(F)(F)F)cc1Oc1ccc(C(F)(F)F)c(F)c1 ; O=[N+]([O-])c1ccc(C(OP(=O)(N2CC2)N2CC2)C(F)(F)F)cc1Oc1ccc(OC(F)(F)F)cc1 ; O=[N+]([O-])c1ccc(C(OP(=O)(N2CC2)N2CC2)C(F)(F)F)cc1Oc1ccc(F)c(C(F)(F)F)c1 ; O=[N+]([O-])c1ccc(C(OP(=O)(N2CC2)N2CC2)C(F)(F)F)cc1Oc1ccc(-c2ccc(F)cc2)c(F)c1 ; O=[N+]([O-])c1ccc(C(OP(=O)(N2CC2)N2CC2)C(F)(F)F)cc1Oc1ccc(-c2ccc(F)cc2F)c(F)c1 ; O=[N+]([O-])c1ccc(C(OP(=O)(N2CC2)N2CC2)C(F)(F)F)cc1Oc1ccc(-c2ccc(F)cc2)nc1]

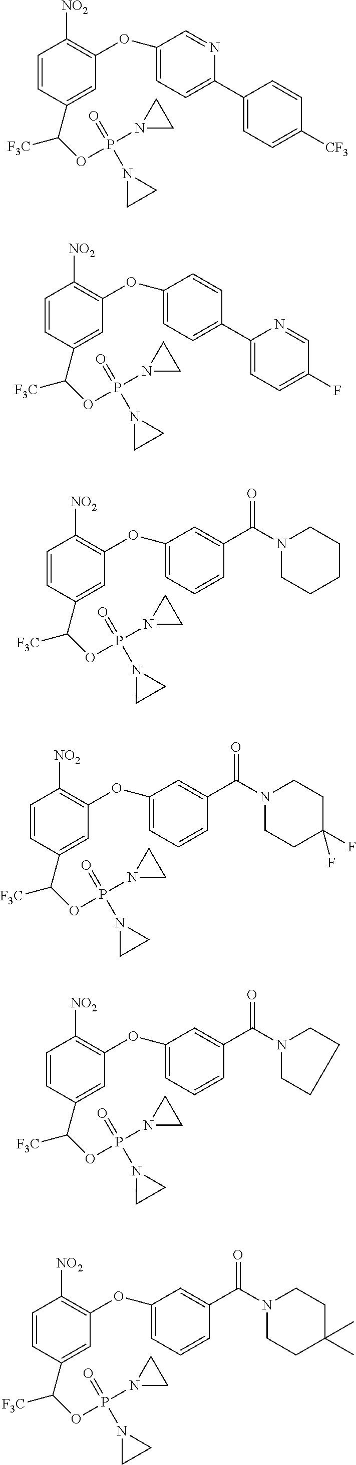 [SMILES: O=[N+]([O-])c1ccc(C(OP(=O)(N2CC2)N2CC2)C(F)(F)F)cc1Oc1ccc(-c2ccc(C(F)(F)F)cc2)nc1 ; O=[N+]([O-])c1ccc(C(OP(=O)(N2CC2)N2CC2)C(F)(F)F)cc1Oc1ccc(-c2ccc(F)cn2)cc1 ; O=C(c1cccc(Oc2cc(C(OP(=O)(N3CC3)N3CC3)C(F)(F)F)ccc2[N+](=O)[O-])c1)N1CCCCC1 ; O=C(c1cccc(Oc2cc(C(OP(=O)(N3CC3)N3CC3)C(F)(F)F)ccc2[N+](=O)[O-])c1)N1CCC(F)(F)CC1 ; O=C(c1cccc(Oc2cc(C(OP(=O)(N3CC3)N3CC3)C(F)(F)F)ccc2[N+](=O)[O-])c1)N1CCCC1 ; CC1(C)CCN(C(=O)c2cccc(Oc3cc(C(OP(=O)(N4CC4)N4CC4)C(F)(F)F)ccc3[N+](=O)[O-])c2)CC1]

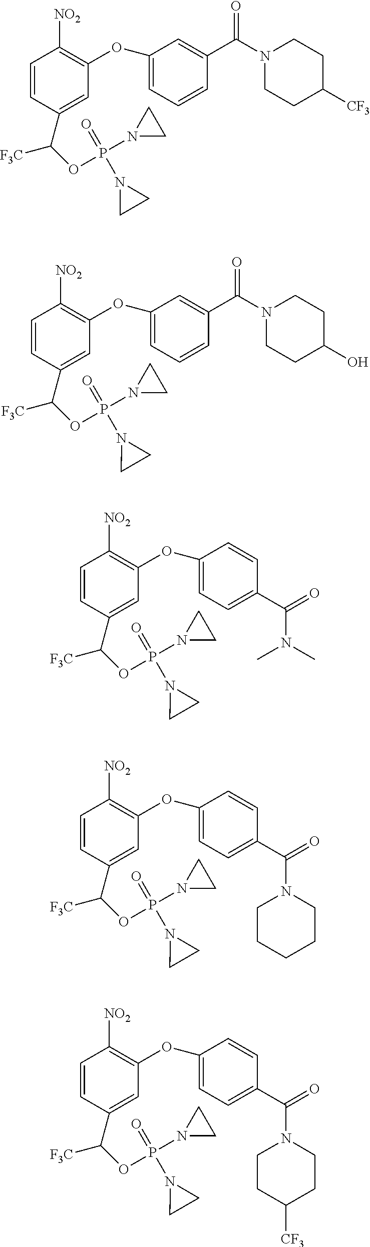 [SMILES: O=C(c1cccc(Oc2cc(C(OP(=O)(N3CC3)N3CC3)C(F)(F)F)ccc2[N+](=O)[O-])c1)N1CCC(C(F)(F)F)CC1 ; O=C(c1cccc(Oc2cc(C(OP(=O)(N3CC3)N3CC3)C(F)(F)F)ccc2[N+](=O)[O-])c1)N1CCC(O)CC1 ; CN(C)C(=O)c1ccc(Oc2cc(C(OP(=O)(N3CC3)N3CC3)C(F)(F)F)ccc2[N+](=O)[O-])cc1 ; O=C(c1ccc(Oc2cc(C(OP(=O)(N3CC3)N3CC3)C(F)(F)F)ccc2[N+](=O)[O-])cc1)N1CCCCC1 ; O=C(c1ccc(Oc2cc(C(OP(=O)(N3CC3)N3CC3)C(F)(F)F)ccc2[N+](=O)[O-])cc1)N1CCC(C(F)(F)F)CC1]

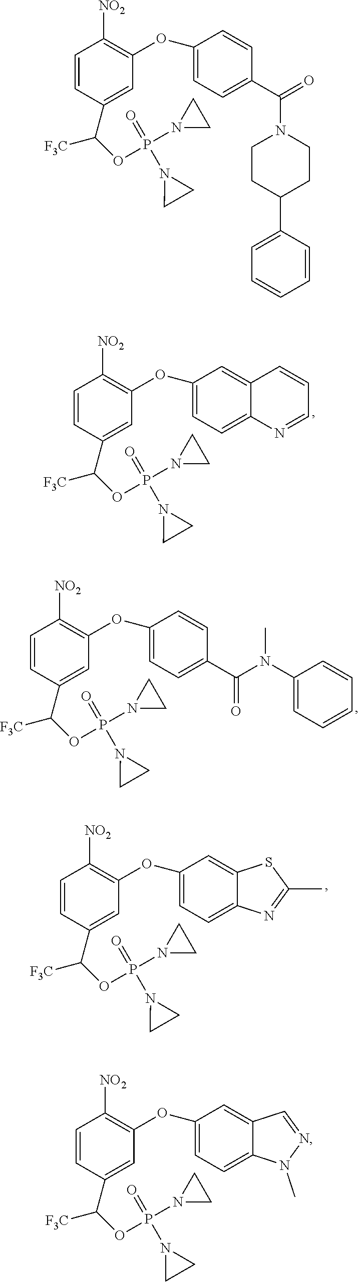 [SMILES: O=C(c1ccc(Oc2cc(C(OP(=O)(N3CC3)N3CC3)C(F)(F)F)ccc2[N+](=O)[O-])cc1)N1CCC(c2ccccc2)CC1 ; O=[N+]([O-])c1ccc(C(OP(=O)(N2CC2)N2CC2)C(F)(F)F)cc1Oc1ccc2ncccc2c1 ; CN(C(=O)c1ccc(Oc2cc(C(OP(=O)(N3CC3)N3CC3)C(F)(F)F)ccc2[N+](=O)[O-])cc1)c1ccccc1 ; Cc1nc2ccc(Oc3cc(C(OP(=O)(N4CC4)N4CC4)C(F)(F)F)ccc3[N+](=O)[O-])cc2s1 ; Cn1ncc2cc(Oc3cc(C(OP(=O)(N4CC4)N4CC4)C(F)(F)F)ccc3[N+](=O)[O-])ccc21]

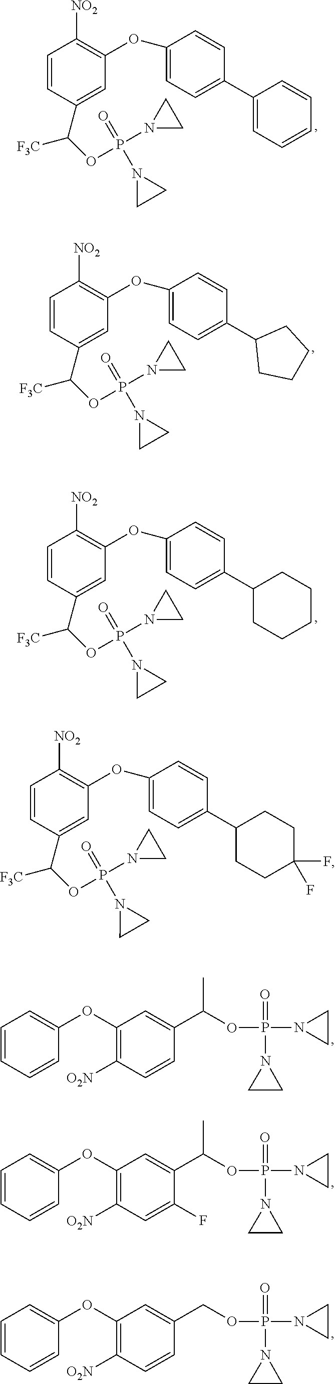

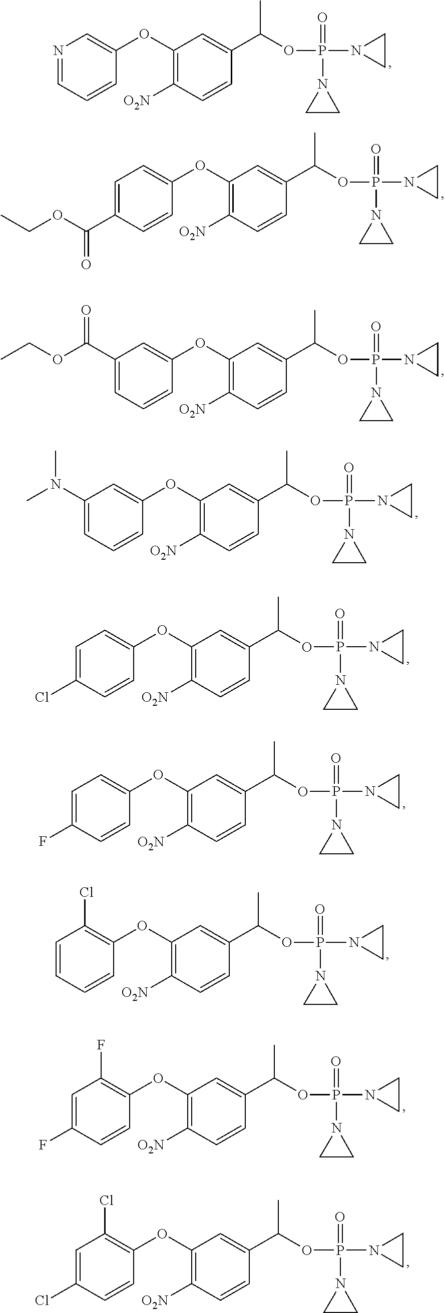

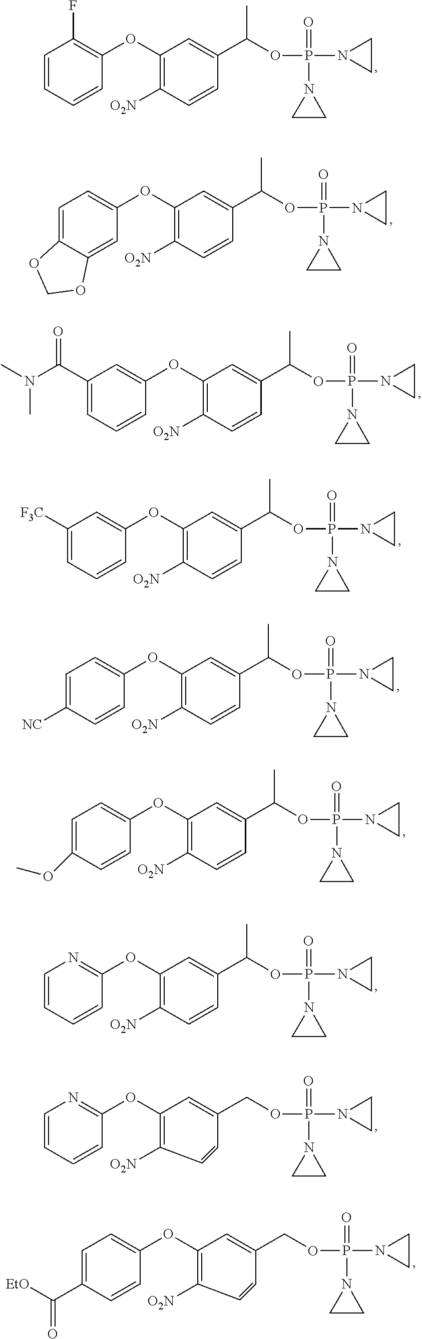

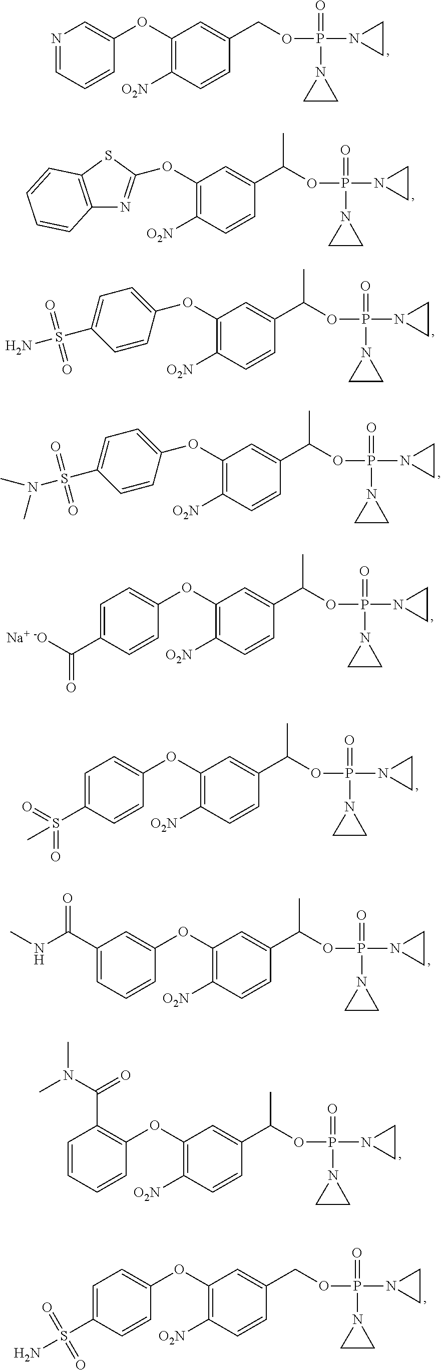

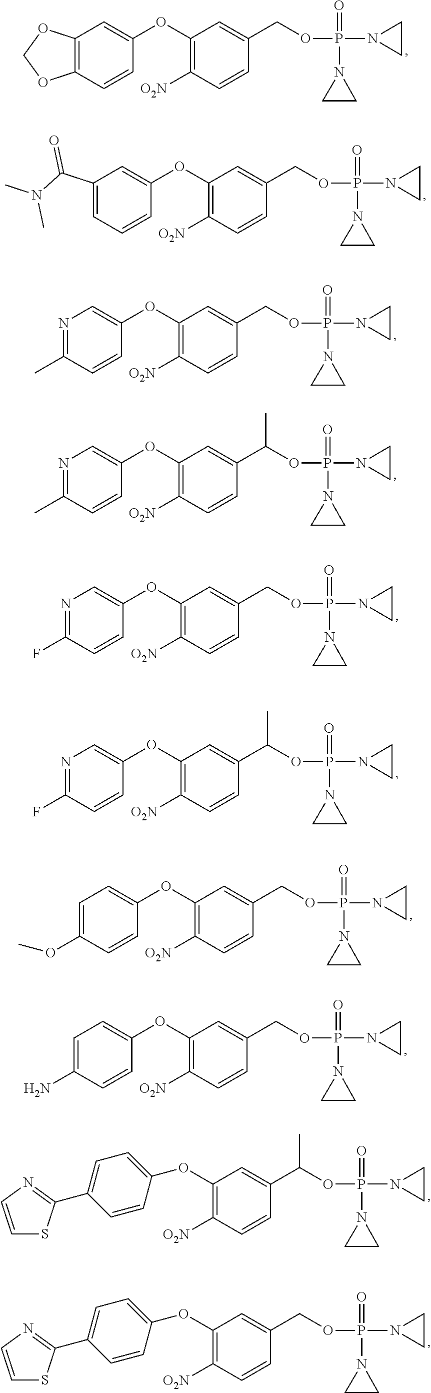

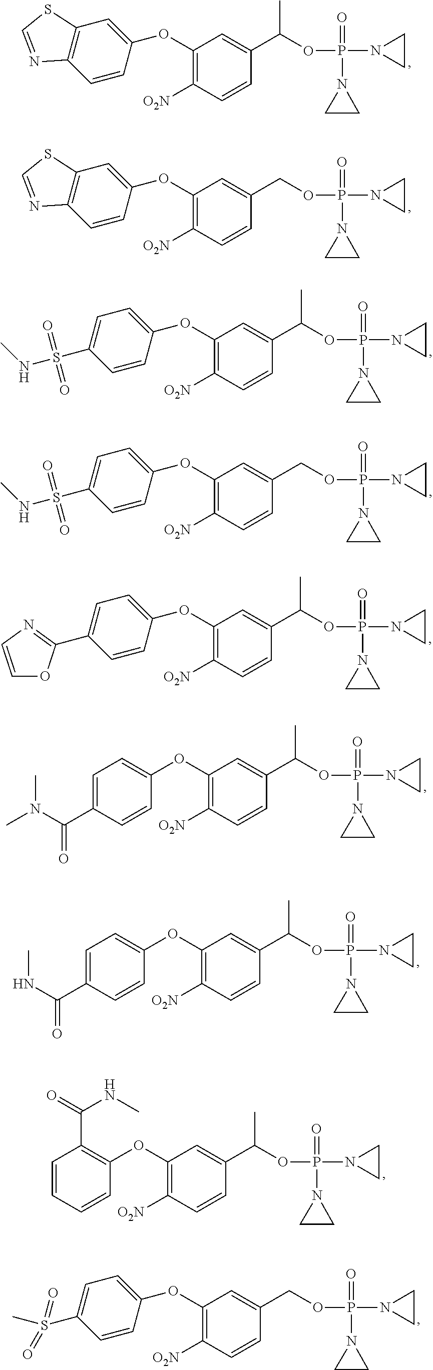

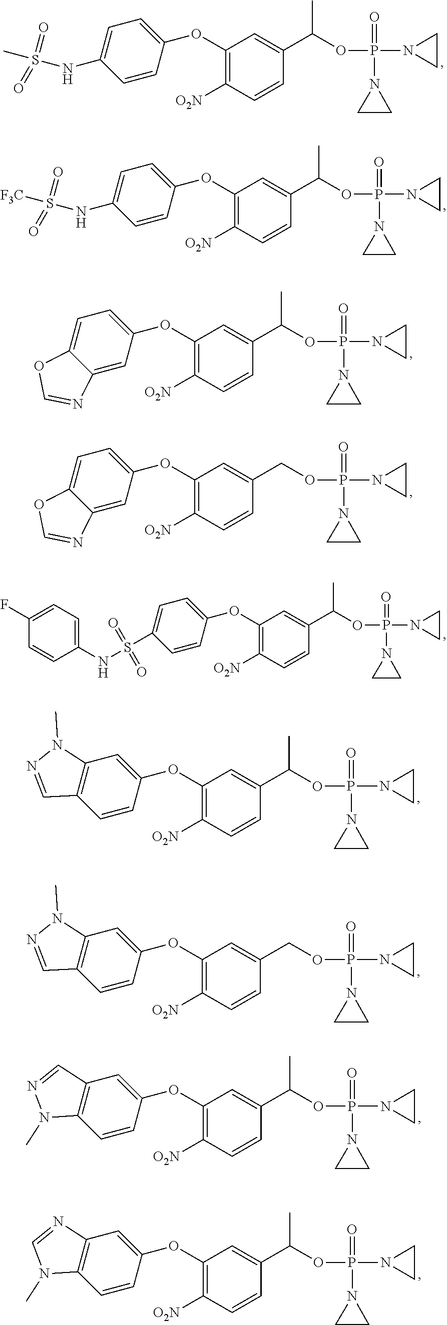

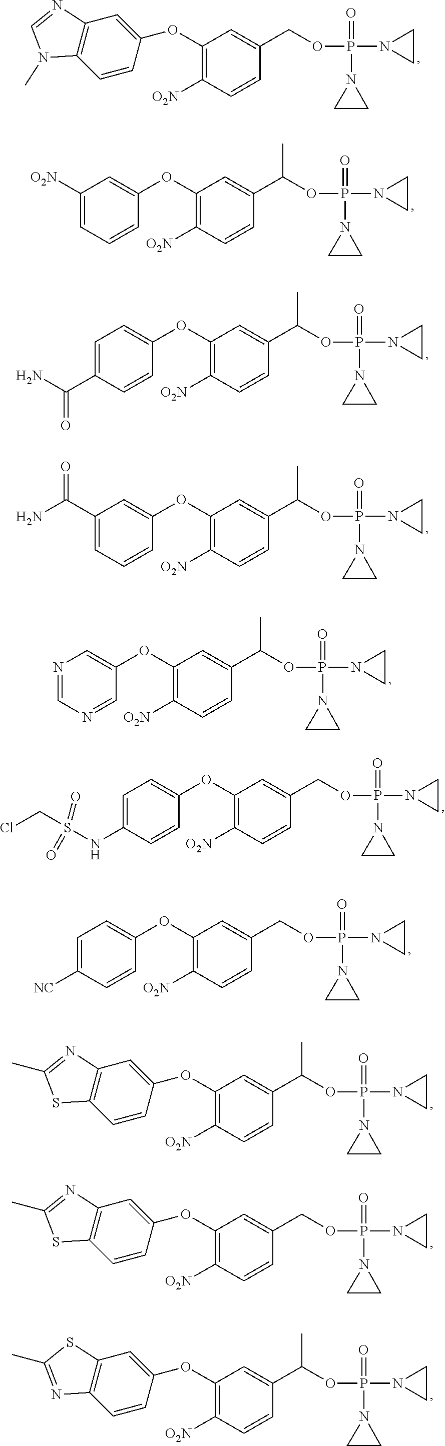

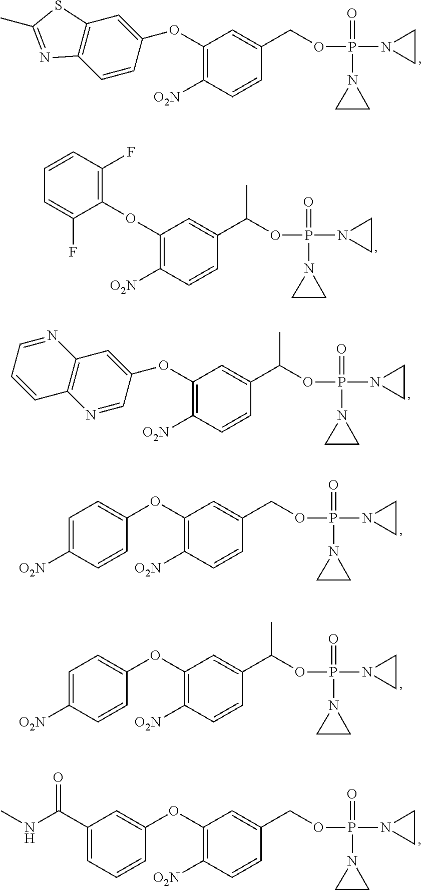

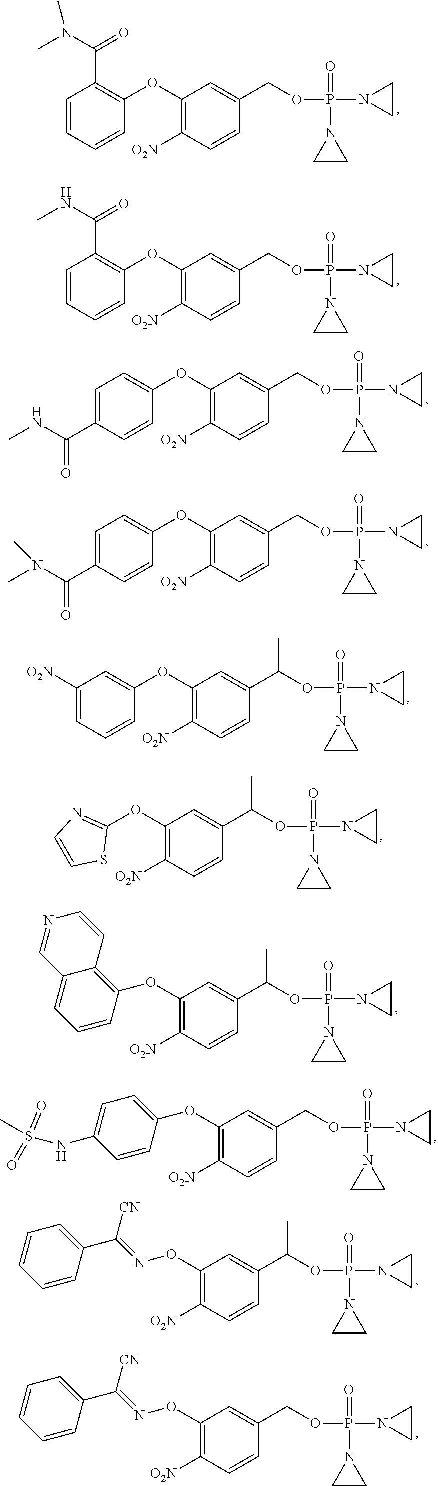

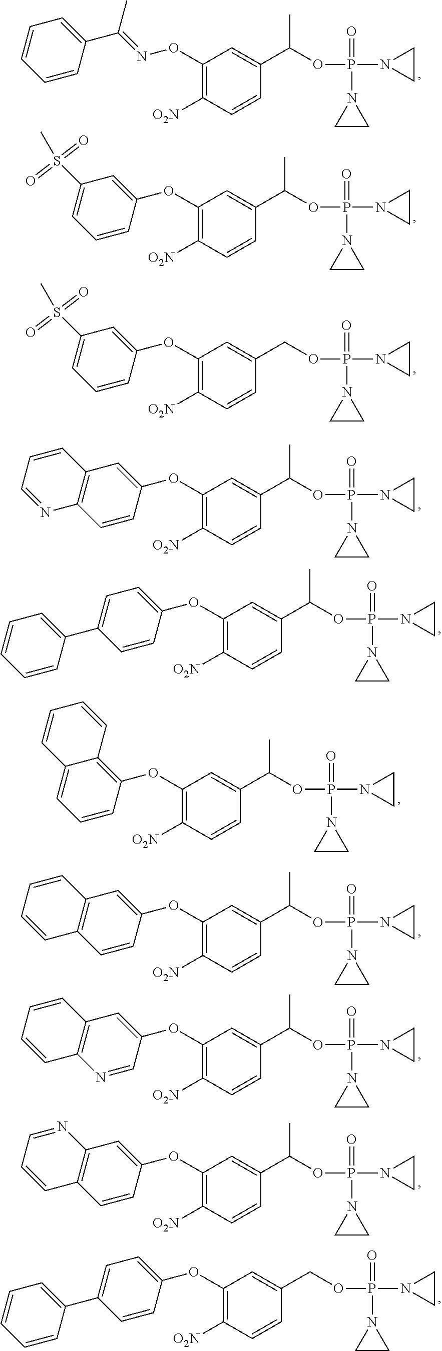

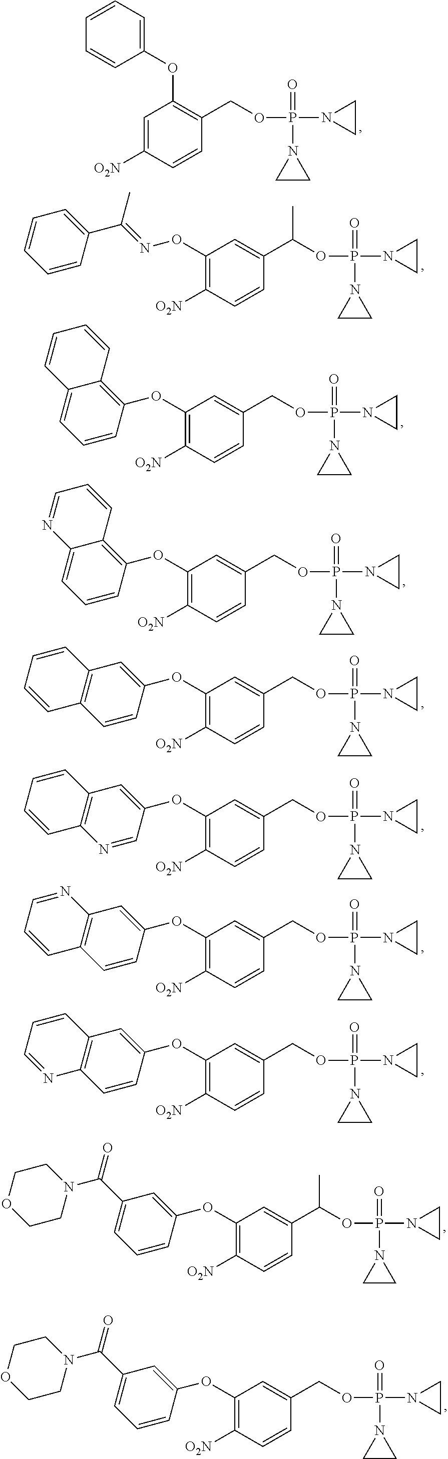

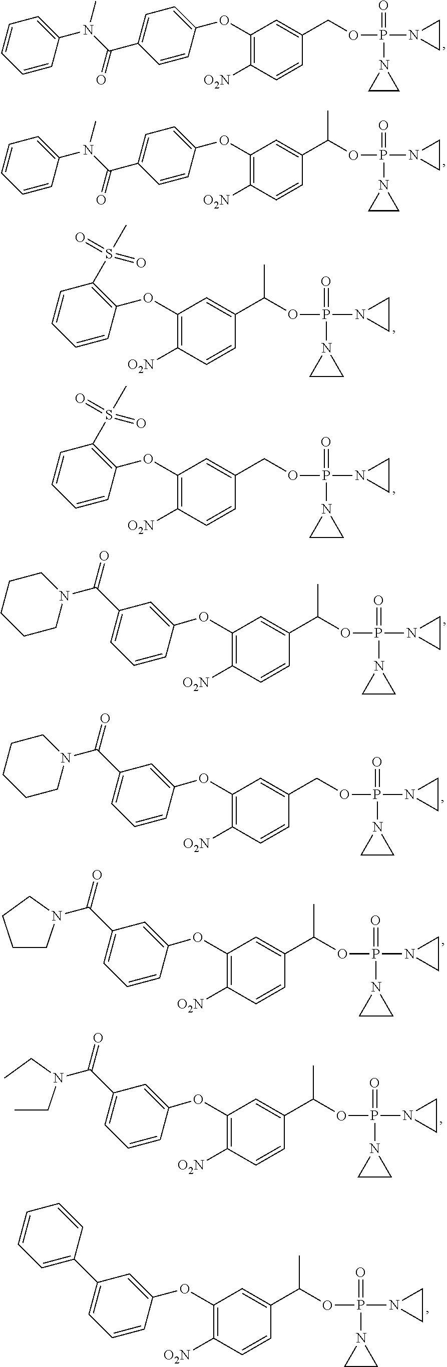

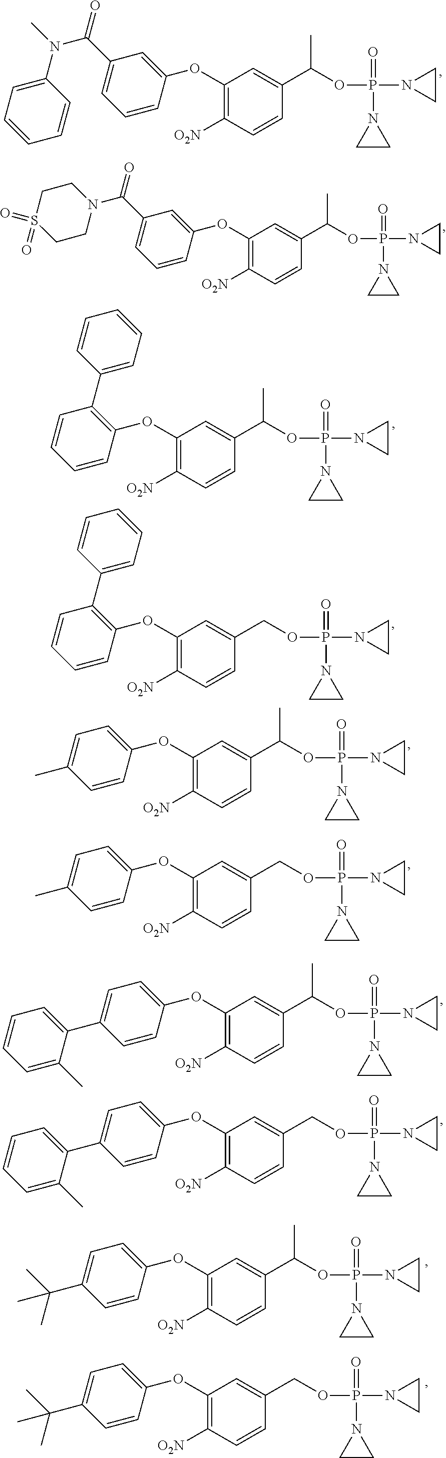

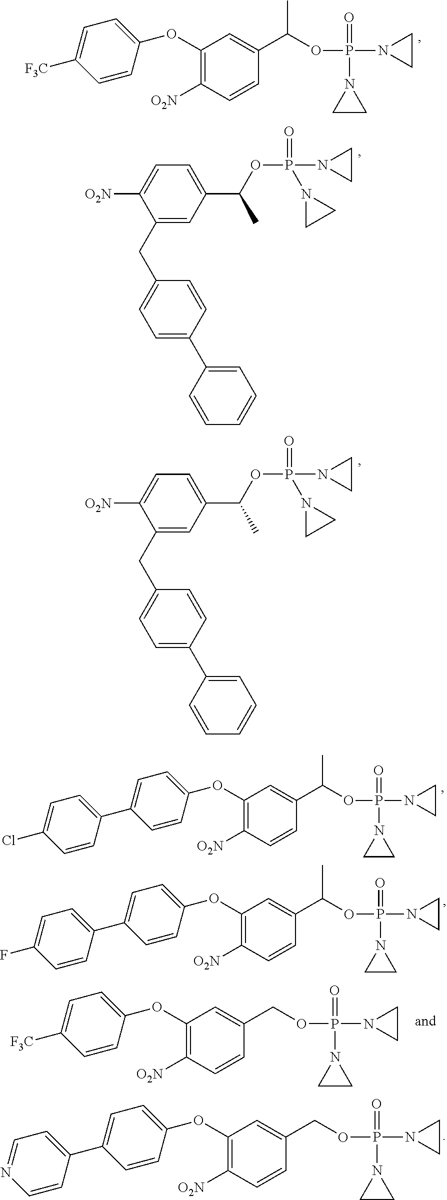

Specific definitions and meanings of the groups, and the preparation methods and spectral data of the compounds have been described in PCT/US2016/021581 with Publication No. WO2016145092A1 (corresponding to Chinese Patent Application No. 2016800150788 with Publication No. CN107530556A); PCT/US2016/062114 with Publication No. WO2017087428A1 (corresponding to Chinese Patent Application No. 2016800446081 with Publication No. CN108290911A); and PCT/CN2020/089692 with Publication No. WO2020228685A1, which are incorporated herein by reference in its entirety.

Evidently, the compounds of Formula (6), similar to AST-3424 or AST, are prodrugs of AST-2660, and can be activated by the AKR1C3 enzyme to form AST-2660 to exert its anticancer efficacy:

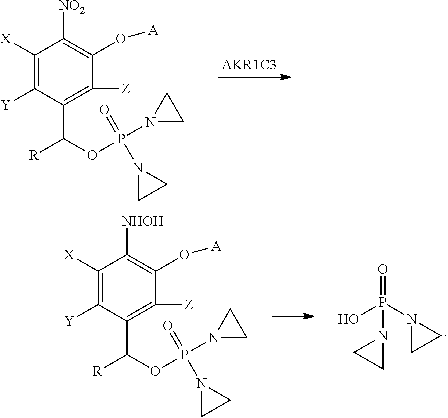

The preparation methods and spectral data of AST-3424 (OBI-3424),

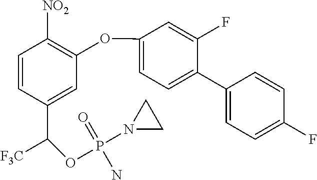 [SMILES: NP(=O)(OC(c1ccc([N+](=O)[O-])c(Oc2ccc(-c3ccc(F)cc3)c(F)c2)c1)C(F)(F)F)N1CC1]

(hereinafter referred to as AST) and

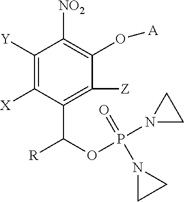

have been disclosed in the following patent applications: PCT/US2016/021581 with Publication No. WO2016145092A1 (corresponding to Chinese Patent Application No. 2016800150788 with Publication No. CN107530556A); PCT/US2016/062114 with Publication No. WO2017087428A1 (corresponding to Chinese Patent Application No. 2016800446081 with Publication No. CN108290911A); and PCT/CN2020/089692 with Publication No. WO2020228685A1; the relevant preparation concentrate injection, and the relevant prescription, preparation method and clinical compatibility and administration method have been described in detail and disclosed by the relevant patents: WO2021008520A1, WO2021043275A1, which are incorporated herein by reference in its entirety.

Monotherapy refers to a single drug therapy. Combination refers to a combined drug therapy. Single drug therapy refers to the use of only one anticancer drug in a course of treatment. Combination therapy refers to the simultaneous or sequential use of two or more anticancer drugs in a course of treatment.

Generally speaking, combination therapy needs to explore different administration dosages and administration cycles according to the characteristics of the disease and the types of drugs to be used in combination, and only according to the above situation, the combination therapy plan obtained from the exploration may achieve better therapeutic effect as compared with single drug therapy.

The administration dosages and administration cycles of drug of both monotherapy and combination therapy regimens need to be explored through clinical trials with reference to the dosage and administration regimens of AST-3424 and analogues thereof and other drugs as described above.

The administration dosages of the monotherapy can be determined with reference to the animal experimental dosages of WO2019062919A1 as well as WO2016145092A1 and WO2017087428A1.

Furthermore, the KRAS mutation is selected from the group consisting of KRAS-G12D mutation, KRAS-G12V mutation and KRAS-G12C mutation.

Mutations in either or both of the genes corresponding to KRAS can be detected and diagnosed with commercially available (companion) diagnostic kits, such as those approved in China:
Amoy Dx, State instrument registration 20153401126, Human KRAS gene mutation detection kit (fluorescent PCR method)
Tellgen, State instrument registration 20163401341, Human K-RAS gene 7 mutations detection kit (PCR fluorescence method).

Evidently, NGS sequencing (YS 450 gene NGS large panel) can also be used to determine the specific KRAS mutation subtype.

More preferably, the KRAS mutation is selected from the KRAS-G12D mutation.

The TMB (Tumor Mutation Load (burden)) level of the described gene mutation is medium.

Since the TMB (Tumor Mutation Load (burden)) is differently high or low between different tumor types: it is generally considered that: a TMB of more than 20 mutations/Mb (Mb represents bases per million) is high; a TMB of less than 10 mutations/Mb is low, and a TMB in the middle is medium. At the World Conference on Lung Cancer in 2017, Squibb Inc. had announced the results of a clinical trial named CheckMate-032. This was a phase II clinical trial that enrolled 401 patients with advanced lung cancer who had failed first-line therapy and were treated with a PD-1 inhibitor alone or in combination with Ipilimumab. The patients were divided into three categories of patients with high TMB, medium TMB, and low TMB according to their TMB levels, then among those who received the combination therapy, the efficiency of the three groups was 62%, 20%, and 23%, respectively, and the efficiency of the group with high TMB was three times higher than the remaining two groups; and the median overall survival of the three groups was: 22.0 months, 3.6 months, and 3.4 months, respectively, with 22.0 months versus 3.4 months, a 6-fold difference! This trial demonstrated that for different cancer treatment drugs, different TMB levels have a significant impact on the efficacy of the drugs.

Further, the prodrug compound is preferably selected from the group consisting of:

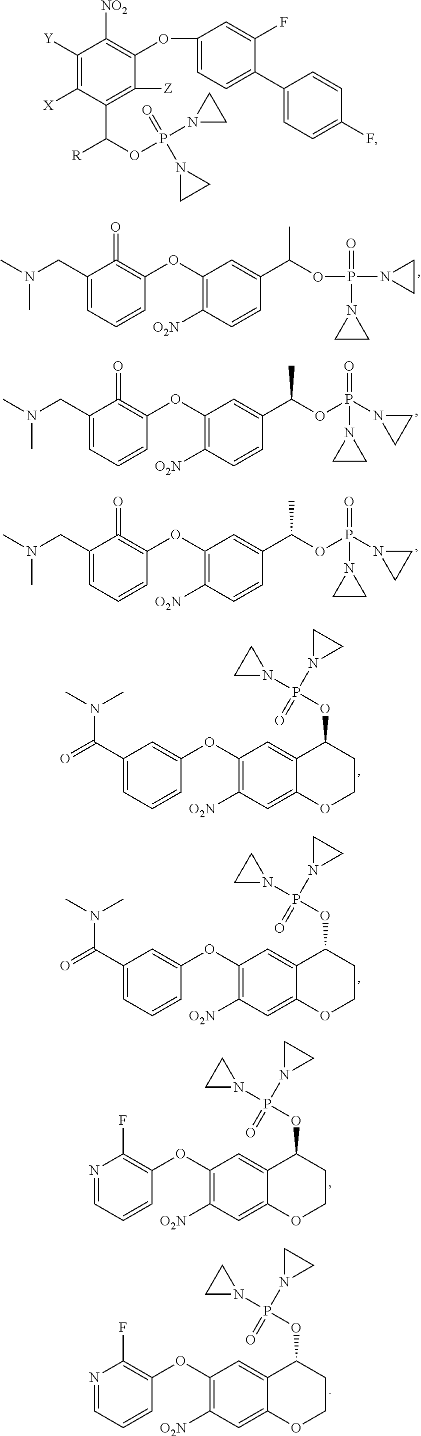

The above-mentioned cancer is selected from the group consisting of ovarian cancer, breast cancer, pancreatic cancer, fallopian tube cancer, primary peritoneal cancer, gastric cancer, prostate cancer, liver cancer, colon cancer, rectal cancer, lung cancer, and bladder cancer.

Other therapeutic drugs are selected from the group consisting of KRAS inhibitors and immunotherapy drugs (immune checkpoint inhibitors).

KRAS inhibitors are substances that inhibit KRAS enzyme activity. For details, please refer to the review document Goebel, Lisa & Müller, Matthias & Goody, Roger & Rauh, Daniel. (2020). KRasG12C inhibitors in clinical trials: A short historical perspective. RSC Medicinal Chemistry. 11. 10.1039/DOMD00096E.

KRAS inhibitors that have entered clinical development or have already been marketed include Sotorasib (AMG510) developed by Amgen in the United States, adagrasib (MRTX849) developed by Mirati Therapeutics, GDC6036 developed by Roche, LY3499446 developed by Lilly, JNJ74699157 (ARS3248) jointly developed by Araxes and Janssen, D-1553 developed by InventisBio Co., Ltd, JAB-3312 developed by Jacobio Pharma, JAB-3068 developed by Jacobio Pharma, GH35 developed by Gen House, and BPI-421286 developed by Betta Pharma, BI17016963 developed by Boehringer-Ingelheim, mRNA-5671 developed by Moderna, and AZD-4785 developed in collaboration with AstraZeneca and Ionis.

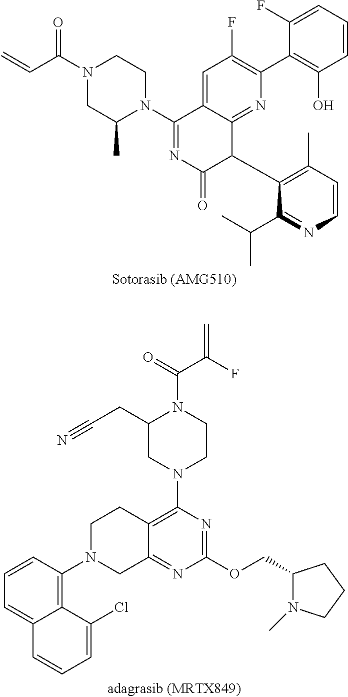

Wherein, KRAS inhibitor is selected from the group consisting of Sotorasib (AMG510), adagrasib (MRTX849), GDC6036, LY3499446, JNJ74699157 (ARS3248), D-1553;

Immune checkpoint molecules are regulatory molecules in the immune system that play an inhibitory role and are essential for maintaining self-tolerance, preventing autoimmune reactions, and minimizing tissue damage by controlling the timing and intensity of the immune response. Immune checkpoint molecules are expressed on immune cells, and will inhibit the function of immune cell, thereby preventing the body from generating an effective anti-tumor immune response, and causing immune escape of tumor. Tumor-related immune checkpoint molecules include PD1, PD-L1, CTLA4, Tim3, and LAG3, etc. Currently, PD1, PD-L1, and CTLA4 are more frequently studied. Immune checkpoint inhibitors are some monoclonal antibody drugs developed for corresponding immune checkpoints. Their main function is to block the interaction between tumor cells expressing immune checkpoints and immune cells, thereby blocking the inhibitory effect of tumor cells on immune cells. Immunotherapy drugs are selected from the group consisting of PD-1 monoclonal antibodies and PD-L1 monoclonal antibodies.

The cancer and tumor are preferably selected from the group consisting of gastric cancer, pancreatic cancer and lung cancer.

In addition to containing corresponding prodrug compounds, the above-mentioned drugs should also be supplemented with pharmaceutically acceptable auxiliaries or excipients based on the specific characteristics of the drug, medicament or formulation. The medicament can be any dosage form for clinical administration, such as tablets, suppositories, dispersible tablets, enteric-coated tablets, chewable tablets, orally disintegrating tablets, capsules, sugar coated agents, granules, dry powders, oral solutions, a small needle for injection, lyophilized powder for injection, or infusion solutions. According to the specific dosage form and the mode of administration, the pharmaceutically acceptable auxiliaries or excipients in the medicament may include one or more of the following: diluent, solubilizer, disintegrant, suspension, lubricant, adhesive, filler, flavoring agent, sweetener, antioxidant, surfactant, preservative, wrapping agent and pigment, etc.

### BRIEF DESCRIPTION OF THE DRAWINGS

FIG. 1 is growth curves of tumor volume in each group of mice in the HuPrime® gastric cancer GA6201 model;

FIG. 2 is relative tumor growth inhibition rate curves in each group of mice in the HuPrime® gastric cancer GA6201 model;

FIG. 3 is body weight curves in each group of mice in the HuPrime® gastric cancer GA6201 model;

FIG. 4 is percentage change curves of body weight in each group of mice in the HuPrime® gastric cancer GA6201 model;

FIG. 5 is growth curves of tumor volume in each group of mice in the HuPrime® pancreatic cancer PA1222 model;

FIG. 6 is relative tumor growth inhibition rate curves in each group of mice in the HuPrime® pancreatic cancer PA1222 model;

FIG. 7 is body weight curves in each group of mice in the HuPrime® pancreatic cancer PA1222 model;

FIG. 8 is percentage change curves of body weight in each group of mice in the HuPrime® pancreatic cancer PA1222 model;

FIG. 9 is growth curves of tumor volume in each group of mice in the HuPrime® lung cancer LU11693 model;

FIG. 10 is relative tumor growth inhibition rate curves in each group of mice in the HuPrime® lung cancer LU11693 model;

FIG. 11 is body weight curves in each group of mice in the HuPrime® lung cancer LU11693 model;

FIG. 12 is percentage change curves of body weight in each group of mice in the HuPrime® lung cancer LU11693 model;

FIG. 13 is growth curves of tumor volume in each group of mice in the human-derived pancreatic cancer HPAF-II subcutaneous xenograft model;

FIG. 14 is percentage change curves of body weight in each group of mice in the human-derived pancreatic cancer HPAF-II subcutaneous xenograft model;

FIG. 15 is growth curves of tumor volume in each group of mice in the HuPrime® lung cancer LU5161 subcutaneous model;

FIG. 16 is percentage change curves of body weight in each group of mice in the HuPrime® lung cancer LU5161 subcutaneous model;

FIG. 17 is growth curves of tumor volume in each group of mice in the HuPrime® intestinal cancer CR3820 subcutaneous model;

FIG. 18 is percentage change curves of body weight in each group of mice in the HuPrime® intestinal cancer CR3820 subcutaneous model;

FIG. 19 is growth curves of tumor volume in each group of mice in the HuPrime® pancreatic cancer PA2637 subcutaneous model;

FIG. 20 is percentage change curves of body weight in each group of mice in the HuPrime® pancreatic cancer PA2637 subcutaneous model;

FIG. 21 is growth curves of tumor volume in each group of mice in the HuPrime® lung cancer LU11873 subcutaneous model;

FIG. 22 is percentage change curves of body weight in each group of mice in the HuPrime® lung cancer LU11873 subcutaneous model;

FIG. 23 is growth curves of tumor volume in each group of mice in the HuPrime® pancreatic cancer PA1383 subcutaneous model;

FIG. 24 is percentage change curves of body weight in each group of mice in the HuPrime® pancreatic cancer PA1383 subcutaneous model;

FIG. 25 is the photographs of the IHC staining results of the GA6201, LU11693, PA1222 models and the control group.

### DETAILED DESCRIPTION OF THE INVENTION

The present invention will be described below with reference to specific embodiments. Those skilled in the art will understand that these examples are only used to illustrate the present invention and do not limit the scope of the present invention in any way.

“Administering” or “administration of” a drug to a patient (and the grammatical equivalents of this phrase) refers to direct administration, which may be administered to a patient by a medical professional or may be self-administered, and/or indirect administration, which may be the act of prescribing a drug. For example, a physician who instructs a patient to self-administer a drug and/or provides a patient with a prescription for a drug is administering the drug to the patient.

“Cancer” refers to leukemias, lymphomas, carcinomas, and other malignant tumors (including solid tumors) with potentially unrestrained growth that can expand locally by invasion and systemically by metastasis. Examples of cancers include, but are not limited to, cancer of the adrenal gland, bone, brain, breast, bronchi, colon and/or rectum, gallbladder, head and neck, kidney, larynx, liver, lung, nervous tissue, pancreas, prostate, parathyroid, skin, stomach and thyroid. Certain other examples of cancers include acute and chronic lymphocytic and granulocytic neoplasms, adenocarcinoma, adenoma, basal cell carcinoma, cervical dysplasia carcinoma and in situ carcinoma, Ewing's sarcoma, epidermoid carcinoma, giant cell carcinoma, glioblastoma multiforme, hairy-cell tumor, intestinal ganglioneuroma, hyperplastic corneal nerve tumor, islet cell carcinoma, Kaposi's sarcoma, leiomyoma, leukemia, lymphoma, malignant carcinoid, malignant melanoma, malignant hypercalcemia, marfanoid habitus tumor, medullary epithelial carcinoma, metastatic skin cancer, mucosal neuroma, myeloma, mycosis fungoides, neuroblastoma, osteosarcomas, osteogenic and other sarcomas, ovarian tumors, pheochromocytoma, polycythemia vera, primary brain tumor, small cell lung cancer, squamous cell carcinoma of both ulcerative and papillary types, hyperplasia, seminoma, soft tissue sarcoma, retinoblastoma, rhabdomyosarcoma, renal cell tumors, topical skin lesion, reticulum cell sarcoma and Wilm's tumor.

“Patient” and “individual” are used interchangeably and refer to a mammal in need of treatment for cancer. Typically, the patient is a human. Typically, the patient is a human diagnosed with cancer. In certain embodiments, a “patient” or “individual” may refer to a non-human mammal, such as a non-human primate, a dog, cat, rabbit, pig, mouse, or rats used for screening, characterizing, and evaluating drugs and therapies.

“Solid tumors” refers to solid tumors including, but not limited to, metastatic tumors in bone, brain, liver, lung, lymph node, pancreas, prostate, skin, and soft tissue (sarcoma).

“Therapeutically effective amount” of a drug refers to an amount of a drug that, when administered to a patient with cancer, will have the intended therapeutic effect (e.g., alleviation, amelioration, palliation, or elimination of one or more clinical manifestations of cancer in the patient). A therapeutic effect does not necessarily occur by administration of one dose, and may occur only after administration of a series of doses. Thus, a therapeutically effective amount may be administered in one or more administrations.

“Treating”, “treatment of” or “therapy of” a condition or a patient refers to taking steps to obtain a beneficial or desired result (including clinical results). For purposes of the present invention, beneficial or desired clinical results include, but are not limited to, alleviation or amelioration of one or more symptoms of cancer; diminishment of extent of disease; delay or slowing of disease progression; amelioration, remission, or stabilization of disease status; or other beneficial results. In some cases, treatment of cancer can result in partial response or stable disease.

“Tumor cells” refers to tumor cells of any appropriate species, e.g., mammalian such as murine, canine, feline, equine or human.

“Patient” and “individual” are used interchangeably and refer to a mammal in need of treatment for cancer. Typically, the patient is a human. Typically, the patient is a human diagnosed with cancer. In certain embodiments, a “patient” or “individual” may refer to a non-human mammal, such as a non-human primate, a dog, cat, rabbit, pig, mouse, or rats used for screening, characterizing, and evaluating drugs and therapies.

“Treatment” or “treatment of a patient” is to administer, use or administer a therapeutic effective amount of a drug to a patient in relation to the present invention.

“Administering” or “administration of” or “use of” a drug to a patient refers to direct administration, which may be administered to a patient by a medical professional or may be self-administered, and/or indirect administration, which may be the act of prescribing a drug. For example, a physician who instructs a patient to self-administer a drug and/or provides a patient with a prescription for a drug is administering the drug to the patient.

“Treating”, “treatment of” or “therapy of” a condition or a patient refers to taking steps to obtain a beneficial or desired result (including clinical results). For purposes of the present invention, beneficial or desired clinical results include, but are not limited to, alleviation or amelioration of one or more symptoms of cancer; diminishment of extent of disease; delay or slowing of disease progression; amelioration, remission, or stabilization of disease status; or other beneficial results. In some cases, treatment of cancer can result in partial response or stable disease.

The experimental methods in the following examples are conventional methods unless specified otherwise. The medicinal raw materials, reagent materials, etc. used in the following examples are all commercially available products unless specified otherwise.

The above description of embodiments of the present invention does not limit the present invention. Those skilled in the art can make various modifications and changes according to the present invention, and any modification and change within the spirit of the present invention shall be covered in the scope of the claims appended to the present invention.

Specific experiments of the present invention are provided below.

### 1. Pharmacodynamics Evaluation of the Test Substances AST, AST-3424 and Ifosfamide in the HuPrime® Gastric Cancer GA6201 Subcutaneous Xenograft Model

The HuPrime® gastric cancer GA6201 PDX model was a model of KRAS pathogenic mutation having G12D amino acid mutation (KRAS-G12D). BALB/c nude mice were subcutaneously inoculated with HuPrime® model GA6201 tumor blocks so as to establish a subcutaneous transplantation tumor model of human gastric cancer. The test was divided into the test drug Ifosfamide 60 mg/kg group, the test drug AST-3424 5 mg/kg group, AST 2.5 mg/kg and 5 mg/kg groups, and physiological saline (pH 7.0-7.6) vehicle control group, with a total of 5 groups and 5 mice in each group. Among them, physiological saline (pH 7.0-7.6) vehicle control group, the test drug AST-3424 5 mg/kg group, AST 2.5 mg/kg group and 5 mg/kg group were administered by tail vein injection once a week for a total of three weeks, and observed for four weeks. The Ifosfamide 60 mg/kg group was administered through intraperitoneal injection continuously for five days a week and discontinued for two days for a total of two weeks, and observed for five weeks. The therapeutic effect was evaluated based on the relative tumor growth inhibition rate (TGI (%)), and the safety was evaluated based on the body weight changes and the death situation of the animals.

The specific administration regimen for each group was shown in Table 1 below.

TABLE 1
Experimental design of anti-tumor effects of test drugs at different doses
in the HuPrime ® gastric cancer GA6201 tumor model

| | The | | | | | |
|---|---|---|---|---|---|---|
| | number | | | | Planned | Actual |
| Group | of | Administration | Dosage | Administration | administration | administration |
| No. | Animals | group | (mg/kg) | method | period | period |
| 1 | 5 | Physiological | 0 | tail vein | Q7D × 3 | Q7D × 3 |
| | | saline, pH 7.0- | | | | |
| | | 7.6 | | | | |
| 2 | 5 | Ifosfamide | 60 | intraperitoneal | QD × 5/week × 2 wks | QD × 5/week × 2 wks |
| 3 | 5 | AST-3424 | 5 | tail vein | Q7D × 3 | Q7D × 3 |
| 4 | 5 | AST | 5 | tail vein | Q7D × 3 | Q7D × 3 |
| 5 | 5 | AST | 2.5 | tail vein | Q7D × 3 | Q7D × 3 |

The tumor volumes of mice in different groups were measured on different days and the mean values were obtained. The results were shown in Table 2 below.

TABLE 2
Changes in tumor volume in each group of mice over treatment time
in the HuPrime ® gastric cancer GA6201 model

| | Tumor Volume (mm³) (X̅ ± S) | | | | |
|---|---|---|---|---|---|
| | Group 1 | | | | |
| | Physiological | Group 2 | Group 3 | Group 4 | Group 5 |
| | saline, | Ifosfamide | AST-3424 | AST | AST |
| Time | pH 7.0-7.6 | 60 mg/kg | 5 mg/kg | 5 mg/kg | 2.5 mg/kg |
| 0 day after initial | 94.22 ± 8.5 | 94.15 ± 6.68 | 94.19 ± 12.46 | 94.36 ± 14.61 | 94.61 ± 11.1 |
| administration | | | | | |
| 3 days after initial | 123.26 ± 13.74 | 122.22 ± 12.32 | 114.98 ± 21.76 | 118.42 ± 20.83 | 120.19 ± 15.57 |
| administration | | | | | |
| 7 days after initial | 161.08 ± 22.55 | 129.39 ± 13.47 | 84.33 ± 17.54 | 101.91 ± 19.72 | 118.33 ± 27.54 |
| administration | | | | | |
| 10 days after initial | 182.31 ± 28.05 | 175 ± 22.09 | 77.04 ± 15.37 | 97.17 ± 25.14 | 118.65 ± 31.45 |
| administration | | | | | |
| 14 days after initial | 242.26 ± 40.75 | 190.74 ± 29.81 | 63.59 ± 10.46 | 74.2 ± 16.99 | 96.08 ± 26.3 |
| administration | | | | | |
| 17 days after initial | 297.28 ± 47.21 | 189.23 ± 29.28 | 52.08 ± 9.25 | 59.77 ± 15.76 | 76.14 ± 18.07 |
| administration | | | | | |
| 21 days after initial | 398.37 ± 87.32 | 217.96 ± 41.06 | 45.81 ± 13.3 | 46.21 ± 13.48 | 71.72 ± 9.08 |
| administration | | | | | |
| 24 days after initial | 479.52 ± 119.81 | 225.66 ± 44.63 | 40.55 ± 10.4 | 44.03 ± 11.57 | 57.94 ± 10.4 |
| administration | | | | | |
| 28 days after initial | 579.9 ± 154.47 | 247.04 ± 50.8 | 30.28 ± 3.78 | 38.5 ± 11 | 45.51 ± 1.64 |
| administration | | | | | |
| 31 days after initial | 648.12 ± 177.44 | 265.76 ± 52.69 | 23.24 ± 7.23 | 35.92 ± 10.45 | 43.44 ± 4.51 |
| administration | | | | | |
| 35 days after initial | 831.15 ± 234.39 | 324.51 ± 73.75 | 18.55 ± 5.77 | 22.7 ± 11.73 | 32.04 ± 4.52 |
| administration | | | | | |
| 38 days after initial | 905.75 ± 252.4 | 371.02 ± 75.96 | 12.65 ± 7.91 | 19.97 ± 10.18 | 28.69 ± 8.2 |
| administration | | | | | |
| 42 days after initial | 1026.01 ± 294.05 | 403.47 ± 72.29 | 13.42 ± 8.22 | 5.05 ± 5.05 | 23.49 ± 6.18 |
| administration | | | | | |
| 45 days after initial | 1130.43 ± 319.13 | 484.83 ± 98.33 | 10.82 ± 7.05 | 11.14 ± 7.04 | 25 ± 6.37 |
| administration | | | | | |
| 49 days after initial | 1283.11 ± 366.66 | 578.6 ± 99.79 | 8.15 ± 5.03 | 9.96 ± 7.12 | 17.72 ± 4.6 |
| administration | | | | | |

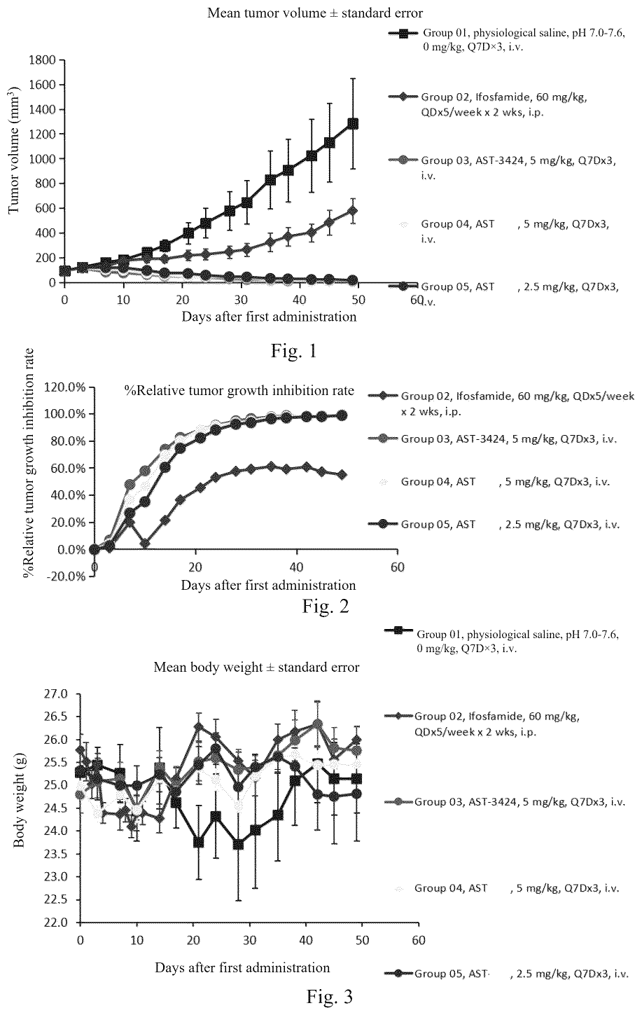

FIG. 2 showing the tumor growth of each treatment group and control group was made according to the data in Table 2.

The drug efficacy evaluation and analysis data was made according to the data in Table 2. The specific data were shown in Table 3.

TABLE 3
Drug efficacy analysis table of each group in the HuPrime ® gastric
cancer GA6201 model

| | Day 3 after the end of administration (i.e., Day 38, Feb. 27, 2020) | | | | |
|---|---|---|---|---|---|
| | | Relative | | | P Value |
| | Tumor volume | tumor volume | TGI | T/C | (comparing with the |
| Experiment group | (X̅ ± S) | (X̅ ± S) | (%) | (%) | control group) |
| Group 1 | 905.75 ± 252.4 | 9.34 ± 2.42 | — | — | — |
| Physiological | | | | | |
| saline, pH 7.0-7.6 | | | | | |
| Group 2 | 371.02 ± 75.96 | 3.85 ± 0.63 | 58.83 | 41.17 | 0.857 |
| Ifosfamide | | | | | |
| 60 mg/kg | | | | | |
| Group 3 | 12.65 ± 7.91 | 0.11 ± 0.07 | 98.79 | 1.21 | 0.00000265 |
| AST-3424 | | | | | |
| 5 mg/kg | | | | | |
| Group 4 | 19.97 ± 10.18 | 0.19 ± 0.08 | 97.96 | 2.04 | 0.0000182 |
| AST | | | | | |
| 5 mg/kg | | | | | |
| Group 5 | 28.69 ± 8.2 | 0.29 ± 0.08 | 96.85 | 3.15 | 0.000135 |
| AST | | | | | |
| 2.5 mg/kg | | | | | |

The relative tumor proliferation rate (T/C %) in Table 3 was the percentage of relative tumor volume or tumor weight between the treatment group and the control group at a certain time point. The calculation formula was as follows:
T/C %=TRTV/CRTV×100% (TRTV: the mean RTV of the treatment group; CRTV: the mean RTV of the vehicle control group; RTV=Vt/V0, V0 was the tumor volume of the animal when grouped, Vt was the tumor volume of the animal after treatment);
The relative tumor growth inhibition rate (TGI (%)) was calculated as follows: TGI %=(1−T/C)×100%. (T and C were the mean relative tumor volumes (RTV) of the treatment group and the control group at a specific time point, respectively).

TABLE 4
Relative tumor growth inhibition rate of each group of tumors in the HuPrime ® gastric cancer GA6201 model

| Group | 0 | 3 | 7 | 10 | 14 | 17 | 23 | 24 | 28 | 31 | 35 | 38 |
|---|---|---|---|---|---|---|---|---|---|---|---|---|
| Group 01 | | | | | | | | | | | | |
| Group 02 | 0.08% | 0.85% | 19.67% | 4.01% | 21.27% | 36.35% | 45.29% | 52.94% | 57.40% | 59.00% | 60.86% | 59.04% |
| Group 03 | 0.04% | 6.72% | 47.65% | 57.74% | 73.75% | 82.48% | 88.50% | 91.54% | 94.78% | 96.41% | 97.77% | 98.60% |
| Group 04 | −0.15% | 3.93% | 36.73% | 46.70% | 69.37% | 79.89% | 88.40% | 90.82% | 93.36% | 94.46% | 97.27% | 97.80% |
| Group 05 | −0.42% | 2.50% | 26.54% | 34.92% | 60.34% | 74.39% | 82.00% | 87.92% | 92.15% | 93.30% | 96.15% | 96.83% |
| Group 06 | −0.02% | 1.19% | 23.52% | 15.82% | 21.09% | 26.03% | 38.10% | 43.33% | 53.57% | 58.24% | 63.99% | 69.40% |

The above Table 4 was made into a curve graph to obtain FIG. 2.

The body weights of mice in different groups were measured on different days and the mean values were obtained. The results were shown in Table 5 below.

TABLE 5
Body weights of mice on different inoculation days in the
HuPrime ® gastric cancer GA6201 model

| Group | 0 | 1 | 2 | 3 | 4 | 7 | 8 | 9 | 10 | 11 |
|---|---|---|---|---|---|---|---|---|---|---|
| Group | 25.3 | | | 25.4 | | 25.3 | | | 24.4 | |
| 01 | | | | | | | | | | |
| Group | 25.8 | 25.5 | 25.0 | 25.5 | 24.4 | 24.4 | 24.5 | 24.1 | 24.4 | 24.4 |
| 02 | | | | | | | | | | |
| Group | 24.8 | | | 25.1 | | 25.1 | | | 24.5 | |
| 03 | | | | | | | | | | |
| Group | 25.0 | | | 24.4 | | 24.8 | | | 24.5 | |
| 04 | | | | | | | | | | |
| Group | 25.3 | | | 25.1 | | 25.0 | | | 25.0 | |
| 05 | | | | | | | | | | |

| Group | 14 | 15 | 16 | 17 | 18 | 21 | 24 | 28 | 31 | 35 | 38 |
|---|---|---|---|---|---|---|---|---|---|---|---|
| Group | 25.4 | | | 24.6 | | 23.8 | 24.3 | 23.7 | 24.0 | 24.4 | 25.1 |
| 01 | | | | | | | | | | | |
| Group | 24.3 | | | 25.1 | | 26.3 | 26.1 | 25.5 | 25.2 | 26.0 | 26.2 |
| 02 | | | | | | | | | | | |
| Group | 25.4 | | | 25.0 | | 25.5 | 25.6 | 25.3 | 25.4 | 25.7 | 26.0 |
| 03 | | | | | | | | | | | |
| Group | 25.2 | | | 24.9 | | 25.4 | 25.1 | 24.6 | 25.2 | 25.6 | 25.7 |
| 04 | | | | | | | | | | | |
| Group | 25.2 | | | 24.9 | | 25.4 | 25.8 | 25.0 | 25.4 | 25.6 | 25.4 |
| 05 | | | | | | | | | | | |

The above table was made into a curve graph to obtain FIG. 3, which was body weight curve in each group of mice in the HuPrime® gastric cancer GA6201 model.

Similarly, the data in Table 5 was processed to obtain the following Table 6.

TABLE 6
Percentage changes in body weight of mice on different inoculation days in the
HuPrime ® gastric cancer GA6201 model (% Group Mean Change =
mean((T − T0)/T0)*100, T represented current value, T0 represented initial value)

| Group | 0 | 1 | 2 | 3 | 4 | 7 | 8 | 9 | 10 | 11 |
|---|---|---|---|---|---|---|---|---|---|---|
| Group | 0.00% | | | 0.72% | | −0.09% | | | −3.34% | |
| 01 | | | | | | | | | | |
| Group | 0.00% | 1.02% | 2.93% | 1.20% | 5.32% | −5.50% | −5.07% | −6.48% | −5.33% | −5.32% |
| 02 | | | | | | | | | | |
| Group | 0.00% | | | 1.21% | | 1.46% | | | −1.11% | |
| 03 | | | | | | | | | | |
| Group | 0.00% | | | 2.44% | | −0.77% | | | −2.14% | |
| 04 | | | | | | | | | | |
| Group | 0.00% | | | 0.70% | | −1.21% | | | −1.22% | |
| 05 | | | | | | | | | | |

| Group | 14 | 15 | 16 | 17 | 18 | 21 | 24 | 28 | 31 | 35 | 38 |
|---|---|---|---|---|---|---|---|---|---|---|---|
| Group | 0.31% | | | −2.58% | | −4.69% | −2.40% | −4.98% | −3.68% | −2.33% | 0.69% |
| 01 | | | | | | | | | | | |
| Group | −5.80% | | | −2.46% | | 1.96% | 1.12% | −0.90% | −2.41% | 0.86% | 1.53% |
| 02 | | | | | | | | | | | |
| Group | 2.52% | | | 0.87% | | 3.03% | 3.31% | 2.27% | 2.52% | 3.58% | 4.87% |
| 03 | | | | | | | | | | | |
| Group | 0.65% | | | −0.46% | | 1.57% | 0.53% | −1.72% | 0.85% | 2.51% | 2.73% |
| 04 | | | | | | | | | | | |
| Group | −0.28% | | | −1.77% | | 0.50% | 1.97% | −1.37% | 0.38% | 1.23% | 0.43% |
| 05 | | | | | | | | | | | |

Group01, Group02, Group03, Group04, and Group05 in the above tables 4/5/6 were the above-mentioned Group 1, Group 2, Group 3, Group 4, Group 5, and Group 6. 0/1/2/3/4/7/8/9/10/11/14/15/16/17/18/21/24/28/31/35/38 was the number of days after inoculation.

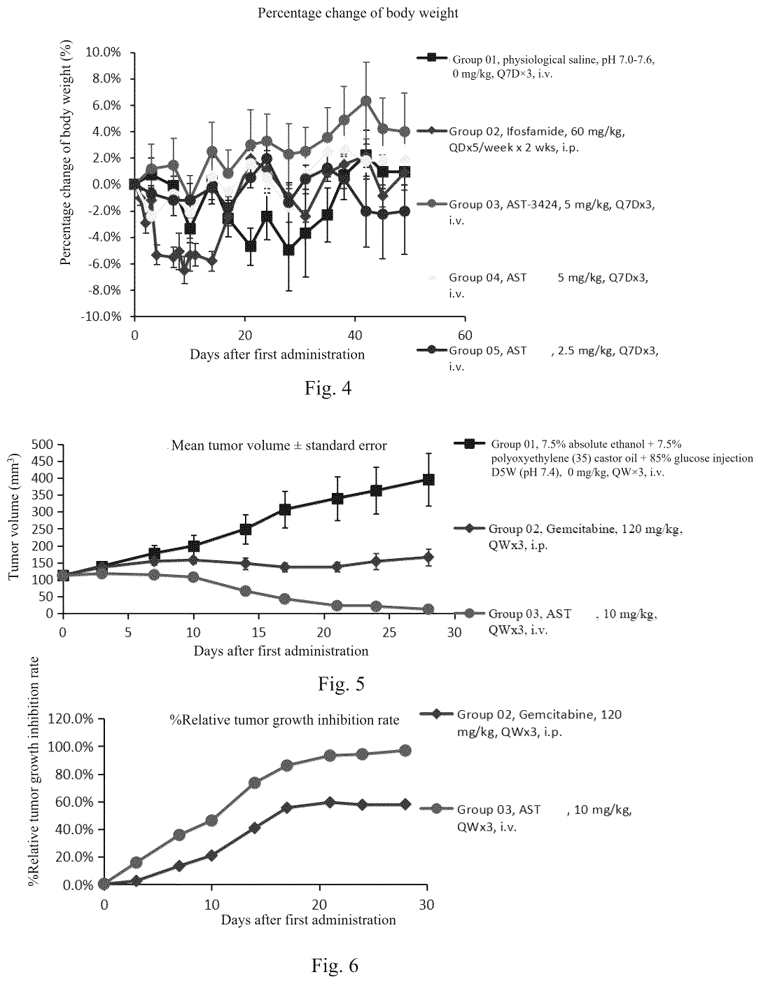

The above table was made into a curve graph to obtain FIG. 4.

It can be known from the analysis of experimental data that in the respect of therapeutic effect:

The test drug AST-3424 at the dose of 5 mg/kg, and the test drug AST at the doses of 2.5 mg/kg and 5 mg/kg, had a significant inhibitory effect on tumor growth of the HuPrime® gastric cancer GA6201, with statistically significant difference compared with the control group.

The test drug Ifosfamide at the dose of 60 mg/kg, had a certain inhibitory effect on tumor growth of HuPrime® gastric cancer GA6201, but no statistically significant difference compared with the control group.

Analysis of the experimental data showed that Ifosfamide, AST-3424, and AST were well tolerated by tumor-bearing mice at the tested doses.

### 2. Pharmacodynamics and Safety Evaluation of the Test Substances AST and Gemcitabine in the HuPrime® Pancreatic Cancer PA1222 Subcutaneous Xenograft Model

The HuPrime® pancreatic cancer PA1222 PDX model was a model of KRAS pathogenic mutation having G12D amino acid mutation (KRAS-G12D). BALB/c nude mice were subcutaneously inoculated with the HuPrime® model PA1222 tumor blocks so as to establish a subcutaneous transplantation tumor model of human pancreatic cancer. The test was divided into the test drug Gemcitabine 120 mg/kg group, the test drug AST 10 mg/kg group and 7.5% absolute ethanol+7.5% polyoxyethylene (35) castor oil+85% glucose injection D5W (pH 7.4) vehicle control group, with a total of 3 groups and 5 mice in each group. Among them, the 7.5% absolute ethanol+7.5% polyoxyethylene (35) castor oil+85% glucose injection D5W (pH 7.4) vehicle control group and the test drug AST 10 mg/kg group were administered by tail vein injection once a week for three consecutive weeks. The test drug Gemcitabine 120 mg/kg group was administered through intraperitoneal injection once a week for three consecutive weeks. The therapeutic efficacy was evaluated based on the relative tumor growth inhibition rate (TGI (%)), and the safety was evaluated based on the body weight changes and the death situation of the animals.

The specific administration regimen for each group was shown in Table 7 below.

TABLE 7
Experimental design of anti-tumor effects of test drugs at different doses
in the HuPrime ® pancreatic cancer PA1222 PDX tumor model

| | The | | | | |
|---|---|---|---|---|---|
| | number | | | | |
| | of | | Dosage | Administration | Administration |
| Group No. | Animals | Administration group | (mg/kg) | method | period |
| 1 | 5 | 7.5% absolute ethanol + 7.5% | — | i.v. | QW × 3 |
| | | polyoxyethylene (35) castor oil + | | | |
| | | 85% glucose injection D5W (pH | | | |
| | | 7.4) | | | |
| 2 | 5 | Gemcitabine | 120 | i.p. | QW × 3 |
| 3 | 5 | AST | 10 | i.v. | QW × 3 |

Note:
1. Dosing volume was 10 μl/g.
2. QW × 3; administered once a week for three weeks

The tumor volumes of mice in different groups were measured on different days and the mean values were obtained. The results were shown in Table 8 below.

TABLE 8
Changes in tumor volume in each group of mice over treatment time
in the HuPrime ® pancreatic cancer PA1222 model

| | Tumor volume(mm³) (X̅ ± S) | | |
|---|---|---|---|
| | Group 1 | | |
| | 7.5% absolute ethanol + 7.5% | | |
| | polyoxyethylene (35) castor oil + | Group 2 | Group 3 |
| | 85% glucose injection D5W (pH | Gemcitabine, 120 | AST, 10 mg/kg, |
| Time | 7.4), 0 mg/kg, QW × 3, i.v. | mg/kg, QW × 3, i.p. | QW × 3, i.v. |
| 0 day after initial | 112.01 ± 7.53 | 111.91 ± 7.78 | 111.50 ± 8.63 |
| administration | | | |
| 3 days after initial | 139.75 ± 12.85 | 136.17 ± 12.64 | 117.62 ± 6.91 |
| administration | | | |
| 7 days after initial | 177.43 ± 24.82 | 153.75 ± 10.18 | 113.91 ± 10.61 |
| administration | | | |
| 10 days after initial | 199.20 ± 31.63 | 157.55 ± 9.68 | 107.17 ± 11.77 |
| administration | | | |
| 14 days after initial | 249.21 ± 43.56 | 147.44 ± 16.31 | 66.08 ± 4.54 |
| administration | | | |
| 17 days after initial | 306.79 ± 53.54 | 136.71 ± 13.80 | 42.94 ± 2.17 |
| administration | | | |
| 21 days after initial | 339.57 ± 63.97 | 138.01 ± 14.81 | 23.22 ± 2.75 |
| administration | | | |
| 24 days after initial | 362.58 ± 69.17 | 153.69 ± 22.96 | 21.20 ± 2.38 |
| administration | | | |
| 28 days after initial | 395.45 ± 76.89 | 166.11 ± 24.40 | 12.51 ± 5.25 |
| administration | | | |

The tumor growth of each treatment group and control group was shown in Table 8 and FIG. 5, and the drug efficacy evaluation was shown in Table 9.

TABLE 9
Drug efficacy analysis table of each group in the HuPrime ® gastric cancer GA6201 model

| | Day 28 after first administration (i.e., Day 28, Dec. 29, 2020) | | | | |
|---|---|---|---|---|---|
| | | Relative | | | P Value |
| | Tumor volume | tumor volume | TGI | T/C | (compared with the |
| Experiment group | (X̅ ± S) | (X̅ ± S) | (%) | (%) | control group) |
| Group 1 | 395.45 ± 76.89 | 3.49 ± 0.62 | — | — | — |
| 7.5% absolute ethanol + | | | | | |
| 7.5% polyoxyethylene (35) | | | | | |
| castor oil + 85% glucose | | | | | |
| injection D5W (pH 7.4), | | | | | |
| 0 mg/kg, QW × 3, i.v. | | | | | |
| Group 2 | 166.11 ± 24.40 | 1.49 ± 0.19 | 57.17% | 42.83% | 0.000778 |
| Gemcitabine, 120 mg/kg, | | | | | |
| QW × 3, i.p. | | | | | |
| Group 3 | 12.51 ± 5.25 | 0.10 ± 0.04 | 97.14% | 2.86% | 0.000000914 |
| AST, 10 mg/kg, QW × 3, i.v. | | | | | |

The relative tumor proliferation rate (T/C %) in Table 9 was the percentage of relative tumor volume or tumor weight between the treatment group and the control group at a certain time point. The calculation formula was as follows:
T/C %=TRTV/CRTV×100% (TRTV: the mean RTV of the treatment group; CRTV: the mean RTV of the vehicle control group; RTV=Vt/V0, V0 was the tumor volume of the animal when grouped, Vt was the tumor volume of the animal after treatment);
The relative tumor growth inhibition rate (TGI (%)) was calculated as follows: TGI %=(1−T/C)×100%. (T and C were the mean relative tumor volumes (RTV) of the treatment group and the control group at a specific time point, respectively).

TABLE 10
Relative tumor growth inhibition rate of each group of
tumors in the HuPrime pancreatic cancer PA1222 model

| | Dates/Study Days | | | | | | | | |
|---|---|---|---|---|---|---|---|---|---|
| | Dec. 1, | Dec. 4, | Dec. 8, | Dec. 11, | Dec. 15, | Dec. 18, | Dec. 22, | Dec. 25, | Dec. 29, |
| | 2020 | 2020 | 2020 | 2020 | 2020 | 2020 | 2020 | 2020 | 2020 |
| Group | 0 | 3 | 7 | 10 | 14 | 17 | 21 | 24 | 28 |
| Group 01 | | | | | | | | | |
| Group 02 | 0.09% | 2.56% | 13.35% | 20.91% | 40.84% | 55.44% | 59.36% | 57.61% | 57.99% |
| Group 03 | 0.45% | 15.84% | 35.80% | 46.20% | 73.48% | 86.00% | 93.16% | 94.15% | 96.84% |

The above table was made into a curve graph to obtain FIG. 6.

The body weights of mice in different groups were measured on different days and the mean values were obtained. The results were shown in Table 11 below.

TABLE 11
Body weight of mice on different inoculation days in the
HuPrime ® pancreatic cancer PA1222 model

| Group | 0 | 3 | 7 | 10 | 14 | 17 | 21 | 24 | 28 |
|---|---|---|---|---|---|---|---|---|---|
| Group | 24.0 | 24.1 | 24.6 | 24.6 | 24.4 | 24.3 | 25.1 | 24.8 | 25.0 |
| 01 | | | | | | | | | |
| Group | 24.1 | 23.9 | 24.4 | 24.3 | 24.6 | 24.2 | 25.1 | 25.1 | 25.4 |
| 02 | | | | | | | | | |
| Group | 24.0 | 23.5 | 24.0 | 23.6 | 23.6 | 23.6 | 24.0 | 24.0 | 24.1 |
| 03 | | | | | | | | | |

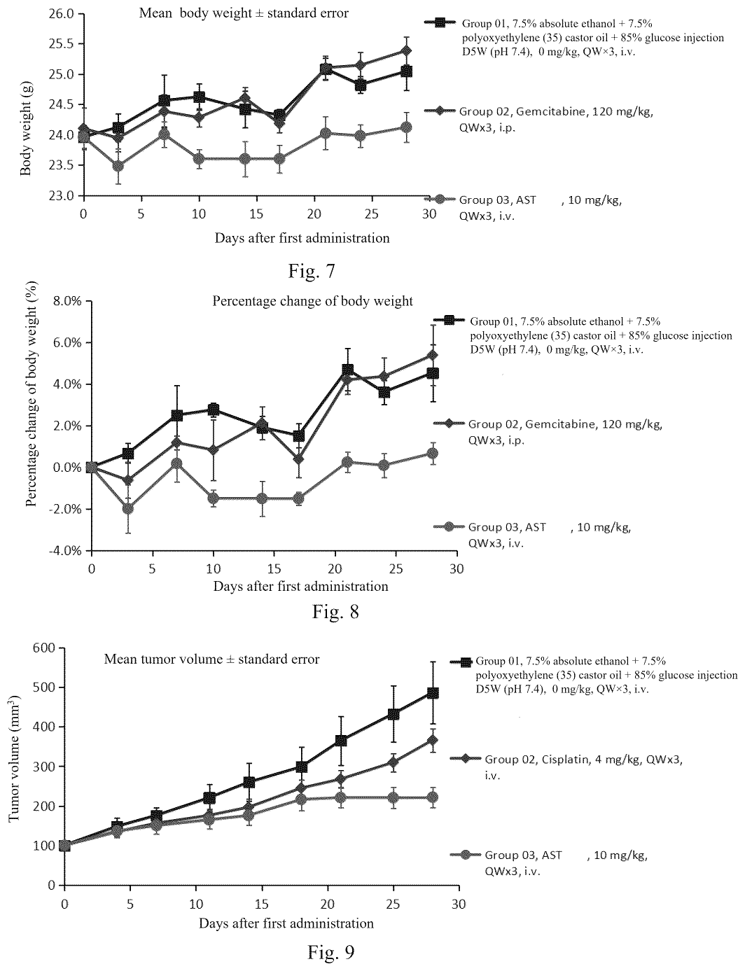

The above table was made into a curve graph to obtain the FIG. 7, which was the body weight curve in each group of mice in the HuPrime® pancreatic cancer PA1222 model.

Similarly, the data in Table 11 was processed to obtain the following Table 12.

TABLE 12
Percentage change in body weight of mice on different inoculation days in the
HuPrime ® pancreatic cancer PA1222 model (% Group mean Change =
mean((T − T0)/T0)*100, T represented current value, T0 represented initial value)

| Group | 0 | 3 | 7 | 10 | 14 | 17 | 21 | 24 | 28 |
|---|---|---|---|---|---|---|---|---|---|
| Group 01 | 0.00% | 0.67% | 2.49% | 2.75% | 1.91% | 1.52% | 4.70% | 3.60% | 4.52% |
| Group 02 | 0.00% | −0.63% | 1.18% | 0.82% | 2.12% | 0.38% | 4.19% | 4.36% | 5.38% |
| Group 03 | 0.00% | −1.99% | 0.18% | −1.50% | −1.50% | −1.51% | 0.24% | 0.09% | 0.66% |

The above table was made into a curve graph to obtain FIG. 8.

It can be known from the analysis of experimental data that in the respect of therapeutic effect:

The test drug Gemcitabine at the dose of 120 mg/kg (Group 2) had a certain inhibitory effect on tumor growth of HuPrime® pancreatic cancer PA1222, with statistically significant difference compared with the control group. The test drug AST at the dose of 10 mg/kg (Group 3) had a significant inhibitory effect on tumor growth of HuPrime® pancreatic cancer PA1222, with statistically significant difference compared with the control group, and two mice in this group had tumors cured, wherein the cure rate both was 40%. The tumor inhibitory effect of the test drug AST 10 mg/kg (Group 3) was significantly better than that of the test drug Gemcitabine (120 mg/kg, Group 2) (p=0.000778).

Analysis of experimental data showed that the mice in the test drug Gemcitabine (120 mg/kg, Group 2) treatment group, AST 10 mg/kg (Group 3) treatment group and control group (Group 1) did not have any obvious weight loss and were well tolerated during the treatment period.

### 3. Pharmacodynamics and Safety Evaluation of the Test Substances AST and Cisplatin in the HuPrime® Lung Cancer LU11693 Subcutaneous Xenograft Model

The HuPrime® lung cancer LU11693 PDX model was a model of KRAS pathogenic mutation having G12C amino acid mutation. BALB/c nude mice were subcutaneously inoculated with HuPrime® model LU11693 tumor blocks so as to establish a subcutaneous transplantation tumor model of human lung cancer. The test was divided into the test drug Cisplatin 4 mg/kg group, the test drug AST 10 mg/kg group and the 7.5% absolute ethanol+7.5% polyoxyethylene (35) castor oil+85% glucose injection D5W (pH 7.4) vehicle control group, with a total of 3 groups and 6 mice in each group. The mice in each group were administered by tail vein injection once a week for three consecutive weeks. The therapeutic efficacy was evaluated based on the relative tumor growth inhibition rate (TGI (%)), and the safety was evaluated based on the body weight changes and the death situation of the animals.

The specific administration regimen for each group was shown in Table 13 below.

TABLE 13
Experimental design of anti-tumor effects of test drugs at different doses
in the HuPrime ® lung cancer LU11693 PDX tumor model

| | The | | | | |
|---|---|---|---|---|---|
| | number | | | | |
| Group | of | | Dosage | Administration | Administration |
| No. | Animals | Administration group | (mg/kg) | method | period |
| 1 | 6 | 7.5% absolute ethanol + 7.5% | 0 | Tail vein | QW × 3 |
| | | polyoxyethylene (35) castor oil + 85% | | | |
| | | glucose injection D5W (pH 7.4) | | | |
| 2 | 6 | Cisplatin | 4 | Tail vein | QW × 3 |
| 3 | 6 | AST | 10 | Tail vein | QW × 3 |

Note:
1. Dosing volume was 10 μl/g
2. QW × 3; administered once a week for three weeks

The tumor volumes of mice in different groups were measured on different days and the mean values were obtained. The results were shown in Table 14 below.

TABLE 14
Changes in tumor volume in each group of mice over treatment
time in the HuPrime ® lung cancer LU11693 model

| | Tumor volume(mm³) (X̅ ± S) | | |
|---|---|---|---|
| | Group 1 | | |
| | 7.5% absolute ethanol + 7.5% | | |
| | polyoxyethylene (35) castor | Group 2 | Group 3 |
| | oil + 85% glucose injection | Cisplatin | AST |
| Time | D5W (pH 7.4) | 4 mg/kg | 10 mg/kg |
| 0 day after initial | 100.73 ± 6.73 | 100.12 ± 6.13 | 100.52 ± 7.48 |
| administration | | | |
| 4 days after initial | 148.49 ± 20.70 | 134.73 ± 12.15 | 136.39 ± 17.11 |
| administration | | | |
| 7 days after initial | 176.25 ± 19.10 | 156.35 ± 14.10 | 150.25 ± 21.20 |
| administration | | | |
| 11 days after initial | 220.66 ± 33.68 | 176.12 ± 15.18 | 164.92 ± 23.46 |
| administration | | | |
| 14 days after initial | 259.63 ± 48.15 | 196.54 ± 20.83 | 175.66 ± 24.12 |
| administration | | | |
| 18 days after initial | 298.53 ± 50.33 | 244.94 ± 21.34 | 216.17 ± 27.04 |
| administration | | | |
| 21 days after initial | 364.53 ± 61.94 | 267.58 ± 22.10 | 221.04 ± 25.74 |
| administration | | | |
| 25 days after initial | 432.25 ± 71.05 | 309.77 ± 23.28 | 220.53 ± 26.00 |
| administration | | | |
| 28 days after initial | 485.88 ± 78.14 | 365.88 ± 29.58 | 221.93 ± 25.52 |
| administration | | | |

The tumor growth of each treatment group and control group was shown in Table 14 and FIG. 9, and the drug efficacy evaluation was shown in Table 15.

TABLE 15
Drug efficacy analysis table of each group in the HuPrime ® lung cancer LU11693 model

| | Day 28 after first administration (i.e., Day 28, Jan. 28, 2021) | | | | |
|---|---|---|---|---|---|
| | | | | | P Value |
| | Tumor volume | Relative tumor | | | (compared with |
| Experiment group | (X̅ ± S) | volume (X̅ ± S) | TGI (%) | T/C (%) | the control group) |
| Group 1 | 485.88 ± 78.14 | 4.79 ± 0.58 | — | — | — |
| 7.5% absolute ethanol + | | | | | |
| 7.5% polyoxyethylene | | | | | |
| (35) castor oil + 85% | | | | | |
| glucose injection D5W | | | | | |
| (pH 7.4) | | | | | |
| Group 2 | 365.88 ± 29.58 | 3.64 ± 0.14 | 23.98 | 76.02 | 0.0152 |
| Cisplatin | | | | | |
| 4 mg/kg | | | | | |
| Group 3 | 221.93 ± 25.52 | 2.17 ± 0.11 | 54.64 | 45.36 | 0.00000337 |
| AST | | | | | |
| 10 mg/kg | | | | | |

The relative tumor proliferation rate (T/C %) in Table 15 was the percentage of relative tumor volume or tumor weight between the treatment group and the control group at a certain time point. The calculation formula was as follows:
T/C %=TRTV/CRTV×100% (TRTV: the mean RTV of the treatment group; CRTV: the mean RTV of the vehicle control group; RTV=Vt/V0, V0 was the tumor volume of the animal when grouped, Vt was the tumor volume of the animal after treatment);
The relative tumor growth inhibition rate (TGI (%)) was calculated as follows: TGI %=(1−T/C)×100%. (T and C were the mean relative tumor volumes (RTV) of the treatment group and the control group at a specific time point, respectively).

TABLE 16
Relative tumor growth inhibition rate of each group of tumors
in the HuPrime ® lung cancer LU11693 model

| | Dates/Study Days | | | | | | | | |
|---|---|---|---|---|---|---|---|---|---|
| | Dec. 31, | Jan. 4, | Jan. 7, | Jan. 11, | Jan. 14, | Jan. 18, | Jan. 21, | Jan. 25, | Jan. 28, |
| | 2020 | 2021 | 2021 | 2021 | 2021 | 2021 | 2021 | 2021 | 2021 |
| Group | 0 | 4 | 7 | 11 | 14 | 18 | 21 | 25 | 28 |
| Group 01 | | | | | | | | | |
| Group 02 | 0.61% | 9.27% | 11.29% | 20.19% | 24.30% | 17.95% | 26.60% | 28.34% | 24.70% |
| Group 03 | 0.21% | 8.15% | 14.75% | 25.26% | 32.34% | 27.59% | 39.36% | 48.98% | 54.32% |

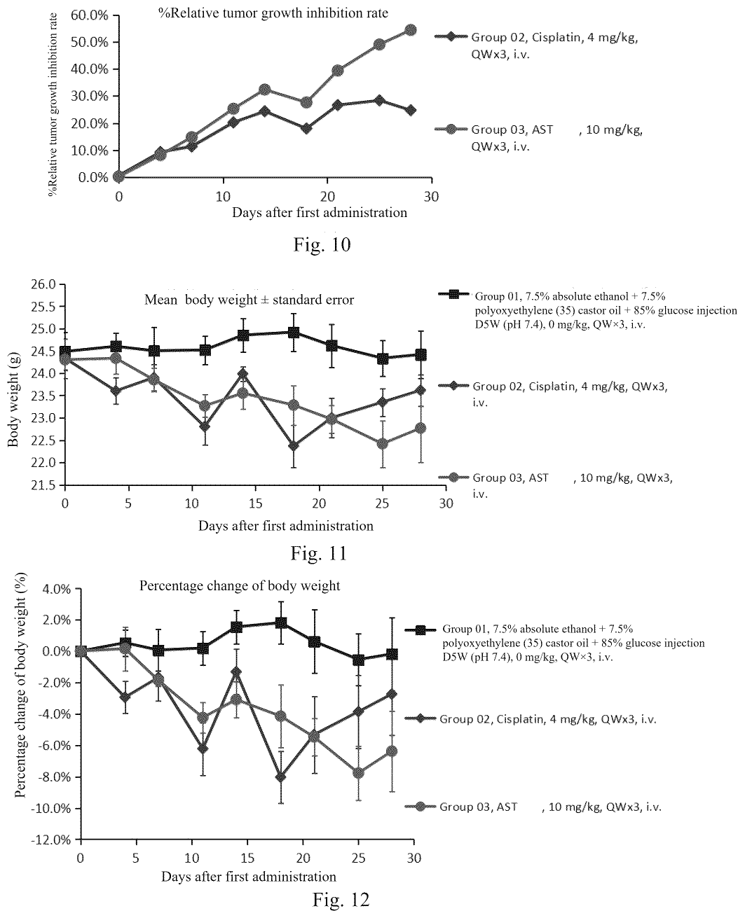

The above table was made into a curve graph to obtain FIG. 10.

The body weights of mice in different groups were measured on different days and the mean values were obtained. The results were shown in Table 17 below.

TABLE 17
Body weight of mice on different inoculation days in
the HuPrime ® lung cancer LU11693 model

| Group | 0 | 4 | 7 | 11 | 14 | 18 | 21 | 25 | 28 |
|---|---|---|---|---|---|---|---|---|---|
| Group | 24.5 | 24.6 | 24.5 | 24.5 | 24.9 | 24.9 | 24.6 | 24.3 | 24.4 |
| 01 | | | | | | | | | |
| Group | 24.3 | 23.6 | 23.9 | 22.8 | 24.0 | 22.4 | 23.0 | 23.4 | 23.6 |
| 02 | | | | | | | | | |
| Group | 24.3 | 24.3 | 23.9 | 23.3 | 23.6 | 23.3 | 23.0 | 22.4 | 22.8 |
| 03 | | | | | | | | | |

The above table was made into a curve graph to obtain FIG. 11, which was the body weight curve in each group of mice in the HuPrime® lung cancer LU11693 model.

Similarly, the data in Table 17 was processed to obtain the following Table 18.

TABLE 18
Percentage change in body weight of mice on different inoculation days in
the HuPrime ® lung cancer LU11693 model (% Group Mean Change =
mean((T − T0)/T0)*100, T represented current value, T0 represented initial value)

| Group | 0 | 4 | 7 | 11 | 14 | 18 | 21 | 25 | 28 |
|---|---|---|---|---|---|---|---|---|---|
| Group 01 | 0.00% | 0.53% | 0.07% | 0.20% | 1.54% | 1.81% | 0.63% | −0.54% | −0.18% |
| Group 02 | 0.00% | −2.95% | −1.68% | −6.23% | −1.32% | −8.04% | −5.33% | −3.86% | −2.74% |
| Group 03 | 0.00% | 0.15% | −1.86% | −4.24% | −3.09% | −4.15% | −5.47% | −7.78% | −6.38% |

The above table was made into a curve graph to obtain FIG. 12.

It can be known from the analysis of experimental data that in the respect of therapeutic effect:

The test drug Cisplatin (4 mg/kg) treatment group showed a certain tumor inhibitory effect on Day 28 after first administration, with a statistically significant difference compared with the control group (p=0.0152), and the relative tumor growth inhibition rate TGI (%) was 23.98%.

The test drug AST (10 mg/kg) treatment group showed a certain tumor inhibitory effect on Day 28 after first administration, with a statistically significant difference compared with the control group (p<0.001), and the relative tumor growth inhibition rate TGI (%) was 54.64%, while the TGI was less than 60% with no obvious tumor inhibitory effect.

Analysis of the experimental data showed that some mice in the test drug AST (10 mg/kg) and Cisplatin (4 mg/kg) treatment groups displayed severe weight loss, which might be related to the potential toxicity of high-dose drugs.

From the above three sets of experimental data, we can draw the following conclusions:
1. AST-3424 and AST both had significant drug efficacy in the KRAS pathogenic mutation models having G12D amino acid mutation: gastric cancer GA6021 and pancreatic cancer PA1222 PDX model, with TGI % greater than 90%;
2. The tumor inhibitory effect of AST in the KRAS pathogenic mutation model having G12C amino acid mutation: lung cancer LU11693 was not obvious, with TGI % less than 60%;
3. Both AST-3424 and AST had relatively good tolerance in each model.

The inventor further found through research that in the KRAS pathogenic mutation model having G12D amino acid mutation, AST-3424 and AST had generally significant therapeutic effects on a variety of cancer indications. In the KRAS pathogenic mutation model having G12C amino acid mutation, the therapeutic effect of AST was not only related to the cancer type, but also related to other factors.

### 4. Anti-Tumor Effect and Safety Evaluation of the Test Substances AST, AST-3424 and Ifosfamide in Human-Derived Pancreatic Cancer HPAF-1H Subcutaneous Xenograft Model

The human-derived pancreatic cancer HPAF-II subcutaneous xenograft model was a CDX model harboring the KRAS G12D pathogenic mutation.

Balb/c nude female mice were subcutaneously inoculated with human-derived pancreatic cancer HPAF-II cells so as to establish a subcutaneous transplantation model of human-derived pancreatic cancer. The test was divided into the following groups: the test drug treatment group Ifosfamide 60 mg/kg monotherapy group (Group 2) was administered intraperitoneally once a day for 5 consecutive days, rested for 2 days, and then administered once a day for 5 consecutive days again; AST 4 mg/kg monotherapy group (Group 3) was administered through the tail vein once a day for 5 consecutive days, rested for 2 days, then rested for 2 weeks, and then administered once a day for 5 consecutive days again; AST 8 mg/kg monotherapy group (Group 4) was administered through the tail vein once a week for a total of 3 weeks; AST-3424 1 mg/kg monotherapy group (Group 5) was administered through the tail vein once a day for 5 consecutive days, rested for 2 days, then rested for 2 weeks, and then administered once a day for 5 consecutive days again; and glucose injection (pH 7.7-8.0) vehicle control group (Group 1) was administered through the tail vein once a day for 5 consecutive days, rested for 2 days, then rested for 2 weeks, and then administered once a day for 5 consecutive days again. There were 5 groups in total and 6 mice in each group in this study. The administration routes, dosages and regimens of the experimental design were shown in Table 19.

TABLE 19
Administration routes, dosages and regimens in human-
derived pancreatic cancer HPAF-II animal model

| | The | | | | |
|---|---|---|---|---|---|
| | number | | | | |
| Group | of | Administration | Dosage | Administration | |
| No. | Animals | group | (mg/kg) | method | Administration period |
| 1 | 6 | glucose injection | — | i.v. | QD × 5, 2 days off, 2 weeks off, QD × 5 |
| | | (pH 7.7-8.0) | | | |
| 2 | 6 | Ifosfamide | 60 | i.p. | QD × 5/week × 2 weeks |
| 3 | 6 | AST | 4 | i.v. | QD × 5, 2 days off, 2 weeks off, QD × 5 |
| 4 | 6 | AST | 8 | i.v. | QW × 3 |
| 5 | 6 | AST-3424 | 1 | i.v. | QD × 5, 2 days off, 2 weeks off, QD × 5 |

In the present application, with respect to the abbreviations related to administration route, i.v. represented tail vein injection, i.p. represented intraperitoneal injection; with respect to the abbreviations related to the dosing cycle, QW represented once a week, QD represented once a day, “QD*5, 2 days off, 2 weeks off, QD*5” represented administering once a day for 5 consecutive days, resting for 2 days, then resting for 2 weeks, and then administering once a day for 5 consecutive days again.

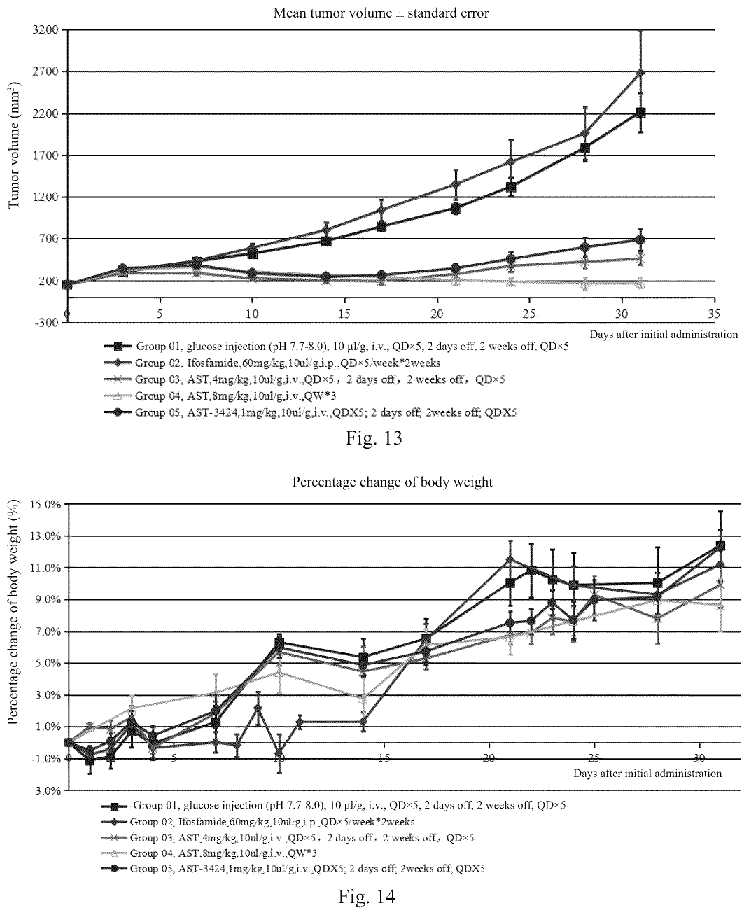

The tumor growth of each treatment group and control group was recorded on different days of the test, as shown in Table 20. The corresponding growth curve of tumor volume in each group of mice was shown in FIG. 13. The therapeutic effect was evaluated based on the relative tumor proliferation rate and relative tumor growth inhibition rate. The drug efficacy analysis of each group was shown in Table 21. The body weight changes of the treatment group and the control group after administration were recorded, and the safety of each group in the human-derived pancreatic cancer HPAF-II subcutaneous xenograft model was studied. The results of body weight changes of mice were shown in Table 22. The curve graph of the percentage change of body weight in each treatment group over time was shown in FIG. 14.

TABLE 20
Changes in tumor volume in each group of mice over treatment time in
the human-derived pancreatic cancer HPAF-II subcutaneous model (mm³)

| Group | Days after inoculation | | | | | | | | | |
|---|---|---|---|---|---|---|---|---|---|---|
| No. | 0 | 3 | 7 | 10 | 14 | 17 | 21 | 24 | 28 | 31 |
| Group 1 | 148.71 | 298.82 | 427.94 | 521.32 | 669.76 | 844.44 | 1064.44 | 1321.14 | 1787.35 | 2212.64 |
| Group 2 | 148.64 | 313.53 | 436.26 | 588.61 | 801.41 | 1041.60 | 1347.90 | 1617.30 | 1959.58 | 2678.00 |
| Group 3 | 148.81 | 286.38 | 287.09 | 223.40 | 199.45 | 189.54 | 274.92 | 373.75 | 422.27 | 457.66 |
| Group 4 | 148.63 | 320.80 | 364.88 | 306.82 | 262.16 | 241.53 | 200.54 | 188.10 | 163.95 | 170.65 |
| Group 5 | 149.44 | 344.77 | 382.62 | 285.47 | 243.00 | 263.64 | 344.86 | 457.08 | 596.76 | 685.85 |

TABLE 21
Drug efficacy analysis table of each group in the human-
derived pancreatic cancer HPAF-II subcutaneous model

| | Day 31 after grouping | | | | |
|---|---|---|---|---|---|
| | | Relative | | | P Value |
| | | tumor | | | (comparing with |
| | Tumor volume | volume | | | the control |
| Experiment group | (X̅ ± S) | (X̅ ± S) | TGI (%) | T/C (%) | group) |
| Group 1 | 2212.64 ± 232.88 | 15.17 ± 1.64 | — | — | — |
| Glucose injection (pH 7.7- | | | | | |
| 8.0), 10 μL/g, i.v., QD × 5, 2 | | | | | |
| days off, 2 weeks off, QD × 5 | | | | | |
| Group 2 | 2678.00 ± 517.46 | 17.38 ± 2.69 | −14.56 | 114.56 | >0.05 |
| Ifosfamide, | | | | | |
| 60 mg/kg, 10 μL/g, i.p., | | | | | |
| QD × 5/week × 2 weeks | | | | | |
| Group 3 | 457.66 ± 69.87 | 3.10 ± 0.40 | 79.55 | 20.45 | <0.001** |
| AST, 4 mg/kg, 10 μL/g, i.v., | | | | | |
| QD × 5, 2 days off, 2 weeks | | | | | |
| off, QD × 5 | | | | | |
| Group 4 | 170.65 ± 56.64 | 1.12 ± 0.32 | 92.64 | 7.36 | <0.001** |
| AST, 8 mg/kg, 10 μL/g, i.v., | | | | | |
| QW × 3 | | | | | |
| Group 5 | 685.85 ± 130.78 | 4.63 ± 0.79 | 69.46 | 30.54 | <0.001 |
| AST-3424, 1 mg/kg, 10 μL/g, | | | | | |
| i.v., QD × 5, 2 days off, 2 | | | | | |
| weeks off, QD × 5 | | | | | |

TABLE 22
Changes in body weight of mice in the human-derived pancreatic cancer HPAF-II subcutaneous model

| Group | | | | | | | | | | | | | | | | | | | |
|---|---|---|---|---|---|---|---|---|---|---|---|---|---|---|---|---|---|---|---|
| No. | 0 | 1 | 2 | 3 | 4 | 7 | 8 | 9 | 10 | 11 | 14 | 17 | 21 | 22 | 23 | 24 | 25 | 28 | 31 |
| Group 1 | 23.9 | 23.6 | 23.7 | 24.0 | 23.8 | 24.2 | — | — | 25.4 | — | 25.1 | 25.4 | 26.3 | 26.4 | 26.3 | 26.2 | — | 26.3 | 26.8 |
| Group 2 | 23.3 | 23.1 | 23.2 | 23.5 | 23.2 | 23.3 | 23.2 | 23.8 | 23.1 | 23.6 | 23.6 | 24.8 | 25.9 | — | — | 25.6 | — | 25.4 | 25.9 |
| Group 3 | 23.4 | 23.7 | 23.6 | 23.8 | 23.4 | 23.9 | — | — | 24.8 | — | 24.5 | 24.7 | 25.0 | 25.1 | 25.3 | 25.2 | 25.6 | 25.3 | 25.8 |
| Group 4 | 23.5 | — | — | 24.0 | — | 24.2 | — | — | 24.5 | — | 24.2 | 25.0 | 25.1 | — | — | 25.3 | — | 25.6 | 25.5 |
| Group 5 | 22.8 | 22.7 | 22.8 | 23.1 | 22.9 | 23.3 | — | — | 24.2 | — | 23.9 | 24.1 | 24.5 | 24.6 | 24.8 | 24.6 | 24.9 | 24.9 | 25.6 |

The mean tumor volume of mice in the vehicle control group was 2212.64 mm³ on Day 31 after the initial administration. The mean tumor volume of the test drug Ifosfamide treatment group (Group 2) at the dose of 60 mg/kg on Day 31 was 2678.00 mm³, and the relative tumor growth inhibition rate TGI (%) was −14.65%, with no statistically significant difference compared with the control group (p>0.05).

The mean tumor volumes of the test drug AST treatment groups (Groups 3 and 4) at the dose of 4 mg/kg (QD×5, 2 days off, 2 weeks off, QD×5) and at the dose of 8 mg/kg (QW×3), and AST-3424 group (Group 5) at the dose of 1 mg/kg (QD×5, 2 days off, 2 weeks off, QD×5) on Day 31 were 457.66, 170.65 and 685.85 mm³, respectively, with statistically significant difference compared with the control group (p<0.05), and the relative tumor growth inhibition rates TGI (%) were 79.55%, 92.64% and 69.46%, respectively.

The above experimental results showed that AST-3424 and AST in the model having KRAS G12D pathogenic mutation, AST at the dose of 8 mg/kg (QW×3), at the dose of 4 mg/kg (QD×5, 2 days off, 2 weeks off, QD×5), and AST-3424 at the dose of 1 mg/kg (QD×5, 2 days off, 2 weeks off, QD×5) had a significant anti-tumor effect on the human-derived pancreatic cancer HPAF-II subcutaneous model. Moreover, the mice in each test drug treatment group did not lose weight and were well tolerated during the treatment period.

### 5. Anti-Tumor Effect and Safety Evaluation of the Test Substances AST, AST-3424 and Ifosfamide Monotherapy in the HuPrime® Lung Cancer LU5161 Subcutaneous Model

The HuPrime® lung cancer LU5161 subcutaneous model was a PDX model harboring the KRAS G12D pathogenic mutation.

Balb/nude female mice were subcutaneously inoculated with HuPrime® lung cancer LU5161 tumor blocks so as to establish a subcutaneous transplantation tumor model of human lung cancer. The test was divided into the following groups: the test drug Ifosfamide 60 mg/kg monotherapy group (Group 2) was administered once a day for 5 consecutive days, rested for 2 days, and then administered once a day for 5 consecutive days again; AST 4 mg/kg monotherapy group (Group 3) and AST 8 mg/kg monotherapy group (Group 4) were administered once a week for a total of 3 weeks; AST 4 mg/kg monotherapy group (Group 5) and AST-3424 1 mg/kg monotherapy group (Group 6) was administered once a day for 5 consecutive days, rested for 2 days, then rested for 2 weeks, and then administered once a day for 5 consecutive days again; and glucose injection (pH 7.7-8.0) vehicle control group (Group 1) was administered once a day for 5 consecutive days, rested for 2 days, then rested for 2 weeks, and then administered once a day for 5 consecutive days again. There were 6 groups in total and 6 mice in each group in this study. Among them, the test drug Ifosfamide was administered intraperitoneally, and the vehicle control group, AST, and AST-3424 were all administered by tail vein injection. The administration routes, dosages and regimens of the experimental design were shown in Table 23.

TABLE 23
Administration routes, dosages and regimens in the HuPrime ® lung
cancer LU5161 subcutaneous model

| | The | | | | |
|---|---|---|---|---|---|
| | number | | | | |
| Group | of | Administration | Dosage | Administration | |
| No. | Animals | group | (mg/kg) | method | Administration period |
| 1 | 6 | Glucose | — | i.v. | QD × 5, 2 days off, 2 weeks off, QD × 5 |
| | | injection | | | |
| | | (pH 7.7-8.0) | | | |
| 2 | 6 | Ifosfamide | 60 | i.p. | QD × 5/week × 2 weeks |
| 3 | 6 | AST | 4 | i.v. | QW × 3 |
| 4 | 6 | AST | 8 | i.v. | QW × 3 |
| 5 | 6 | AST | 4 | i.v. | QD × 5, 2 days off, 2 weeks off, QD × 5 |
| 6 | 6 | AST-3424 | 1 | i.v. | QD × 5, 2 days off, 2 weeks off, QD × 5 |

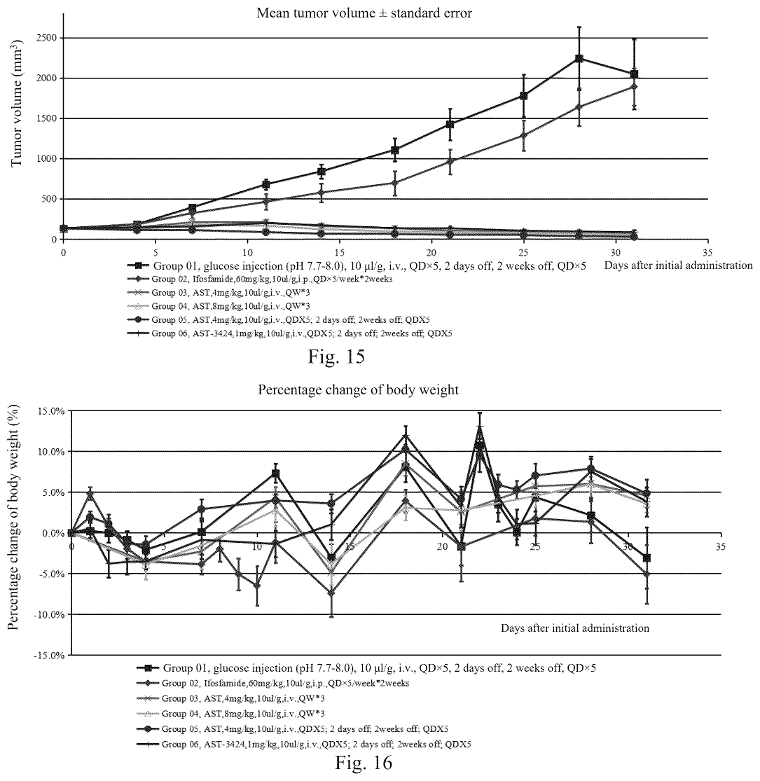

The tumor growth of each treatment group and control group was recorded on different days of the test, as shown in Table 24. The corresponding growth curve of tumor volume in each group of mice was shown in FIG. 15. The therapeutic effect was evaluated based on the relative tumor G proliferation rate and relative tumor growth inhibition rate. The drug efficacy analysis of each group was shown in Table 25. The body weight changes of the treatment group and the control group after administration was recorded, and the safety of each group in the HuPrime® lung cancer LU5161 subcutaneous xenograft model was studied. The results of body weight changes of mice were shown in Table 26. The curve graph of the percentage changes of body weight in each treatment group over time was shown in FIG. 16.

TABLE 24
Changes in tumor volume in each group of mice over treatment time in the
HuPrime ® lung cancer LU5161 subcutaneous model (mm³)

| Group | Days after inoculation | | | | | | | | | |
|---|---|---|---|---|---|---|---|---|---|---|
| No. | 0 | 4 | 7 | 11 | 14 | 18 | 21 | 25 | 28 | 31 |
| 1 | 132.27 | 185.36 | 393.39 | 678.36 | 839.01 | 1106.30 | 1423.45 | 1777.59 | 2238.97 | 2045.61 |
| 2 | 132.21 | 182.89 | 322.31 | 462.34 | 577.36 | 696.39 | 957.80 | 1284.49 | 1636.39 | 1887.60 |
| 3 | 132.11 | 144.28 | 209.41 | 206.30 | 161.45 | 136.83 | 103.27 | 88.21 | 71.72 | 58.51 |
| 4 | 133.19 | 124.17 | 179.20 | 165.66 | 122.07 | 95.27 | 86.00 | 75.80 | 68.18 | 49.88 |
| 5 | 133.15 | 111.35 | 110.86 | 86.51 | 66.85 | 62.20 | 53.86 | 47.74 | 37.14 | 27.78 |
| 6 | 133.17 | 145.58 | 155.12 | 199.04 | 171.71 | 134.63 | 134.55 | 103.78 | 94.42 | 85.23 |

TABLE 25
Drug efficacy analysis table of each group in the HuPrime ® lung
cancer LU5161 subcutaneous model

| | Day 28 | | | | | |
|---|---|---|---|---|---|---|
| | | Relative | | | P Value | Tumor |
| | | tumor | | | (compared | clearance |
| | Tumor volume | volume | TGI | T/C | with the | rate |
| Experiment group | (X̅ ± S) | (X̅ ± S) | (%) | (%) | control group) | Day 31 |
| Group 1 | 2238.97 ± 391.43 | 17.05 ± 3.10 | — | — | — | 0/6 |
| Glucose injection (pH | | | | | | |
| 7.7-8.0), 10 μL/g, i.v., | | | | | | |
| QD × 5, 2 days off, 2 | | | | | | |
| weeks off, QD × 5 | | | | | | |
| Group 2 | 1636.39 ± 234.40 | 12.38 ± 1.21 | 27.39 | 72.61 | >0.05 | 0/6 |
| Ifosfamide, 60 mg/kg, | | | | | | |
| 10 μL/g, i.p., | | | | | | |
| QD × 5/week × 2 weeks | | | | | | |
| Group 3 | 71.72 ± 21.03 | 0.53 ± 0.16 | 96.92 | 3.08 | <0.001*** | 1/6 |
| AST, 4 mg/kg, 10 | | | | | | |
| μL/g, i.v., QW × 3 | | | | | | |
| Group 4 | 68.18 ± 18.97 | 0.50 ± 0.11 | 97.05 | 2.95 | <0.001*** | 1/6 |
| AST, 8 mg/kg, 10 | | | | | | |
| μL/g, i.v., QW × 3 | | | | | | |
| Group 5 | 37.14 ± 6.33 | 0.28 ± 0.04 | 98.38 | 1.62 | <0.001*** | 1/6 |
| AST, 4 mg/kg, 10 μL/g, | | | | | | |
| i.v., QD × 5, 2 days | | | | | | |
| off, 2 weeks off, QD × 5 | | | | | | |
| Group 6 | 94.42 ± 23.39 | 0.75 ± 0.21 | 95.62 | 4.38 | <0.001*** | 1/6 |
| AST-3424, 1 mg/kg, 10 | | | | | | |
| μL/g, i.v., QD × 5, 2 | | | | | | |
| days off, 2 weeks off, | | | | | | |
| QD × 5 | | | | | | |

TABLE 26
Percentage change in body weight of mice in the HuPrime ® lung
cancer LU5161 subcutaneous model

| Group | Days after administration | | | | | | | |
|---|---|---|---|---|---|---|---|---|
| No. | 0 | 1 | 2 | 3 | 4 | 7 | 11 | 14 |
| 1 | 0.00% | 0.24% | −0.03% | −0.88% | −2.09% | 0.11% | 7.31% | −3.03% |
| 2 | 0.00% | 4.81% | 0.76% | −1.96% | −3.53% | −3.86% | −1.15% | −7.43% |
| 3 | 0.00% | — | — | — | −3.51% | −2.32% | 4.33% | −4.89% |
| 4 | 0.00% | — | — | — | −3.88% | −1.58% | 2.80% | −3.82% |
| 5 | 0.00% | 1.92% | 1.12% | −1.35% | −1.45% | 2.90% | 3.96% | 3.59% |
| 6 | 0.00% | 0.49% | −3.79% | −3.57% | −3.51% | −0.88% | −1.37% | 1.02% |

| | |
|---|---|
| Group | Days after administration |

| No. | 18 | 21 | 22 | 23 | 24 | 25 | 28 | 31 |
|---|---|---|---|---|---|---|---|---|
| 1 | 8.10% | −1.66% | 10.74% | 3.54% | 0.09% | 4.36% | 2.14% | −3.04% |
| 2 | 3.92% | −1.71% | — | — | — | 1.76% | 1.34% | −5.07% |
| 3 | 8.46% | 2.57% | — | — | — | 5.72% | 5.99% | 4.57% |
| 4 | 3.09% | 2.73% | — | — | — | 4.54% | 5.95% | 3.58% |
| 5 | 10.22% | 4.17% | 9.52% | 5.89% | 5.29% | 7.03% | 7.88% | 4.83% |
| 6 | 12.01% | 3.21% | 13.16% | 4.38% | 1.05% | 1.17% | 7.51% | 3.83% |

The mean tumor volume of mice in the vehicle control group was 2238.97 mm³ on Day 28 after the initial administration. The mean tumor volume of the test drug Ifosfamide treatment group (Group 2) at the dose of 60 mg/kg on Day 28 was 1636.39 mm³, and the relative tumor growth inhibition rate TGI (%) was 27.39%, with no statistically significant difference compared with the control group (p>0.05).

The mean tumor volumes of the test drug AST treatment groups at the dose of 4 mg/kg (QW×3, Group 3), at the dose of 8 mg/kg (QW×3, Group 4), at the dose of 4 mg/kg (QD×5, 2 days off, 2 weeks off, QD×5, Group 5), and AST-3424 group (Group 6) at the dose of 1 mg/kg (QD×5, 2 days off, 2 weeks off, QD×5) on Day 28 were 71.72 mm³, 68.18 mm³, 37.14 mm³ and 94.42 mm³, respectively, with a statistically significant difference compared with the control group (p<0.001), and the relative tumor growth inhibition rates TGI (%) were 96.92%, 97.05%, 98.38% and 95.62%, respectively. Each one mouse in all treatment groups of test drugs AST and AST-3424 had tumors cleared, with a clearance rate of 16.7%.

The above experimental results showed that AST-3424 and AST in the model having KRAS G12D pathogenic mutation, AST treatment groups (Group 3, Group 4 and Group 5) at the dose of 4 mg/kg (QW×3), at the dose of 8 mg/kg (QW×3), at the dose of 4 mg/kg (QD×5, 2 days off, 2 weeks off, QD×5), and AST-3424 group (Group 6) at the dose of 1 mg/kg (QD×5, 2 days off, 2 weeks off, QD×5) had a significant anti-tumor effect on the HuPrime® lung cancer LU5161 subcutaneous model under the tested dosage and dosing frequency of this study. Ifosfamide administration group had no tumor inhibitory effect. The mice in each test drug treatment group were well tolerated during the treatment period.

### 6. Anti-Tumor Effect and Safety Evaluation of the Test Substances AST and Ifosfamide Monotherapy in the HuPrime® Intestinal Cancer CR3820 Subcutaneous Model

The HuPrime® intestinal cancer CR3820 subcutaneous model was a PDX model harboring the KRAS G12D pathogenic mutation.

NOD.SCID female mice were subcutaneously inoculated with HuPrime® intestinal cancer CR3820 tumor blocks so as to establish a subcutaneous transplantation tumor model of human intestinal cancer. The test was divided into the following groups: the test drug Ifosfamide 60 mg/kg monotherapy group (QD×5/week×2 weeks, Group 2) was administered intraperitoneally once a day for 5 consecutive days, rested for 2 days, and then administrated once a day for 5 consecutive days again; AST 8 mg/kg monotherapy group (QW×3, Group 3) was administered through the tail vein once a week for a total of 3 weeks; AST 4 mg/kg monotherapy group (QD×5, 2 days off, 2 weeks off, QD×5, Group 4) and the vehicle control group glucose injection (pH 7.7-8.0, Group 1) both were administered through the tail vein, and both had the same dosing cycle: administering once a day for 5 consecutive days, resting for 2 days, then resting for 2 weeks, and then administering once a day for 5 consecutive days again. There are 4 groups in total and 6 mice in each group in this experiment. The administration routes, dosages and regimens of the experimental design were shown in Table 27.

TABLE 27
Administration routes, dosages and regimens in the HuPrime ® intestinal
cancer CR3820 subcutaneous model

| | The | | | | |
|---|---|---|---|---|---|
| | number | | | | |
| Group | of | Administration | Dosage | Administration | |
| No. | Animals | group | (mg/kg) | method | Administration period |
| 1 | 6 | Glucose injection | — | i.v. | QD × 5, 2 days off, 2 weeks off, QD × 5 |
| | | (pH 7.7-8.0) | | | QD × 5/week × 2 weeks |
| 2 | 6 | Ifosfamide | 60 | i.p. | |
| 3 | 6 | AST | 8 | i.v. | QW × 3 |
| 4 | 6 | AST | 4 | i.v. | QD × 5, 2 days off, 2 weeks off, QD × 5 |

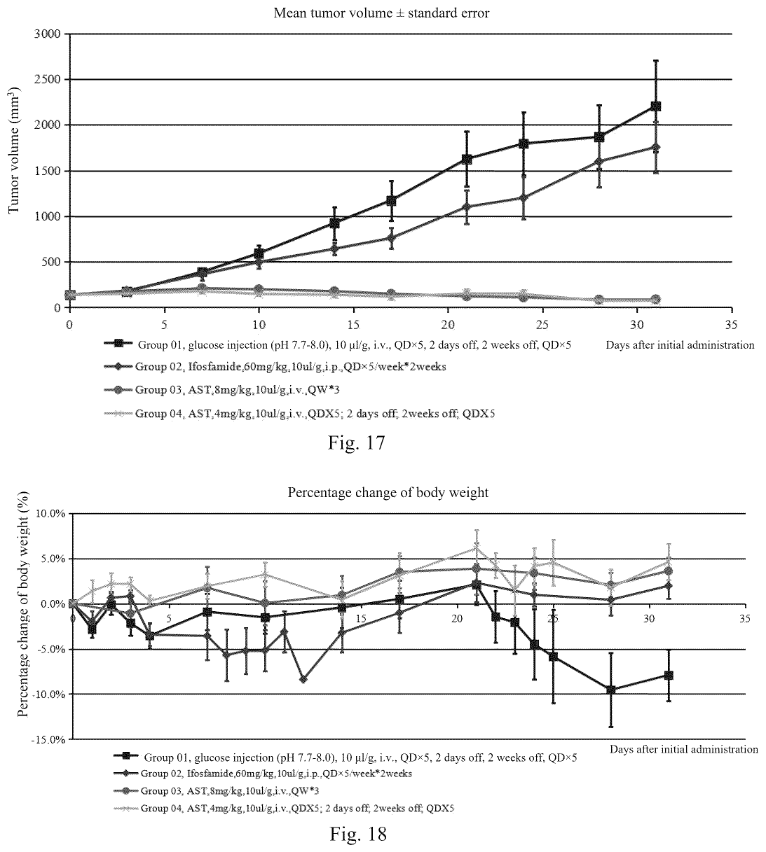

The tumor growth of each treatment group and control group was recorded on different days of the test, as shown in Table 28. The corresponding growth curve of tumor volume in each group of mice was shown in FIG. 17. The therapeutic effect was evaluated based on the relative tumor proliferation rate and relative tumor growth inhibition rate. The drug efficacy analysis of each group was shown in Table 29. The body weight changes of the treatment group and the control group after administration were recorded, and the safety of each group in the HuPrime® intestinal cancer CR3820 subcutaneous xenograft model was studied. The results of body weight changes of the mice were shown in Table 30. Correspondingly, the curve graph of the percentage changes of body weight in each treatment group over time was shown in FIG. 18.

TABLE 28
Changes in tumor volume in each group of mice over treatment time in the
HuPrime ® intestinal cancer CR3820 subcutaneous model (mm³)

| | Days after initial administration | | | | | | | | | |
|---|---|---|---|---|---|---|---|---|---|---|
| Group | 0 | 3 | 7 | 10 | 14 | 17 | 21 | 24 | 28 | 31 |
| 01 | 137.43 | 171.88 | 387.71 | 591.57 | 923.47 | 1171.58 | 1624.45 | 1792.37 | 1865.61 | 2203.47 |
| 02 | 137.64 | 183.78 | 362.76 | 495.03 | 640.83 | 758.49 | 1099.76 | 1199.09 | 1596.60 | 1753.12 |
| 03 | 137.23 | 172.55 | 209.91 | 197.67 | 176.74 | 148.74 | 121.71 | 110.59 | 85.22 | 88.44 |
| 04 | 137.49 | 148.90 | 177.26 | 147.06 | 137.48 | 117.53 | 152.25 | 146.58 | 73.81 | 75.21 |

TABLE 29
Drug efficacy analysis table of each group in the HuPrime ® intestinal
cancer CR3820 subcutaneous model

| | Day 24 | | | | |
|---|---|---|---|---|---|
| | | Relative | | | P Value |
| | | tumor | | | (compared with |
| | Tumor volume | volume | | | the control |
| Experiment group | (X̅ ± S) = | (X̅ ± S) | TGI (%) | T/C (%) | group) |
| Group 1 | 1792.37 ± 347.13 | 13.58 ± 7.27 | — | — | — |
| Glucose injection | | | | | |
| (pH 7.7-8.0), 10 μL/g, i.v., | | | | | |
| QD × 5; 2 days off; 2 weeks | | | | | |
| off; QD × 5 | | | | | |
| Group 2 | 1199.09 ± 228.15 | 8.46 ± 2.85 | 37.73 | 62.27 | >0.05 |
| Ifosfamide, 60 mg/kg, | | | | | |
| 10 μL/g, i.p., | | | | | |
| QD × 5/week × 2 weeks | | | | | |
| Group 3 | 110.59 ± 13.60 | 0.80 ± 0.24 | 94.08 | 5.92 | <0.001** |
| AST, 8 mg/kg, 10 μL/g, | | | | | |
| i.v., QW × 3 | | | | | |
| Group 4 | 146.58 ± 44.99 | 1.03 ± 0.71 | 92.44 | 7.56 | <0.001*** |
| AST, 4 mg/kg, 10 μL/g, | | | | | |
| i.v., QD × 5; 2 days off; | | | | | |
| 2 weeks off; QD × 5 | | | | | |

TABLE 30
Changes in body weight of mice in the HuPrime ® intestinal cancer CR3820 subcutaneous model

| | Days after initial administration | | | | | | | | | | | | | | | |
|---|---|---|---|---|---|---|---|---|---|---|---|---|---|---|---|---|
| Group | 0 | 1 | 2 | 3 | 4 | 7 | 10 | 14 | 17 | 21 | 22 | 23 | 24 | 25 | 28 | 31 |
| 01 | 24.5 | 23.8 | 24.5 | 24.0 | 23.6 | 24.3 | 24.1 | 24.4 | 24.6 | 25.0 | 24.1 | 24.0 | 23.4 | 23.1 | 22.2 | 22.5 |
| 02 | 25.0 | 24.5 | 25.1 | 25.2 | 24.1 | 24.1 | 23.7 | 24.1 | 24.7 | 25.5 | — | — | 25.2 | — | 25.1 | 25.4 |
| 03 | 25.5 | — | — | 25.2 | — | 25.9 | 25.5 | 25.7 | 26.4 | 26.5 | — | — | 26.4 | — | 26.0 | 26.4 |
| 04 | 24.8 | 25.1 | 25.3 | 25.3 | 24.9 | 25.3 | 25.6 | 24.9 | 25.5 | 26.3 | 25.8 | 25.1 | 25.8 | 25.9 | 25.2 | 25.9 |

The mean tumor volume of mice in the vehicle control group was 1792.37 mm³ on Day 24 after the initial administration. The mean tumor volume of the test drug Ifosfamide treatment group (QD×5/week×2 weeks, Group 2) at the dose of 60 mg/kg on Day 24 was 1199.09 mm³, and the relative tumor growth inhibition rate TGI (%) was 37.73%, with no statistically significant difference compared with the control group (p>0.05).

The mean tumor volumes of the test drug AST treatment groups at the dose of 8 mg/kg (QW×3, Group 3), at the dose of 4 mg/kg (QD×5, 2 days off, 2 weeks off, QD×5, Group 4) on Day 24 were 110.59 mm³ and 146.58 mm³, and the relative tumor growth inhibition rates TGI (%) were 94.08% and 92.44%, respectively, with statistically significant difference compared with the control group (p<0.05).

The above experimental results showed that AST in the model having KRAS G12D pathogenic mutation, the test drug AST 8 mg/kg (QW×3, Group 3), 4 mg/kg (QD×5, 2 days off, 2 weeks off, QD×5, Group 4) had a statistically significant anti-tumor effect on the HuPrime® intestinal cancer CR3820 subcutaneous model at the tested dosage and dosing frequency of this experiment. The test drugs Ifosfamide and AST were well tolerated at the tested doses in this study.

### 7. Anti-Tumor Effect and Safety Evaluation of the Test Substances AST, AST-3424 and Ifosfamide Monotherapy in the HuPrime® Pancreatic Cancer PA2637 Subcutaneous Model

The HuPrime® pancreatic cancer PA2637 subcutaneous model was a PDX model harboring the KRAS G12D pathogenic mutation.

NOD.SCID female mice were subcutaneously inoculated with the HuPrime® pancreatic cancer PA2637 tumor blocks so as to establish a subcutaneous transplantation tumor model of human pancreatic cancer. The test was divided into the following groups: the test drug Ifosfamide 60 mg/kg monotherapy treatment group (QD×5/week×2 weeks, Group 2) was administered intraperitoneally once a day for 5 consecutive days, rested for 2 days, and then administered once a day for 5 consecutive days again; AST 8 mg/kg monotherapy group (QW×3, Group 3) was administered through the tail vein once a week for a total of 3 weeks; AST 4 mg/kg monotherapy group (QD×5, 2 days off, 2 weeks off, QD×5, Group 4), AST-3424 1 mg/kg monotherapy group (QD×5, 2 days off, 2 weeks off, QD×5, Group 5) and the vehicle control group glucose injection (pH 7.7-8.0, Group 1) both were administered through the tail vein, and both had the same dosing cycle: administering once a day for 5 consecutive days, resting for 2 days, then resting for 2 weeks, and then administering once a day for 5 consecutive days again. There were 5 groups in total and 6 mice in each group in this experiment. The administration routes, dosages and regimens of the experimental design were shown in Table 31.

TABLE 31
Administration routes, dosages and regimens of the HuPrime ® pancreatic
cancer PA2637 subcutaneous model

| | The | | | | |
|---|---|---|---|---|---|
| | number | | | | |
| Group | of | Administration | Dosage | Administration | |
| No. | Animals | group | (mg/kg) | method | Administration period |
| 1 | 6 | Glucose | — | i.v. | QD × 5, 2 days off, 2 weeks off, QD × 5 |
| | | injection | | | |
| | | (pH 7.7-8.0) | | | |
| 2 | 6 | Ifosfamide | 60 | i.p. | QD × 5/week × 2 weeks |
| 3 | 6 | AST | 8 | i.v. | QW × 3 |
| 4 | 6 | AST | 4 | i.v. | QD × 5, 2 days off, 2 weeks off, QD × 5 |
| 5 | 6 | AST-3424 | 1 | i.v. | QD × 5, 2 days off, 2 weeks off, QD × 5 |

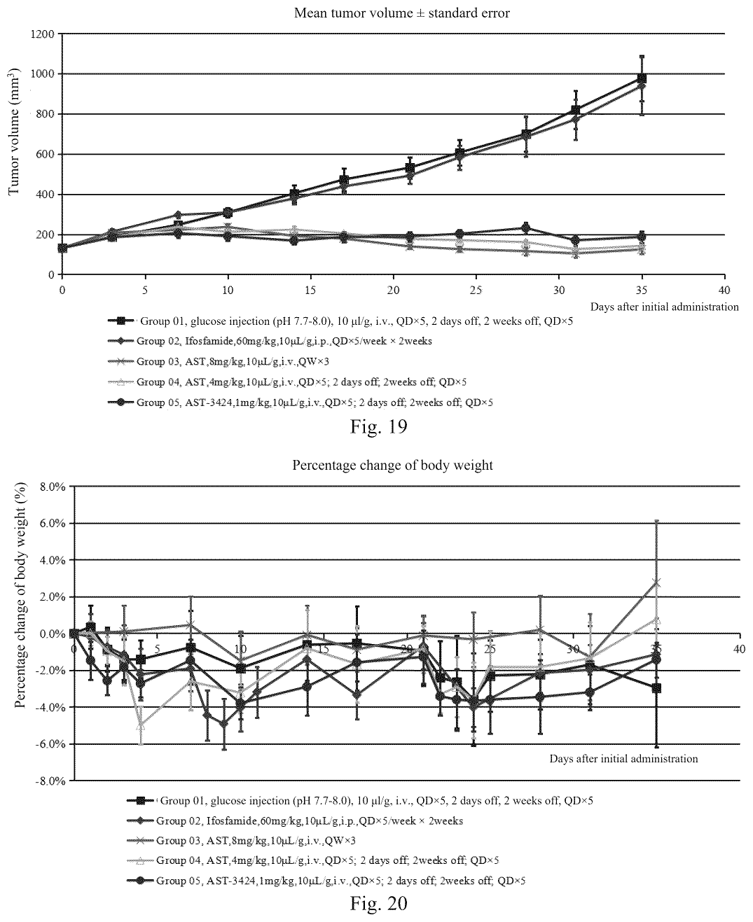

The tumor growth of each treatment group and control group was recorded on different days of the test, as shown in Table 32. The corresponding growth curve of tumor volume in each group of mice was shown in FIG. 19. The therapeutic effect was evaluated based on the relative tumor proliferation rate and relative tumor growth inhibition rate. The drug efficacy analysis of each group was shown in Table 33. The body weight changes of the treatment group and the control group after administration were recorded, and the safety of each group in the HuPrime® pancreatic cancer PA2637 subcutaneous xenograft model was studied. The results of body weight changes of the mice were shown in Table 34. Correspondingly, the curve graph of the percentage changes of body weight in each treatment group over time was shown in FIG. 20.

TABLE 32
Changes in tumor volume in each group of mice over treatment time in the
HuPrime ® pancreatic cancer PA2637 subcutaneous model (mm³)

| | Days after initial administration | | | | | | | | | | |
|---|---|---|---|---|---|---|---|---|---|---|---|
| Group | 0 | 3 | 7 | 10 | 14 | 17 | 21 | 24 | 28 | 31 | 35 |
| Group 01 | 130.37 | 186.35 | 246.28 | 308.00 | 402.92 | 472.45 | 531.56 | 606.41 | 700.31 | 820.04 | 977.46 |
| Group 02 | 130.24 | 211.69 | 295.87 | 307.84 | 377.51 | 437.58 | 491.24 | 582.72 | 684.36 | 771.40 | 938.33 |
| Group 03 | 130.35 | 206.38 | 221.16 | 235.81 | 190.59 | 178.00 | 138.24 | 125.43 | 115.08 | 103.87 | 123.47 |
| Group 04 | 130.55 | 184.14 | 235.94 | 210.81 | 223.54 | 204.85 | 175.78 | 170.26 | 160.17 | 125.16 | 141.48 |
| Group 05 | 130.32 | 184.00 | 204.51 | 190.00 | 167.36 | 185.97 | 188.97 | 201.89 | 230.64 | 169.27 | 186.08 |

TABLE 33
Drug efficacy analysis table of each group in the HuPrime ® pancreatic
cancer PA2637 subcutaneous model

| | Day 35 | | | | |
|---|---|---|---|---|---|
| | | Relative | | | |
| | | tumor | | | P Value |
| | Tumor volume | volume | | | (compared with the |
| Experiment group | (X̅ ± S) | (X̅ ± S) | TGI (%) | T/C (%) | control group) |
| Group 1 | 977.46 ± 113.44 | 7.45 ± 0.73 | — | — | — |
| Glucose injection | | | | | |
| ((pH 7.7-8.0), 10 μL/g, | | | | | |
| i.v., QD × 5; 2 days off; | | | | | |
| 2 weeks off; QD × 5 | | | | | |
| Group 2 | 938.33 ± 143.94 | 7.25 ± 1.18 | 2.74 | 97.26 | >0.05 |
| Ifosfamide, 60 mg/kg, | | | | | |
| 10 μL/g, i.p., | | | | | |
| QD × 5/week × 2 weeks | | | | | |
| Group 3 | 123.47 ± 21.61 | 0.95 ± 0.16 | 87.28 | 12.72 | <0.001*** |
| AST, 8 mg/kg, 10 μL/g, i.v., | | | | | |
| QW × 3 | | | | | |
| Group 4 | 141.48 ± 13.32 | 1.08 ± 0.10 | 85.46 | 14.54 | <0.001 |
| AST, 4 mg/kg, 10 μL/g, i.v., | | | | | |
| QD × 5; 2 days off; 2 weeks | | | | | |
| off; QD × 5 | | | | | |
| Group 5 | 186.08 ± 29.94 | 1.43 ± 0.23 | 80.78 | 19.22 | <0.001** |
| AST-3424, 1 mg/kg, | | | | | |
| 10 μL/g, i.v., QD × 5; 2 days | | | | | |
| off; 2 weeks off; QD × 5 | | | | | |

TABLE 34
Percentage changes in body weight of mice in the HuPrime ® pancreatic
cancer PA2637 subcutaneous model

| | Days after initial administration | | | | | | | |
|---|---|---|---|---|---|---|---|---|
| Group | 0 | 1 | 2 | 3 | 4 | 7 | 10 | 14 |
| 01 | 0.00% | 0.35% | −0.87% | −1.42% | −1.42% | −0.76% | −1.90% | −0.63% |
| 02 | 0.00% | −0.20% | −0.74% | −1.17% | −2.24% | −1.92% | −4.05% | −1.42% |
| 03 | 0.00% | — | — | 0.09% | — | 0.45% | −1.49% | −0.09% |
| 04 | 0.00% | 0.00% | −0.97% | −1.51% | −4.99% | −2.61% | −3.22% | −0.81% |
| 05 | 0.00% | −1.47% | −2.58% | −1.85% | −2.73% | −1.48% | −3.80% | −2.90% |

| | Days after initial administration | | | | | | | |
|---|---|---|---|---|---|---|---|---|
| Group | 17 | 21 | 22 | 23 | 24 | 28 | 31 | 35 |
| 01 | −0.55% | −0.96% | −2.41% | −2.65% | −3.61% | −2.23% | −1.71% | −2.96% |
| 02 | −3.36% | −0.68% | — | — | −4.03% | −2.16% | −1.99% | −1.15% |
| 03 | −0.89% | −0.12% | — | — | −0.33% | 0.19% | −1.29% | 2.76% |
| 04 | −1.66% | −0.85% | −3.32% | −2.91% | −3.63% | −1.82% | −1.36% | 0.78% |
| 05 | −1.58% | −1.29% | −3.42% | −3.61% | −3.68% | −3.46% | −3.20% | −1.43% |

The mean tumor volume of mice in the vehicle control group was 977.46 mm³ on Day 35 after the initial administration. The mean tumor volume of the test drug Ifosfamide treatment group (Group 2) at the dose of 60 mg/kg on Day 35 was 938.33 mm³, and the relative tumor growth inhibition rate TGI (%) was 2.74%, with no statistically significant difference compared with the control group (p>0.05).

The mean tumor volumes of the test drug AST treatment groups at the dose of 8 mg/kg (QW×3, Group 3), at the dose of 4 mg/kg (QD×5, 2 days off, 2 weeks off, QD×5, Group 4), and the test drug AST-3424 at the dose of 1 mg/kg (QD×5, 2 days off, 2 weeks off, QD×5, Group 5) on Day 35 were 123.47 mm³, 141.48 mm³ and 186.08 mm³, and the relative tumor growth inhibition rates TGI (%) were 87.28%, 85.46% and 80.78%, respectively, with statistically significant difference compared with the control group (p<0.001).

The above experimental results showed that AST and AST-3424 in the model having KRAS G12D pathogenic mutation, the test drug AST 8 mg/kg (QW×3, Group 3), 4 mg/kg (QD×5, 2 days off, 2 weeks off, QD×5, Group 4) and the test drug AST-3424 1 mg/kg (QD×5, 2 days off, 2 weeks off, QD×5, Group 5) had a statistically significant anti-tumor effect on the HuPrime® pancreatic cancer PA2637 subcutaneous model at the tested dosage and dosing frequency of this experiment. During the experiment, the mice in each test drug treatment group were well tolerated during the treatment period.

### 8. Anti-Tumor Effect and Safety Evaluation of the Test Substances AST and Ifosfamide Monotherapy in the HuPrime® Lung Cancer LU11873 Subcutaneous Model

The HuPrime® lung cancer LU11873 subcutaneous model was a PDX model harboring the KRAS G12C pathogenic mutation.

NOD.SCID female mice were subcutaneously inoculated with the HuPrime® lung cancer LU11873 tumor blocks so as to establish a subcutaneous transplantation tumor model of human lung cancer. The test was divided into the following groups: the test drug Ifosfamide 60 mg/kg monotherapy group (Group 2) was administered once a day for 5 consecutive days, rested for 2 days, and then administered once a day for 5 consecutive days again; AST 4 mg/kg monotherapy group (Group 5) was administered once a day for 5 consecutive days, rested for 2 days, then rested for 2 weeks, and then administered once a day for 5 consecutive days again; and glucose injection (pH 7.7-8.0) vehicle control group (Group 1) was administered once a day for 5 consecutive days, rested for 2 days, then rested for 2 weeks, and then administered once a day for 5 consecutive days again. There were 3 groups in total and 6 mice in each group in this experiment. The test drug Ifosfamide was administered intraperitoneally. The vehicle control group and each AST group were administered by tail vein injection. The administration routes, dosages and regimens of the experimental design were shown in Table 35.

TABLE 35
Administration routes, doses and regimens in the HuPrime ® lung
cancer LU11873 subcutaneous model

| | The | | | | |
|---|---|---|---|---|---|
| | number | | | | |
| Group | of | Administration | Dosage | Administration | |
| No. | Animals | group | (mg/kg) | method | Administration period |
| 1 | 6 | Glucose | — | i.v. | QD × 5, 2 days off, 2 weeks off, QD × 5 |
| | | injection | | | |
| | | (pH 7.7-8.0) | | | |
| 2 | 6 | Ifosfamide | 60 | i.p. | QD × 5/week × 2 weeks |
| 5 | 6 | AST | 4 | i.v. | QD × 5, 2 days off, 2 weeks off, QD × 5 |

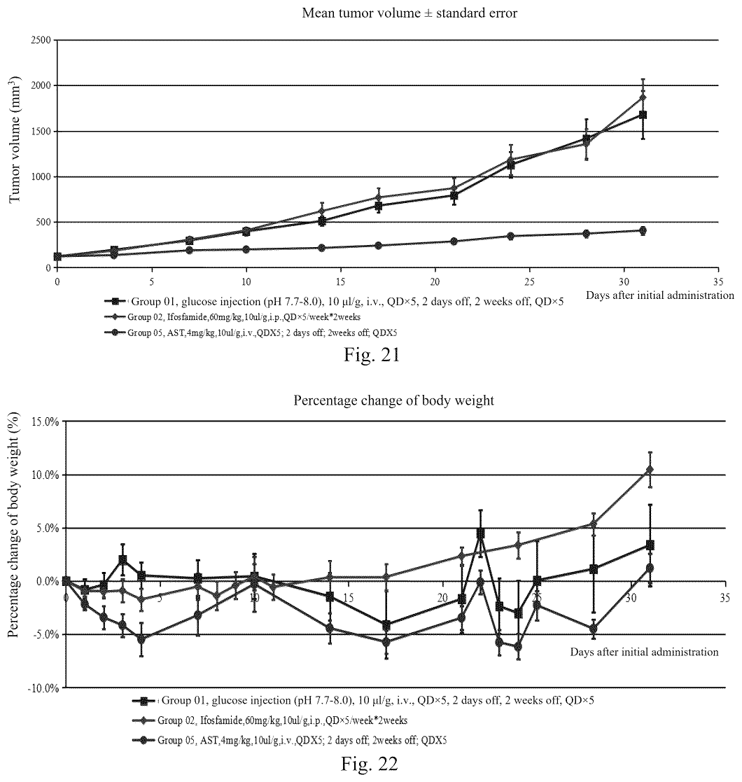

The tumor growth of each treatment group and control group was recorded on different days of the test, as shown in Table 36. The corresponding growth curve of tumor volume in each group of mice was shown in FIG. 21. The therapeutic effect was evaluated based on the relative tumor proliferation rate and relative tumor growth inhibition rate. The drug efficacy analysis of each group was shown in Table 37. The body weight changes of the treatment group and the control group after administration were recorded, and the safety of each group in the HuPrime® lung cancer LU11873 subcutaneous xenograft model was studied. The results of body weight changes of the mice were shown in Table 38. Correspondingly, the curve graph of the percentage changes of body weight in each treatment group over time was shown in FIG. 22.

TABLE 36
Changes in tumor volume in each group of mice over treatment time in the
HuPrime ® lung cancer LU11873 subcutaneous model (mm³)

| | Days after initial administration | | | | | | | | | |
|---|---|---|---|---|---|---|---|---|---|---|
| Group | 0 | 3 | 7 | 10 | 14 | 17 | 21 | 24 | 28 | 31 |
| Group 01 | 121.41 | 194.85 | 296.16 | 396.17 | 509.62 | 678.47 | 790.84 | 1125.75 | 1415.78 | 1677.89 |
| Group 02 | 121.82 | 183.55 | 305.91 | 406.78 | 619.15 | 768.80 | 870.60 | 1183.70 | 1354.36 | 1866.37 |
| Group 05 | 121.48 | 135.18 | 188.07 | 197.93 | 214.44 | 240.28 | 286.32 | 344.68 | 371.66 | 406.40 |

TABLE 37
Drug efficacy analysis table of each group in the HuPrime ® lung cancer LU11873 subcutaneous model

| | Day 31 after initial administration | | | | |
|---|---|---|---|---|---|
| | | Relative | | | |
| | | tumor | | | P Value |
| | Tumor volume | volume | | | (compared with |
| Experiment group | (X̅ ± S) | (X̅ ± S) | TGI (%) | T/C (%) | the control group) |
| Group 1 | 1677.89 ± 262.51 | 13.77 ± 1.89 | — | — | — |
| Glucose injection (pH 7.7-8.0), | | | | | |
| 10 μL/g, i.v., QD × 5, 2 days off, | | | | | |
| 2 weeks off, QD × 5 | | | | | |
| Group 2 | 1866.37 ± 203.06 | 15.16 ± 1.26 | −10.08 | 110.08 | >0.05 |
| Ifosfamide, 60 mg/kg, 10 μL/g, | | | | | |
| i.p., QD × 5/week × 2 weeks | | | | | |
| Group 5 | 406.40 ± 43.43 | 3.42 ± 0.44 | 75.16 | 24.84 | <0.001*** |
| AST, 4 mg/kg, 10 μL/g, i.v., | | | | | |
| QD × 5, 2 days off, 2 weeks | | | | | |
| off, QD × 5 | | | | | |

TABLE 38
Percentage change in body weight of mice in the HuPrime ® lung
cancer LU11873 subcutaneous model

| Group | 0 | 1 | 2 | 3 | 4 | 7 | 10 | 14 |
|---|---|---|---|---|---|---|---|---|
| 01 | 0.00% | −0.82% | −0.37% | 2.00% | 0.51% | 0.24% | 0.43% | −1.45% |
| 02 | 0.00% | −0.95% | −1.01% | −0.90% | −1.74% | −0.52% | 0.39% | 0.34% |
| 05 | 0.00% | −2.18% | −3.41% | −4.15% | −5.47% | −3.20% | −0.29% | −4.42% |

| Group | 17 | 21 | 22 | 23 | 24 | 25 | 28 | 31 |
|---|---|---|---|---|---|---|---|---|
| 01 | −4.10% | −1.68% | 4.47% | −2.36% | −3.01% | 0.05% | 1.13% | 3.37% |
| 02 | 0.38% | 2.33% | — | — | 3.36% | — | 5.36% | 10.46% |
| 05 | −5.73% | −3.45% | −0.11% | −5.76% | −6.17% | −2.27% | −4.48% | 1.21% |

The mean tumor volume of mice in the vehicle control group was 1677.89 mm³ on Day 31 after the initial administration. The mean tumor volume of the test drug Ifosfamide treatment group (Group 2) at the dose of 60 mg/kg on Day 31 was 1866.37 mm³, and the relative tumor growth inhibition TGI (%) was −10.08%, with no statistically significant difference compared with the control group (p>0.05). The mean tumor volume of the test drug AST treatment group (Group 5) at the dose of 4 mg/kg (QD×5, 2 days off, 2 weeks off, QD×5) on Day 31 was 406.40 mm³, with statistically significant difference compared with the control group (p<0.05), and the relative tumor growth inhibition rate TGI (%) was 75.16%.

The above experimental results showed that AST in the model having KRAS G12C pathogenic mutation, the test drug AST treatment group (Group 5) at the dose of 4 mg/kg (QD×5, 2 days off, 2 weeks off, QD×5) had a significant anti-tumor effect on the HuPrime® lung cancer LU11873 subcutaneous model at the tested dosage and dosing frequency of this study. Ifosfamide administration group had no tumor inhibitory effect. During the experiment, the mice in each test drug treatment group did not have any weight loss and were well tolerated during the treatment period.

Specifically, the applicant found that in Example 3, AST in the KRAS pathogenic mutation model lung cancer LU11693 having G12C amino acid mutation had no significant anti-tumor effect, with TGI % of 54.64% at the dose of 10 mg/kg; while in this example, AST in the KRAS pathogenic mutation model lung cancer LU11873 having G12C amino acid mutation had a significant anti-tumor effect, with TGI % of 75.16% at the dose of 4 mg/kg. The difference between the two models was significant, indicating that there might be some sort of difference between these two PDX models. It can be seen from data about the source of the models:
LU11693 originated from a 58-year-old female patient who showed cachexia and mild ulcers clinically;
LU11873 originated from a 51-year-old male patient who showed slight weight loss and mild ulcers clinically.

### 9. Antitumor Effect and Safety Evaluation of Test Substances AST and Ifosfamide Monotherapy in the HuPrime® Pancreatic Cancer PA1383 Subcutaneous Model

The HuPrime® pancreatic cancer PA1383 subcutaneous model was a PDX model harboring KRAS G12C pathogenic mutation.

Balb/nude female mice were subcutaneously inoculated with HuPrime® pancreatic cancer PA1383 tumor blocks so as to establish a subcutaneous transplantation tumor model of human pancreatic cancer. The test was divided into the following groups: the test drug Ifosfamide 60 mg/kg monotherapy group (Group 2) was administered once a day for 5 consecutive days, rested for 2 days, and then administered once a day for 5 consecutive days again; AST 8 mg/kg monotherapy group (Group 4) was administered once a week for a total of 3 weeks; AST 4 mg/kg monotherapy group (Group 5) was administered once a day for 5 consecutive days, rested for 2 days, then rested for 2 weeks, and then administered once a day for 5 consecutive days again; and glucose injection (pH 7.7-8.0) vehicle control group (Group 1) was administered once a day for 5 consecutive days, rested for 2 days, then rested for 2 weeks, and then administered once a day for 5 consecutive days again. There were 4 groups in total and 6 mice in each group in this study. Among them, the test drug Ifosfamide was administered intraperitoneally. The vehicle control group and each AST group were administered by tail vein injection. The administration routes, dosages and regimens of the experimental design were shown in Table 39.

TABLE 39
Administration routes, doses and regimens in the HuPrime ® pancreatic
cancer PA1383 subcutaneous model

| | The | | | | |
|---|---|---|---|---|---|
| Group | number of | Administration | Dosage | Administration | |
| No. | animals | group | (mg/kg) | method | Administration period |
| 1 | 6 | glucose injection | — | i.v. | QD × 5, 2 days off, 2 weeks off, QD × 5 |
| | | (pH 7.7-8.0) | | | |
| 2 | 6 | Ifosfamide | 60 | i.p. | QD × 5/week × 2 weeks |
| 4 | 6 | AST | 8 | i.v. | QW × 3 |
| 5 | 6 | AST | 4 | i.v. | QD × 5, 2 days off, 2 weeks off, QD × 5 |

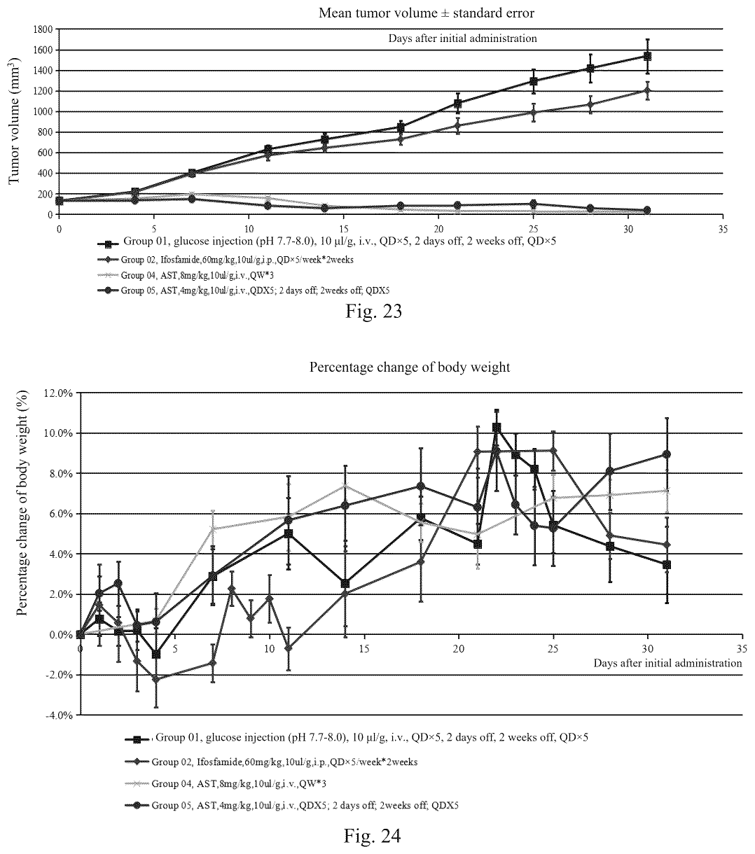

The tumor growth of each treatment group and control group was recorded on different days of the test, as shown in Table 40. The corresponding growth curve of tumor volume in each group of mice was shown in FIG. 23. The therapeutic effect was evaluated based on the relative tumor proliferation rate and relative tumor growth inhibition rate. The drug efficacy analysis of each group was shown in Table 41. The body weight changes of the treatment group and the control group after administration were recorded, and the safety of each group in the HuPrime® pancreatic cancer PA1383 subcutaneous xenograft model was studied. The results of body weight changes of the mice were shown in Table 42. Correspondingly, the curve graph of the percentage changes of body weight in each treatment group over time was shown in FIG. 24.

TABLE 40
Changes in tumor volume in each group of mice over treatment time in the
HuPrime ® pancreatic cancer PA1383 subcutaneous model (mm³)

| | Days after initial administration | | | | | | | | | |
|---|---|---|---|---|---|---|---|---|---|---|
| Group | 0 | 4 | 7 | 11 | 14 | 18 | 21 | 25 | 28 | 31 |
| 01 | 131.14 | 220.07 | 402.23 | 630.24 | 726.31 | 847.35 | 1077.74 | 1291.64 | 1417.06 | 1536.48 |
| 02 | 131.04 | 215.30 | 391.66 | 570.57 | 643.25 | 727.85 | 859.10 | 986.71 | 1064.57 | 1202.01 |
| 04 | 131.08 | 152.43 | 195.20 | 156.69 | 84.43 | 45.14 | 31.21 | 25.71 | 23.93 | 18.57 |
| 05 | 131.05 | 134.98 | 147.15 | 82.79 | 57.51 | 82.53 | 87.39 | 100.47 | 58.54 | 39.94 |

TABLE 41
Drug efficacy analysis table of each group in the HuPrime ® pancreatic
cancer PA1383 subcutaneous model

| | Day 31 after initial administration | | | | |
|---|---|---|---|---|---|
| | | Relative | | | P Value |
| | Tumor | tumor | | | (compared |
| | volume | volume | | | with the |
| Experiment group | (X̅ ± S) | (X̅ ± S) | TGI (%) | T/C (%) | control group) |
| Group 1 | 1536.48 ± 165.08 | 11.74 ± 1.31 | — | — | — |
| glucose injection (pH 7.7- | | | | | |
| 8.0), 10 μL/g, i.v., QD × 5, | | | | | |
| 2 days off, 2 weeks off, | | | | | |
| QD × 5 | | | | | |
| Group 2 | 1202.01 ± 85.63 | 9.18 ± 0.54 | 21.84 | 78.16 | >0.05 |
| Ifosfamide, 60 mg/kg, 10 | | | | | |
| μL/g, i.p., QD × 5/week × 2 | | | | | |
| weeks | | | | | |
| Group 4 | 18.57 ± 7.43 | 0.15 ± 0.06 | 98.72 | 1.28 | <0.001*** |
| AST, 8 mg/kg, 10 μL/g, i.v., | | | | | |
| QW × 3 | | | | | |
| Group 5 | 39.94 ± 20.41 | 0.30 ± 0.15 | 97.46 | 2.54 | <0.001*** |
| AST, 4 mg/kg, 10 μL/g, i.v., | | | | | |
| QD × 5, 2 days off, 2 weeks | | | | | |
| off, QD × 5 | | | | | |

TABLE 42
Percentage change in body weight of mice in the HuPrime ® pancreatic
cancer PA1383 subcutaneous model

| | Days after initial administration | | | | | | | |
|---|---|---|---|---|---|---|---|---|
| Group | 0 | 1 | 2 | 3 | 4 | 7 | 11 | 14 |
| 1 | 0.00% | 0.76% | 0.16% | 0.19% | −0.99% | 2.88% | 5.00% | 2.54% |
| 2 | 0.00% | 1.45% | 0.57% | −1.33% | −2.25% | −1.43% | −0.70% | 2.01% |
| 4 | 0.00% | — | — | — | 0.68% | 5.22% | 5.83% | 7.37% |
| 5 | 0.00% | 2.03% | 2.53% | 0.44% | 0.61% | 2.91% | 5.66% | 6.39% |

| | Days after initial administration | | | | | | | |
|---|---|---|---|---|---|---|---|---|
| Group | 18 | 21 | 22 | 23 | 24 | 25 | 28 | 31 |
| 1 | 5.77% | 4.49% | 10.28% | 8.94% | 8.20% | 5.41% | 4.37% | 3.46% |
| 2 | 3.59% | 9.06% | — | — | — | 9.12% | 4.90% | 4.44% |
| 4 | 5.55% | 4.97% | — | — | — | 6.77% | 6.91% | 7.13% |
| 5 | 7.35% | 6.31% | 9.09% | 6.44% | 5.39% | 5.27% | 8.10% | 8.93% |

The mean tumor volume of mice in the vehicle control group was 1536.48 mm³ on Day 31 after the initial administration. The mean tumor volume of the test drug Ifosfamide treatment group (Group 2) at the dose of 60 mg/kg on Day 31 was 1202.01 mm³, and the relative tumor growth inhibition TGI (%) was 21.84%, with no statistically significant difference compared with the control group (p>0.05).

The mean tumor volumes of the test drug AST treatment groups (Group 4 and Group 5) at the dose of 8 mg/kg (QD×3) and at the dose of 4 mg/kg (QD×5, 2 days off, 2 weeks off, QD×5) on Day 31 were 18.57 mm³ and 39.94 mm³, respectively, with statistically significant difference compared with the control group (p<0.05), and the relative tumor growth inhibition rates TGI (%) were 98.72% and 97.46%, respectively. Two mice in each group had tumor completely cleared, with a clearance rate of 33.3%.

The above experimental results showed that AST in the model having KRAS G12C pathogenic mutation, AST treatment groups (Group 4 and Group 5) at the dose of 8 mg/kg (QD×3) and at the dose of 4 mg/kg (QD×5, 2 days off, 2 weeks off, QD×5) had a significant anti-tumor effect on the HuPrime® pancreatic cancer PA1383 subcutaneous model at the tested dosage and dosing frequency of this study. Ifosfamide administration group had no tumor inhibitory effect. The mice in each test drug treatment group did not have any weight loss and were well tolerated during the treatment period.

### 10. Detection of AKR1C3 RNA Expression Level and Enzyme Content in Tissues

### A. FPKM Detection of AKR1C3 RNA Expression Level

According to the method described in the document (Meng, F., Li, W. F., Jung, D., Wang, C. C., Qi, T., Shia, C. S., Hsu, R. Y., Hsieh, Y. C., & Duan, J. (2021). A novel selective AKR1C3-activated prodrug AST-3424/OBI-3424 exhibits broad anti-tumor activity. American journal of cancer research, 11(7), 3645-3659), AKR1C3 RNA expression levels in tissues of gastric cancer GA6021, pancreatic cancer PA1222, and lung cancer LU11693 as described above were analyzed with RNA-Seq and quantified by Log 2 FPKM. The results were as follows:
AKR1C3 LOG 2 (FPKM) was detected for GA6201 as 6.78, for LU11693 as 11.14, for PA1222 as 7.39, for HPAF-II as 8.31, for LU5161 as 11.56, for CR3820 as 8.34, for PA2637 as 9.12, for LU11873 as 10.26, and for PA1383 as 9.57 (see Table 43).

According to the above document, AKR1C3 RNA in all the nine tumor tissues was highly expressed.

### B. IHC Method Detection and H-SCORE of AKR1C3 Protein Content

The AKR1C3 protein contents of the three tissues were determined according to the customary IHC (immunohistochemistry) staining (commercial IHC reagents was used, the first antibody was Rabbit IgG mAb from Abcam, and the second antibody was Bond Polymer Refine Detection from Leica; staining conditions: antigen retrieval 100° C., pH 9.0 EDTA buffer 20 min, dilution ratio: 1:800), and the staining results were performed for H-SCORE:

The immunohistochemical staining intensity was divided into 0 (negative), 1+(weak staining), 2+(medium staining), and 3+(strong staining). The thresholds for weak staining, medium staining, and strong staining were manually set on the scoring instrument, and then color recognition was performed on the staining sample photographs using image processing software. For staining photographs of all the samples, the corresponding staining of a certain cell was scored according to a unified standard by the scoring software: 0/1/2/3. Then the percentage of positive cells with different staining intensities to the total cells in the slice was counted. H-Score was calculated as the score of IHC results for each sample using the following formula. The H-score would be between 0 and 300, and the higher the score, the higher the expression level of the corresponding target of the antibody (AKR1C3 enzyme protein) in the sample. The calculation formula was as follows:

$$H - Score = ( \% at 0 ) \times 0 + ( \% at 1 ) \times 1 + ( \% at 2 ) \times 2 + ( \% at 3 ) \times 3$$

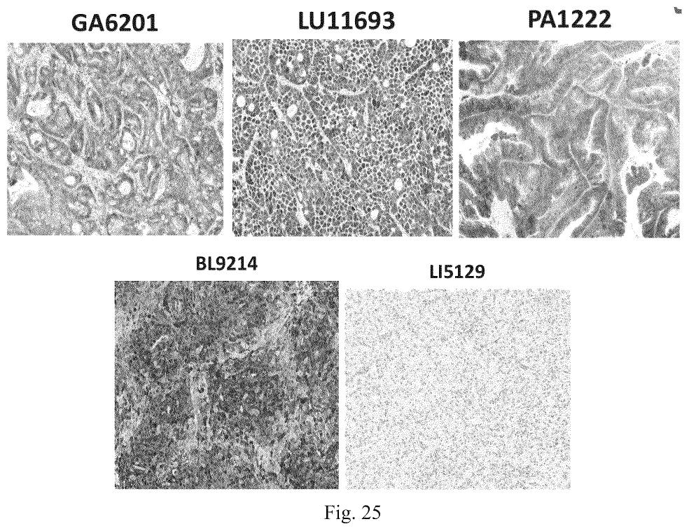

The staining results of GA6201, LU11693, PA1222, and two control groups were shown in FIG. 25, and the scoring results were shown in Table 43 below:

TABLE 43
IHC and RNA analysis results of nine models and control groups

| | | | AKR1C3 |
|---|---|---|---|
| | | AKR1C3 | LOG₂ |
| Model ID | Cancer type | H-Score | (FPKM) |
| GA6201 | Gastric Cancer | 248.75 | 6.78 |
| LU11693 | Lung Cancer | 269.10 | 11.14 |
| PA1222 | Pancreatic Cancer | 204.28 | 7.39 |
| HPAF-II | Pancreatic Cancer | high | 8.31 |
| | | expression | |
| LU5161 | Lung Cancer | 272.78 | 11.56 |
| CR3820 | Intestinal Cancer | 274.88 | 8.34 |
| PA2637 | Pancreatic Cancer | 238.23 | 9.12 |
| LU11873 | Lung Cancer | 236.69 | 10.26 |
| PA1383 | Pancreatic Cancer | 252.92 | 9.57 |
| BL9214 | Bladder Cancer | 265.86 | 10.86 |
| (positive control) | | | |
| LI5129 | Liver Cancer | 0.00 | −2.00 |
| (negative control) | | | |

Specific staining statistical results of models GA6201, LU11693, PA1222, and control groups were shown in Table 44 below:

TABLE 44
IHC results and H-SCORE of models GA6201,
LU11693, PA1222, and control groups

| | | AKR1C3 | | | | |
|---|---|---|---|---|---|---|
| Model ID | Cancer type | 0 | 1 | 2 | 3 | H-Score |
| GA6201 | Gastric Cancer | 1.07 | 8.75 | 30.54 | 59.63 | 248.75 |
| LU11693 | Lung Cancer | 1.70 | 5.51 | 14.79 | 78.00 | 269.10 |
| PA1222 | Pancreatic Cancer | 6.82 | 24.13 | 27.01 | 42.04 | 204.28 |
| BL9214 | Bladder Cancer | 1.47 | 7.23 | 15.26 | 76.04 | 265.86 |
| (positive | | | | | | |
| control) | | | | | | |
| LI5129 | Liver Cancer | 100.00 | 0.00 | 0.00 | 0.00 | 0.00 |
| (negative | | | | | | |
| control) | | | | | | |

In the above staining results, the positive and negative control results within the control range indicated that the H-SCORE results of this IHC staining were reliable.

According to the above results, the corresponding AKR1C3 protein in all the nine tissues was highly expressed.

It can be seen from comprehensive pharmacodynamic experiment results of the above nine models, together with the fact that the tissues used in the nine models were all tumor tissues with high expression of human AKR1C3 that: AST-3424 and AST have generally significant therapeutic effect on cancer with high expression of AKR1C3 and having the KRAS pathogenic mutation of G12D amino acid mutation; AST may have significant therapeutic effect on cancer with high expression of AKR1C3 and having the KRAS pathogenic mutation of G12C amino acid mutation. This means that high expression of AKR1C3 in certain tumors may be associated with KRAS (pathogenic) mutation subtypes, that is, high expression or overexpression of AKR1C3 often coexists with certain subtypes of KRAS (pathogenic) mutation in certain tumors, which leads to higher sensitivity of tumor models with these characteristics to AST-3424 or AST.

According to the document (Meng, F., Li, W. F., Jung, D., Wang, C. C., Qi, T., Shia, C. S., Hsu, R. Y., Hsieh, Y. C., & Duan, J. (2021). A novel selective AKR1C3-activated prodrug AST-3424/OBI-3424 exhibits broad anti-tumor activity. American journal of cancer research, 11(7), 3645-3659; Evans, K., Duan, J., Pritchard, T., Jones, C. D., McDermott, L., Gu, Z., Toscan, C. E., El-Zein, N., Mayoh, C., Erickson, S. W., Guo, Y., Meng, F., Jung, D., Rathi, K. S., Roberts, K. G., Mullighan, C. G., Shia, C. S., Pearce, T., Teicher, B. A., Smith, M. A., . . . Lock, R. B. (2019). OBI-3424, a Novel AKR1C3-Activated Prodrug, Exhibits Potent Efficacy against Preclinical Models of T-ALL. Clinical cancer research: an official journal of the American Association for Cancer Research, 25(14), 4493-4503; Yanlan Wang, Yue Liu, Changhua Zhou, Chunnian Wang, Ning Zhang, Donglin Cao, Qing Li & Zhong Wang (2020) An AKR1C3-specific prodrug with potent anti-tumor activities against T-ALL, Leukemia & Lymphoma, 61(7), 1660-1668) and corresponding patent applications:
PCT/US2016/021581 with Publication No. WO2016145092A1 (corresponding to Chinese Patent Application No. 2016800150788 with Publication No. CN107530556A),
PCT/US2016/062114 with Publication No. WO2017087428A1 (corresponding to Chinese Patent Application No. 2016800446081 with Publication No. CN108290911A),
PCT/CN2020/089692 with Publication No. WO2020228685A1;
PCT/NZ2019/050030 with Publication No. WO2019190331A1 (corresponding to Chinese Patent Application No. 2019800234236 with Publication No. CN111918864A);
PCT/CN2020/120281 with Publication No. WO2021068952A1,

These compounds in these patent applications, similar to compounds AST-3424 and AST, are AKR1C3-activated anticancer prodrugs, which are cleaved into the DNA alkylating agent

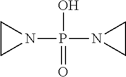

or nitrogen mustard structure after AKR1C3 activation.

Thus, in combination with the above experimental results of AST-3424 and AST, it can be inferred that AKR1C3-activated DNA alkylating agent prodrugs monotherapy or in combination with other therapeutic drugs have significant therapeutic effect on cancer and tumor patients having KRAS mutations, especially those with KRAS-G12D subtype mutation.

1. A treatment method which uses a drug monotherapy containing an AKR1C3-activated DNA alkylating agent prodrug compound or in combination with other therapeutic drugs for treating cancer and tumor patients having KRAS mutations.

2. The treatment method according to claim 1, wherein the compound is selected from structural Formulae 1/2/3/4/5/6 and salts, esters, solvates, isotopic isomers thereof: 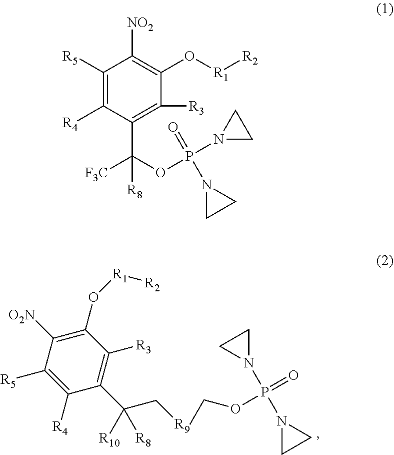
wherein the definitions of R₁, R₂, R₃, R₄, R₅, R₈, R₉, and R₁₀ are described in the claims of Patent Application PCT/CN2020/089692 with Publication No. WO2020228685A1; 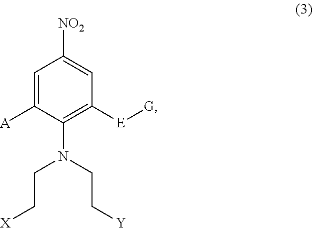
wherein the definitions of A, E, G, X and Y are described in the claims of Patent Application PCT/NZ2019/050030 with Publication No. WO2019190331A1 (corresponding to Chinese Patent Application No. 2019800234236 with Publication No. CN111918864A); 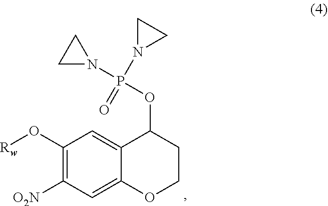
wherein the definition of R_{w} is described in the claims of Patent Application PCT/CN2020/120281 with Publication No. WO2021068952A1; 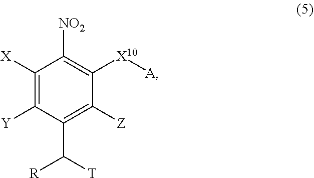
wherein the definitions of X, Y, Z, R, T, A, and X¹⁰ are described in the claims of Patent Application PCT/US2016/062114 with Publication No. WO2017087428A1 (corresponding to Chinese Patent Application No. 2016800200132 with Publication No. CN108136214A); 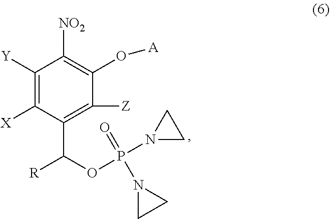
wherein:
A is a substituted or unsubstituted C₆-C₁₀ aryl, biaryl or substituted biaryl, 5-15 membered heteroaryl, or —N═CR¹R², wherein the substituents are selected from the group consisting of halogeno, —CN, —NO₂, —O—(CH₂)—O—, —CO₂H and salts thereof, —OR¹⁰⁰, —CO₂R¹⁰⁰, —CONR¹⁰¹R¹⁰², —NR¹⁰¹R¹⁰², —NR¹⁰⁰SO₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₅ alkyl, or C₆-C₁₂ aryl; or R¹⁰¹ and R¹⁰² together with the nitrogen atom to which they are attached form a 5-7 membered heterocycle;
wherein the alkyl group and the aryl group are each substituted by 1-3 halogeno groups or 1-3 C₁-C₆ alkyl groups;
R¹ and R² are each independently phenyl or methyl;
X, Y and Z are each independently hydrogen or halogeno; and
R is hydrogen or C₁-C₆ alkyl or halogen-substituted alkyl.

3. The treatment method according to claim 1, wherein the cancer is selected from the group consisting of ovarian cancer, breast cancer, pancreatic cancer, fallopian tube cancer, primary peritoneal cancer, gastric cancer, prostate cancer, liver cancer, colon cancer, rectal cancer, lung cancer, and bladder cancer.

4. The treatment method according to claim 1 or 2, wherein the KRAS mutation is selected from the group consisting of KRAS-G12D mutation, KRAS-G12V mutation and KRAS-G12C mutation;
preferably, the KRAS mutation is selected from the KRAS-G12D mutation.

5. The treatment method according to claim 4, wherein the TMB (Tumor Mutation Load (burden)) level of the mutation is medium.

6. The treatment method according to claim 1 or 2, wherein the other therapeutic drugs are selected from the group consisting of KRAS inhibitors and immunotherapy drugs, wherein the KRAS inhibitors are selected from the group consisting of sotorasib (AMG510), adagrasib (MRTX849), GDC6036, LY3499446, JNJ74699157 (ARS3248) and D-1553, and the immunotherapy drugs are selected from the group consisting of PD-1 monoclonal antibodies and PD-L1 monoclonal antibodies.

7. The treatment method according to claim 2, wherein the compounds of Formulae (1) and (2) are selected from the group consisting of: 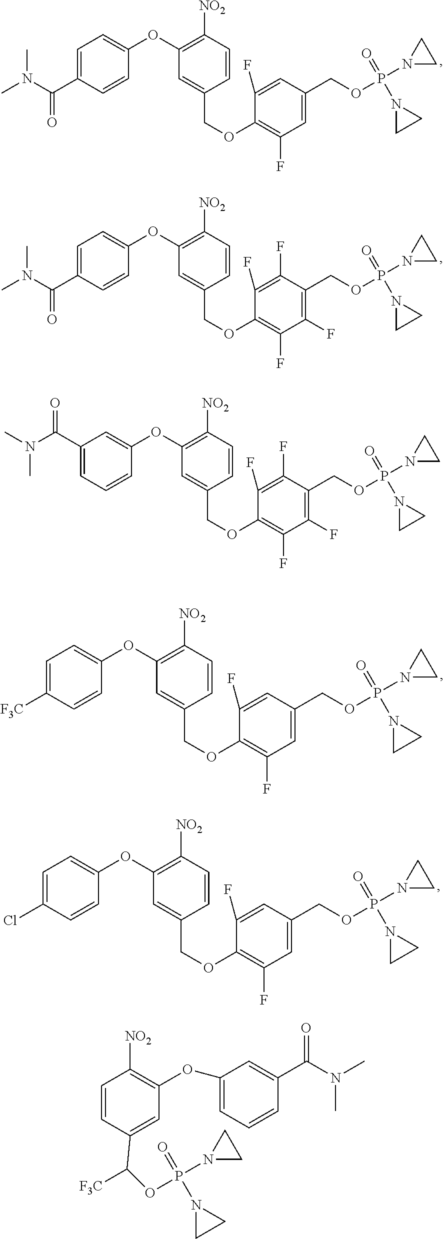 [SMILES: CN(C)C(=O)c1ccc(Oc2cc(COc3c(F)cc(COP(=O)(N4CC4)N4CC4)cc3F)ccc2[N+](=O)[O-])cc1 ; CN(C)C(=O)c1ccc(Oc2cc(COc3c(F)c(F)c(COP(=O)(N4CC4)N4CC4)c(F)c3F)ccc2[N+](=O)[O-])cc1 ; CN(C)C(=O)c1cccc(Oc2cc(COc3c(F)c(F)c(COP(=O)(N4CC4)N4CC4)c(F)c3F)ccc2[N+](=O)[O-])c1 ; O=[N+]([O-])c1ccc(COc2c(F)cc(COP(=O)(N3CC3)N3CC3)cc2F)cc1Oc1ccc(C(F)(F)F)cc1 ; O=[N+]([O-])c1ccc(COc2c(F)cc(COP(=O)(N3CC3)N3CC3)cc2F)cc1Oc1ccc(Cl)cc1 ; CN(C)C(=O)c1cccc(Oc2cc(C(OP(=O)(N3CC3)N3CC3)C(F)(F)F)ccc2[N+](=O)[O-])c1] 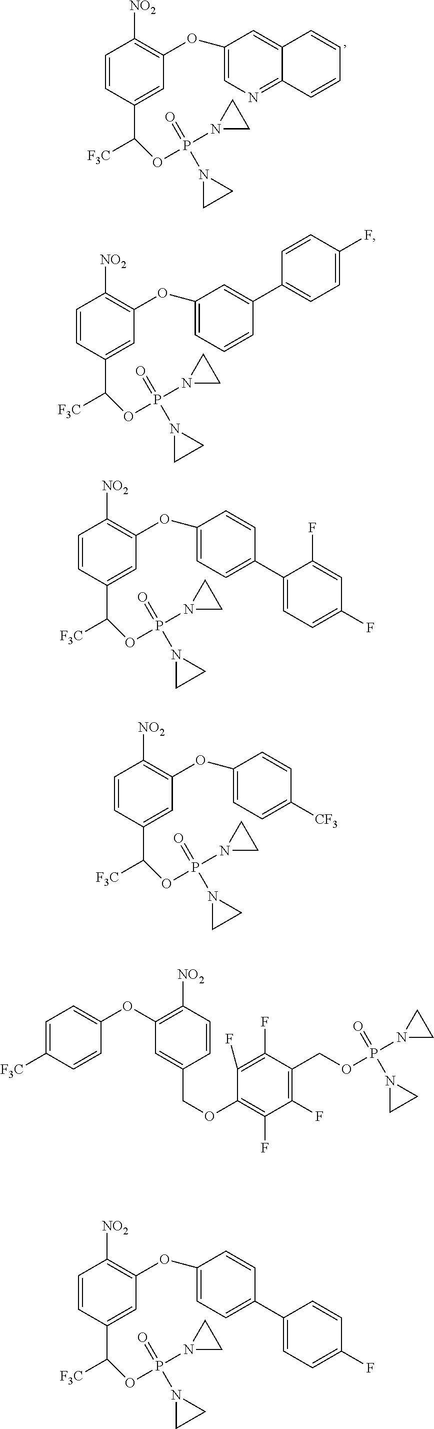 [SMILES: O=[N+]([O-])c1ccc(C(OP(=O)(N2CC2)N2CC2)C(F)(F)F)cc1Oc1cnc2ccccc2c1 ; O=[N+]([O-])c1ccc(C(OP(=O)(N2CC2)N2CC2)C(F)(F)F)cc1Oc1cccc(-c2ccc(F)cc2)c1 ; O=[N+]([O-])c1ccc(C(OP(=O)(N2CC2)N2CC2)C(F)(F)F)cc1Oc1ccc(-c2ccc(F)cc2F)cc1 ; O=[N+]([O-])c1ccc(C(OP(=O)(N2CC2)N2CC2)C(F)(F)F)cc1Oc1ccc(C(F)(F)F)cc1 ; O=[N+]([O-])c1ccc(COc2c(F)c(F)c(COP(=O)(N3CC3)N3CC3)c(F)c2F)cc1Oc1ccc(C(F)(F)F)cc1 ; O=[N+]([O-])c1ccc(C(OP(=O)(N2CC2)N2CC2)C(F)(F)F)cc1Oc1ccc(-c2ccc(F)cc2)cc1] 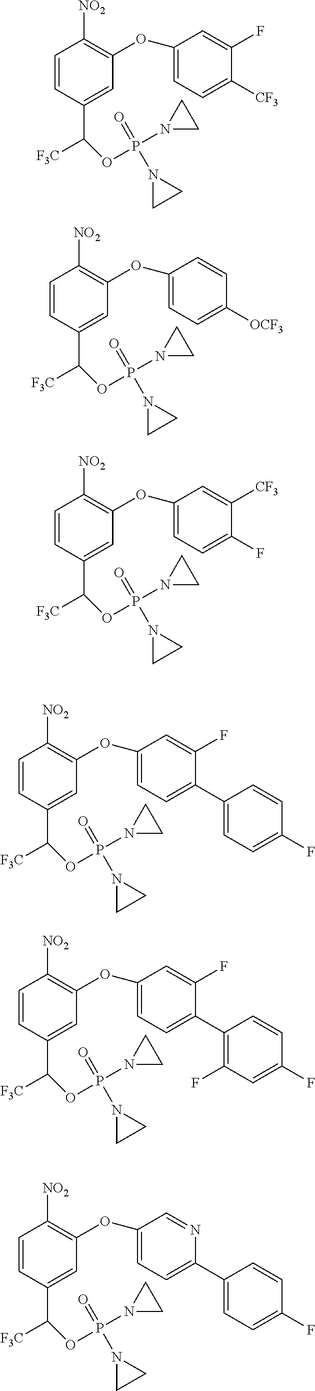 [SMILES: O=[N+]([O-])c1ccc(C(OP(=O)(N2CC2)N2CC2)C(F)(F)F)cc1Oc1ccc(C(F)(F)F)c(F)c1 ; O=[N+]([O-])c1ccc(C(OP(=O)(N2CC2)N2CC2)C(F)(F)F)cc1Oc1ccc(OC(F)(F)F)cc1 ; O=[N+]([O-])c1ccc(C(OP(=O)(N2CC2)N2CC2)C(F)(F)F)cc1Oc1ccc(F)c(C(F)(F)F)c1 ; O=[N+]([O-])c1ccc(C(OP(=O)(N2CC2)N2CC2)C(F)(F)F)cc1Oc1ccc(-c2ccc(F)cc2)c(F)c1 ; O=[N+]([O-])c1ccc(C(OP(=O)(N2CC2)N2CC2)C(F)(F)F)cc1Oc1ccc(-c2ccc(F)cc2F)c(F)c1 ; O=[N+]([O-])c1ccc(C(OP(=O)(N2CC2)N2CC2)C(F)(F)F)cc1Oc1ccc(-c2ccc(F)cc2)nc1] 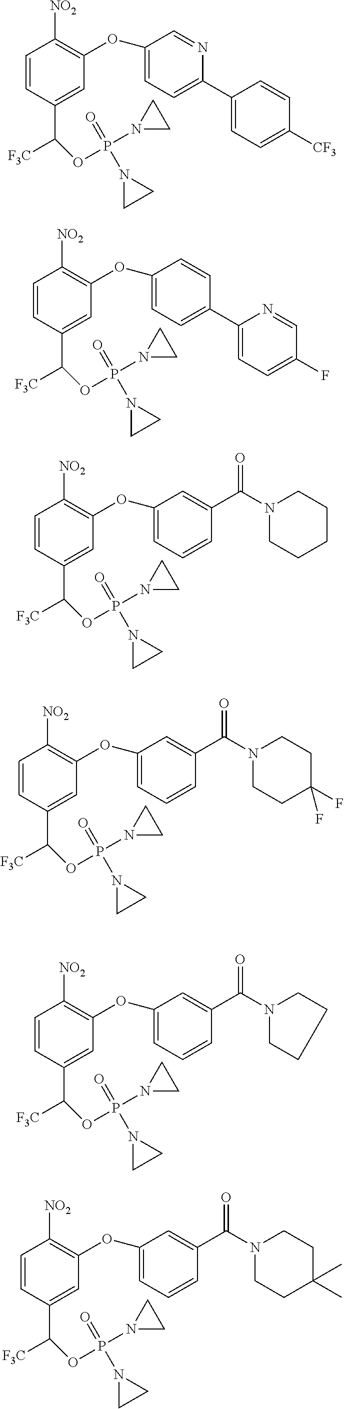 [SMILES: O=[N+]([O-])c1ccc(C(OP(=O)(N2CC2)N2CC2)C(F)(F)F)cc1Oc1ccc(-c2ccc(C(F)(F)F)cc2)nc1 ; O=[N+]([O-])c1ccc(C(OP(=O)(N2CC2)N2CC2)C(F)(F)F)cc1Oc1ccc(-c2ccc(F)cn2)cc1 ; O=C(c1cccc(Oc2cc(C(OP(=O)(N3CC3)N3CC3)C(F)(F)F)ccc2[N+](=O)[O-])c1)N1CCCCC1 ; O=C(c1cccc(Oc2cc(C(OP(=O)(N3CC3)N3CC3)C(F)(F)F)ccc2[N+](=O)[O-])c1)N1CCC(F)(F)CC1 ; O=C(c1cccc(Oc2cc(C(OP(=O)(N3CC3)N3CC3)C(F)(F)F)ccc2[N+](=O)[O-])c1)N1CCCC1 ; CC1(C)CCN(C(=O)c2cccc(Oc3cc(C(OP(=O)(N4CC4)N4CC4)C(F)(F)F)ccc3[N+](=O)[O-])c2)CC1] 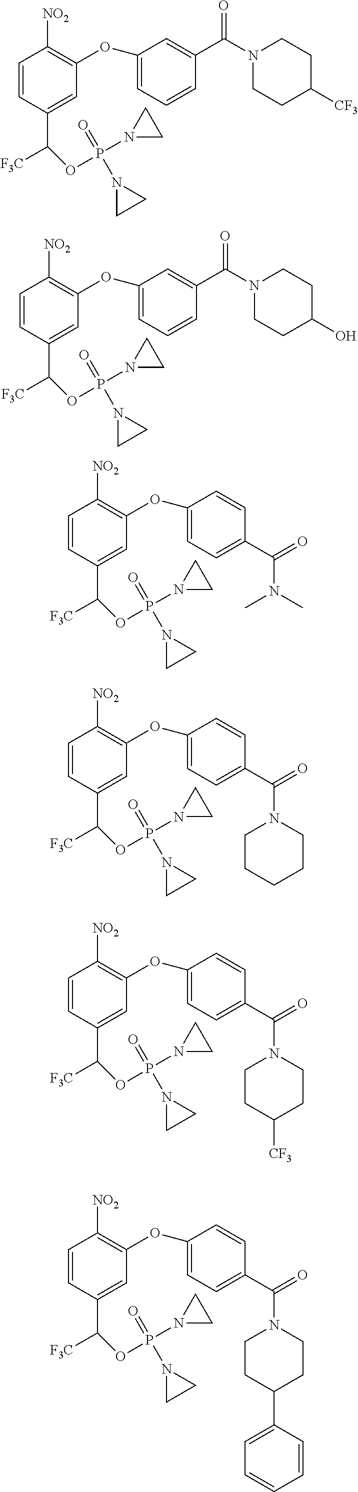 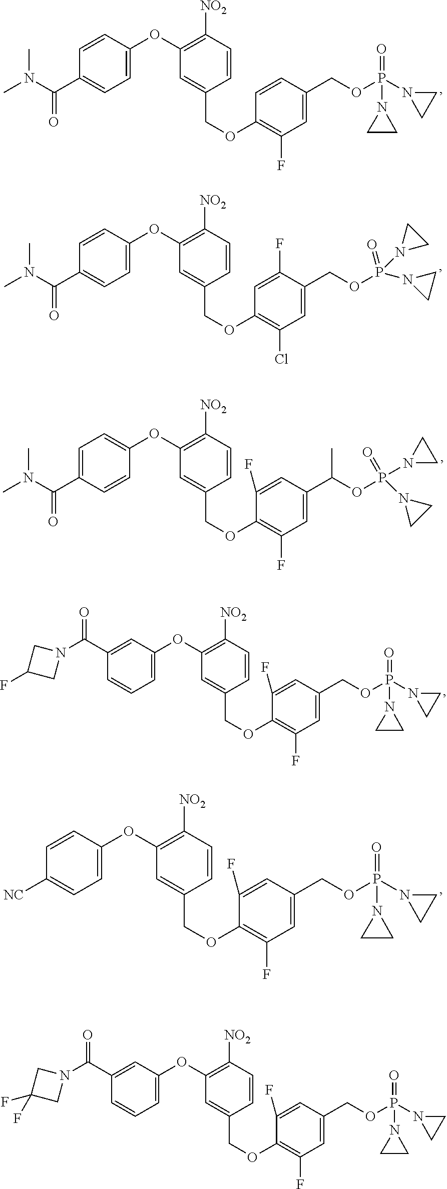 [SMILES: CN(C)C(=O)c1ccc(Oc2cc(COc3ccc(COP(=O)(N4CC4)N4CC4)cc3F)ccc2[N+](=O)[O-])cc1 ; CN(C)C(=O)c1ccc(Oc2cc(COc3cc(F)c(COP(=O)(N4CC4)N4CC4)cc3Cl)ccc2[N+](=O)[O-])cc1 ; CC(OP(=O)(N1CC1)N1CC1)c1cc(F)c(OCc2ccc([N+](=O)[O-])c(Oc3ccc(C(=O)N(C)C)cc3)c2)c(F)c1 ; O=C(c1cccc(Oc2cc(COc3c(F)cc(COP(=O)(N4CC4)N4CC4)cc3F)ccc2[N+](=O)[O-])c1)N1CC(F)C1 ; N#Cc1ccc(Oc2cc(COc3c(F)cc(COP(=O)(N4CC4)N4CC4)cc3F)ccc2[N+](=O)[O-])cc1 ; O=C(c1cccc(Oc2cc(COc3c(F)cc(COP(=O)(N4CC4)N4CC4)cc3F)ccc2[N+](=O)[O-])c1)N1CC(F)(F)C1] 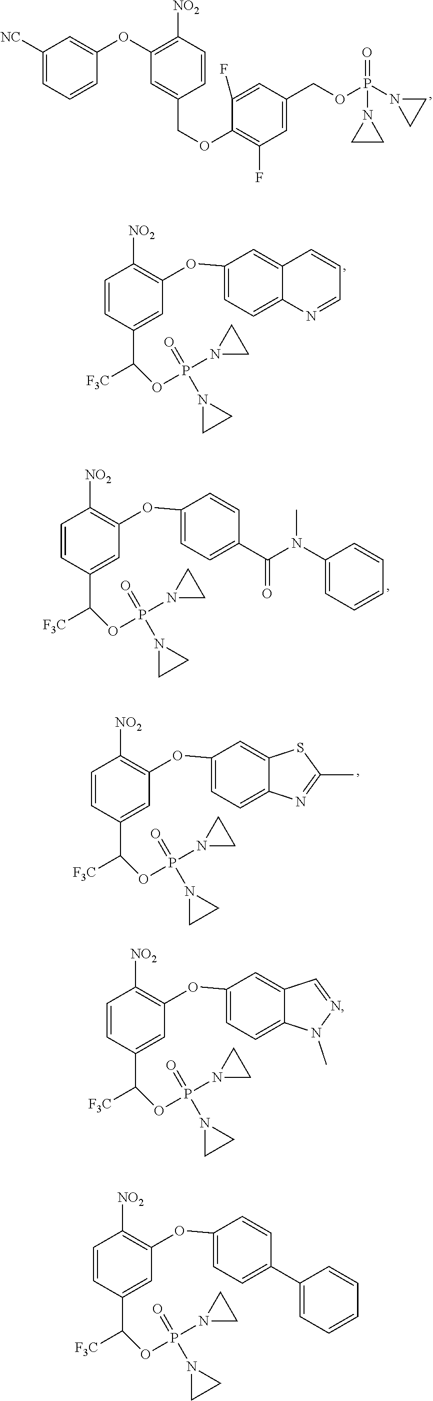 [SMILES: N#Cc1cccc(Oc2cc(COc3c(F)cc(COP(=O)(N4CC4)N4CC4)cc3F)ccc2[N+](=O)[O-])c1 ; O=[N+]([O-])c1ccc(C(OP(=O)(N2CC2)N2CC2)C(F)(F)F)cc1Oc1ccc2ncccc2c1 ; CN(C(=O)c1ccc(Oc2cc(C(OP(=O)(N3CC3)N3CC3)C(F)(F)F)ccc2[N+](=O)[O-])cc1)c1ccccc1 ; Cc1nc2ccc(Oc3cc(C(OP(=O)(N4CC4)N4CC4)C(F)(F)F)ccc3[N+](=O)[O-])cc2s1 ; Cn1ncc2cc(Oc3cc(C(OP(=O)(N4CC4)N4CC4)C(F)(F)F)ccc3[N+](=O)[O-])ccc21 ; O=[N+]([O-])c1ccc(C(OP(=O)(N2CC2)N2CC2)C(F)(F)F)cc1Oc1ccc(-c2ccccc2)cc1] 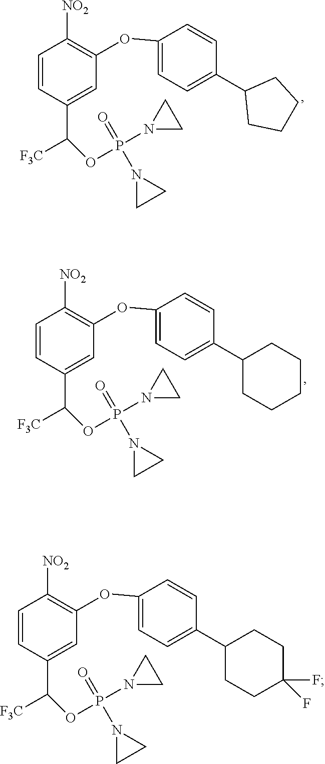 [SMILES: O=[N+]([O-])c1ccc(C(OP(=O)(N2CC2)N2CC2)C(F)(F)F)cc1Oc1ccc(C2CCCC2)cc1 ; O=[N+]([O-])c1ccc(C(OP(=O)(N2CC2)N2CC2)C(F)(F)F)cc1Oc1ccc(C2CCCCC2)cc1 ; O=[N+]([O-])c1ccc(C(OP(=O)(N2CC2)N2CC2)C(F)(F)F)cc1Oc1ccc(C2CCC(F)(F)CC2)cc1]
the compound of Formula (3) is selected from the group consisting of: 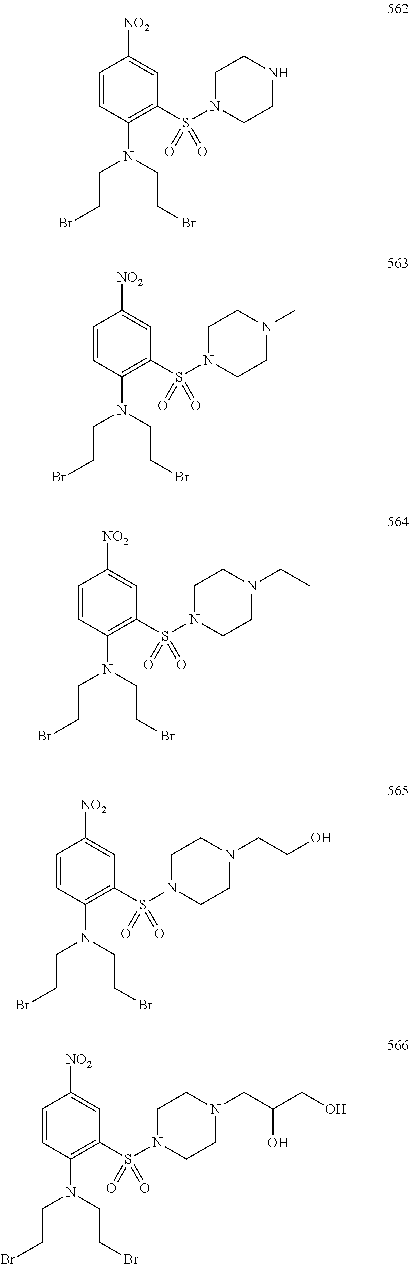 [SMILES: O=[N+]([O-])c1ccc(N(CCBr)CCBr)c(S(=O)(=O)N2CCNCC2)c1 ; CN1CCN(S(=O)(=O)c2cc([N+](=O)[O-])ccc2N(CCBr)CCBr)CC1 ; CCN1CCN(S(=O)(=O)c2cc([N+](=O)[O-])ccc2N(CCBr)CCBr)CC1 ; O=[N+]([O-])c1ccc(N(CCBr)CCBr)c(S(=O)(=O)N2CCN(CCO)CC2)c1 ; O=[N+]([O-])c1ccc(N(CCBr)CCBr)c(S(=O)(=O)N2CCN(CC(O)CO)CC2)c1] 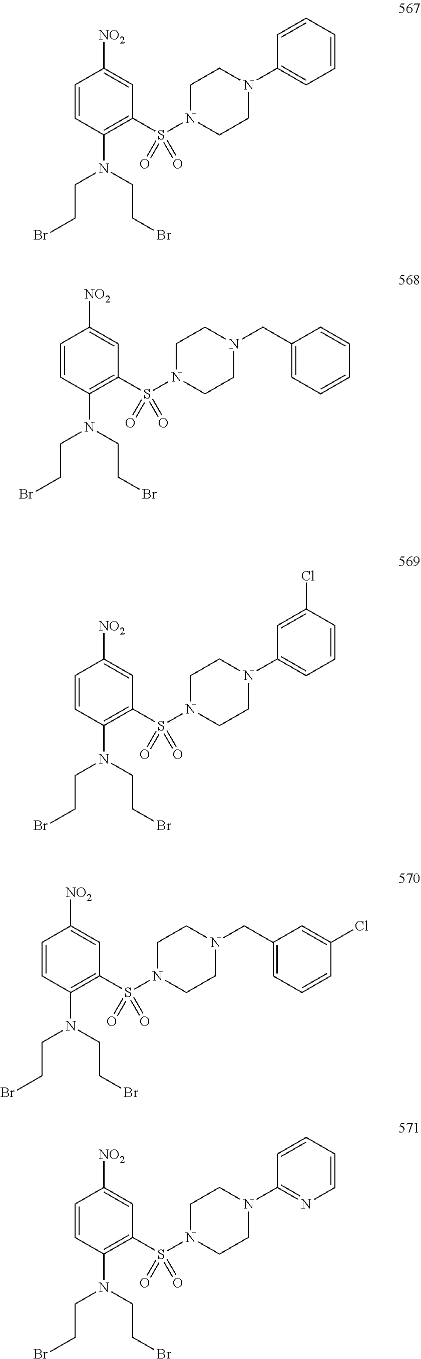 [SMILES: O=[N+]([O-])c1ccc(N(CCBr)CCBr)c(S(=O)(=O)N2CCN(c3ccccc3)CC2)c1 ; O=[N+]([O-])c1ccc(N(CCBr)CCBr)c(S(=O)(=O)N2CCN(Cc3ccccc3)CC2)c1 ; O=[N+]([O-])c1ccc(N(CCBr)CCBr)c(S(=O)(=O)N2CCN(c3cccc(Cl)c3)CC2)c1 ; O=[N+]([O-])c1ccc(N(CCBr)CCBr)c(S(=O)(=O)N2CCN(Cc3cccc(Cl)c3)CC2)c1 ; O=[N+]([O-])c1ccc(N(CCBr)CCBr)c(S(=O)(=O)N2CCN(c3ccccn3)CC2)c1] 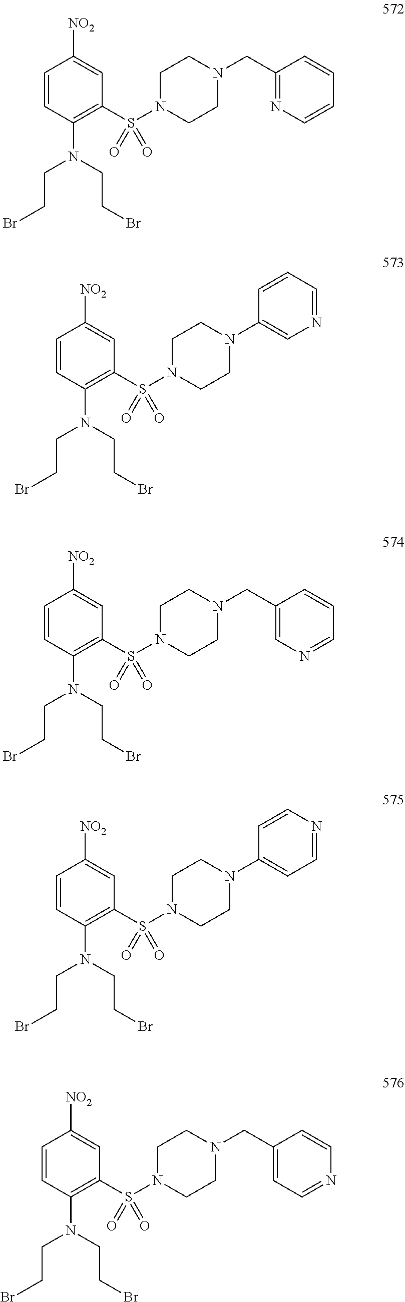 [SMILES: O=[N+]([O-])c1ccc(N(CCBr)CCBr)c(S(=O)(=O)N2CCN(Cc3ccccn3)CC2)c1 ; O=[N+]([O-])c1ccc(N(CCBr)CCBr)c(S(=O)(=O)N2CCN(c3cccnc3)CC2)c1 ; O=[N+]([O-])c1ccc(N(CCBr)CCBr)c(S(=O)(=O)N2CCN(Cc3cccnc3)CC2)c1 ; O=[N+]([O-])c1ccc(N(CCBr)CCBr)c(S(=O)(=O)N2CCN(c3ccncc3)CC2)c1 ; O=[N+]([O-])c1ccc(N(CCBr)CCBr)c(S(=O)(=O)N2CCN(Cc3ccncc3)CC2)c1] 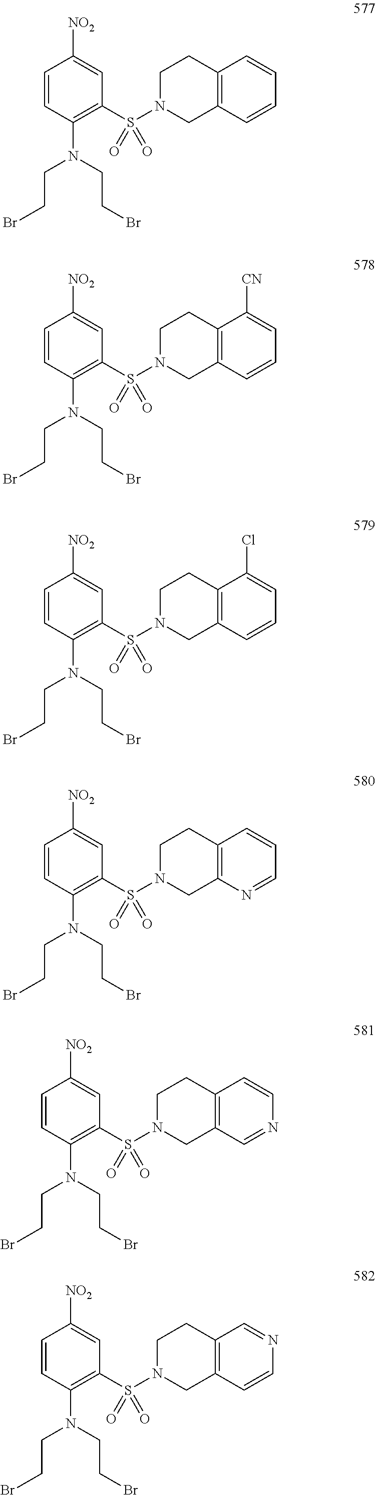 [SMILES: O=[N+]([O-])c1ccc(N(CCBr)CCBr)c(S(=O)(=O)N2CCc3ccccc3C2)c1 ; N#Cc1cccc2c1CCN(S(=O)(=O)c1cc([N+](=O)[O-])ccc1N(CCBr)CCBr)C2 ; O=[N+]([O-])c1ccc(N(CCBr)CCBr)c(S(=O)(=O)N2CCc3c(Cl)cccc3C2)c1 ; O=[N+]([O-])c1ccc(N(CCBr)CCBr)c(S(=O)(=O)N2CCc3cccnc3C2)c1 ; O=[N+]([O-])c1ccc(N(CCBr)CCBr)c(S(=O)(=O)N2CCc3ccncc3C2)c1 ; O=[N+]([O-])c1ccc(N(CCBr)CCBr)c(S(=O)(=O)N2CCc3cnccc3C2)c1] 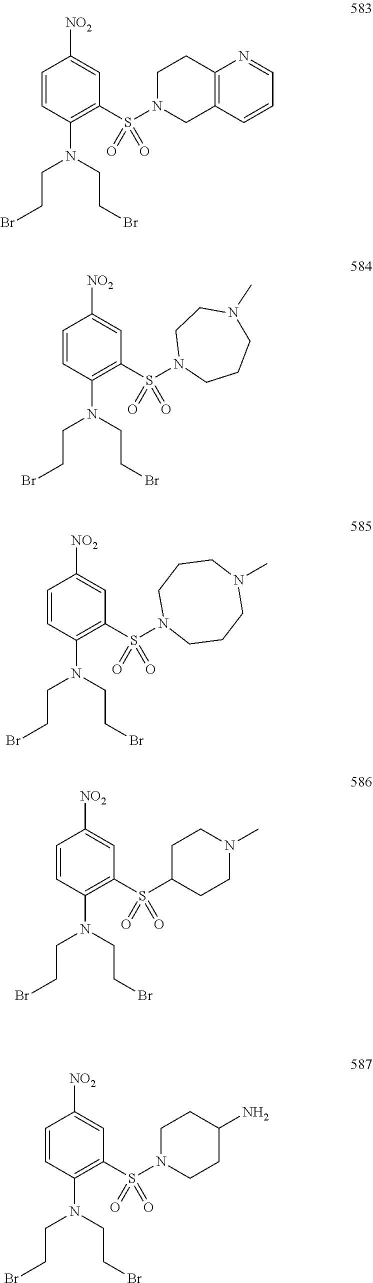 [SMILES: O=[N+]([O-])c1ccc(N(CCBr)CCBr)c(S(=O)(=O)N2CCc3ncccc3C2)c1 ; CN1CCCN(S(=O)(=O)c2cc([N+](=O)[O-])ccc2N(CCBr)CCBr)CC1 ; CN1CCCN(S(=O)(=O)c2cc([N+](=O)[O-])ccc2N(CCBr)CCBr)CCC1 ; CN1CCC(S(=O)(=O)c2cc([N+](=O)[O-])ccc2N(CCBr)CCBr)CC1 ; NC1CCN(S(=O)(=O)c2cc([N+](=O)[O-])ccc2N(CCBr)CCBr)CC1] 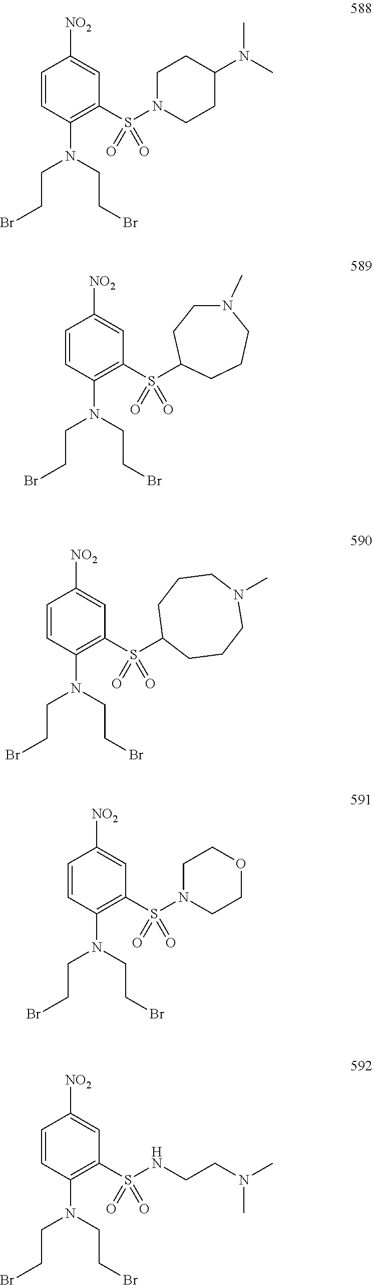 [SMILES: CN(C)C1CCN(S(=O)(=O)c2cc([N+](=O)[O-])ccc2N(CCBr)CCBr)CC1 ; CN1CCCC(S(=O)(=O)c2cc([N+](=O)[O-])ccc2N(CCBr)CCBr)CC1 ; CN1CCCC(S(=O)(=O)c2cc([N+](=O)[O-])ccc2N(CCBr)CCBr)CCC1 ; O=[N+]([O-])c1ccc(N(CCBr)CCBr)c(S(=O)(=O)N2CCOCC2)c1 ; CN(C)CCNS(=O)(=O)c1cc([N+](=O)[O-])ccc1N(CCBr)CCBr] 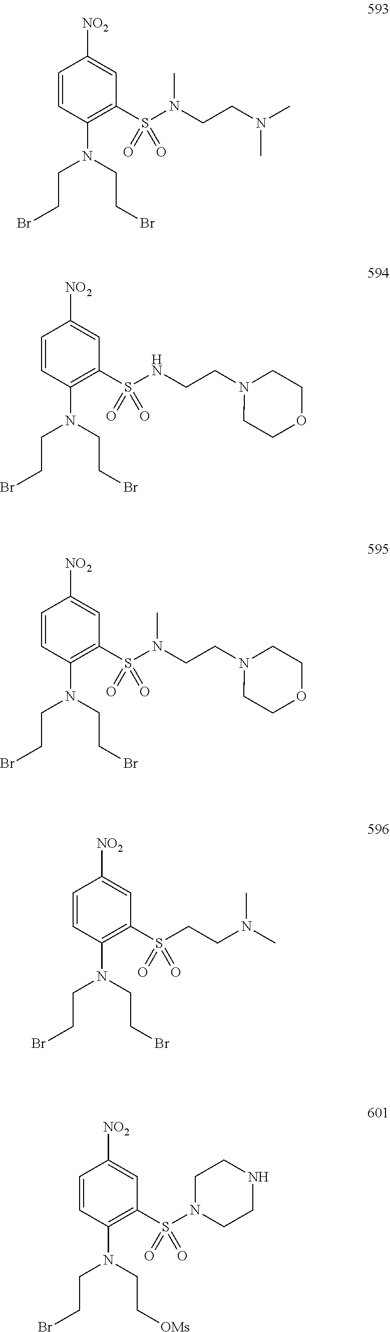 [SMILES: CN(C)CCN(C)S(=O)(=O)c1cc([N+](=O)[O-])ccc1N(CCBr)CCBr ; O=[N+]([O-])c1ccc(N(CCBr)CCBr)c(S(=O)(=O)NCCN2CCOCC2)c1 ; CN(CCN1CCOCC1)S(=O)(=O)c1cc([N+](=O)[O-])ccc1N(CCBr)CCBr ; CN(C)CCS(=O)(=O)c1cc([N+](=O)[O-])ccc1N(CCBr)CCBr ; CS(=O)(=O)OCCN(CCBr)c1ccc([N+](=O)[O-])cc1S(=O)(=O)N1CCNCC1] 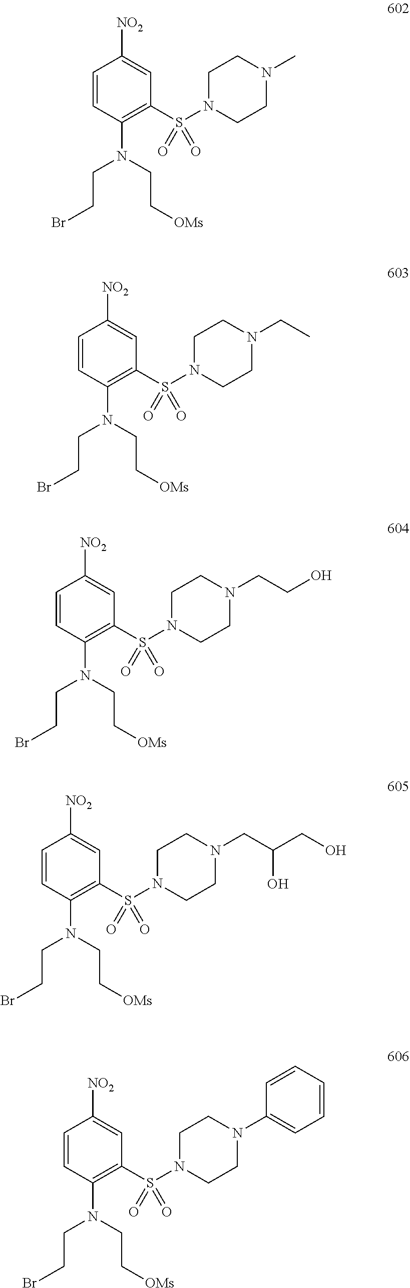 [SMILES: CN1CCN(S(=O)(=O)c2cc([N+](=O)[O-])ccc2N(CCBr)CCOS(C)(=O)=O)CC1 ; CCN1CCN(S(=O)(=O)c2cc([N+](=O)[O-])ccc2N(CCBr)CCOS(C)(=O)=O)CC1 ; CS(=O)(=O)OCCN(CCBr)c1ccc([N+](=O)[O-])cc1S(=O)(=O)N1CCN(CCO)CC1 ; CS(=O)(=O)OCCN(CCBr)c1ccc([N+](=O)[O-])cc1S(=O)(=O)N1CCN(CC(O)CO)CC1 ; CS(=O)(=O)OCCN(CCBr)c1ccc([N+](=O)[O-])cc1S(=O)(=O)N1CCN(c2ccccc2)CC1] 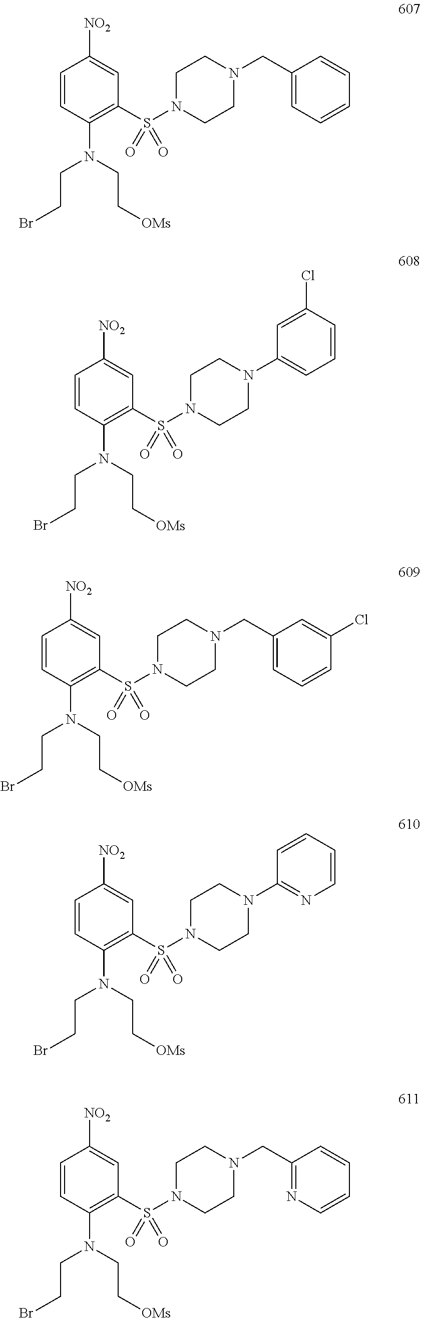 [SMILES: CS(=O)(=O)OCCN(CCBr)c1ccc([N+](=O)[O-])cc1S(=O)(=O)N1CCN(Cc2ccccc2)CC1 ; CS(=O)(=O)OCCN(CCBr)c1ccc([N+](=O)[O-])cc1S(=O)(=O)N1CCN(c2cccc(Cl)c2)CC1 ; CS(=O)(=O)OCCN(CCBr)c1ccc([N+](=O)[O-])cc1S(=O)(=O)N1CCN(Cc2cccc(Cl)c2)CC1 ; CS(=O)(=O)OCCN(CCBr)c1ccc([N+](=O)[O-])cc1S(=O)(=O)N1CCN(c2ccccn2)CC1 ; CS(=O)(=O)OCCN(CCBr)c1ccc([N+](=O)[O-])cc1S(=O)(=O)N1CCN(Cc2ccccn2)CC1] 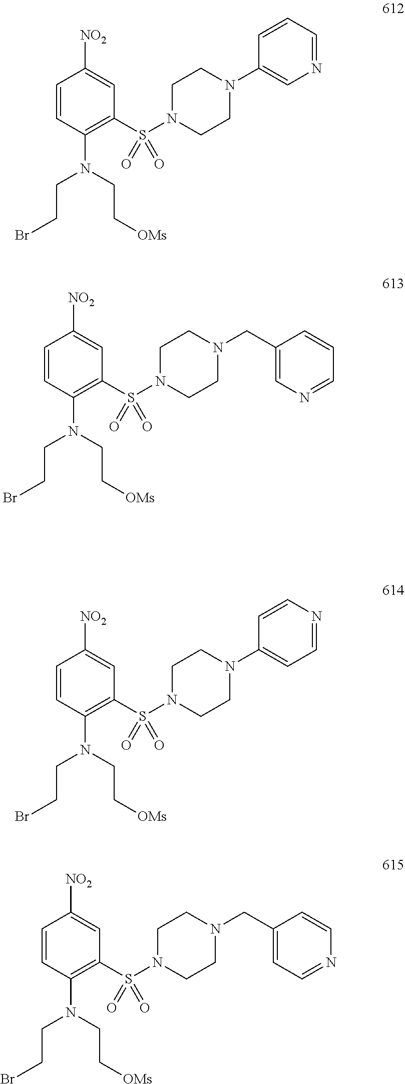 [SMILES: CS(=O)(=O)OCCN(CCBr)c1ccc([N+](=O)[O-])cc1S(=O)(=O)N1CCN(c2cccnc2)CC1 ; CS(=O)(=O)OCCN(CCBr)c1ccc([N+](=O)[O-])cc1S(=O)(=O)N1CCN(Cc2cccnc2)CC1 ; CS(=O)(=O)OCCN(CCBr)c1ccc([N+](=O)[O-])cc1S(=O)(=O)N1CCN(c2ccncc2)CC1 ; CS(=O)(=O)OCCN(CCBr)c1ccc([N+](=O)[O-])cc1S(=O)(=O)N1CCN(Cc2ccncc2)CC1] 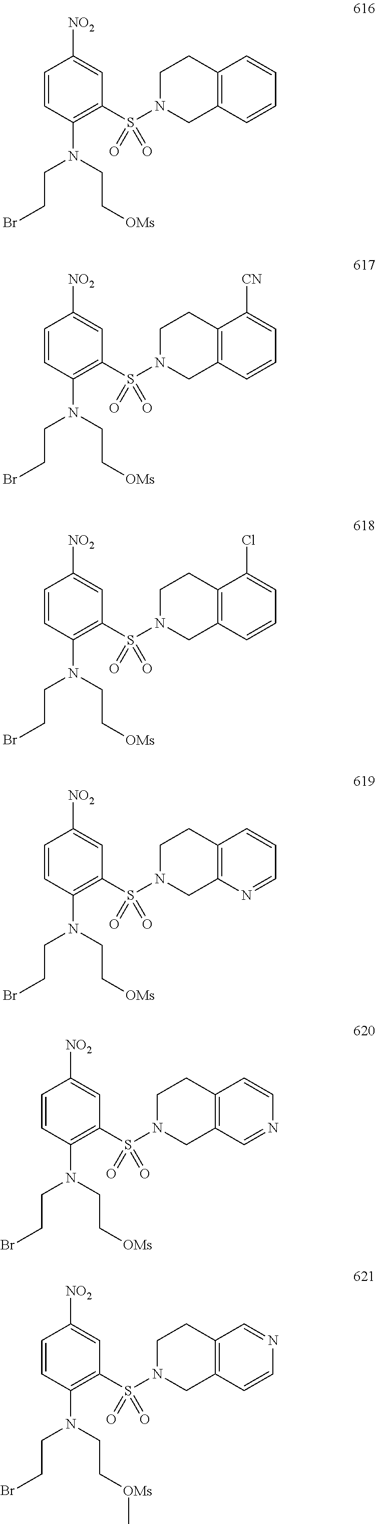 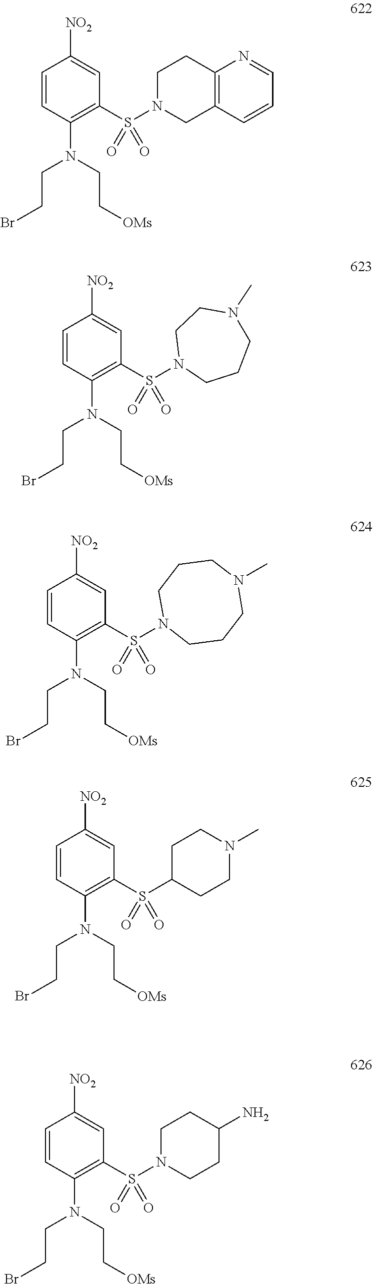 [SMILES: CS(=O)(=O)OCCN(CCBr)c1ccc([N+](=O)[O-])cc1S(=O)(=O)N1CCc2ncccc2C1 ; CN1CCCN(S(=O)(=O)c2cc([N+](=O)[O-])ccc2N(CCBr)CCOS(C)(=O)=O)CC1 ; CN1CCCN(S(=O)(=O)c2cc([N+](=O)[O-])ccc2N(CCBr)CCOS(C)(=O)=O)CCC1 ; CN1CCC(S(=O)(=O)c2cc([N+](=O)[O-])ccc2N(CCBr)CCOS(C)(=O)=O)CC1 ; CS(=O)(=O)OCCN(CCBr)c1ccc([N+](=O)[O-])cc1S(=O)(=O)N1CCC(N)CC1] 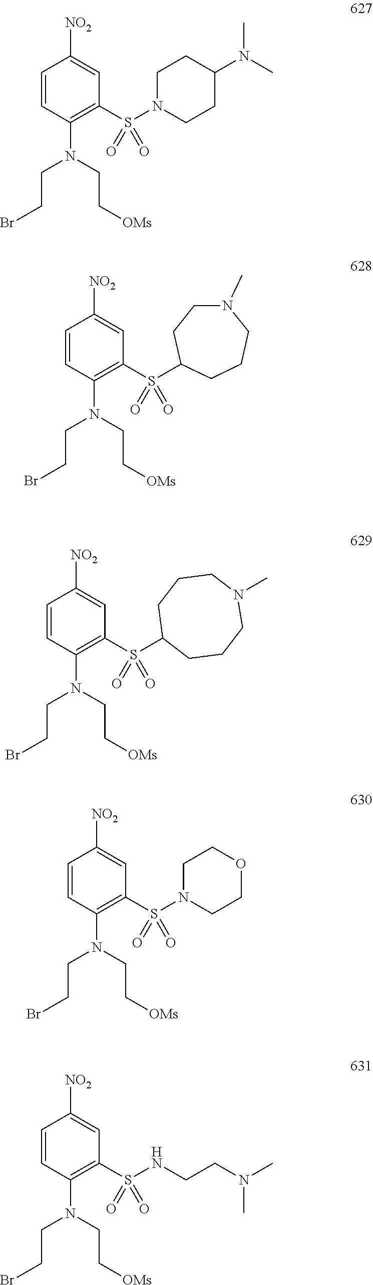 [SMILES: CN(C)C1CCN(S(=O)(=O)c2cc([N+](=O)[O-])ccc2N(CCBr)CCOS(C)(=O)=O)CC1 ; CN1CCCC(S(=O)(=O)c2cc([N+](=O)[O-])ccc2N(CCBr)CCOS(C)(=O)=O)CC1 ; CN1CCCC(S(=O)(=O)c2cc([N+](=O)[O-])ccc2N(CCBr)CCOS(C)(=O)=O)CCC1 ; CS(=O)(=O)OCCN(CCBr)c1ccc([N+](=O)[O-])cc1S(=O)(=O)N1CCOCC1 ; CN(C)CCNS(=O)(=O)c1cc([N+](=O)[O-])ccc1N(CCBr)CCOS(C)(=O)=O] 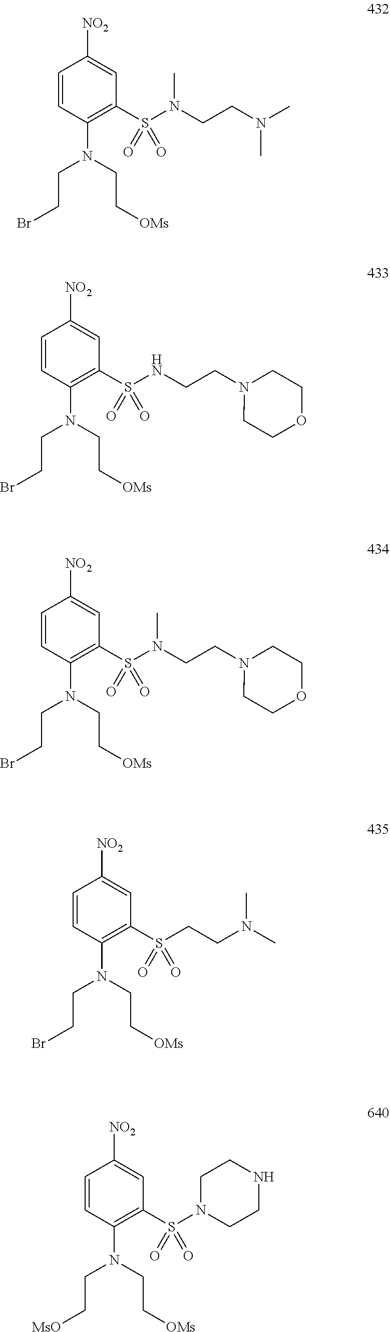 [SMILES: CN(C)CCN(C)S(=O)(=O)c1cc([N+](=O)[O-])ccc1N(CCBr)CCOS(C)(=O)=O ; CS(=O)(=O)OCCN(CCBr)c1ccc([N+](=O)[O-])cc1S(=O)(=O)NCCN1CCOCC1 ; CN(CCN1CCOCC1)S(=O)(=O)c1cc([N+](=O)[O-])ccc1N(CCBr)CCOS(C)(=O)=O ; CN(C)CCS(=O)(=O)c1cc([N+](=O)[O-])ccc1N(CCBr)CCOS(C)(=O)=O ; CS(=O)(=O)OCCN(CCOS(C)(=O)=O)c1ccc([N+](=O)[O-])cc1S(=O)(=O)N1CCNCC1] 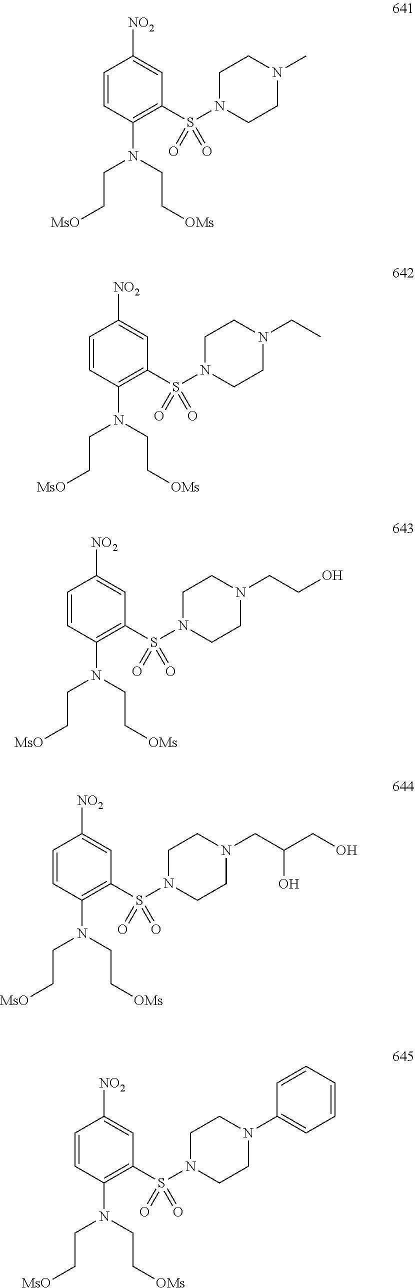 [SMILES: CN1CCN(S(=O)(=O)c2cc([N+](=O)[O-])ccc2N(CCOS(C)(=O)=O)CCOS(C)(=O)=O)CC1 ; CCN1CCN(S(=O)(=O)c2cc([N+](=O)[O-])ccc2N(CCOS(C)(=O)=O)CCOS(C)(=O)=O)CC1 ; CS(=O)(=O)OCCN(CCOS(C)(=O)=O)c1ccc([N+](=O)[O-])cc1S(=O)(=O)N1CCN(CCO)CC1 ; CS(=O)(=O)OCCN(CCOS(C)(=O)=O)c1ccc([N+](=O)[O-])cc1S(=O)(=O)N1CCN(CC(O)CO)CC1 ; CS(=O)(=O)OCCN(CCOS(C)(=O)=O)c1ccc([N+](=O)[O-])cc1S(=O)(=O)N1CCN(c2ccccc2)CC1] 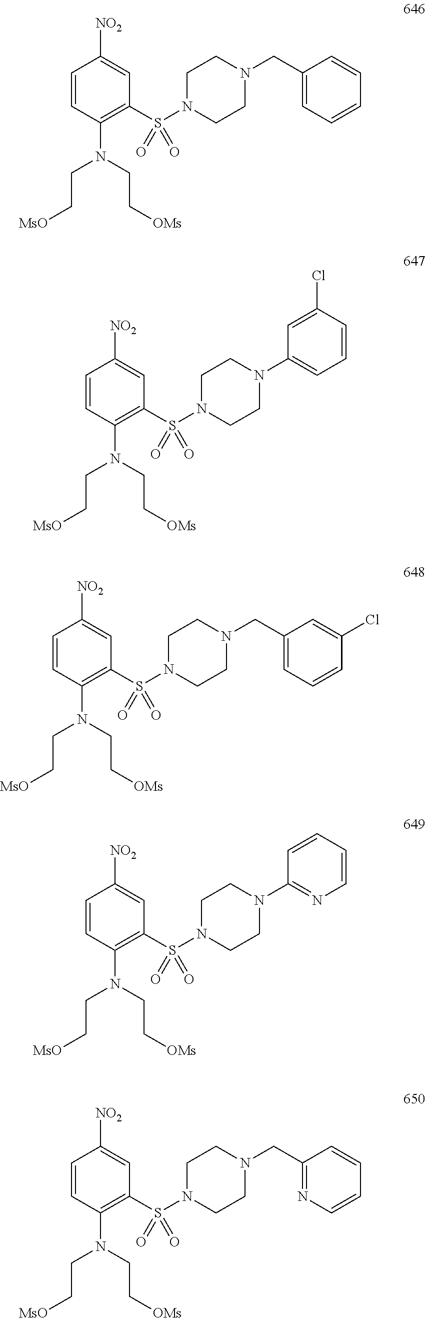 [SMILES: CS(=O)(=O)OCCN(CCOS(C)(=O)=O)c1ccc([N+](=O)[O-])cc1S(=O)(=O)N1CCN(Cc2ccccc2)CC1 ; CS(=O)(=O)OCCN(CCOS(C)(=O)=O)c1ccc([N+](=O)[O-])cc1S(=O)(=O)N1CCN(c2cccc(Cl)c2)CC1 ; CS(=O)(=O)OCCN(CCOS(C)(=O)=O)c1ccc([N+](=O)[O-])cc1S(=O)(=O)N1CCN(Cc2cccc(Cl)c2)CC1 ; CS(=O)(=O)OCCN(CCOS(C)(=O)=O)c1ccc([N+](=O)[O-])cc1S(=O)(=O)N1CCN(c2ccccn2)CC1 ; CS(=O)(=O)OCCN(CCOS(C)(=O)=O)c1ccc([N+](=O)[O-])cc1S(=O)(=O)N1CCN(Cc2ccccn2)CC1] 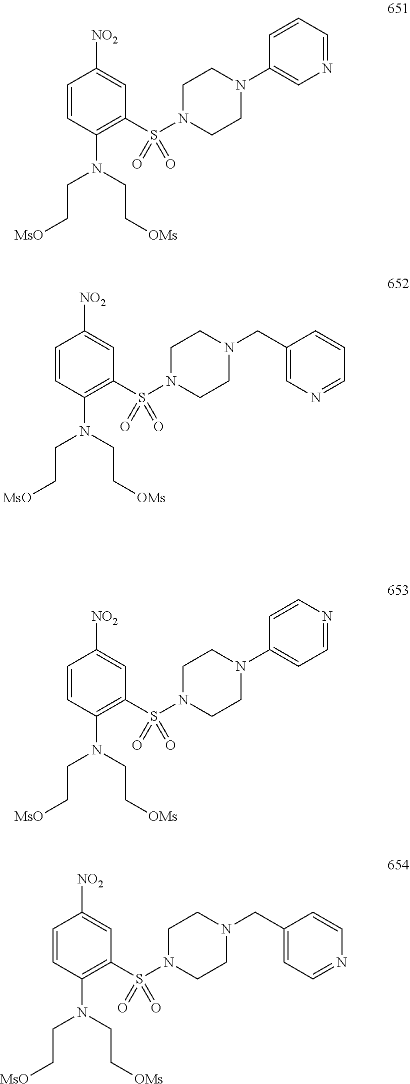 [SMILES: CS(=O)(=O)OCCN(CCOS(C)(=O)=O)c1ccc([N+](=O)[O-])cc1S(=O)(=O)N1CCN(c2cccnc2)CC1 ; CS(=O)(=O)OCCN(CCOS(C)(=O)=O)c1ccc([N+](=O)[O-])cc1S(=O)(=O)N1CCN(Cc2cccnc2)CC1 ; CS(=O)(=O)OCCN(CCOS(C)(=O)=O)c1ccc([N+](=O)[O-])cc1S(=O)(=O)N1CCN(c2ccncc2)CC1 ; CS(=O)(=O)OCCN(CCOS(C)(=O)=O)c1ccc([N+](=O)[O-])cc1S(=O)(=O)N1CCN(Cc2ccncc2)CC1] 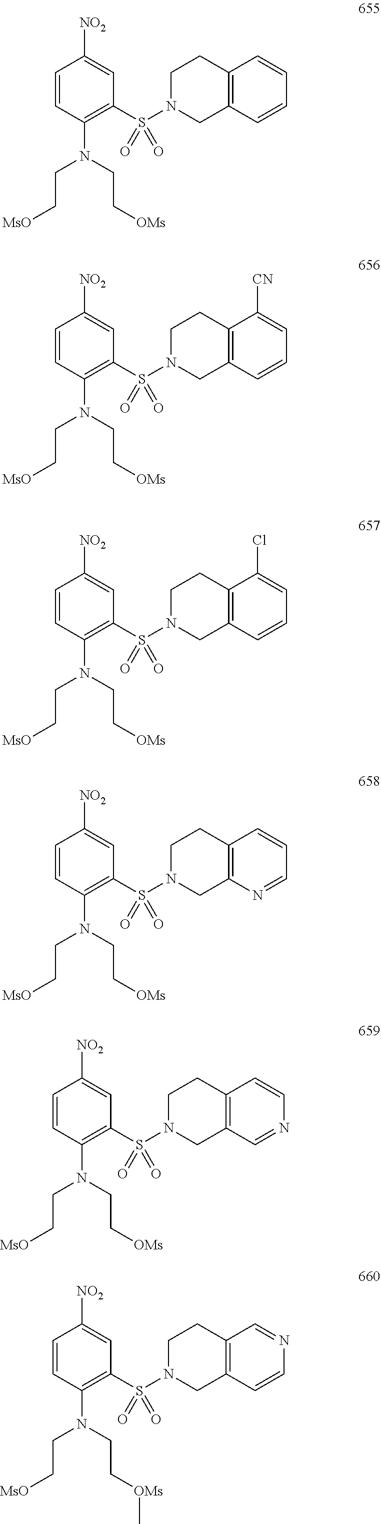 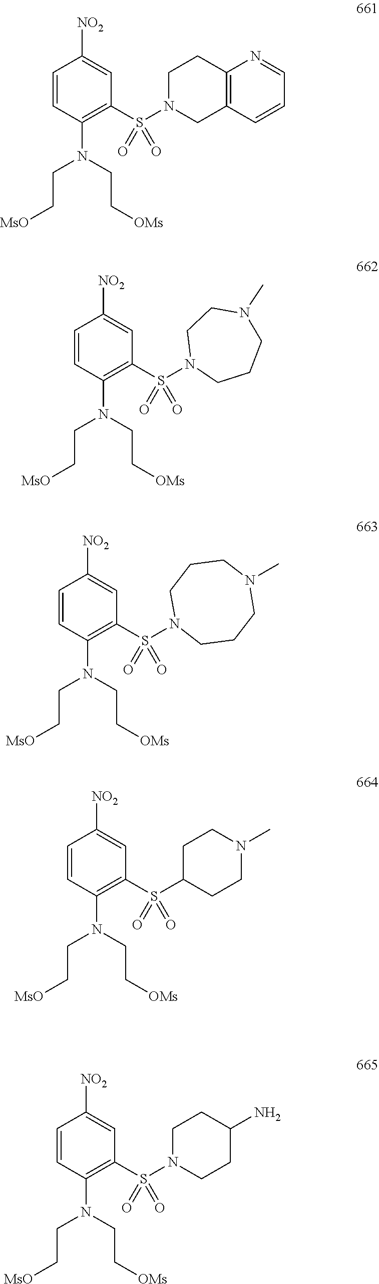 [SMILES: CS(=O)(=O)OCCN(CCOS(C)(=O)=O)c1ccc([N+](=O)[O-])cc1S(=O)(=O)N1CCc2ncccc2C1 ; CN1CCCN(S(=O)(=O)c2cc([N+](=O)[O-])ccc2N(CCOS(C)(=O)=O)CCOS(C)(=O)=O)CC1 ; CN1CCCN(S(=O)(=O)c2cc([N+](=O)[O-])ccc2N(CCOS(C)(=O)=O)CCOS(C)(=O)=O)CCC1 ; CN1CCC(S(=O)(=O)c2cc([N+](=O)[O-])ccc2N(CCOS(C)(=O)=O)CCOS(C)(=O)=O)CC1 ; CS(=O)(=O)OCCN(CCOS(C)(=O)=O)c1ccc([N+](=O)[O-])cc1S(=O)(=O)N1CCC(N)CC1] 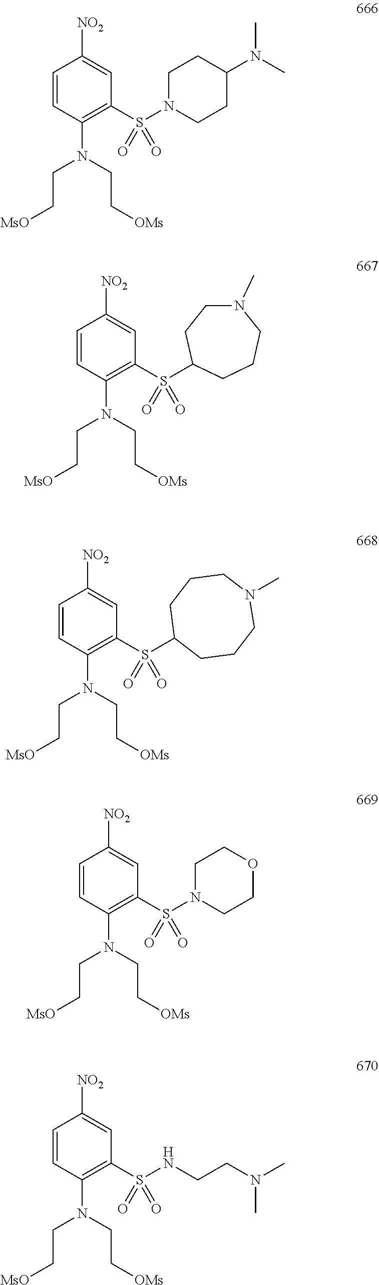 [SMILES: CN(C)C1CCN(S(=O)(=O)c2cc([N+](=O)[O-])ccc2N(CCOS(C)(=O)=O)CCOS(C)(=O)=O)CC1 ; CN1CCCC(S(=O)(=O)c2cc([N+](=O)[O-])ccc2N(CCOS(C)(=O)=O)CCOS(C)(=O)=O)CC1 ; CN1CCCC(S(=O)(=O)c2cc([N+](=O)[O-])ccc2N(CCOS(C)(=O)=O)CCOS(C)(=O)=O)CCC1 ; CS(=O)(=O)OCCN(CCOS(C)(=O)=O)c1ccc([N+](=O)[O-])cc1S(=O)(=O)N1CCOCC1 ; CN(C)CCNS(=O)(=O)c1cc([N+](=O)[O-])ccc1N(CCOS(C)(=O)=O)CCOS(C)(=O)=O] 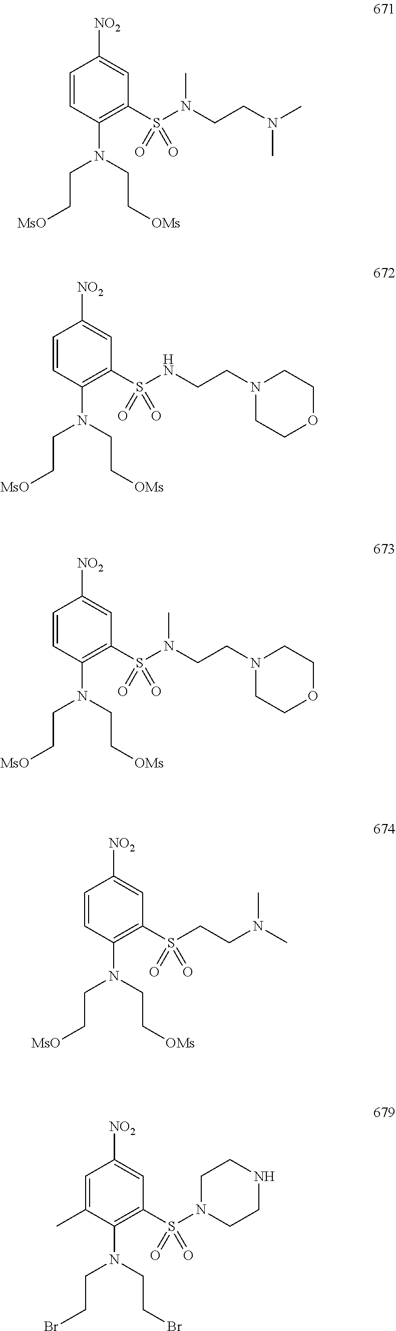 [SMILES: CN(C)CCN(C)S(=O)(=O)c1cc([N+](=O)[O-])ccc1N(CCOS(C)(=O)=O)CCOS(C)(=O)=O ; CS(=O)(=O)OCCN(CCOS(C)(=O)=O)c1ccc([N+](=O)[O-])cc1S(=O)(=O)NCCN1CCOCC1 ; CN(CCN1CCOCC1)S(=O)(=O)c1cc([N+](=O)[O-])ccc1N(CCOS(C)(=O)=O)CCOS(C)(=O)=O ; CN(C)CCS(=O)(=O)c1cc([N+](=O)[O-])ccc1N(CCOS(C)(=O)=O)CCOS(C)(=O)=O ; Cc1cc([N+](=O)[O-])cc(S(=O)(=O)N2CCNCC2)c1N(CCBr)CCBr] 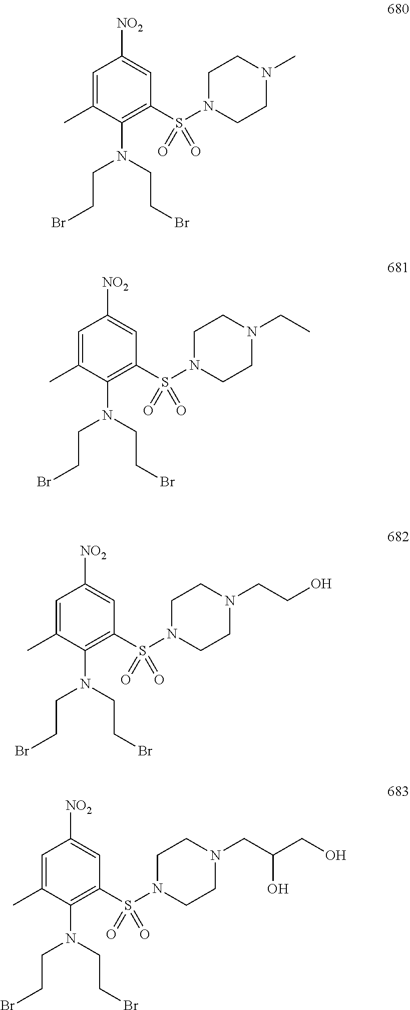 [SMILES: Cc1cc([N+](=O)[O-])cc(S(=O)(=O)N2CCN(C)CC2)c1N(CCBr)CCBr ; CCN1CCN(S(=O)(=O)c2cc([N+](=O)[O-])cc(C)c2N(CCBr)CCBr)CC1 ; Cc1cc([N+](=O)[O-])cc(S(=O)(=O)N2CCN(CCO)CC2)c1N(CCBr)CCBr ; Cc1cc([N+](=O)[O-])cc(S(=O)(=O)N2CCN(CC(O)CO)CC2)c1N(CCBr)CCBr] 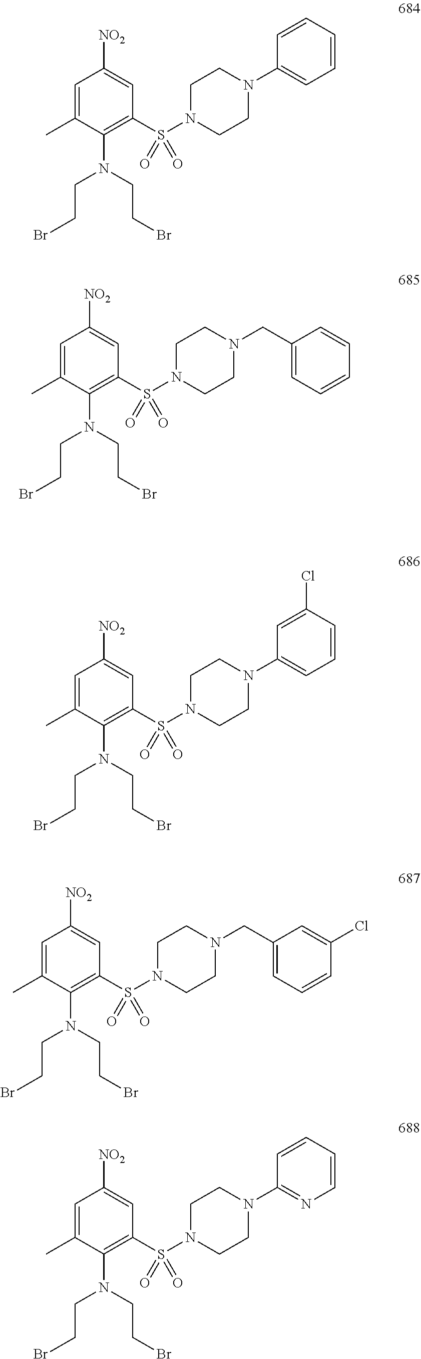 [SMILES: Cc1cc([N+](=O)[O-])cc(S(=O)(=O)N2CCN(c3ccccc3)CC2)c1N(CCBr)CCBr ; Cc1cc([N+](=O)[O-])cc(S(=O)(=O)N2CCN(Cc3ccccc3)CC2)c1N(CCBr)CCBr ; Cc1cc([N+](=O)[O-])cc(S(=O)(=O)N2CCN(c3cccc(Cl)c3)CC2)c1N(CCBr)CCBr ; Cc1cc([N+](=O)[O-])cc(S(=O)(=O)N2CCN(Cc3cccc(Cl)c3)CC2)c1N(CCBr)CCBr ; Cc1cc([N+](=O)[O-])cc(S(=O)(=O)N2CCN(c3ccccn3)CC2)c1N(CCBr)CCBr] 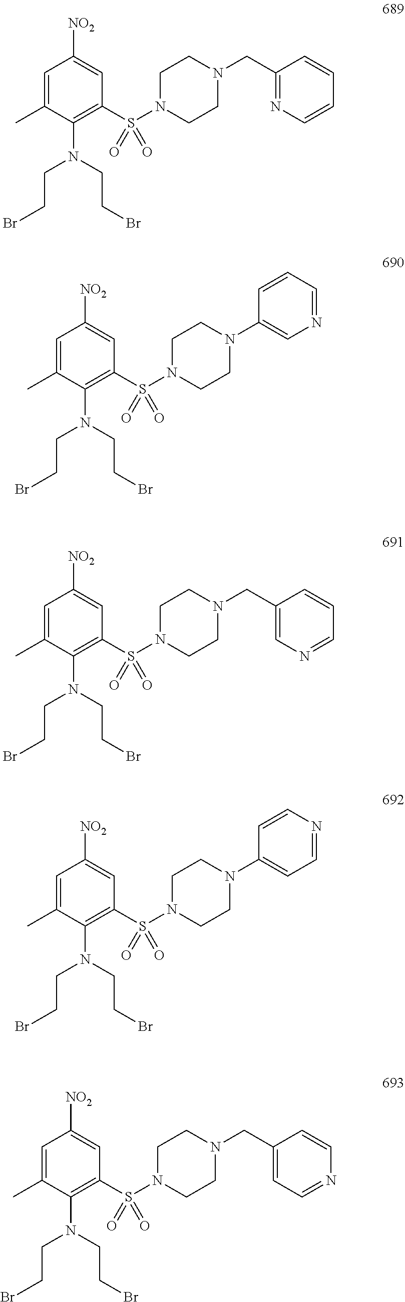 [SMILES: Cc1cc([N+](=O)[O-])cc(S(=O)(=O)N2CCN(Cc3ccccn3)CC2)c1N(CCBr)CCBr ; Cc1cc([N+](=O)[O-])cc(S(=O)(=O)N2CCN(c3cccnc3)CC2)c1N(CCBr)CCBr ; Cc1cc([N+](=O)[O-])cc(S(=O)(=O)N2CCN(Cc3cccnc3)CC2)c1N(CCBr)CCBr ; Cc1cc([N+](=O)[O-])cc(S(=O)(=O)N2CCN(c3ccncc3)CC2)c1N(CCBr)CCBr ; Cc1cc([N+](=O)[O-])cc(S(=O)(=O)N2CCN(Cc3ccncc3)CC2)c1N(CCBr)CCBr] 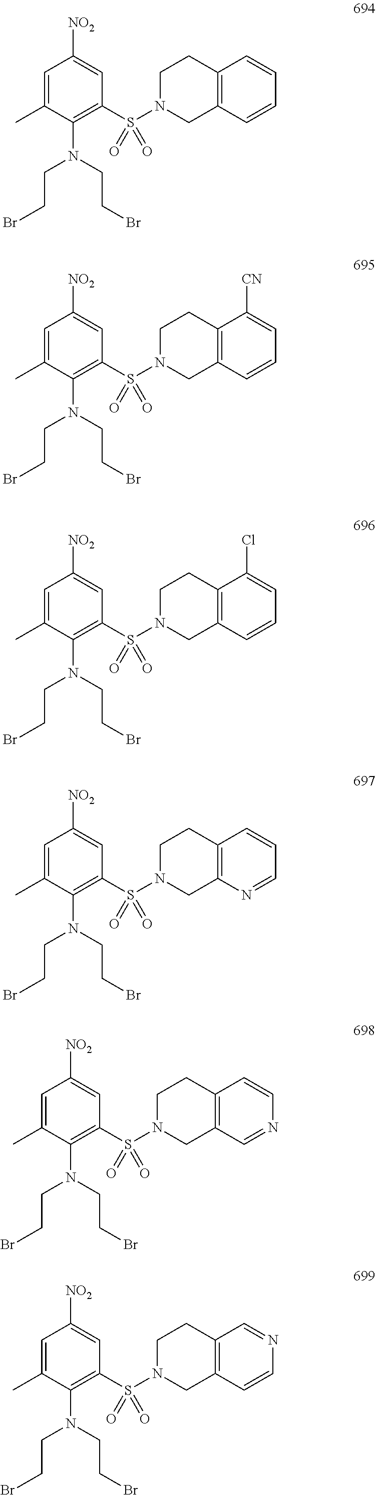 [SMILES: Cc1cc([N+](=O)[O-])cc(S(=O)(=O)N2CCc3ccccc3C2)c1N(CCBr)CCBr ; Cc1cc([N+](=O)[O-])cc(S(=O)(=O)N2CCc3c(C#N)cccc3C2)c1N(CCBr)CCBr ; Cc1cc([N+](=O)[O-])cc(S(=O)(=O)N2CCc3c(Cl)cccc3C2)c1N(CCBr)CCBr ; Cc1cc([N+](=O)[O-])cc(S(=O)(=O)N2CCc3cccnc3C2)c1N(CCBr)CCBr ; Cc1cc([N+](=O)[O-])cc(S(=O)(=O)N2CCc3ccncc3C2)c1N(CCBr)CCBr ; Cc1cc([N+](=O)[O-])cc(S(=O)(=O)N2CCc3cnccc3C2)c1N(CCBr)CCBr] 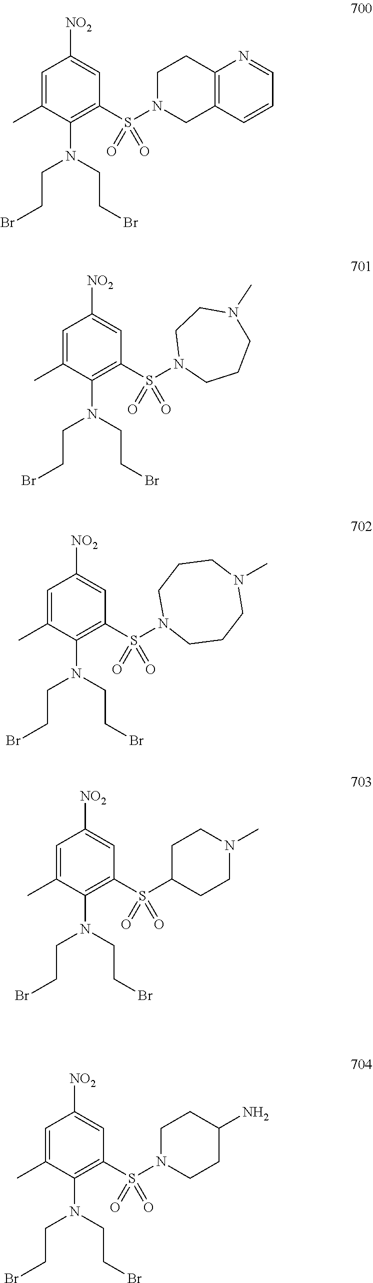 [SMILES: Cc1cc([N+](=O)[O-])cc(S(=O)(=O)N2CCc3ncccc3C2)c1N(CCBr)CCBr ; Cc1cc([N+](=O)[O-])cc(S(=O)(=O)N2CCCN(C)CC2)c1N(CCBr)CCBr ; Cc1cc([N+](=O)[O-])cc(S(=O)(=O)N2CCCN(C)CCC2)c1N(CCBr)CCBr ; Cc1cc([N+](=O)[O-])cc(S(=O)(=O)C2CCN(C)CC2)c1N(CCBr)CCBr ; Cc1cc([N+](=O)[O-])cc(S(=O)(=O)N2CCC(N)CC2)c1N(CCBr)CCBr] 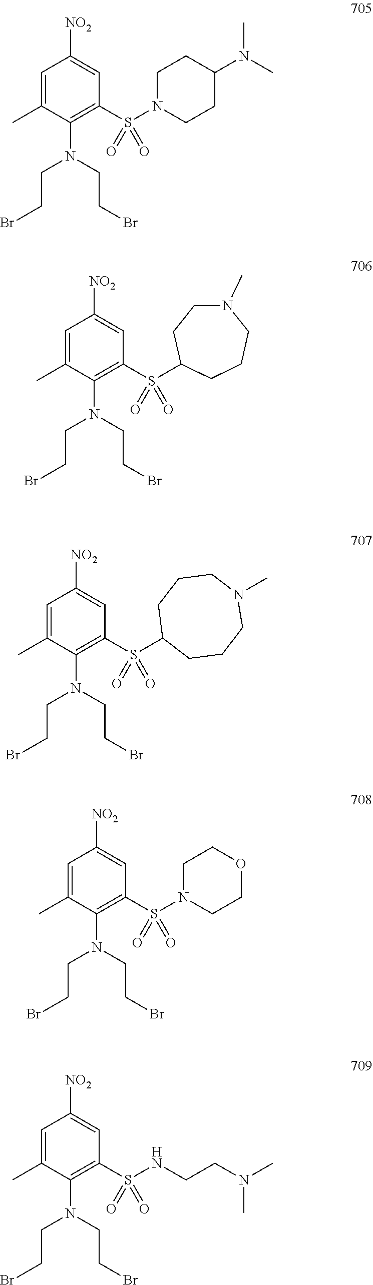 [SMILES: Cc1cc([N+](=O)[O-])cc(S(=O)(=O)N2CCC(N(C)C)CC2)c1N(CCBr)CCBr ; Cc1cc([N+](=O)[O-])cc(S(=O)(=O)C2CCCN(C)CC2)c1N(CCBr)CCBr ; Cc1cc([N+](=O)[O-])cc(S(=O)(=O)C2CCCN(C)CCC2)c1N(CCBr)CCBr ; Cc1cc([N+](=O)[O-])cc(S(=O)(=O)N2CCOCC2)c1N(CCBr)CCBr ; Cc1cc([N+](=O)[O-])cc(S(=O)(=O)NCCN(C)C)c1N(CCBr)CCBr] 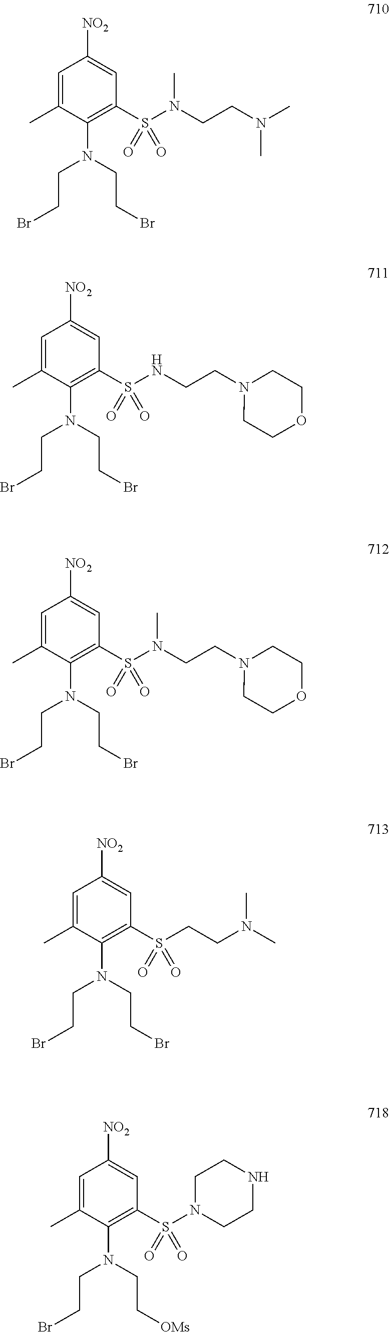 [SMILES: Cc1cc([N+](=O)[O-])cc(S(=O)(=O)N(C)CCN(C)C)c1N(CCBr)CCBr ; Cc1cc([N+](=O)[O-])cc(S(=O)(=O)NCCN2CCOCC2)c1N(CCBr)CCBr ; Cc1cc([N+](=O)[O-])cc(S(=O)(=O)N(C)CCN2CCOCC2)c1N(CCBr)CCBr ; Cc1cc([N+](=O)[O-])cc(S(=O)(=O)CCN(C)C)c1N(CCBr)CCBr ; Cc1cc([N+](=O)[O-])cc(S(=O)(=O)N2CCNCC2)c1N(CCBr)CCOS(C)(=O)=O] 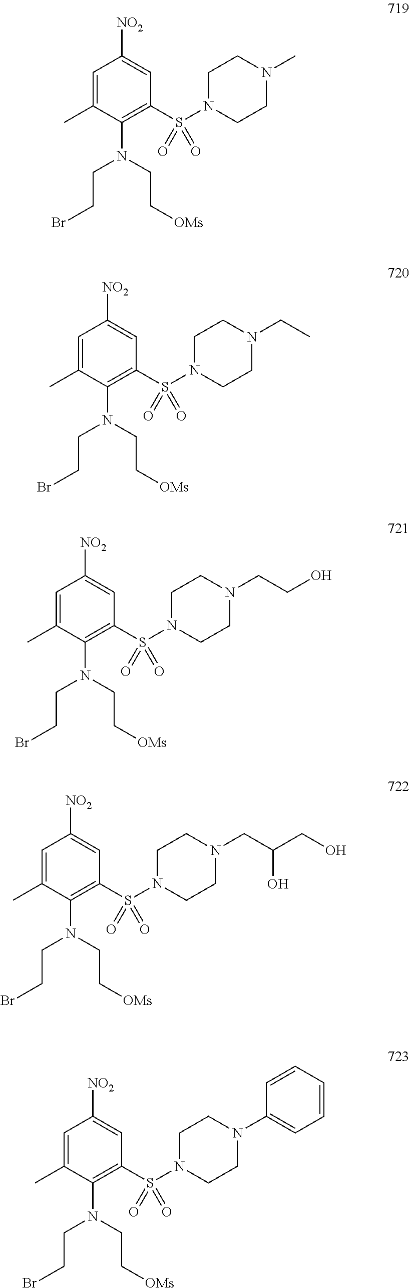 [SMILES: Cc1cc([N+](=O)[O-])cc(S(=O)(=O)N2CCN(C)CC2)c1N(CCBr)CCOS(C)(=O)=O ; CCN1CCN(S(=O)(=O)c2cc([N+](=O)[O-])cc(C)c2N(CCBr)CCOS(C)(=O)=O)CC1 ; Cc1cc([N+](=O)[O-])cc(S(=O)(=O)N2CCN(CCO)CC2)c1N(CCBr)CCOS(C)(=O)=O ; Cc1cc([N+](=O)[O-])cc(S(=O)(=O)N2CCN(CC(O)CO)CC2)c1N(CCBr)CCOS(C)(=O)=O ; Cc1cc([N+](=O)[O-])cc(S(=O)(=O)N2CCN(c3ccccc3)CC2)c1N(CCBr)CCOS(C)(=O)=O] 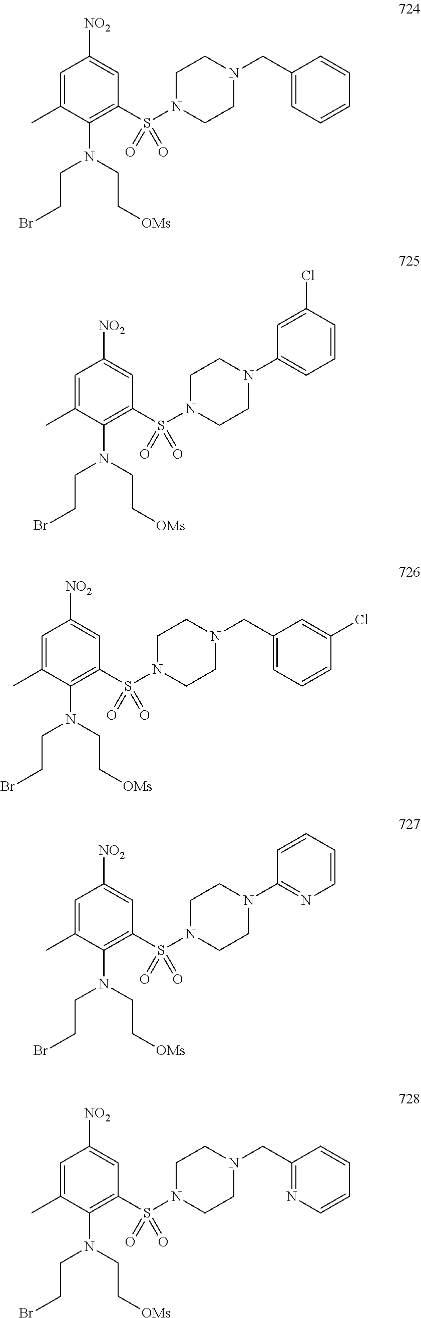 [SMILES: Cc1cc([N+](=O)[O-])cc(S(=O)(=O)N2CCN(Cc3ccccc3)CC2)c1N(CCBr)CCOS(C)(=O)=O ; Cc1cc([N+](=O)[O-])cc(S(=O)(=O)N2CCN(c3cccc(Cl)c3)CC2)c1N(CCBr)CCOS(C)(=O)=O ; Cc1cc([N+](=O)[O-])cc(S(=O)(=O)N2CCN(Cc3cccc(Cl)c3)CC2)c1N(CCBr)CCOS(C)(=O)=O ; Cc1cc([N+](=O)[O-])cc(S(=O)(=O)N2CCN(c3ccccn3)CC2)c1N(CCBr)CCOS(C)(=O)=O ; Cc1cc([N+](=O)[O-])cc(S(=O)(=O)N2CCN(Cc3ccccn3)CC2)c1N(CCBr)CCOS(C)(=O)=O] 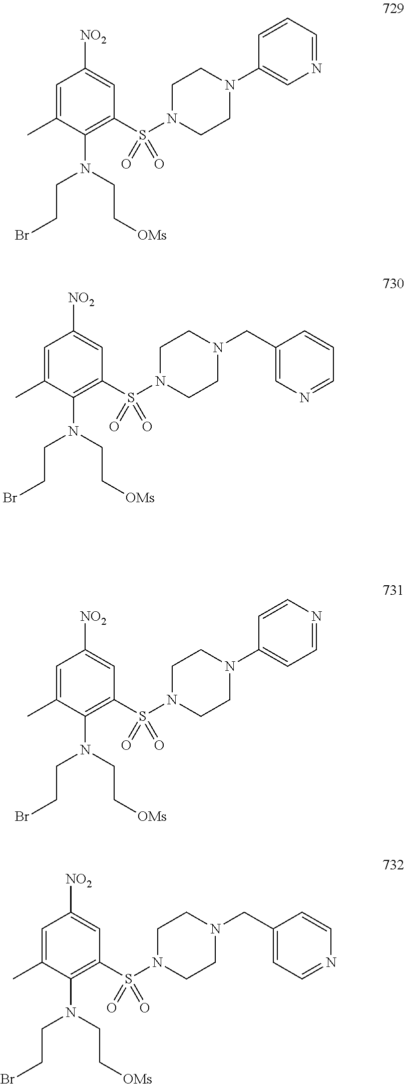 [SMILES: Cc1cc([N+](=O)[O-])cc(S(=O)(=O)N2CCN(c3cccnc3)CC2)c1N(CCBr)CCOS(C)(=O)=O ; Cc1cc([N+](=O)[O-])cc(S(=O)(=O)N2CCN(Cc3cccnc3)CC2)c1N(CCBr)CCOS(C)(=O)=O ; Cc1cc([N+](=O)[O-])cc(S(=O)(=O)N2CCN(c3ccncc3)CC2)c1N(CCBr)CCOS(C)(=O)=O ; Cc1cc([N+](=O)[O-])cc(S(=O)(=O)N2CCN(Cc3ccncc3)CC2)c1N(CCBr)CCOS(C)(=O)=O] 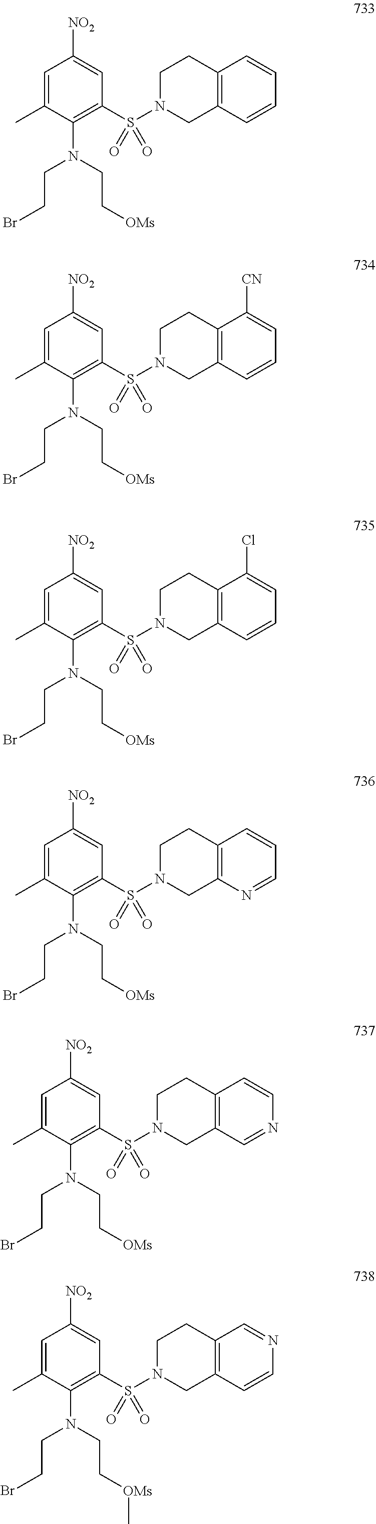 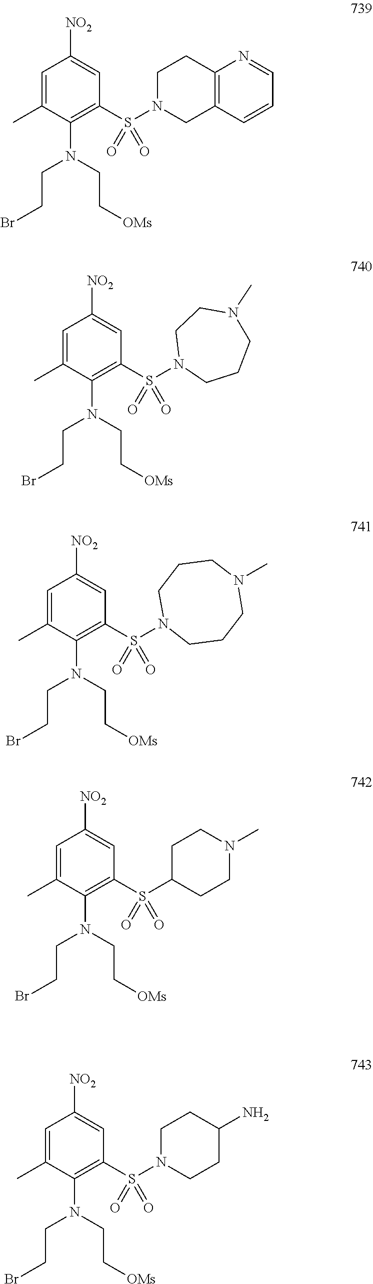 [SMILES: Cc1cc([N+](=O)[O-])cc(S(=O)(=O)N2CCc3ncccc3C2)c1N(CCBr)CCOS(C)(=O)=O ; Cc1cc([N+](=O)[O-])cc(S(=O)(=O)N2CCCN(C)CC2)c1N(CCBr)CCOS(C)(=O)=O ; Cc1cc([N+](=O)[O-])cc(S(=O)(=O)N2CCCN(C)CCC2)c1N(CCBr)CCOS(C)(=O)=O ; Cc1cc([N+](=O)[O-])cc(S(=O)(=O)C2CCN(C)CC2)c1N(CCBr)CCOS(C)(=O)=O ; Cc1cc([N+](=O)[O-])cc(S(=O)(=O)N2CCC(N)CC2)c1N(CCBr)CCOS(C)(=O)=O] 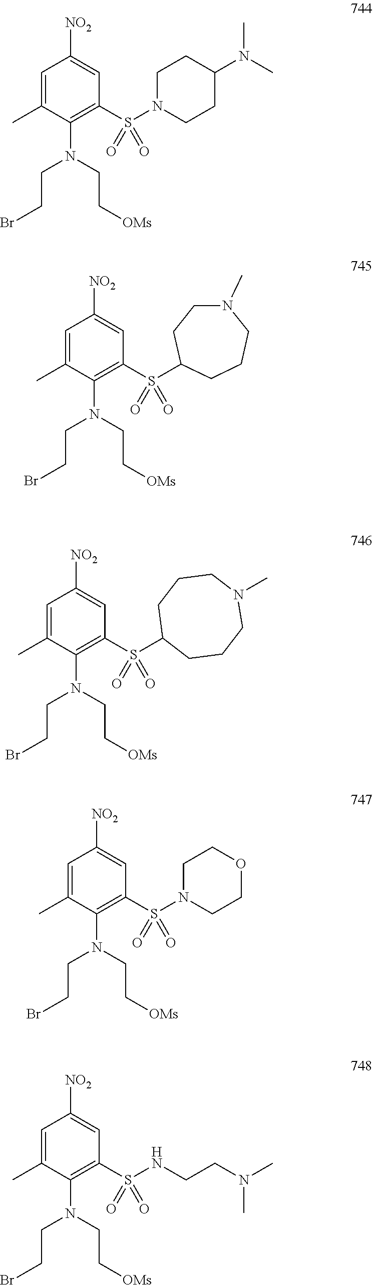 [SMILES: Cc1cc([N+](=O)[O-])cc(S(=O)(=O)N2CCC(N(C)C)CC2)c1N(CCBr)CCOS(C)(=O)=O ; Cc1cc([N+](=O)[O-])cc(S(=O)(=O)C2CCCN(C)CC2)c1N(CCBr)CCOS(C)(=O)=O ; Cc1cc([N+](=O)[O-])cc(S(=O)(=O)C2CCCN(C)CCC2)c1N(CCBr)CCOS(C)(=O)=O ; Cc1cc([N+](=O)[O-])cc(S(=O)(=O)N2CCOCC2)c1N(CCBr)CCOS(C)(=O)=O ; Cc1cc([N+](=O)[O-])cc(S(=O)(=O)NCCN(C)C)c1N(CCBr)CCOS(C)(=O)=O] 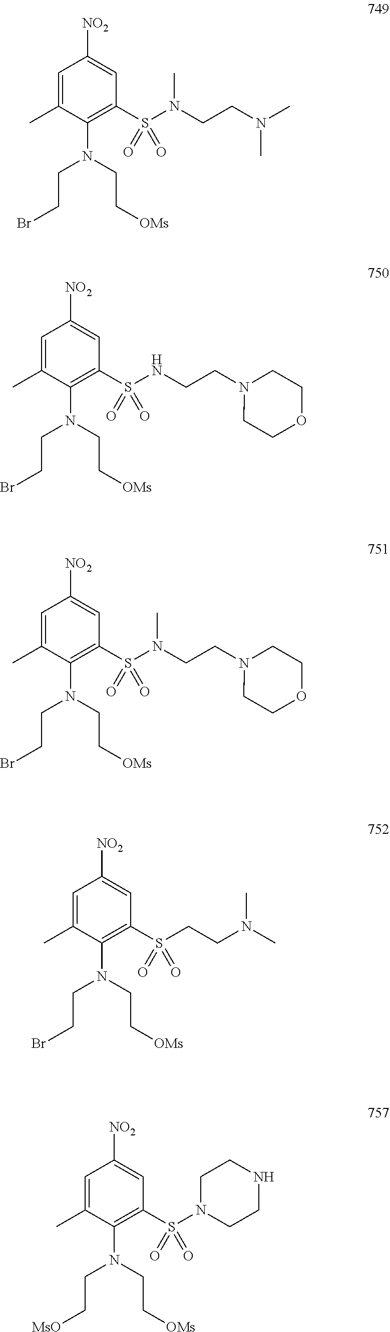 [SMILES: Cc1cc([N+](=O)[O-])cc(S(=O)(=O)N(C)CCN(C)C)c1N(CCBr)CCOS(C)(=O)=O ; Cc1cc([N+](=O)[O-])cc(S(=O)(=O)NCCN2CCOCC2)c1N(CCBr)CCOS(C)(=O)=O ; Cc1cc([N+](=O)[O-])cc(S(=O)(=O)N(C)CCN2CCOCC2)c1N(CCBr)CCOS(C)(=O)=O ; Cc1cc([N+](=O)[O-])cc(S(=O)(=O)CCN(C)C)c1N(CCBr)CCOS(C)(=O)=O ; Cc1cc([N+](=O)[O-])cc(S(=O)(=O)N2CCNCC2)c1N(CCOS(C)(=O)=O)CCOS(C)(=O)=O] 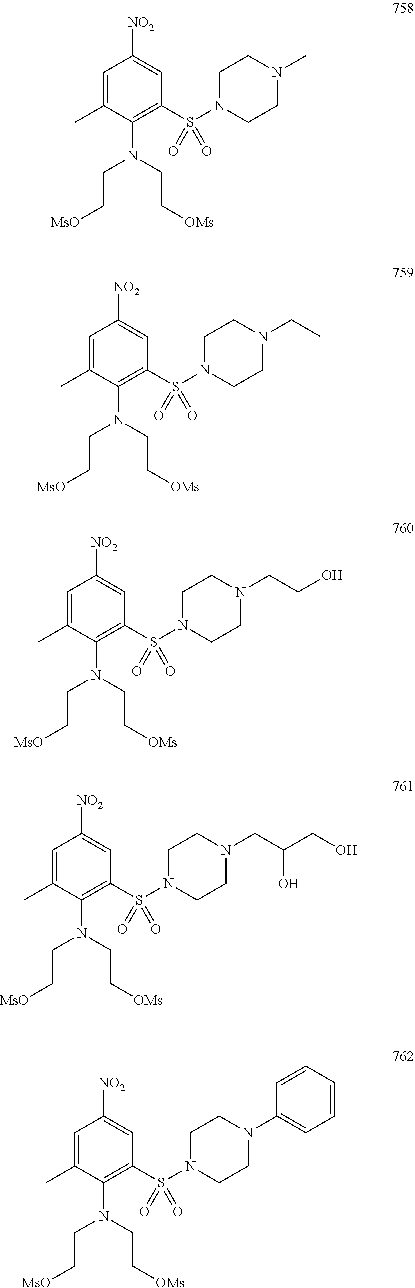 [SMILES: Cc1cc([N+](=O)[O-])cc(S(=O)(=O)N2CCN(C)CC2)c1N(CCOS(C)(=O)=O)CCOS(C)(=O)=O ; CCN1CCN(S(=O)(=O)c2cc([N+](=O)[O-])cc(C)c2N(CCOS(C)(=O)=O)CCOS(C)(=O)=O)CC1 ; Cc1cc([N+](=O)[O-])cc(S(=O)(=O)N2CCN(CCO)CC2)c1N(CCOS(C)(=O)=O)CCOS(C)(=O)=O ; Cc1cc([N+](=O)[O-])cc(S(=O)(=O)N2CCN(CC(O)CO)CC2)c1N(CCOS(C)(=O)=O)CCOS(C)(=O)=O ; Cc1cc([N+](=O)[O-])cc(S(=O)(=O)N2CCN(c3ccccc3)CC2)c1N(CCOS(C)(=O)=O)CCOS(C)(=O)=O] 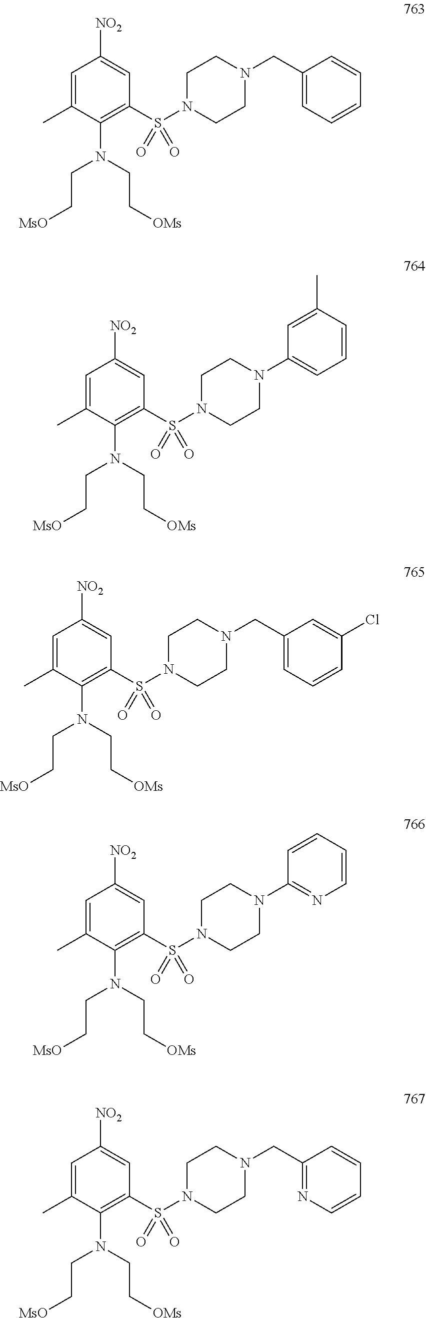 [SMILES: Cc1cc([N+](=O)[O-])cc(S(=O)(=O)N2CCN(Cc3ccccc3)CC2)c1N(CCOS(C)(=O)=O)CCOS(C)(=O)=O ; Cc1cccc(N2CCN(S(=O)(=O)c3cc([N+](=O)[O-])cc(C)c3N(CCOS(C)(=O)=O)CCOS(C)(=O)=O)CC2)c1 ; Cc1cc([N+](=O)[O-])cc(S(=O)(=O)N2CCN(Cc3cccc(Cl)c3)CC2)c1N(CCOS(C)(=O)=O)CCOS(C)(=O)=O ; Cc1cc([N+](=O)[O-])cc(S(=O)(=O)N2CCN(c3ccccn3)CC2)c1N(CCOS(C)(=O)=O)CCOS(C)(=O)=O ; Cc1cc([N+](=O)[O-])cc(S(=O)(=O)N2CCN(Cc3ccccn3)CC2)c1N(CCOS(C)(=O)=O)CCOS(C)(=O)=O] 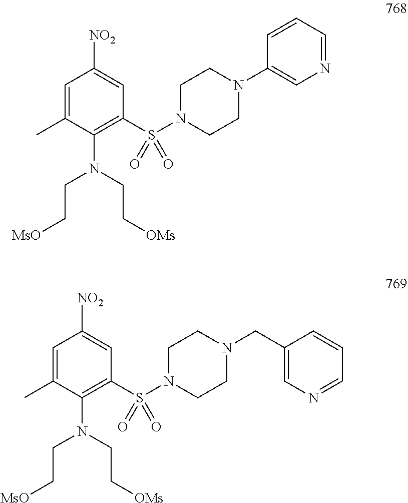 [SMILES: Cc1cc([N+](=O)[O-])cc(S(=O)(=O)N2CCN(c3cccnc3)CC2)c1N(CCOS(C)(=O)=O)CCOS(C)(=O)=O ; Cc1cc([N+](=O)[O-])cc(S(=O)(=O)N2CCN(Cc3cccnc3)CC2)c1N(CCOS(C)(=O)=O)CCOS(C)(=O)=O] 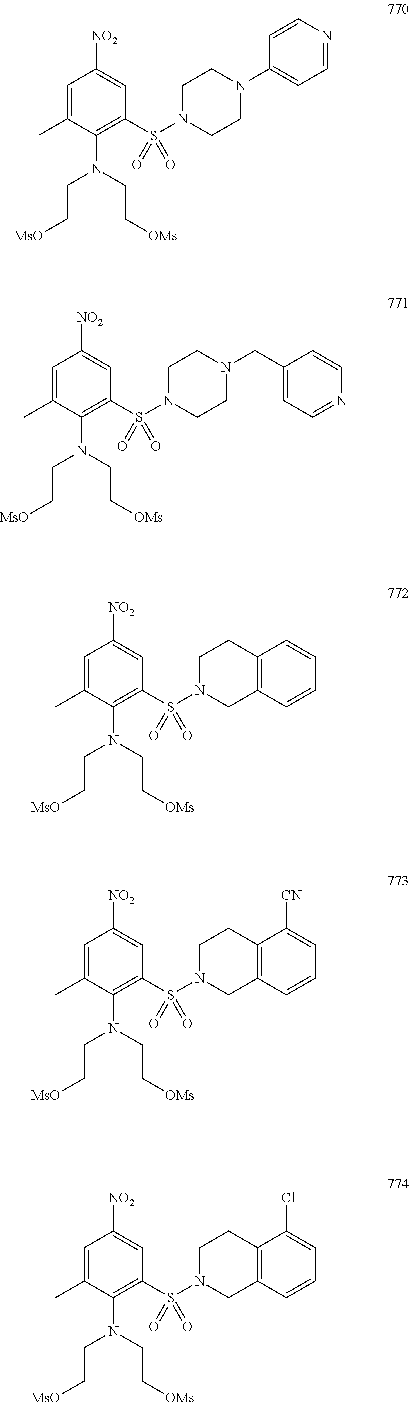 [SMILES: Cc1cc([N+](=O)[O-])cc(S(=O)(=O)N2CCN(c3ccncc3)CC2)c1N(CCOS(C)(=O)=O)CCOS(C)(=O)=O ; Cc1cc([N+](=O)[O-])cc(S(=O)(=O)N2CCN(Cc3ccncc3)CC2)c1N(CCOS(C)(=O)=O)CCOS(C)(=O)=O ; Cc1cc([N+](=O)[O-])cc(S(=O)(=O)N2CCc3ccccc3C2)c1N(CCOS(C)(=O)=O)CCOS(C)(=O)=O ; Cc1cc([N+](=O)[O-])cc(S(=O)(=O)N2CCc3c(C#N)cccc3C2)c1N(CCOS(C)(=O)=O)CCOS(C)(=O)=O ; Cc1cc([N+](=O)[O-])cc(S(=O)(=O)N2CCc3c(Cl)cccc3C2)c1N(CCOS(C)(=O)=O)CCOS(C)(=O)=O] 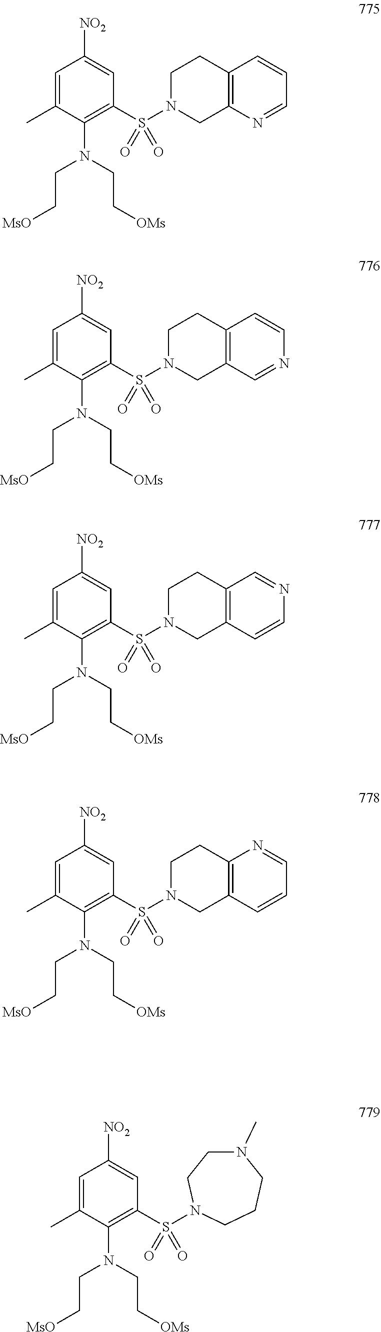 [SMILES: Cc1cc([N+](=O)[O-])cc(S(=O)(=O)N2CCc3cccnc3C2)c1N(CCOS(C)(=O)=O)CCOS(C)(=O)=O ; Cc1cc([N+](=O)[O-])cc(S(=O)(=O)N2CCc3ccncc3C2)c1N(CCOS(C)(=O)=O)CCOS(C)(=O)=O ; Cc1cc([N+](=O)[O-])cc(S(=O)(=O)N2CCc3cnccc3C2)c1N(CCOS(C)(=O)=O)CCOS(C)(=O)=O ; Cc1cc([N+](=O)[O-])cc(S(=O)(=O)N2CCc3ncccc3C2)c1N(CCOS(C)(=O)=O)CCOS(C)(=O)=O ; Cc1cc([N+](=O)[O-])cc(S(=O)(=O)N2CCCN(C)CC2)c1N(CCOS(C)(=O)=O)CCOS(C)(=O)=O] 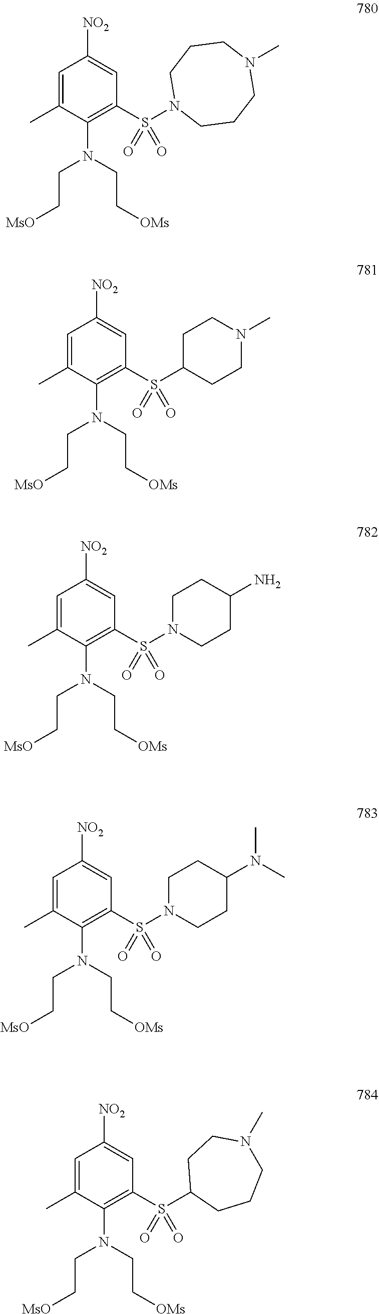 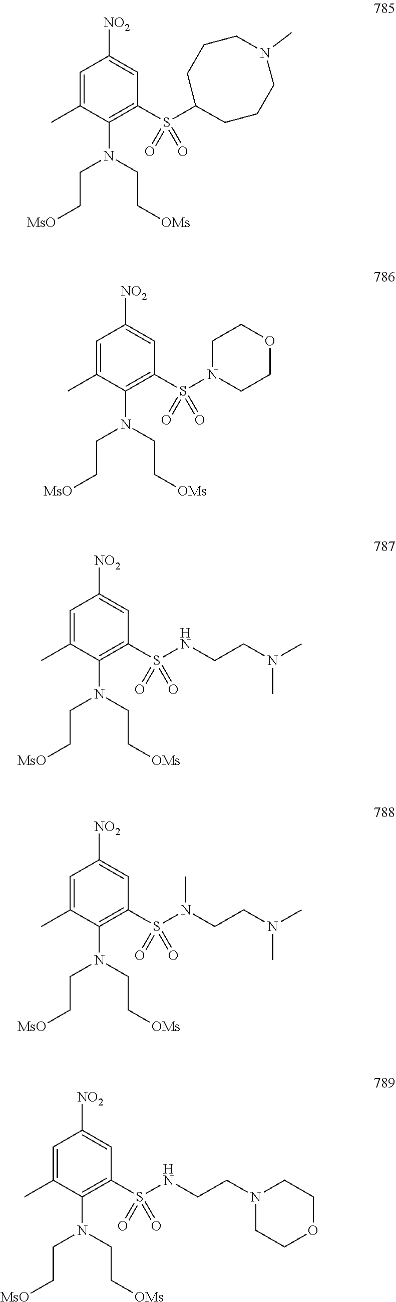 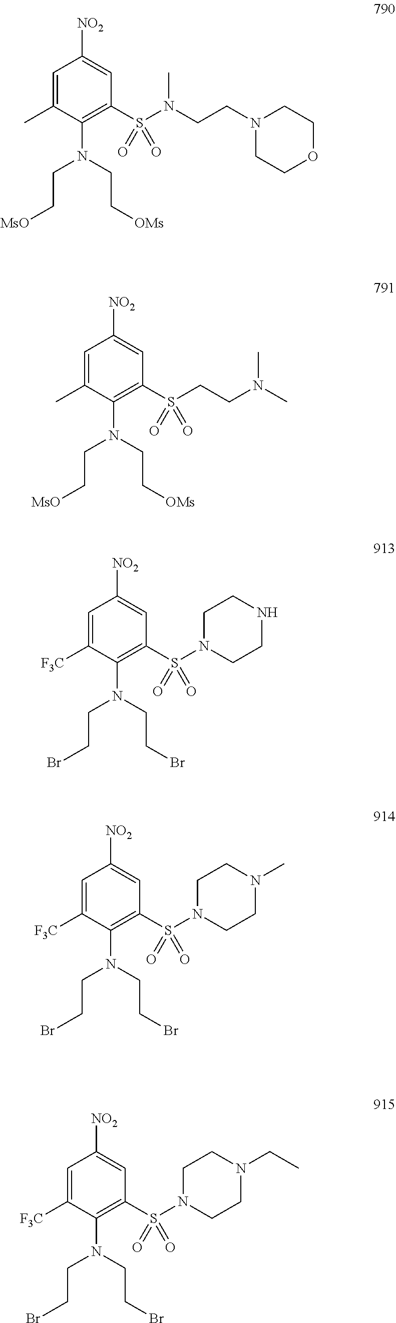 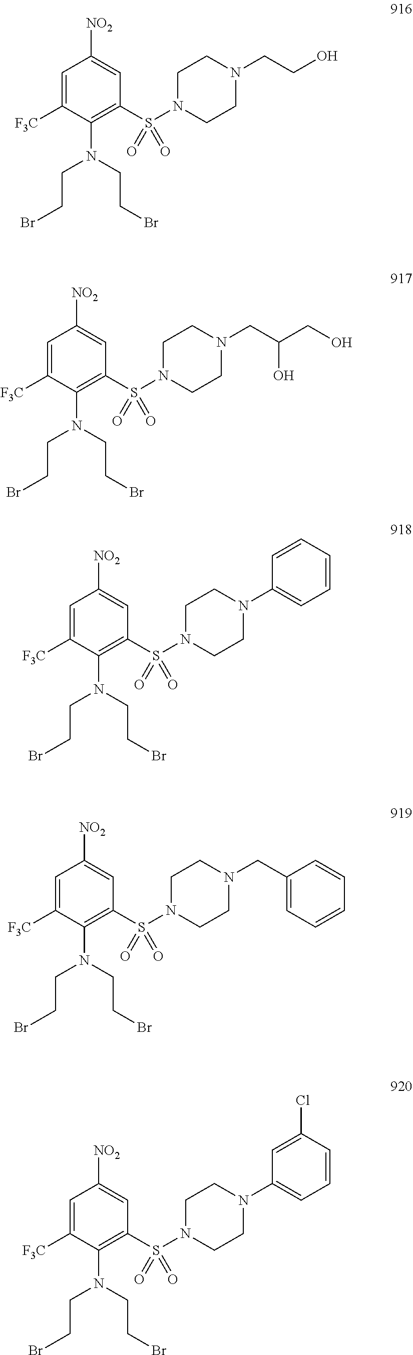 [SMILES: O=[N+]([O-])c1cc(C(F)(F)F)c(N(CCBr)CCBr)c(S(=O)(=O)N2CCN(CCO)CC2)c1 ; O=[N+]([O-])c1cc(C(F)(F)F)c(N(CCBr)CCBr)c(S(=O)(=O)N2CCN(CC(O)CO)CC2)c1 ; O=[N+]([O-])c1cc(C(F)(F)F)c(N(CCBr)CCBr)c(S(=O)(=O)N2CCN(c3ccccc3)CC2)c1 ; O=[N+]([O-])c1cc(C(F)(F)F)c(N(CCBr)CCBr)c(S(=O)(=O)N2CCN(Cc3ccccc3)CC2)c1 ; O=[N+]([O-])c1cc(C(F)(F)F)c(N(CCBr)CCBr)c(S(=O)(=O)N2CCN(c3cccc(Cl)c3)CC2)c1] 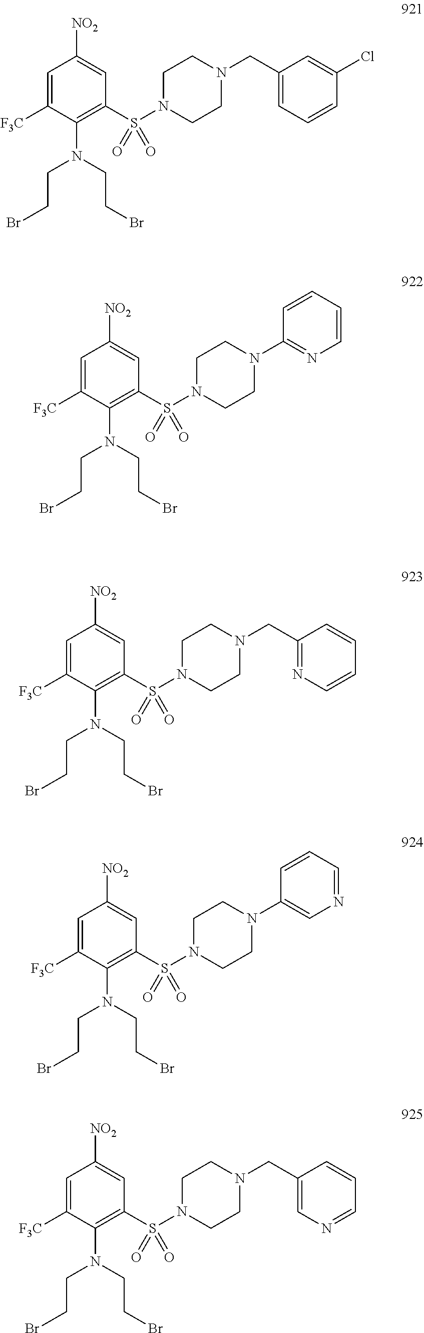 [SMILES: O=[N+]([O-])c1cc(C(F)(F)F)c(N(CCBr)CCBr)c(S(=O)(=O)N2CCN(Cc3cccc(Cl)c3)CC2)c1 ; O=[N+]([O-])c1cc(C(F)(F)F)c(N(CCBr)CCBr)c(S(=O)(=O)N2CCN(c3ccccn3)CC2)c1 ; O=[N+]([O-])c1cc(C(F)(F)F)c(N(CCBr)CCBr)c(S(=O)(=O)N2CCN(Cc3ccccn3)CC2)c1 ; O=[N+]([O-])c1cc(C(F)(F)F)c(N(CCBr)CCBr)c(S(=O)(=O)N2CCN(c3cccnc3)CC2)c1 ; O=[N+]([O-])c1cc(C(F)(F)F)c(N(CCBr)CCBr)c(S(=O)(=O)N2CCN(Cc3cccnc3)CC2)c1] 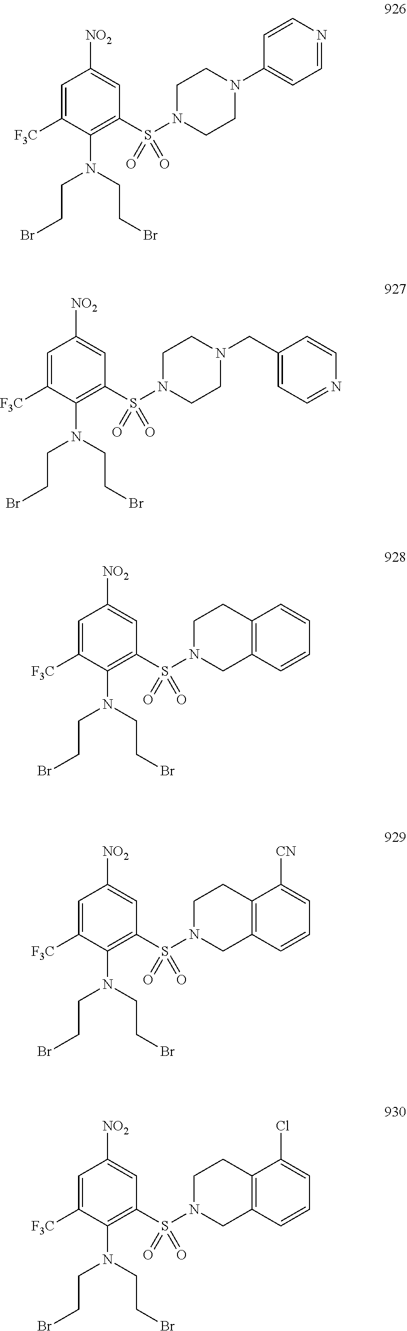 [SMILES: O=[N+]([O-])c1cc(C(F)(F)F)c(N(CCBr)CCBr)c(S(=O)(=O)N2CCN(c3ccncc3)CC2)c1 ; O=[N+]([O-])c1cc(C(F)(F)F)c(N(CCBr)CCBr)c(S(=O)(=O)N2CCN(Cc3ccncc3)CC2)c1 ; O=[N+]([O-])c1cc(C(F)(F)F)c(N(CCBr)CCBr)c(S(=O)(=O)N2CCc3ccccc3C2)c1 ; N#Cc1cccc2c1CCN(S(=O)(=O)c1cc([N+](=O)[O-])cc(C(F)(F)F)c1N(CCBr)CCBr)C2 ; O=[N+]([O-])c1cc(C(F)(F)F)c(N(CCBr)CCBr)c(S(=O)(=O)N2CCc3c(Cl)cccc3C2)c1] 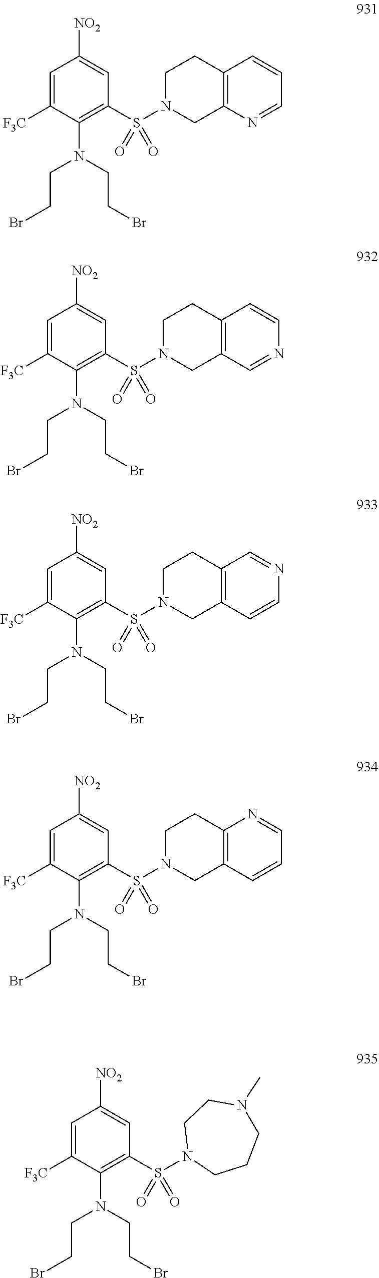 [SMILES: O=[N+]([O-])c1cc(C(F)(F)F)c(N(CCBr)CCBr)c(S(=O)(=O)N2CCc3cccnc3C2)c1 ; O=[N+]([O-])c1cc(C(F)(F)F)c(N(CCBr)CCBr)c(S(=O)(=O)N2CCc3ccncc3C2)c1 ; O=[N+]([O-])c1cc(C(F)(F)F)c(N(CCBr)CCBr)c(S(=O)(=O)N2CCc3cnccc3C2)c1 ; O=[N+]([O-])c1cc(C(F)(F)F)c(N(CCBr)CCBr)c(S(=O)(=O)N2CCc3ncccc3C2)c1 ; CN1CCCN(S(=O)(=O)c2cc([N+](=O)[O-])cc(C(F)(F)F)c2N(CCBr)CCBr)CC1] 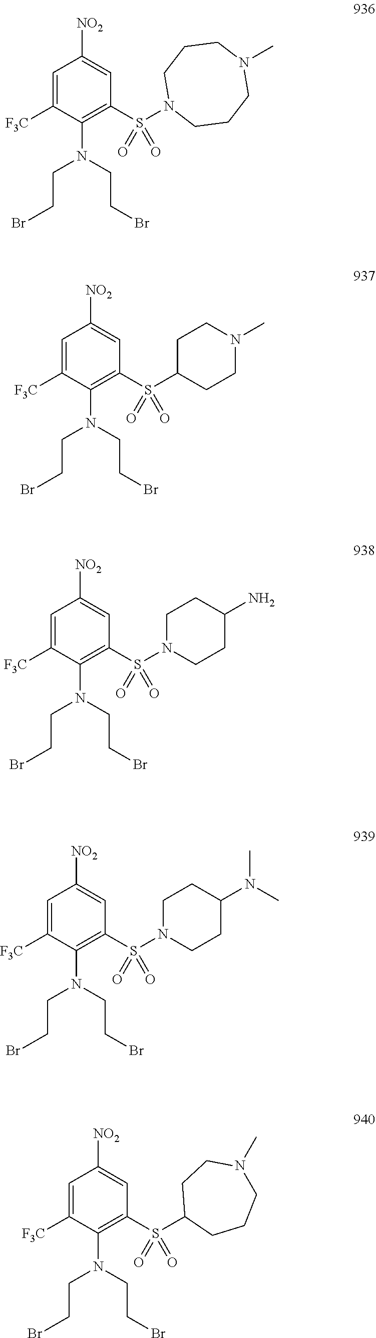 [SMILES: CN1CCCN(S(=O)(=O)c2cc([N+](=O)[O-])cc(C(F)(F)F)c2N(CCBr)CCBr)CCC1 ; CN1CCC(S(=O)(=O)c2cc([N+](=O)[O-])cc(C(F)(F)F)c2N(CCBr)CCBr)CC1 ; NC1CCN(S(=O)(=O)c2cc([N+](=O)[O-])cc(C(F)(F)F)c2N(CCBr)CCBr)CC1 ; CN(C)C1CCN(S(=O)(=O)c2cc([N+](=O)[O-])cc(C(F)(F)F)c2N(CCBr)CCBr)CC1 ; CN1CCCC(S(=O)(=O)c2cc([N+](=O)[O-])cc(C(F)(F)F)c2N(CCBr)CCBr)CC1] 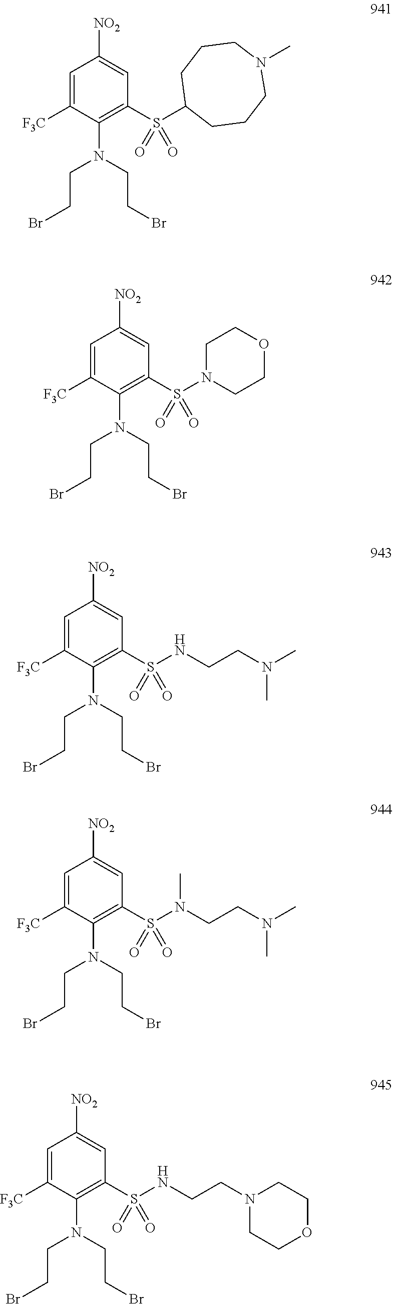 [SMILES: CN1CCCC(S(=O)(=O)c2cc([N+](=O)[O-])cc(C(F)(F)F)c2N(CCBr)CCBr)CCC1 ; O=[N+]([O-])c1cc(C(F)(F)F)c(N(CCBr)CCBr)c(S(=O)(=O)N2CCOCC2)c1 ; CN(C)CCNS(=O)(=O)c1cc([N+](=O)[O-])cc(C(F)(F)F)c1N(CCBr)CCBr ; CN(C)CCN(C)S(=O)(=O)c1cc([N+](=O)[O-])cc(C(F)(F)F)c1N(CCBr)CCBr ; O=[N+]([O-])c1cc(C(F)(F)F)c(N(CCBr)CCBr)c(S(=O)(=O)NCCN2CCOCC2)c1] 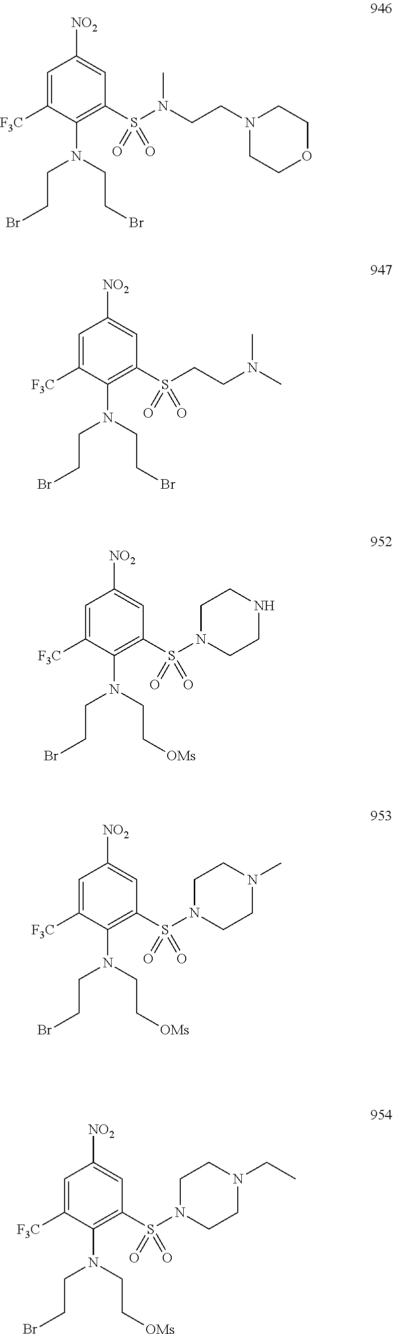 [SMILES: CN(CCN1CCOCC1)S(=O)(=O)c1cc([N+](=O)[O-])cc(C(F)(F)F)c1N(CCBr)CCBr ; CN(C)CCS(=O)(=O)c1cc([N+](=O)[O-])cc(C(F)(F)F)c1N(CCBr)CCBr ; CS(=O)(=O)OCCN(CCBr)c1c(C(F)(F)F)cc([N+](=O)[O-])cc1S(=O)(=O)N1CCNCC1 ; CN1CCN(S(=O)(=O)c2cc([N+](=O)[O-])cc(C(F)(F)F)c2N(CCBr)CCOS(C)(=O)=O)CC1 ; CCN1CCN(S(=O)(=O)c2cc([N+](=O)[O-])cc(C(F)(F)F)c2N(CCBr)CCOS(C)(=O)=O)CC1] 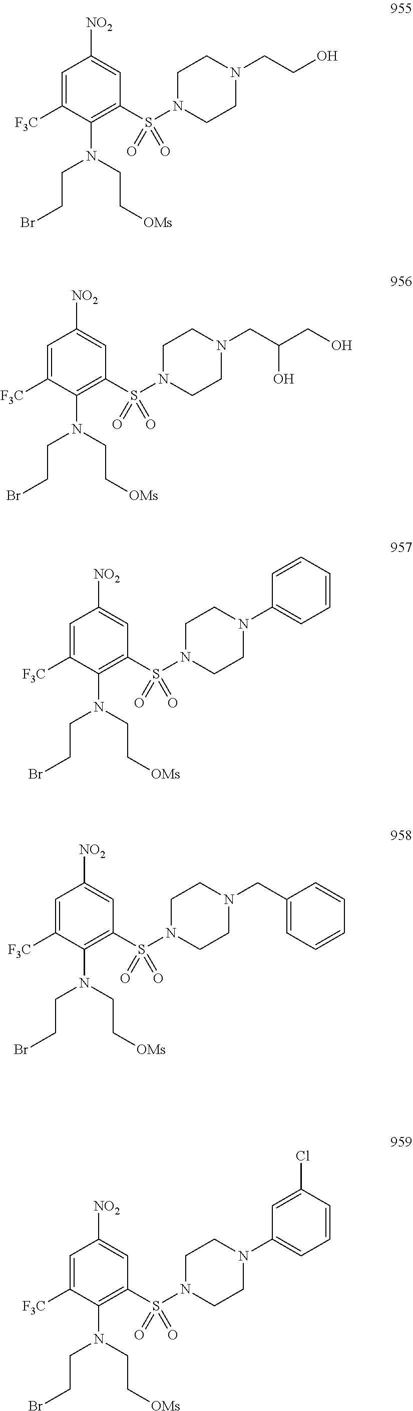 [SMILES: CS(=O)(=O)OCCN(CCBr)c1c(C(F)(F)F)cc([N+](=O)[O-])cc1S(=O)(=O)N1CCN(CCO)CC1 ; CS(=O)(=O)OCCN(CCBr)c1c(C(F)(F)F)cc([N+](=O)[O-])cc1S(=O)(=O)N1CCN(CC(O)CO)CC1 ; CS(=O)(=O)OCCN(CCBr)c1c(C(F)(F)F)cc([N+](=O)[O-])cc1S(=O)(=O)N1CCN(c2ccccc2)CC1 ; CS(=O)(=O)OCCN(CCBr)c1c(C(F)(F)F)cc([N+](=O)[O-])cc1S(=O)(=O)N1CCN(Cc2ccccc2)CC1 ; CS(=O)(=O)OCCN(CCBr)c1c(C(F)(F)F)cc([N+](=O)[O-])cc1S(=O)(=O)N1CCN(c2cccc(Cl)c2)CC1] 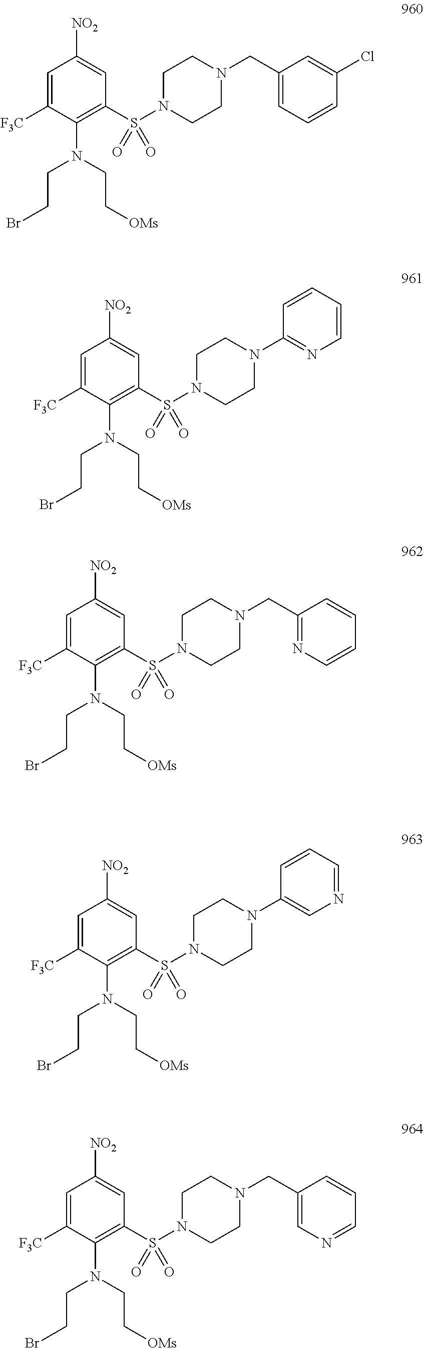 [SMILES: CS(=O)(=O)OCCN(CCBr)c1c(C(F)(F)F)cc([N+](=O)[O-])cc1S(=O)(=O)N1CCN(Cc2cccc(Cl)c2)CC1 ; CS(=O)(=O)OCCN(CCBr)c1c(C(F)(F)F)cc([N+](=O)[O-])cc1S(=O)(=O)N1CCN(c2ccccn2)CC1 ; CS(=O)(=O)OCCN(CCBr)c1c(C(F)(F)F)cc([N+](=O)[O-])cc1S(=O)(=O)N1CCN(Cc2ccccn2)CC1 ; CS(=O)(=O)OCCN(CCBr)c1c(C(F)(F)F)cc([N+](=O)[O-])cc1S(=O)(=O)N1CCN(c2cccnc2)CC1 ; CS(=O)(=O)OCCN(CCBr)c1c(C(F)(F)F)cc([N+](=O)[O-])cc1S(=O)(=O)N1CCN(Cc2cccnc2)CC1] 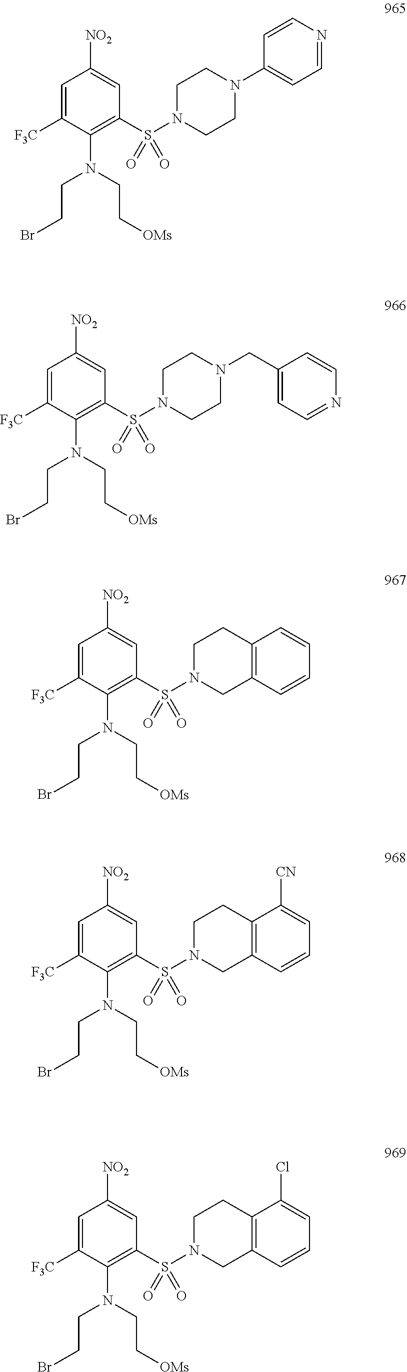 [SMILES: CS(=O)(=O)OCCN(CCBr)c1c(C(F)(F)F)cc([N+](=O)[O-])cc1S(=O)(=O)N1CCN(c2ccncc2)CC1 ; CS(=O)(=O)OCCN(CCBr)c1c(C(F)(F)F)cc([N+](=O)[O-])cc1S(=O)(=O)N1CCN(Cc2ccncc2)CC1 ; CS(=O)(=O)OCCN(CCBr)c1c(C(F)(F)F)cc([N+](=O)[O-])cc1S(=O)(=O)N1CCc2ccccc2C1 ; CS(=O)(=O)OCCN(CCBr)c1c(C(F)(F)F)cc([N+](=O)[O-])cc1S(=O)(=O)N1CCc2c(C#N)cccc2C1 ; CS(=O)(=O)OCCN(CCBr)c1c(C(F)(F)F)cc([N+](=O)[O-])cc1S(=O)(=O)N1CCc2c(Cl)cccc2C1] 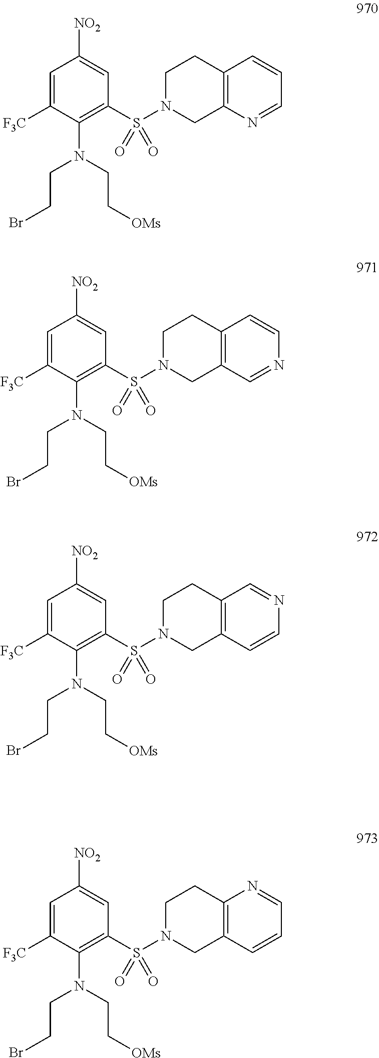 [SMILES: CS(=O)(=O)OCCN(CCBr)c1c(C(F)(F)F)cc([N+](=O)[O-])cc1S(=O)(=O)N1CCc2cccnc2C1 ; CS(=O)(=O)OCCN(CCBr)c1c(C(F)(F)F)cc([N+](=O)[O-])cc1S(=O)(=O)N1CCc2ccncc2C1 ; CS(=O)(=O)OCCN(CCBr)c1c(C(F)(F)F)cc([N+](=O)[O-])cc1S(=O)(=O)N1CCc2cnccc2C1 ; CS(=O)(=O)OCCN(CCBr)c1c(C(F)(F)F)cc([N+](=O)[O-])cc1S(=O)(=O)N1CCc2ncccc2C1] 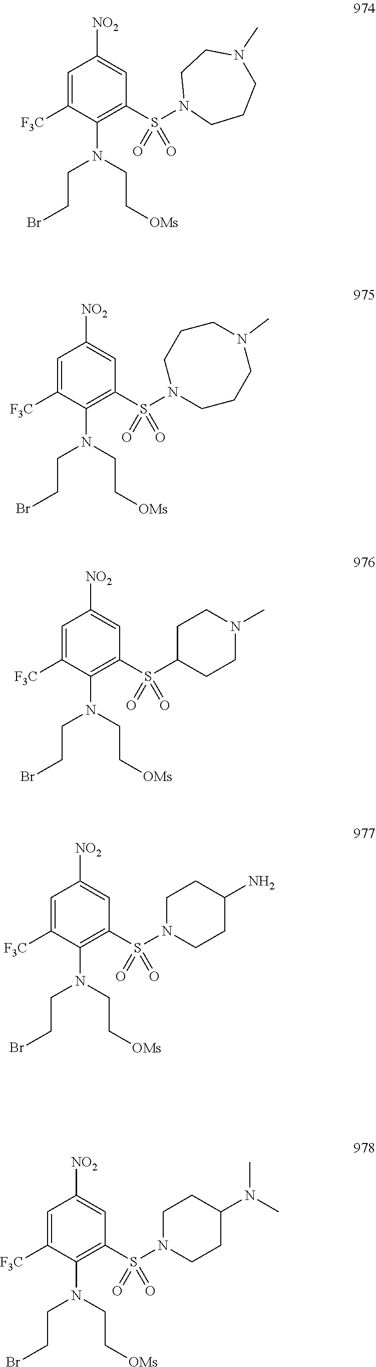 [SMILES: CN1CCCN(S(=O)(=O)c2cc([N+](=O)[O-])cc(C(F)(F)F)c2N(CCBr)CCOS(C)(=O)=O)CC1 ; CN1CCCN(S(=O)(=O)c2cc([N+](=O)[O-])cc(C(F)(F)F)c2N(CCBr)CCOS(C)(=O)=O)CCC1 ; CN1CCC(S(=O)(=O)c2cc([N+](=O)[O-])cc(C(F)(F)F)c2N(CCBr)CCOS(C)(=O)=O)CC1 ; CS(=O)(=O)OCCN(CCBr)c1c(C(F)(F)F)cc([N+](=O)[O-])cc1S(=O)(=O)N1CCC(N)CC1 ; CN(C)C1CCN(S(=O)(=O)c2cc([N+](=O)[O-])cc(C(F)(F)F)c2N(CCBr)CCOS(C)(=O)=O)CC1] 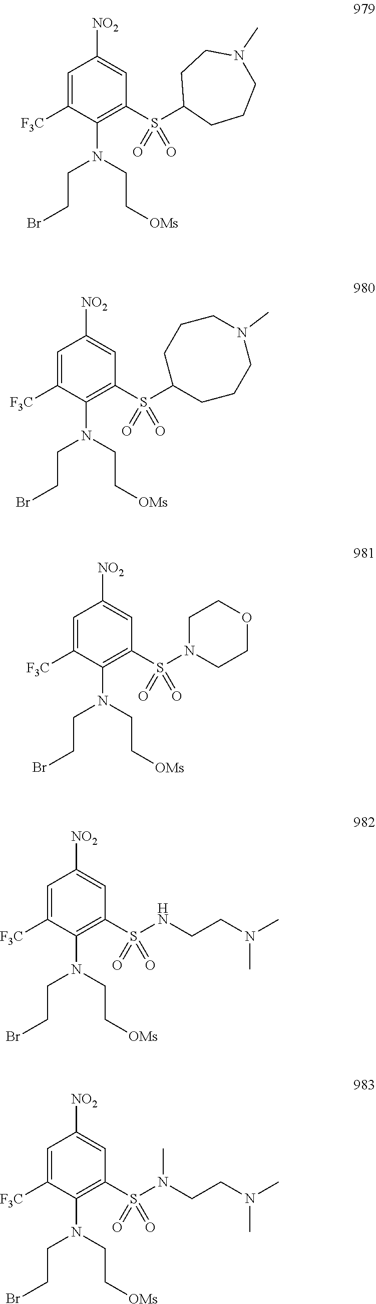 [SMILES: CN1CCCC(S(=O)(=O)c2cc([N+](=O)[O-])cc(C(F)(F)F)c2N(CCBr)CCOS(C)(=O)=O)CC1 ; CN1CCCC(S(=O)(=O)c2cc([N+](=O)[O-])cc(C(F)(F)F)c2N(CCBr)CCOS(C)(=O)=O)CCC1 ; CS(=O)(=O)OCCN(CCBr)c1c(C(F)(F)F)cc([N+](=O)[O-])cc1S(=O)(=O)N1CCOCC1 ; CN(C)CCNS(=O)(=O)c1cc([N+](=O)[O-])cc(C(F)(F)F)c1N(CCBr)CCOS(C)(=O)=O ; CN(C)CCN(C)S(=O)(=O)c1cc([N+](=O)[O-])cc(C(F)(F)F)c1N(CCBr)CCOS(C)(=O)=O] 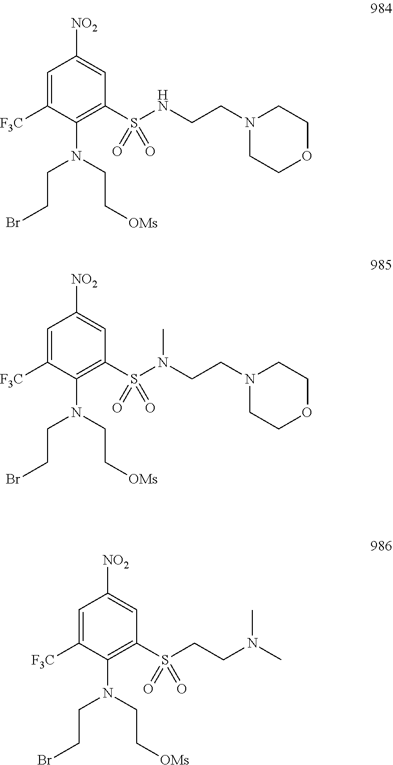 [SMILES: CS(=O)(=O)OCCN(CCBr)c1c(C(F)(F)F)cc([N+](=O)[O-])cc1S(=O)(=O)NCCN1CCOCC1 ; CN(CCN1CCOCC1)S(=O)(=O)c1cc([N+](=O)[O-])cc(C(F)(F)F)c1N(CCBr)CCOS(C)(=O)=O ; CN(C)CCS(=O)(=O)c1cc([N+](=O)[O-])cc(C(F)(F)F)c1N(CCBr)CCOS(C)(=O)=O] 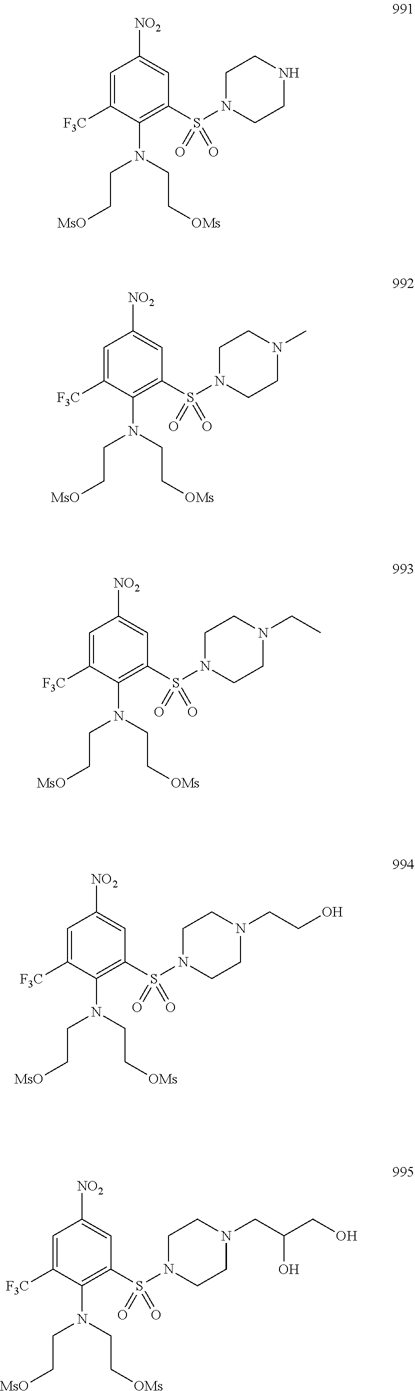 [SMILES: CS(=O)(=O)OCCN(CCOS(C)(=O)=O)c1c(C(F)(F)F)cc([N+](=O)[O-])cc1S(=O)(=O)N1CCNCC1 ; CN1CCN(S(=O)(=O)c2cc([N+](=O)[O-])cc(C(F)(F)F)c2N(CCOS(C)(=O)=O)CCOS(C)(=O)=O)CC1 ; CCN1CCN(S(=O)(=O)c2cc([N+](=O)[O-])cc(C(F)(F)F)c2N(CCOS(C)(=O)=O)CCOS(C)(=O)=O)CC1 ; CS(=O)(=O)OCCN(CCOS(C)(=O)=O)c1c(C(F)(F)F)cc([N+](=O)[O-])cc1S(=O)(=O)N1CCN(CCO)CC1 ; CS(=O)(=O)OCCN(CCOS(C)(=O)=O)c1c(C(F)(F)F)cc([N+](=O)[O-])cc1S(=O)(=O)N1CCN(CC(O)CO)CC1] 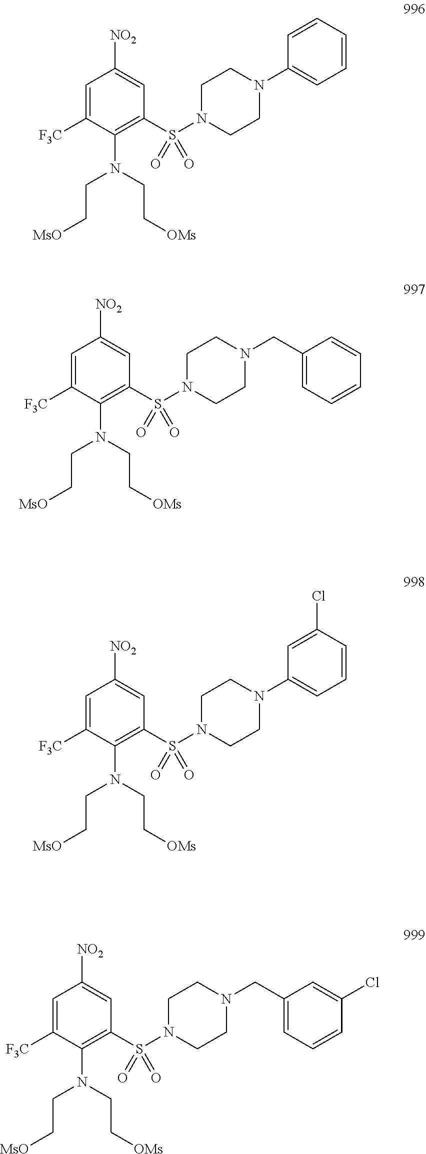 [SMILES: CS(=O)(=O)OCCN(CCOS(C)(=O)=O)c1c(C(F)(F)F)cc([N+](=O)[O-])cc1S(=O)(=O)N1CCN(c2ccccc2)CC1 ; CS(=O)(=O)OCCN(CCOS(C)(=O)=O)c1c(C(F)(F)F)cc([N+](=O)[O-])cc1S(=O)(=O)N1CCN(Cc2ccccc2)CC1 ; CS(=O)(=O)OCCN(CCOS(C)(=O)=O)c1c(C(F)(F)F)cc([N+](=O)[O-])cc1S(=O)(=O)N1CCN(c2cccc(Cl)c2)CC1 ; CS(=O)(=O)OCCN(CCOS(C)(=O)=O)c1c(C(F)(F)F)cc([N+](=O)[O-])cc1S(=O)(=O)N1CCN(Cc2cccc(Cl)c2)CC1] 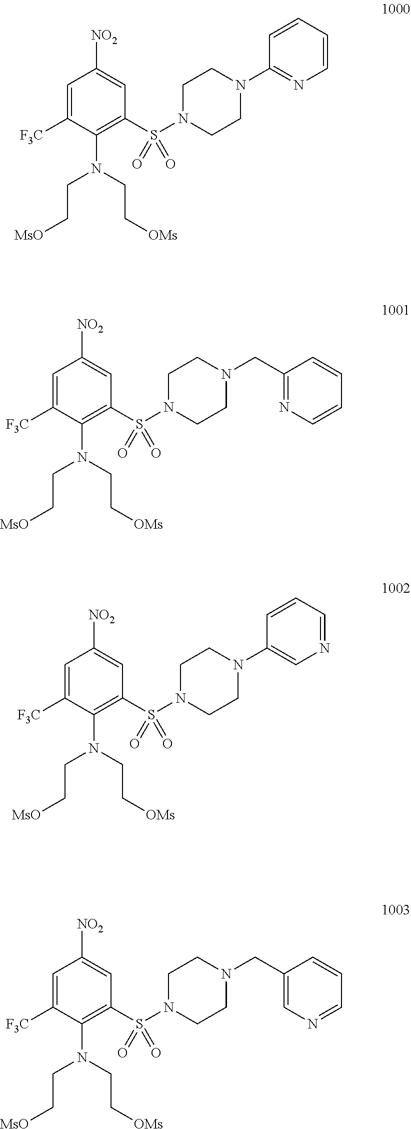 [SMILES: CS(=O)(=O)OCCN(CCOS(C)(=O)=O)c1c(C(F)(F)F)cc([N+](=O)[O-])cc1S(=O)(=O)N1CCN(c2ccccn2)CC1 ; CS(=O)(=O)OCCN(CCOS(C)(=O)=O)c1c(C(F)(F)F)cc([N+](=O)[O-])cc1S(=O)(=O)N1CCN(Cc2ccccn2)CC1 ; CS(=O)(=O)OCCN(CCOS(C)(=O)=O)c1c(C(F)(F)F)cc([N+](=O)[O-])cc1S(=O)(=O)N1CCN(c2cccnc2)CC1 ; CS(=O)(=O)OCCN(CCOS(C)(=O)=O)c1c(C(F)(F)F)cc([N+](=O)[O-])cc1S(=O)(=O)N1CCN(Cc2cccnc2)CC1] 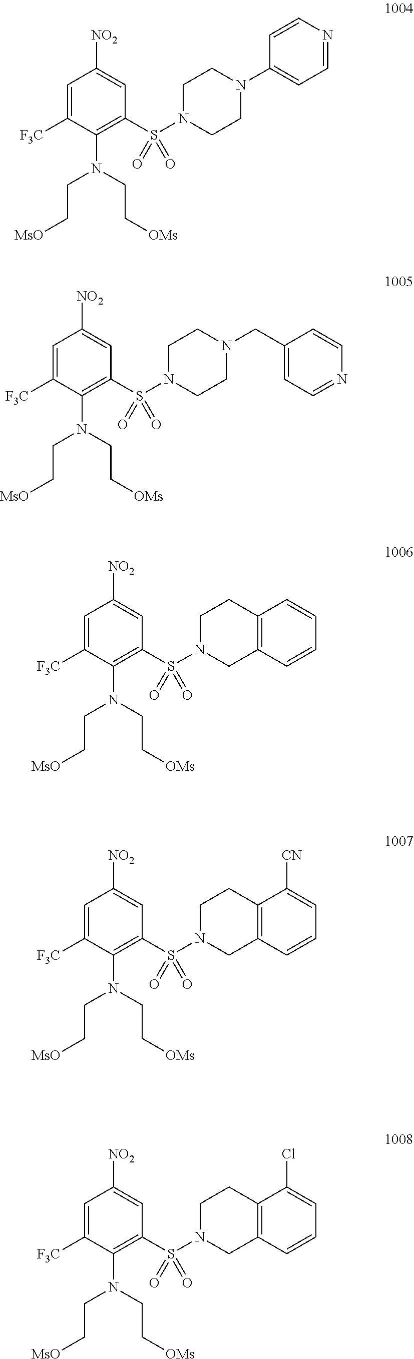 [SMILES: CS(=O)(=O)OCCN(CCOS(C)(=O)=O)c1c(C(F)(F)F)cc([N+](=O)[O-])cc1S(=O)(=O)N1CCN(c2ccncc2)CC1 ; CS(=O)(=O)OCCN(CCOS(C)(=O)=O)c1c(C(F)(F)F)cc([N+](=O)[O-])cc1S(=O)(=O)N1CCN(Cc2ccncc2)CC1 ; CS(=O)(=O)OCCN(CCOS(C)(=O)=O)c1c(C(F)(F)F)cc([N+](=O)[O-])cc1S(=O)(=O)N1CCc2ccccc2C1 ; CS(=O)(=O)OCCN(CCOS(C)(=O)=O)c1c(C(F)(F)F)cc([N+](=O)[O-])cc1S(=O)(=O)N1CCc2c(C#N)cccc2C1 ; CS(=O)(=O)OCCN(CCOS(C)(=O)=O)c1c(C(F)(F)F)cc([N+](=O)[O-])cc1S(=O)(=O)N1CCc2c(Cl)cccc2C1] 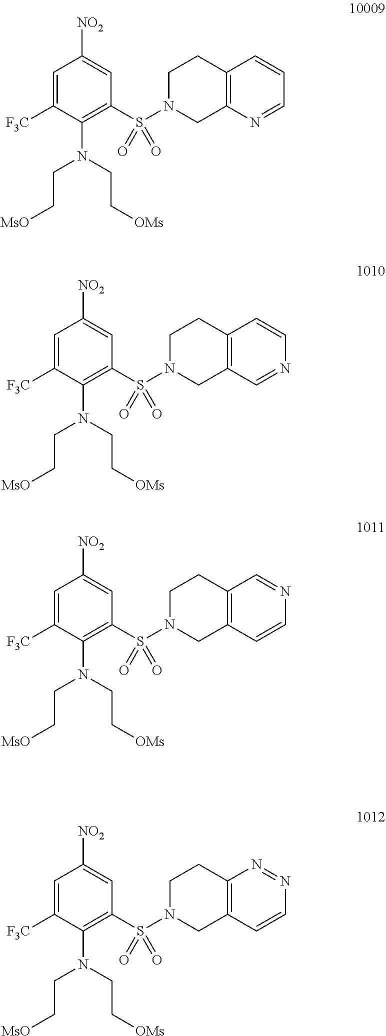 [SMILES: CS(=O)(=O)OCCN(CCOS(C)(=O)=O)c1c(C(F)(F)F)cc([N+](=O)[O-])cc1S(=O)(=O)N1CCc2cccnc2C1 ; CS(=O)(=O)OCCN(CCOS(C)(=O)=O)c1c(C(F)(F)F)cc([N+](=O)[O-])cc1S(=O)(=O)N1CCc2ccncc2C1 ; CS(=O)(=O)OCCN(CCOS(C)(=O)=O)c1c(C(F)(F)F)cc([N+](=O)[O-])cc1S(=O)(=O)N1CCc2cnccc2C1 ; CS(=O)(=O)OCCN(CCOS(C)(=O)=O)c1c(C(F)(F)F)cc([N+](=O)[O-])cc1S(=O)(=O)N1CCc2nnccc2C1] 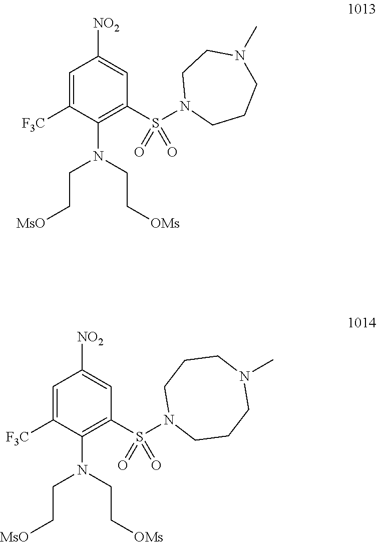 [SMILES: CN1CCCN(S(=O)(=O)c2cc([N+](=O)[O-])cc(C(F)(F)F)c2N(CCOS(C)(=O)=O)CCOS(C)(=O)=O)CC1 ; CN1CCCN(S(=O)(=O)c2cc([N+](=O)[O-])cc(C(F)(F)F)c2N(CCOS(C)(=O)=O)CCOS(C)(=O)=O)CCC1] 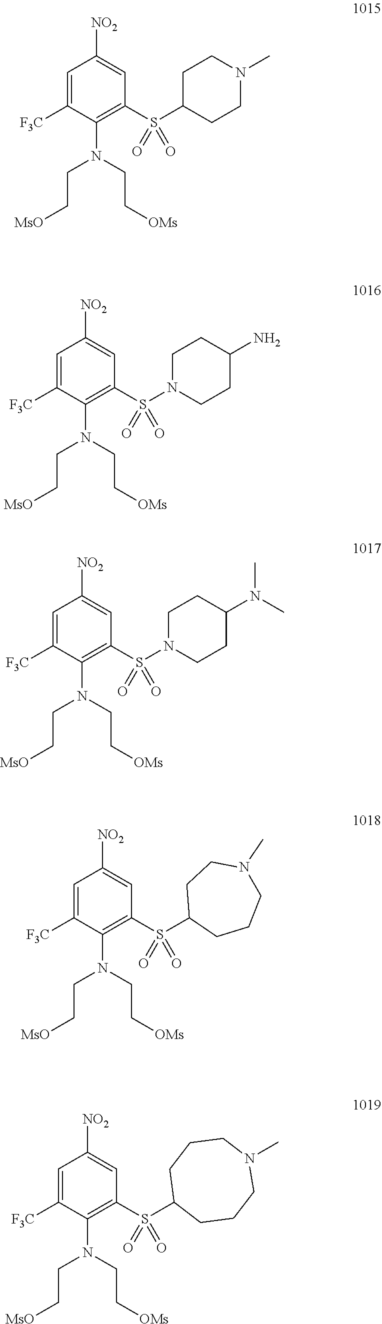 [SMILES: CN1CCC(S(=O)(=O)c2cc([N+](=O)[O-])cc(C(F)(F)F)c2N(CCOS(C)(=O)=O)CCOS(C)(=O)=O)CC1 ; CS(=O)(=O)OCCN(CCOS(C)(=O)=O)c1c(C(F)(F)F)cc([N+](=O)[O-])cc1S(=O)(=O)N1CCC(N)CC1 ; CN(C)C1CCN(S(=O)(=O)c2cc([N+](=O)[O-])cc(C(F)(F)F)c2N(CCOS(C)(=O)=O)CCOS(C)(=O)=O)CC1 ; CN1CCCC(S(=O)(=O)c2cc([N+](=O)[O-])cc(C(F)(F)F)c2N(CCOS(C)(=O)=O)CCOS(C)(=O)=O)CC1 ; CN1CCCC(S(=O)(=O)c2cc([N+](=O)[O-])cc(C(F)(F)F)c2N(CCOS(C)(=O)=O)CCOS(C)(=O)=O)CCC1] 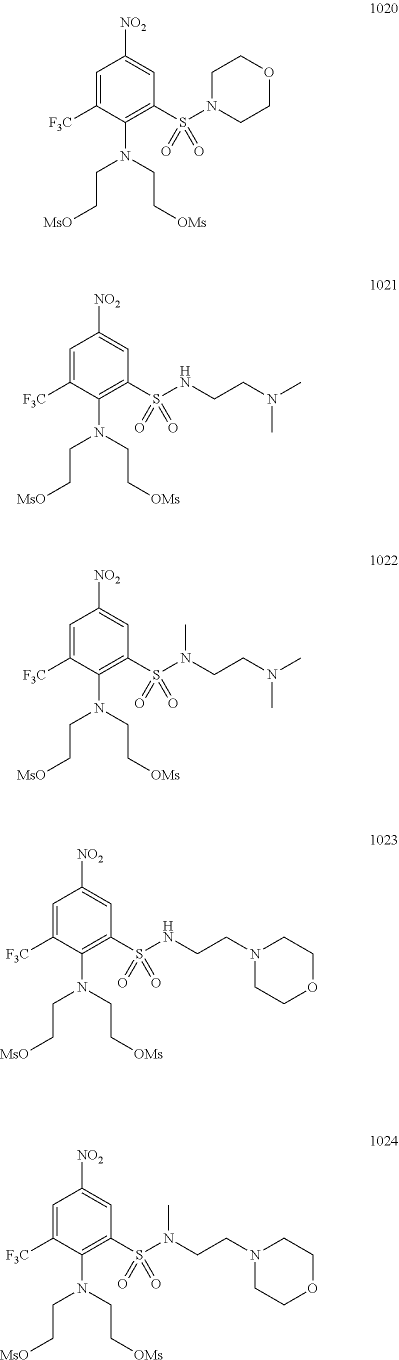 [SMILES: CS(=O)(=O)OCCN(CCOS(C)(=O)=O)c1c(C(F)(F)F)cc([N+](=O)[O-])cc1S(=O)(=O)N1CCOCC1 ; CN(C)CCNS(=O)(=O)c1cc([N+](=O)[O-])cc(C(F)(F)F)c1N(CCOS(C)(=O)=O)CCOS(C)(=O)=O ; CN(C)CCN(C)S(=O)(=O)c1cc([N+](=O)[O-])cc(C(F)(F)F)c1N(CCOS(C)(=O)=O)CCOS(C)(=O)=O ; CS(=O)(=O)OCCN(CCOS(C)(=O)=O)c1c(C(F)(F)F)cc([N+](=O)[O-])cc1S(=O)(=O)NCCN1CCOCC1 ; CN(CCN1CCOCC1)S(=O)(=O)c1cc([N+](=O)[O-])cc(C(F)(F)F)c1N(CCOS(C)(=O)=O)CCOS(C)(=O)=O] 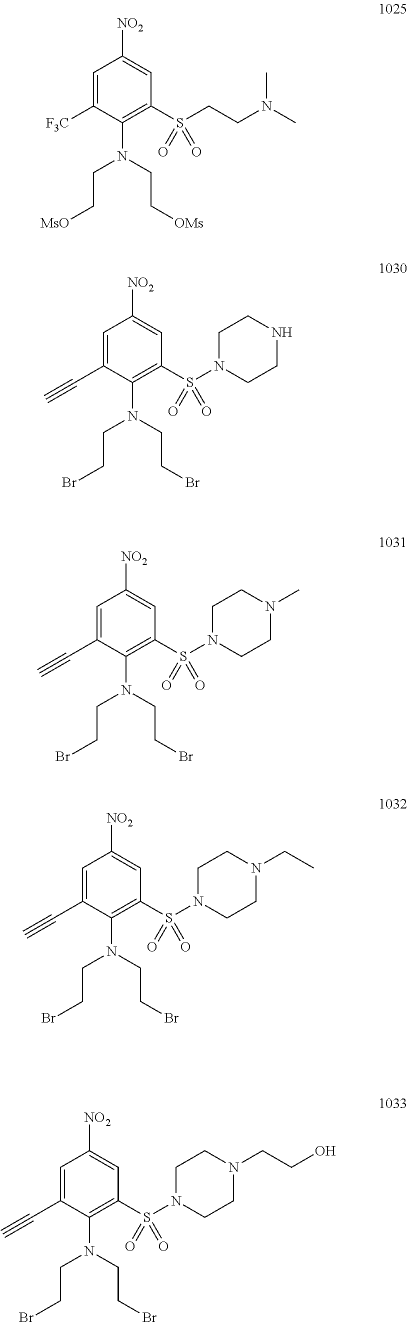 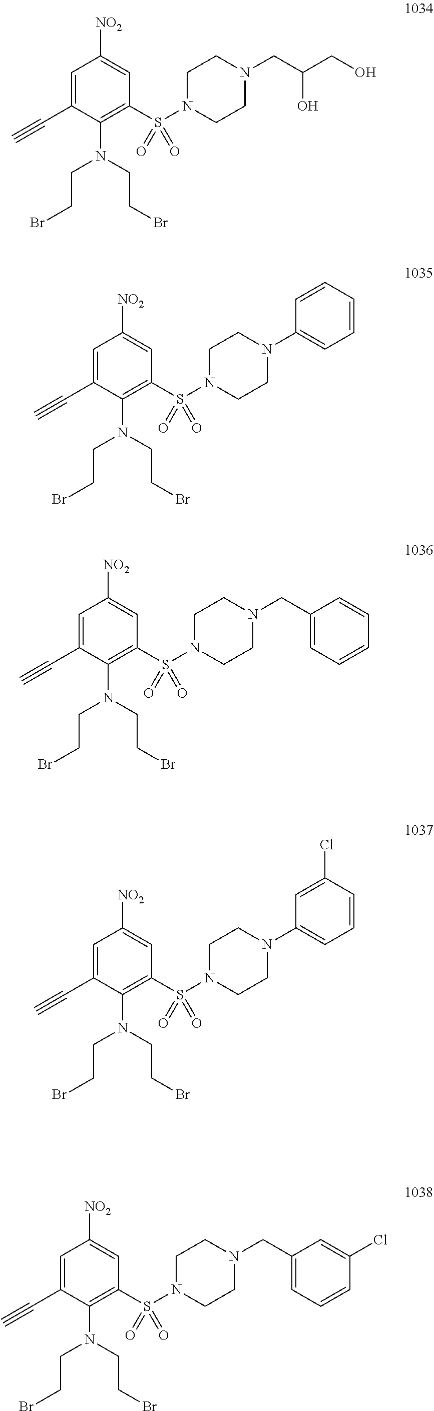 [SMILES: C#Cc1cc([N+](=O)[O-])cc(S(=O)(=O)N2CCN(CC(O)CO)CC2)c1N(CCBr)CCBr ; C#Cc1cc([N+](=O)[O-])cc(S(=O)(=O)N2CCN(c3ccccc3)CC2)c1N(CCBr)CCBr ; C#Cc1cc([N+](=O)[O-])cc(S(=O)(=O)N2CCN(Cc3ccccc3)CC2)c1N(CCBr)CCBr ; C#Cc1cc([N+](=O)[O-])cc(S(=O)(=O)N2CCN(c3cccc(Cl)c3)CC2)c1N(CCBr)CCBr ; C#Cc1cc([N+](=O)[O-])cc(S(=O)(=O)N2CCN(Cc3cccc(Cl)c3)CC2)c1N(CCBr)CCBr] 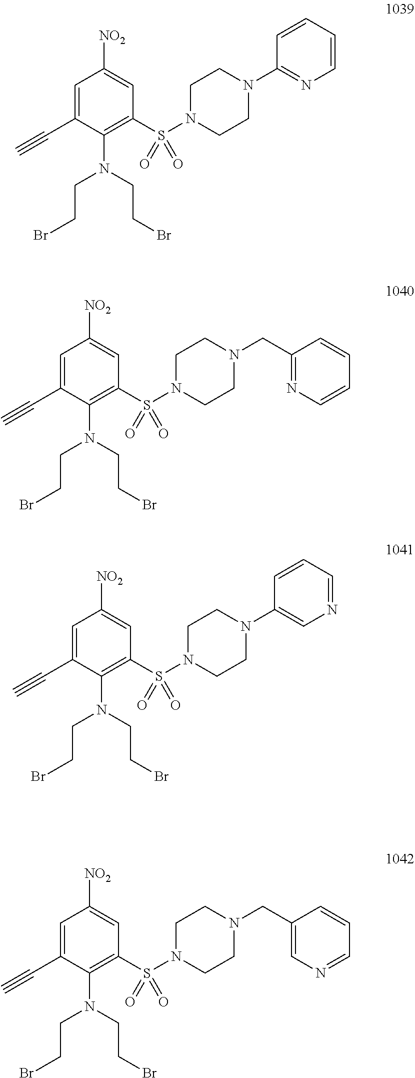 [SMILES: C#Cc1cc([N+](=O)[O-])cc(S(=O)(=O)N2CCN(c3ccccn3)CC2)c1N(CCBr)CCBr ; C#Cc1cc([N+](=O)[O-])cc(S(=O)(=O)N2CCN(Cc3ccccn3)CC2)c1N(CCBr)CCBr ; C#Cc1cc([N+](=O)[O-])cc(S(=O)(=O)N2CCN(c3cccnc3)CC2)c1N(CCBr)CCBr ; C#Cc1cc([N+](=O)[O-])cc(S(=O)(=O)N2CCN(Cc3cccnc3)CC2)c1N(CCBr)CCBr] 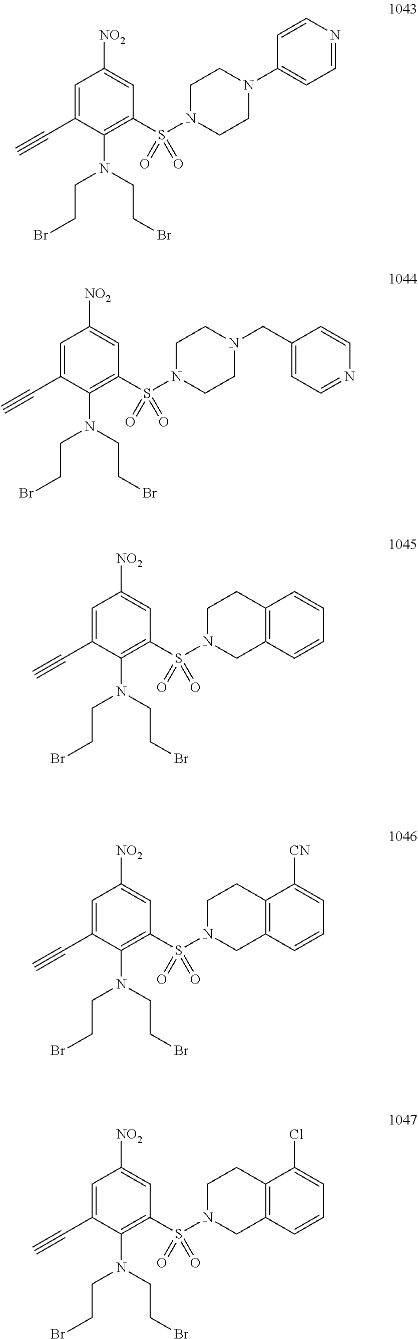 [SMILES: C#Cc1cc([N+](=O)[O-])cc(S(=O)(=O)N2CCN(c3ccncc3)CC2)c1N(CCBr)CCBr ; C#Cc1cc([N+](=O)[O-])cc(S(=O)(=O)N2CCN(Cc3ccncc3)CC2)c1N(CCBr)CCBr ; C#Cc1cc([N+](=O)[O-])cc(S(=O)(=O)N2CCc3ccccc3C2)c1N(CCBr)CCBr ; C#Cc1cc([N+](=O)[O-])cc(S(=O)(=O)N2CCc3c(C#N)cccc3C2)c1N(CCBr)CCBr ; C#Cc1cc([N+](=O)[O-])cc(S(=O)(=O)N2CCc3c(Cl)cccc3C2)c1N(CCBr)CCBr] 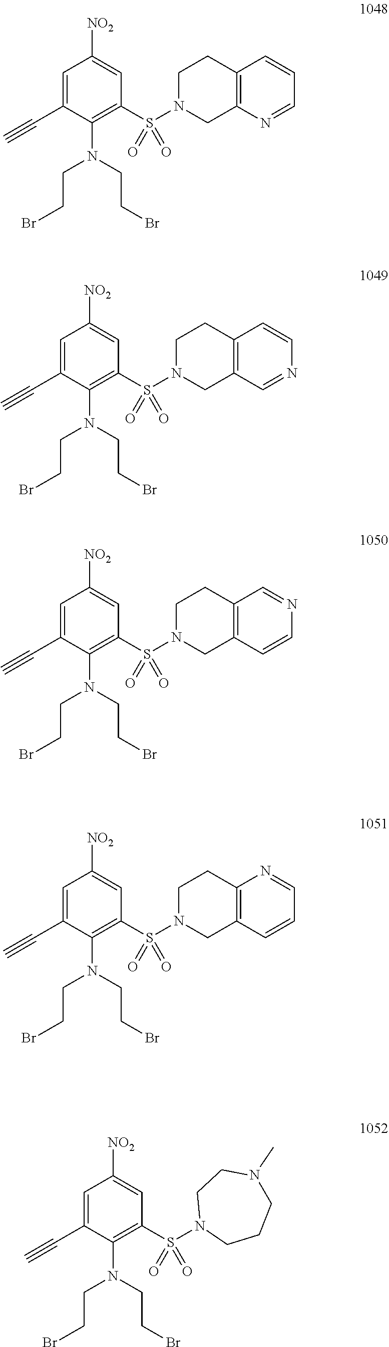 [SMILES: C#Cc1cc([N+](=O)[O-])cc(S(=O)(=O)N2CCc3cccnc3C2)c1N(CCBr)CCBr ; C#Cc1cc([N+](=O)[O-])cc(S(=O)(=O)N2CCc3ccncc3C2)c1N(CCBr)CCBr ; C#Cc1cc([N+](=O)[O-])cc(S(=O)(=O)N2CCc3cnccc3C2)c1N(CCBr)CCBr ; C#Cc1cc([N+](=O)[O-])cc(S(=O)(=O)N2CCc3ncccc3C2)c1N(CCBr)CCBr ; C#Cc1cc([N+](=O)[O-])cc(S(=O)(=O)N2CCCN(C)CC2)c1N(CCBr)CCBr] 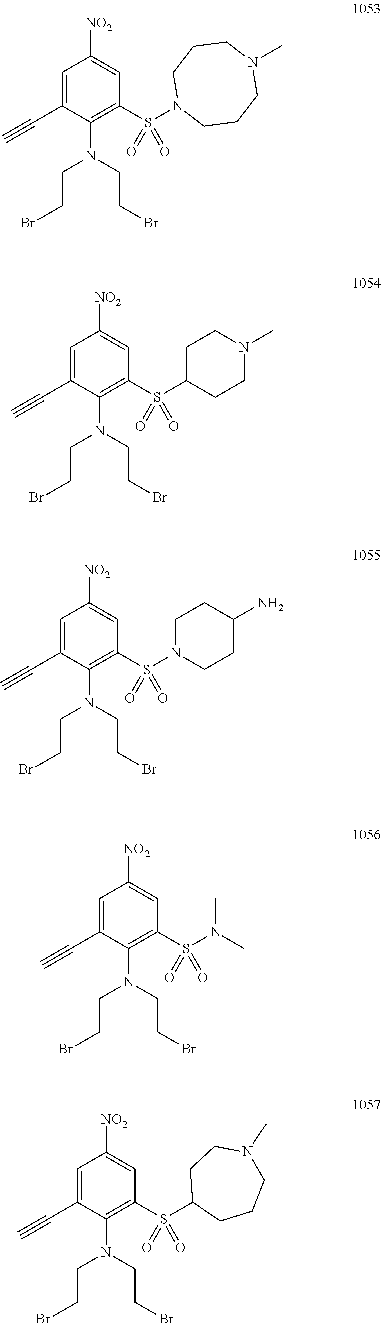 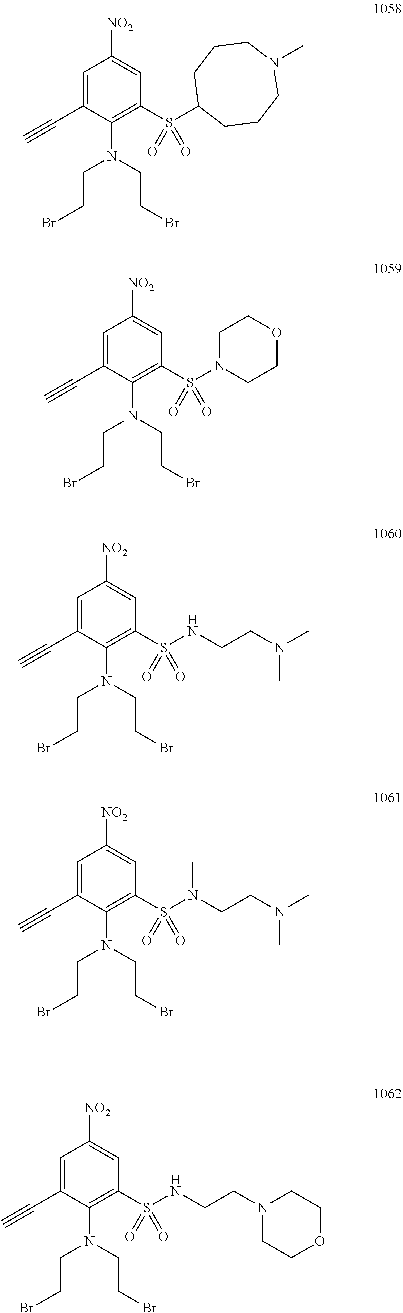 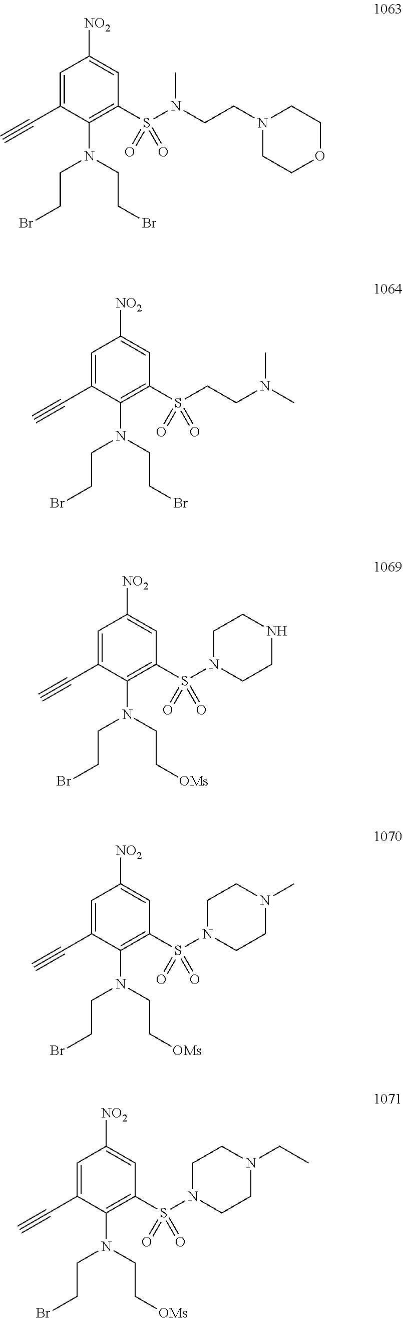 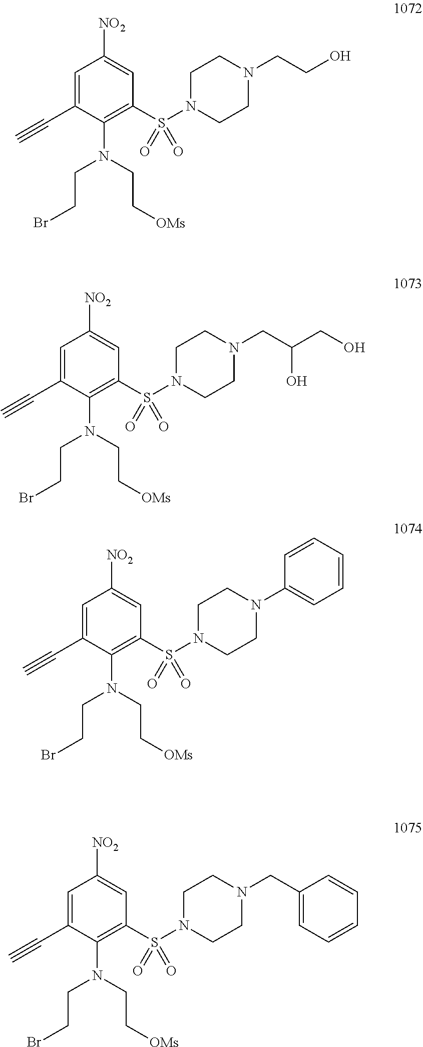 [SMILES: C#Cc1cc([N+](=O)[O-])cc(S(=O)(=O)N2CCN(CCO)CC2)c1N(CCBr)CCOS(C)(=O)=O ; C#Cc1cc([N+](=O)[O-])cc(S(=O)(=O)N2CCN(CC(O)CO)CC2)c1N(CCBr)CCOS(C)(=O)=O ; C#Cc1cc([N+](=O)[O-])cc(S(=O)(=O)N2CCN(c3ccccc3)CC2)c1N(CCBr)CCOS(C)(=O)=O ; C#Cc1cc([N+](=O)[O-])cc(S(=O)(=O)N2CCN(Cc3ccccc3)CC2)c1N(CCBr)CCOS(C)(=O)=O] 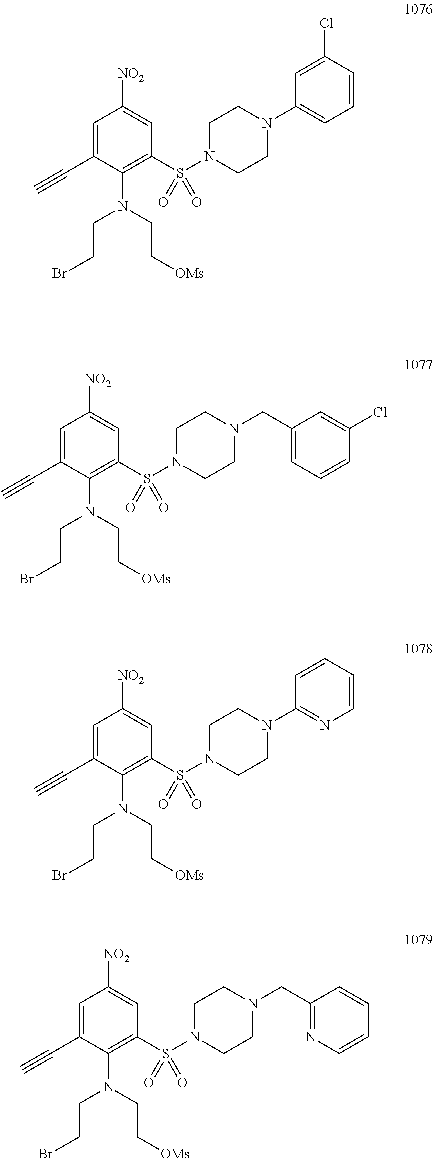 [SMILES: C#Cc1cc([N+](=O)[O-])cc(S(=O)(=O)N2CCN(c3cccc(Cl)c3)CC2)c1N(CCBr)CCOS(C)(=O)=O ; C#Cc1cc([N+](=O)[O-])cc(S(=O)(=O)N2CCN(Cc3cccc(Cl)c3)CC2)c1N(CCBr)CCOS(C)(=O)=O ; C#Cc1cc([N+](=O)[O-])cc(S(=O)(=O)N2CCN(c3ccccn3)CC2)c1N(CCBr)CCOS(C)(=O)=O ; C#Cc1cc([N+](=O)[O-])cc(S(=O)(=O)N2CCN(Cc3ccccn3)CC2)c1N(CCBr)CCOS(C)(=O)=O] 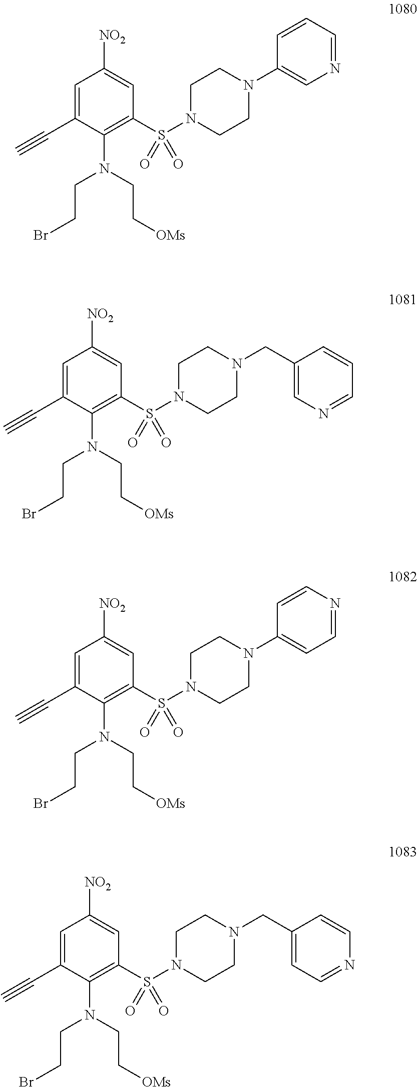 [SMILES: C#Cc1cc([N+](=O)[O-])cc(S(=O)(=O)N2CCN(c3cccnc3)CC2)c1N(CCBr)CCOS(C)(=O)=O ; C#Cc1cc([N+](=O)[O-])cc(S(=O)(=O)N2CCN(Cc3cccnc3)CC2)c1N(CCBr)CCOS(C)(=O)=O ; C#Cc1cc([N+](=O)[O-])cc(S(=O)(=O)N2CCN(c3ccncc3)CC2)c1N(CCBr)CCOS(C)(=O)=O ; C#Cc1cc([N+](=O)[O-])cc(S(=O)(=O)N2CCN(Cc3ccncc3)CC2)c1N(CCBr)CCOS(C)(=O)=O] 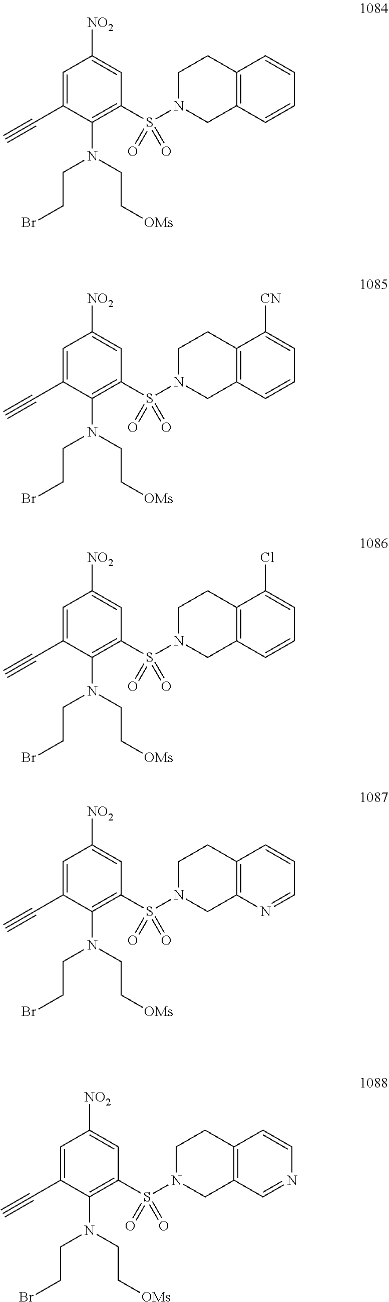 [SMILES: C#Cc1cc([N+](=O)[O-])cc(S(=O)(=O)N2CCc3ccccc3C2)c1N(CCBr)CCOS(C)(=O)=O ; C#Cc1cc([N+](=O)[O-])cc(S(=O)(=O)N2CCc3c(C#N)cccc3C2)c1N(CCBr)CCOS(C)(=O)=O ; C#Cc1cc([N+](=O)[O-])cc(S(=O)(=O)N2CCc3c(Cl)cccc3C2)c1N(CCBr)CCOS(C)(=O)=O ; C#Cc1cc([N+](=O)[O-])cc(S(=O)(=O)N2CCc3cccnc3C2)c1N(CCBr)CCOS(C)(=O)=O ; C#Cc1cc([N+](=O)[O-])cc(S(=O)(=O)N2CCc3ccncc3C2)c1N(CCBr)CCOS(C)(=O)=O] 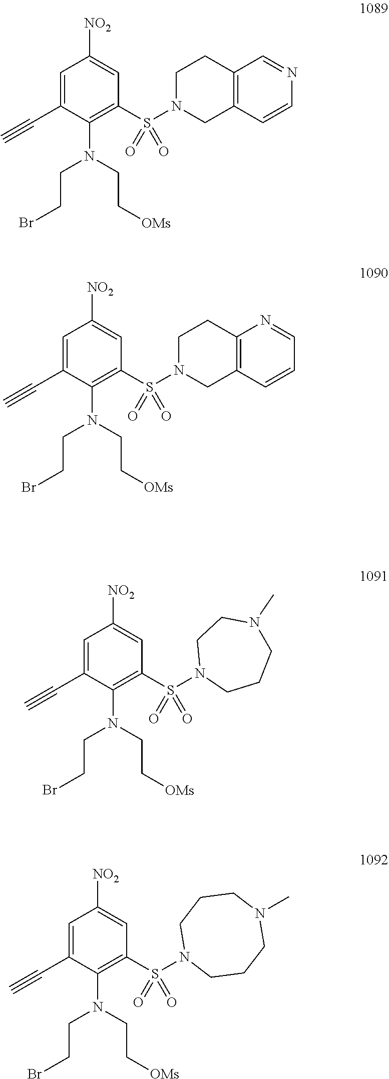 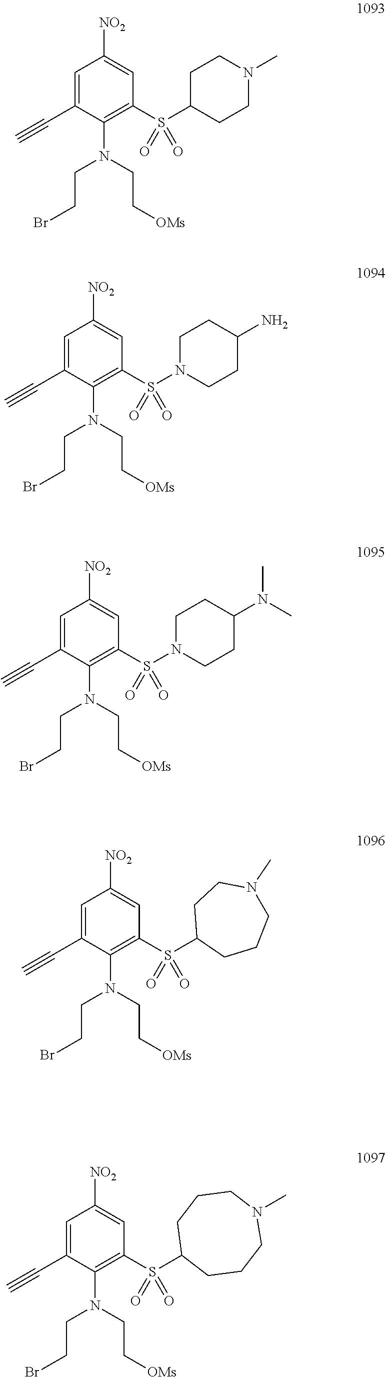 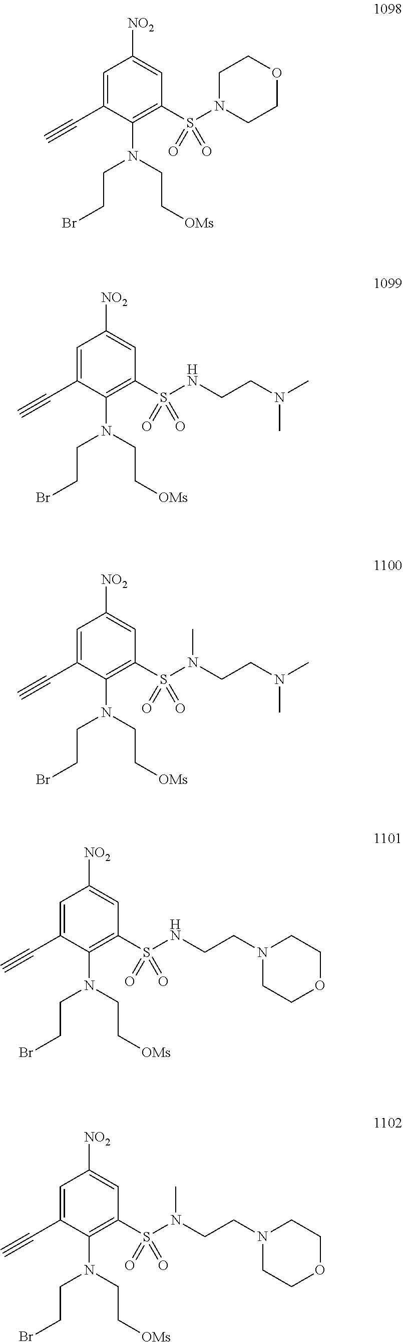 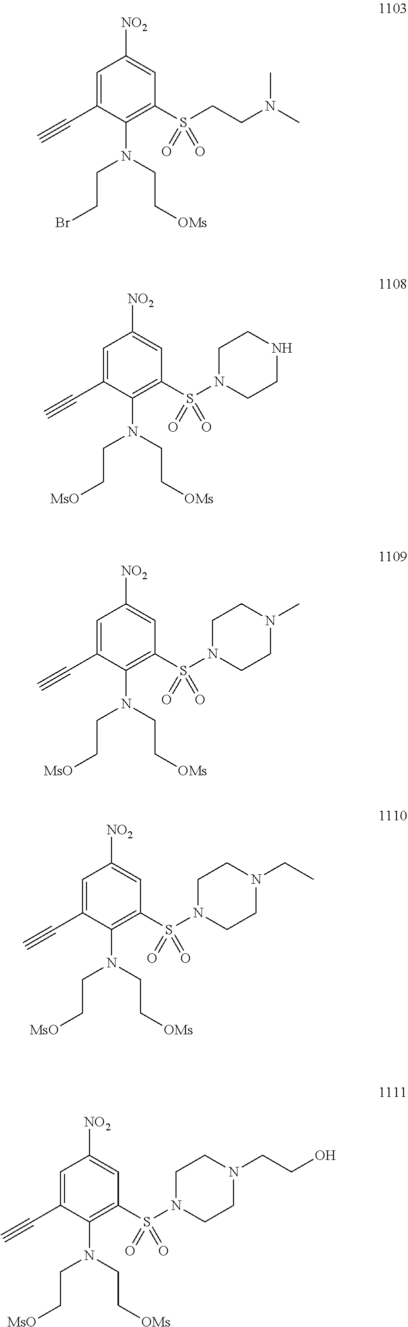 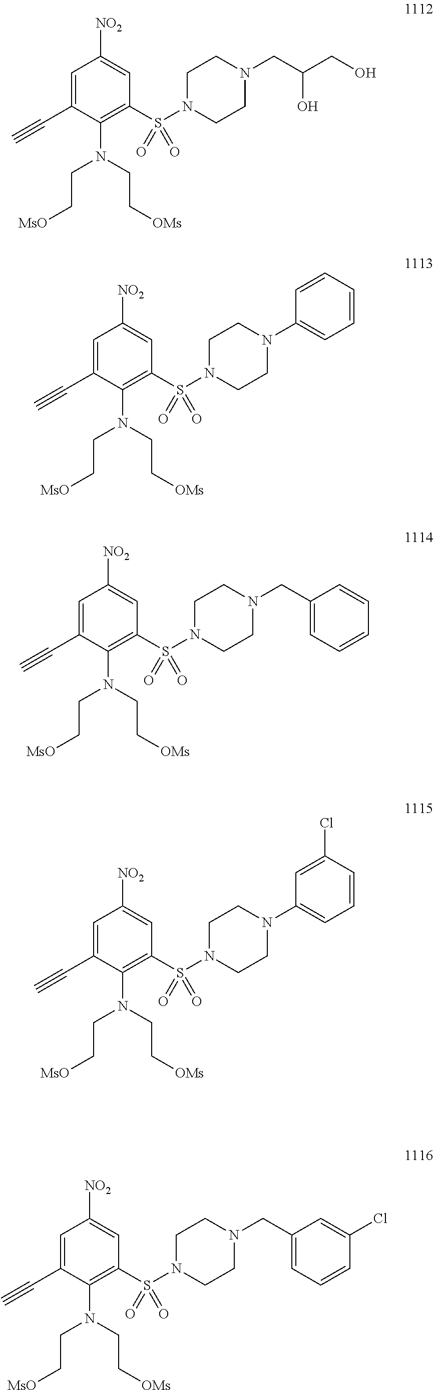 [SMILES: C#Cc1cc([N+](=O)[O-])cc(S(=O)(=O)N2CCN(CC(O)CO)CC2)c1N(CCOS(C)(=O)=O)CCOS(C)(=O)=O ; C#Cc1cc([N+](=O)[O-])cc(S(=O)(=O)N2CCN(c3ccccc3)CC2)c1N(CCOS(C)(=O)=O)CCOS(C)(=O)=O ; C#Cc1cc([N+](=O)[O-])cc(S(=O)(=O)N2CCN(Cc3ccccc3)CC2)c1N(CCOS(C)(=O)=O)CCOS(C)(=O)=O ; C#Cc1cc([N+](=O)[O-])cc(S(=O)(=O)N2CCN(c3cccc(Cl)c3)CC2)c1N(CCOS(C)(=O)=O)CCOS(C)(=O)=O ; C#Cc1cc([N+](=O)[O-])cc(S(=O)(=O)N2CCN(Cc3cccc(Cl)c3)CC2)c1N(CCOS(C)(=O)=O)CCOS(C)(=O)=O] 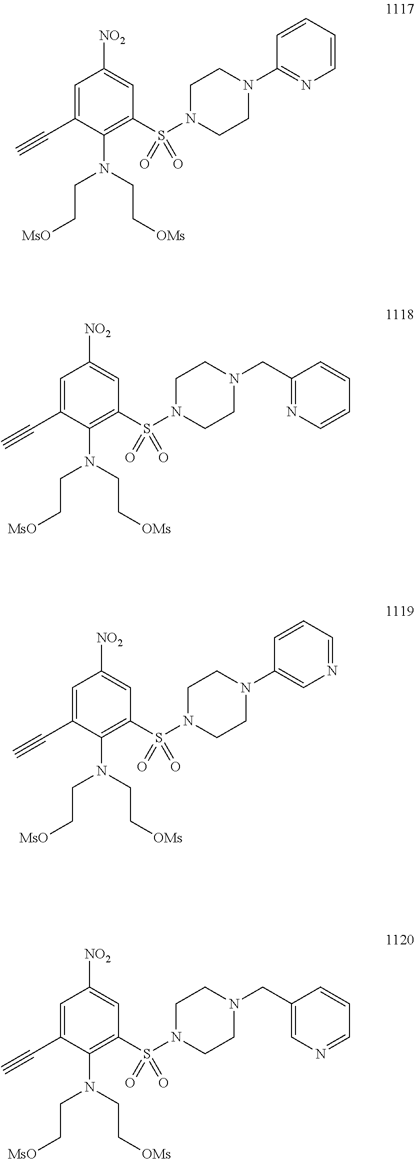 [SMILES: C#Cc1cc([N+](=O)[O-])cc(S(=O)(=O)N2CCN(c3ccccn3)CC2)c1N(CCOS(C)(=O)=O)CCOS(C)(=O)=O ; C#Cc1cc([N+](=O)[O-])cc(S(=O)(=O)N2CCN(Cc3ccccn3)CC2)c1N(CCOS(C)(=O)=O)CCOS(C)(=O)=O ; C#Cc1cc([N+](=O)[O-])cc(S(=O)(=O)N2CCN(c3cccnc3)CC2)c1N(CCOS(C)(=O)=O)CCOS(C)(=O)=O ; C#Cc1cc([N+](=O)[O-])cc(S(=O)(=O)N2CCN(Cc3cccnc3)CC2)c1N(CCOS(C)(=O)=O)CCOS(C)(=O)=O] 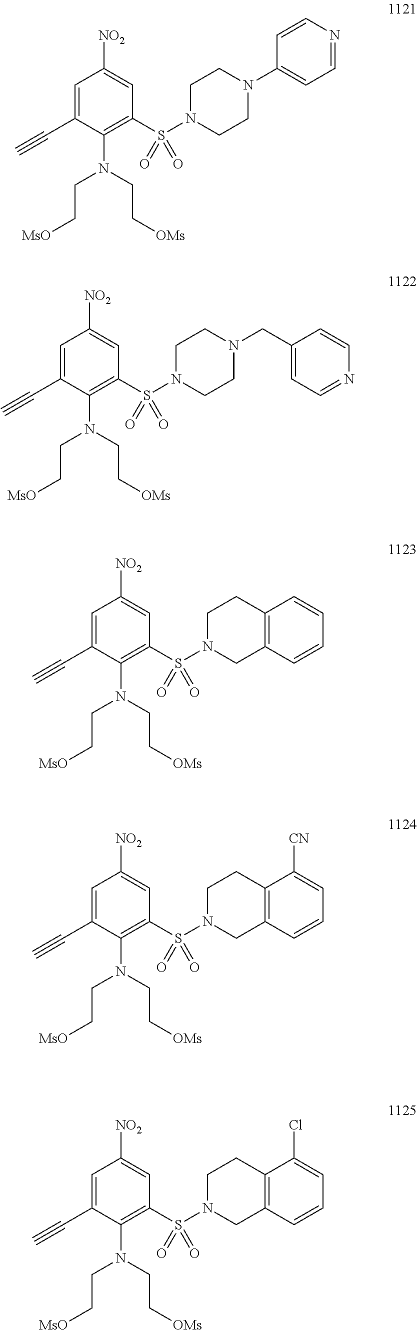 [SMILES: C#Cc1cc([N+](=O)[O-])cc(S(=O)(=O)N2CCN(c3ccncc3)CC2)c1N(CCOS(C)(=O)=O)CCOS(C)(=O)=O ; C#Cc1cc([N+](=O)[O-])cc(S(=O)(=O)N2CCN(Cc3ccncc3)CC2)c1N(CCOS(C)(=O)=O)CCOS(C)(=O)=O ; C#Cc1cc([N+](=O)[O-])cc(S(=O)(=O)N2CCc3ccccc3C2)c1N(CCOS(C)(=O)=O)CCOS(C)(=O)=O ; C#Cc1cc([N+](=O)[O-])cc(S(=O)(=O)N2CCc3c(C#N)cccc3C2)c1N(CCOS(C)(=O)=O)CCOS(C)(=O)=O ; C#Cc1cc([N+](=O)[O-])cc(S(=O)(=O)N2CCc3c(Cl)cccc3C2)c1N(CCOS(C)(=O)=O)CCOS(C)(=O)=O] 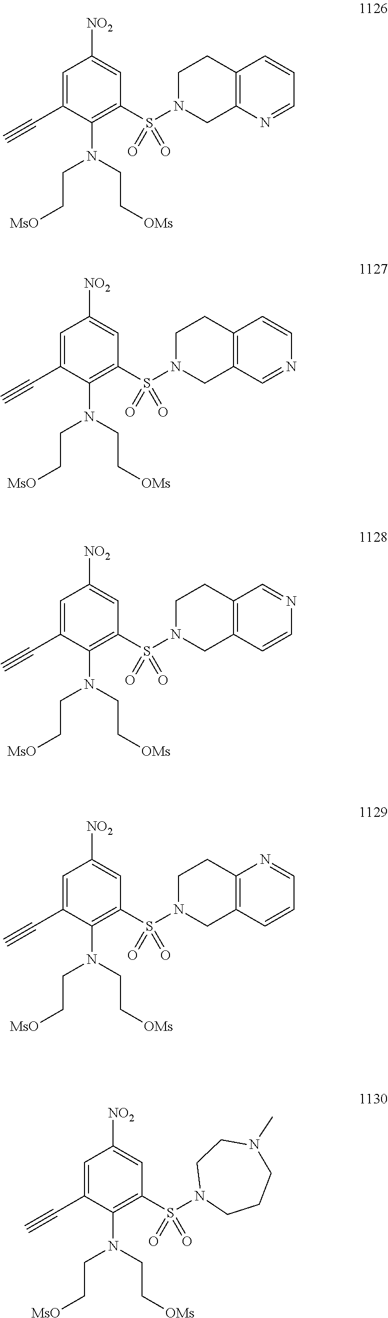 [SMILES: C#Cc1cc([N+](=O)[O-])cc(S(=O)(=O)N2CCc3cccnc3C2)c1N(CCOS(C)(=O)=O)CCOS(C)(=O)=O ; C#Cc1cc([N+](=O)[O-])cc(S(=O)(=O)N2CCc3ccncc3C2)c1N(CCOS(C)(=O)=O)CCOS(C)(=O)=O ; C#Cc1cc([N+](=O)[O-])cc(S(=O)(=O)N2CCc3cnccc3C2)c1N(CCOS(C)(=O)=O)CCOS(C)(=O)=O ; C#Cc1cc([N+](=O)[O-])cc(S(=O)(=O)N2CCc3ncccc3C2)c1N(CCOS(C)(=O)=O)CCOS(C)(=O)=O ; C#Cc1cc([N+](=O)[O-])cc(S(=O)(=O)N2CCCN(C)CC2)c1N(CCOS(C)(=O)=O)CCOS(C)(=O)=O] 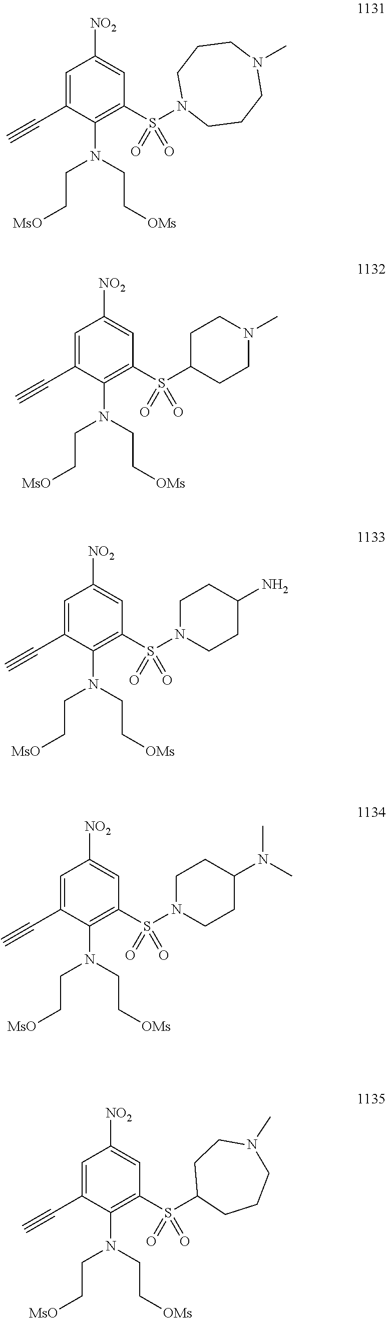 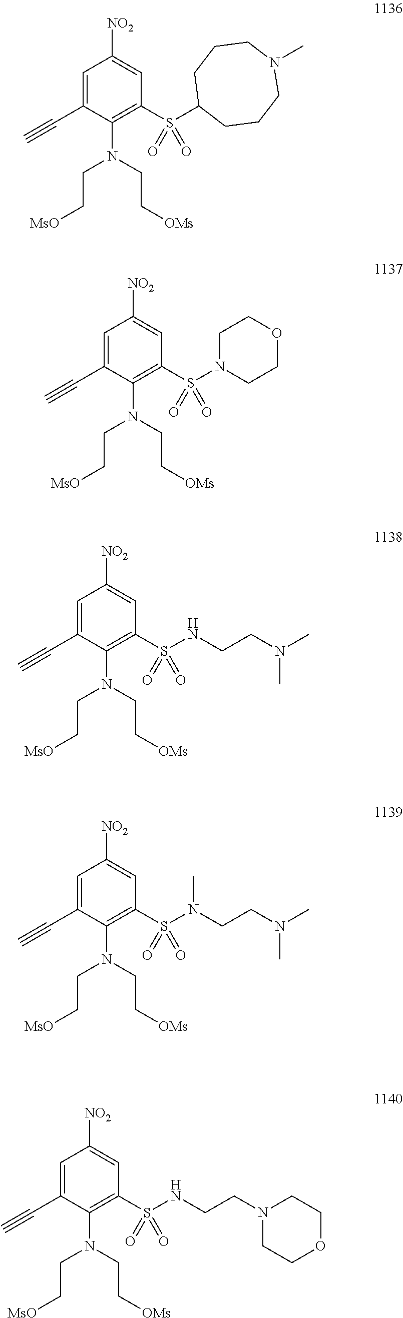 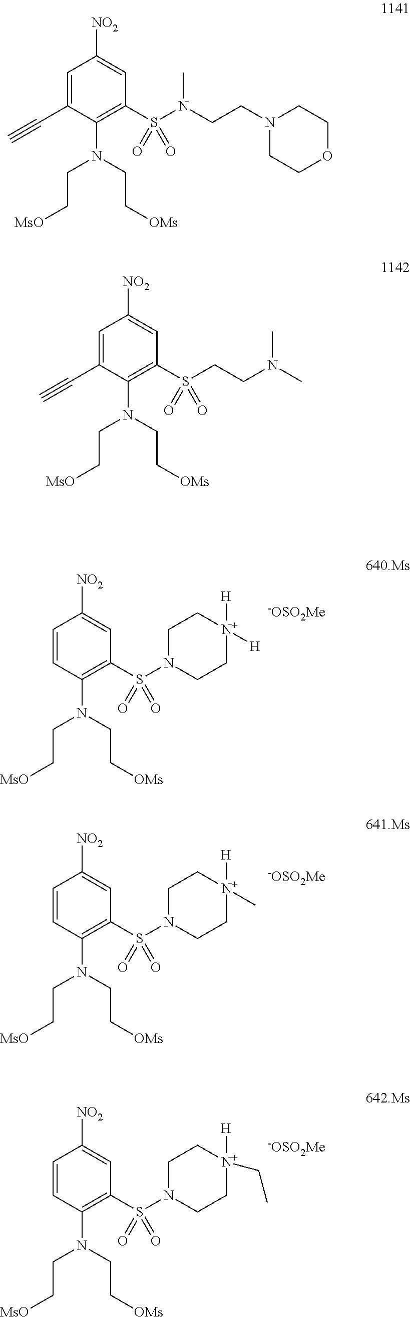 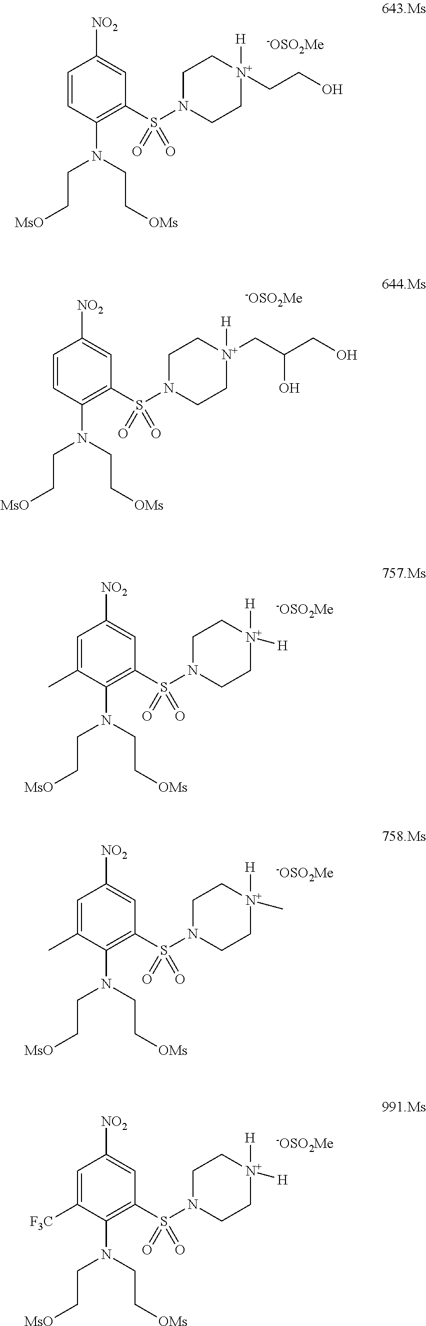 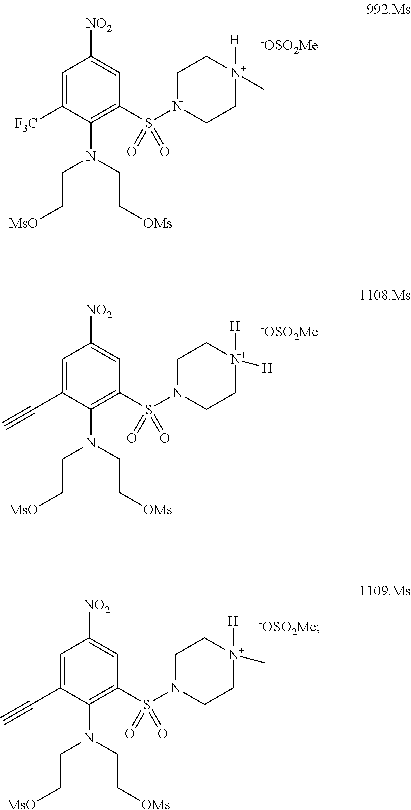
the compound of Formula (4) is selected from the group consisting of: 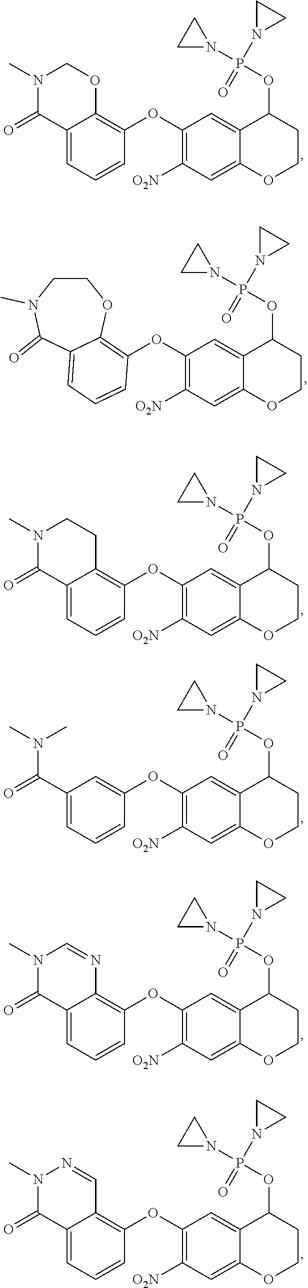 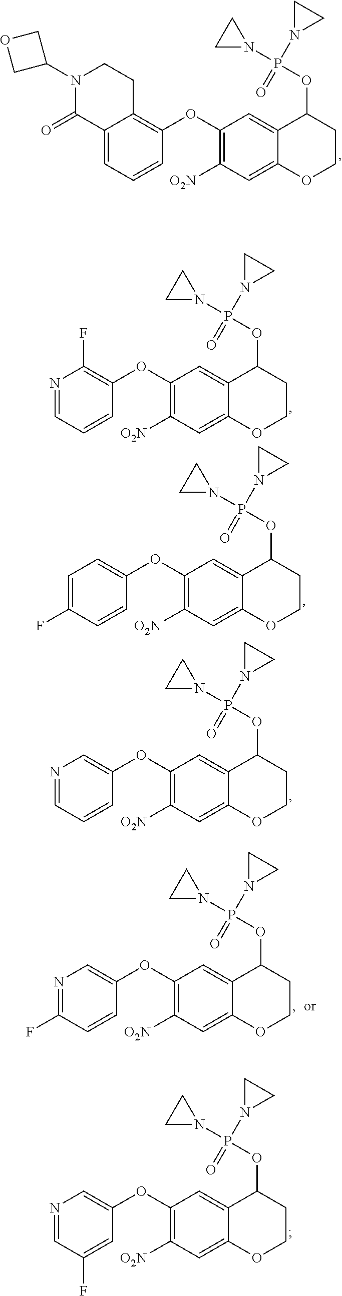
the compound of Formula (5) is selected from the group consisting of: 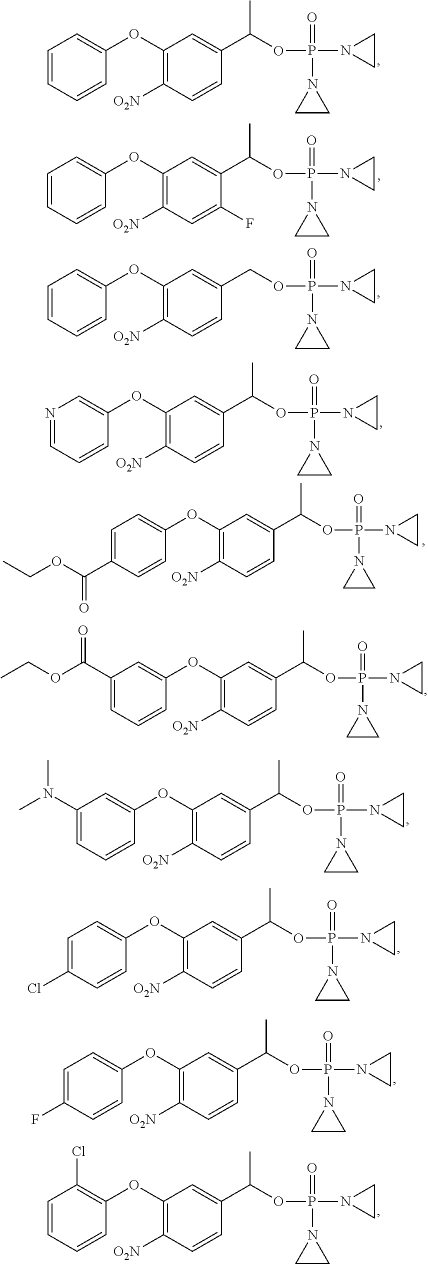 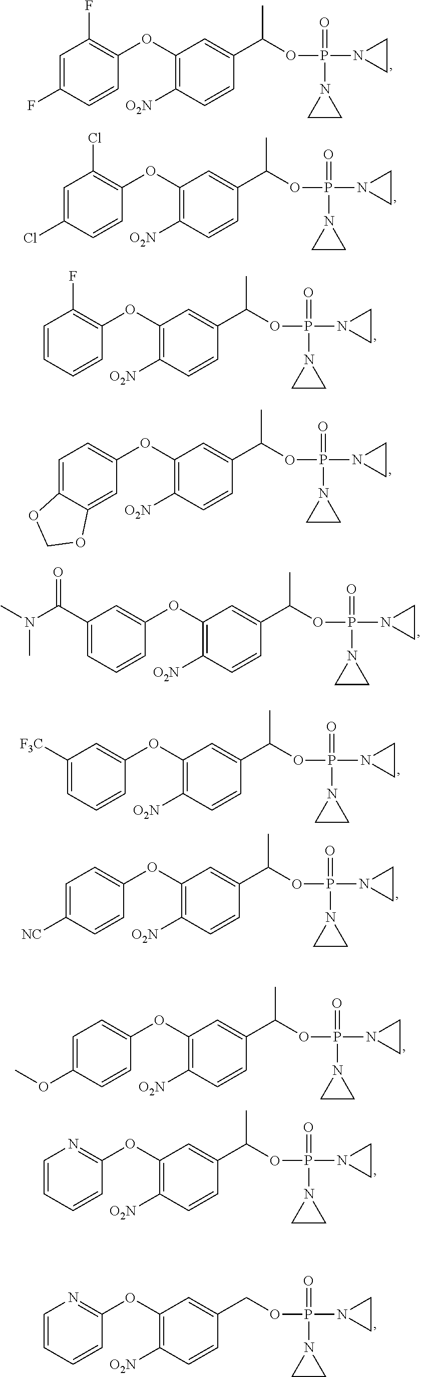 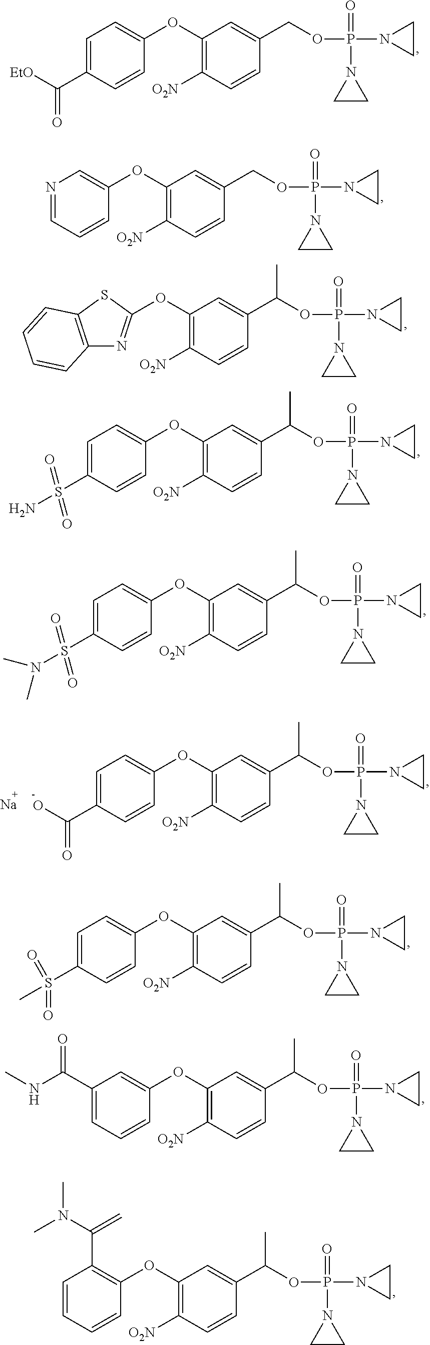 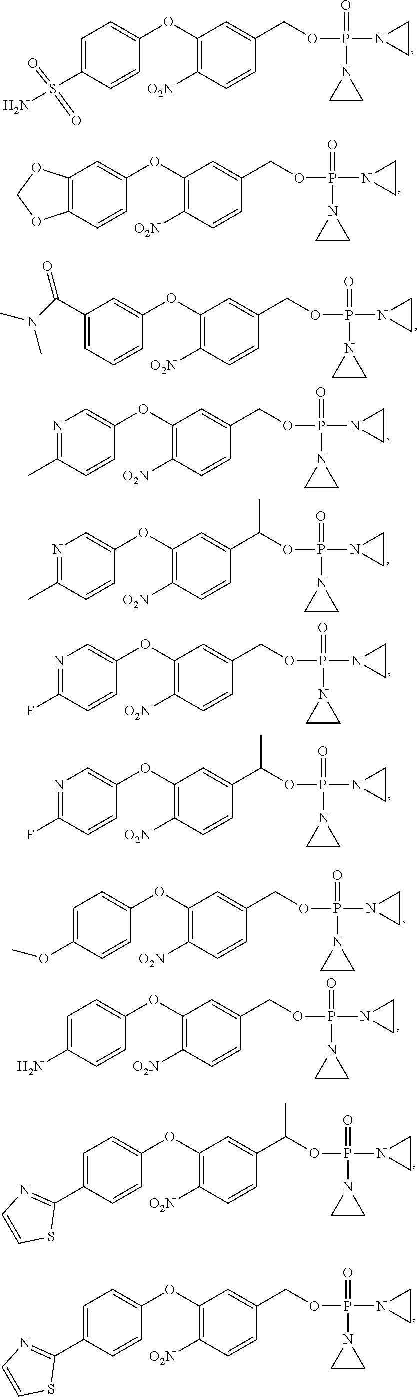 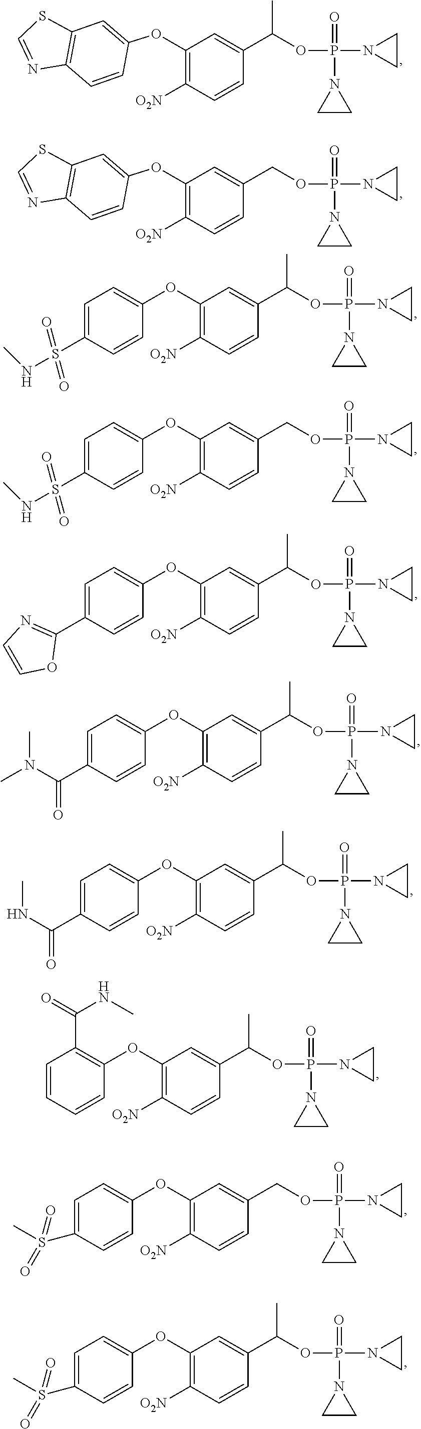 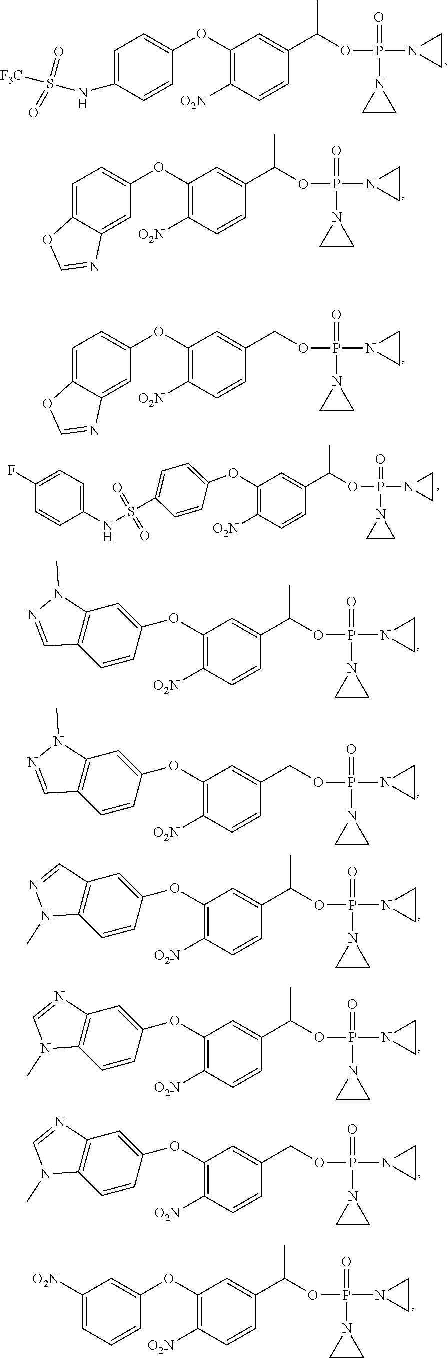 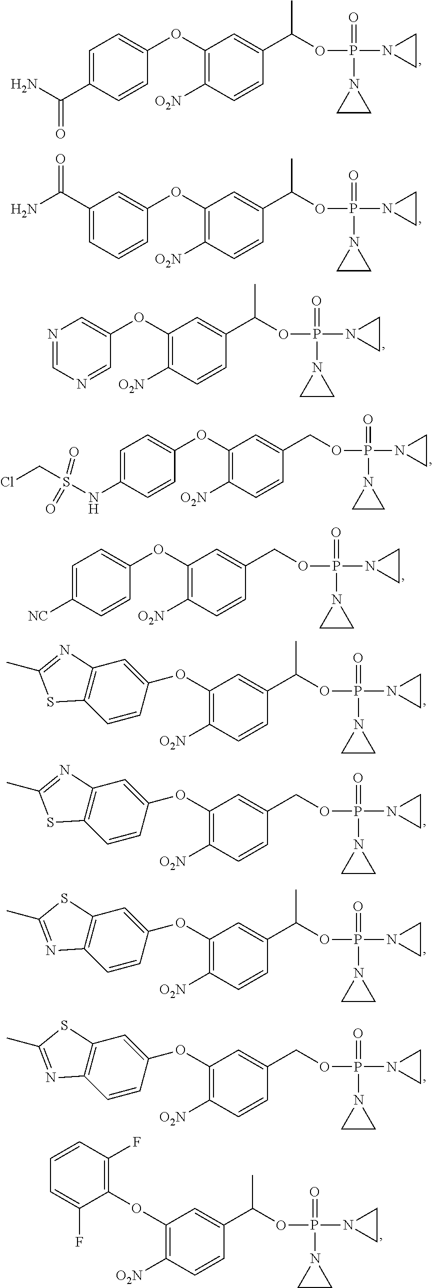 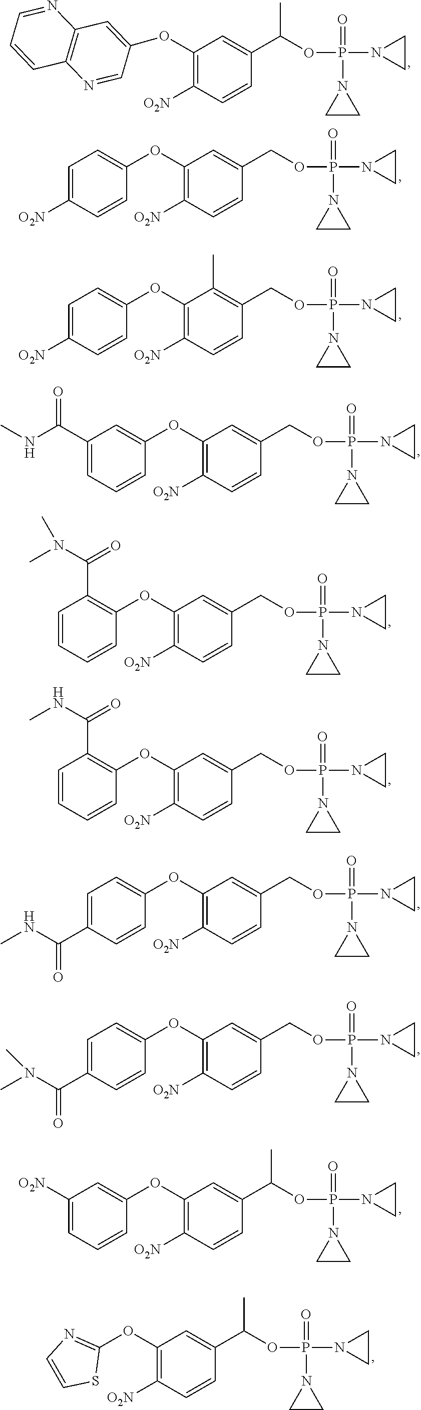 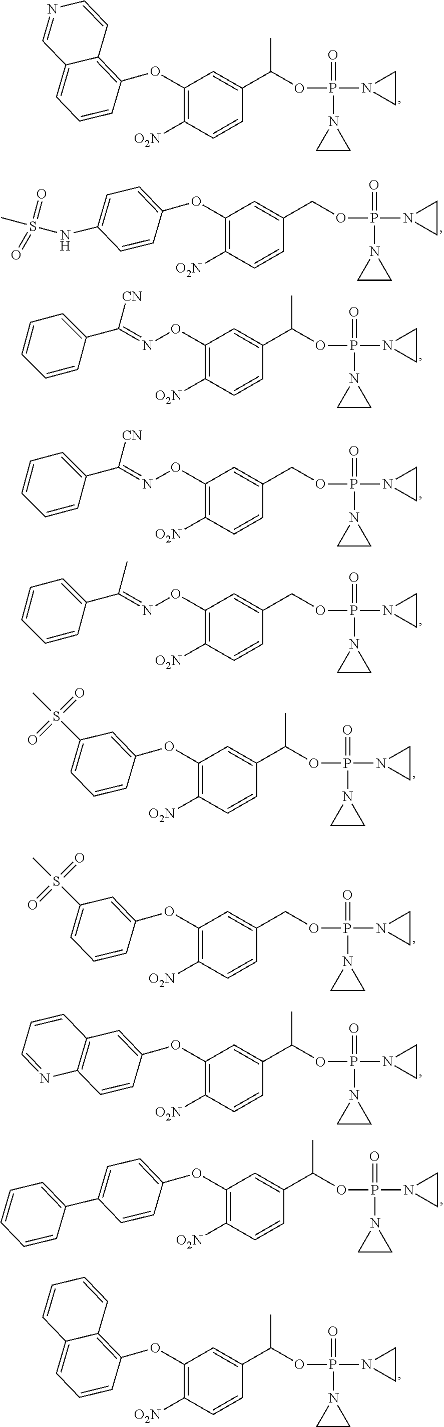 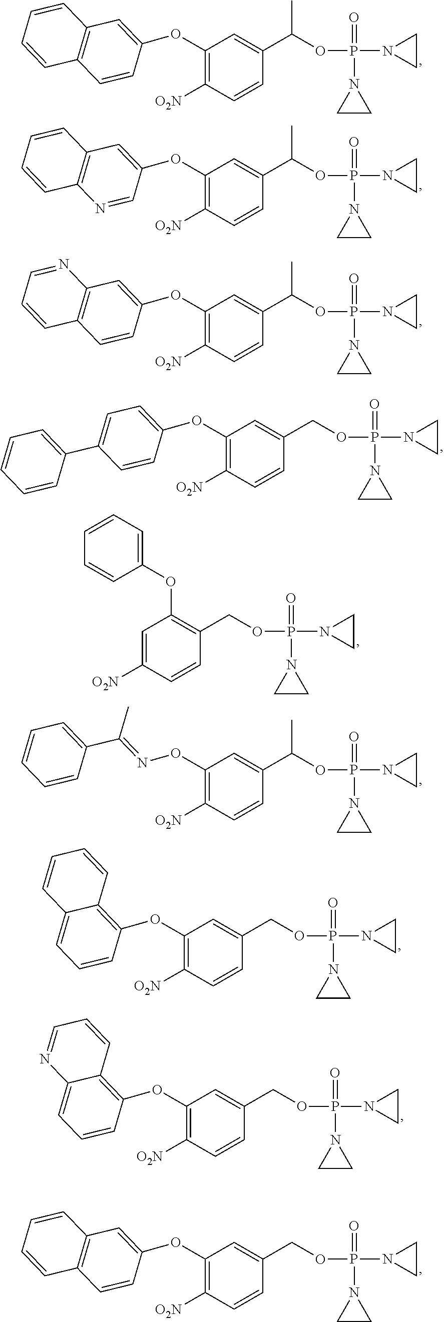 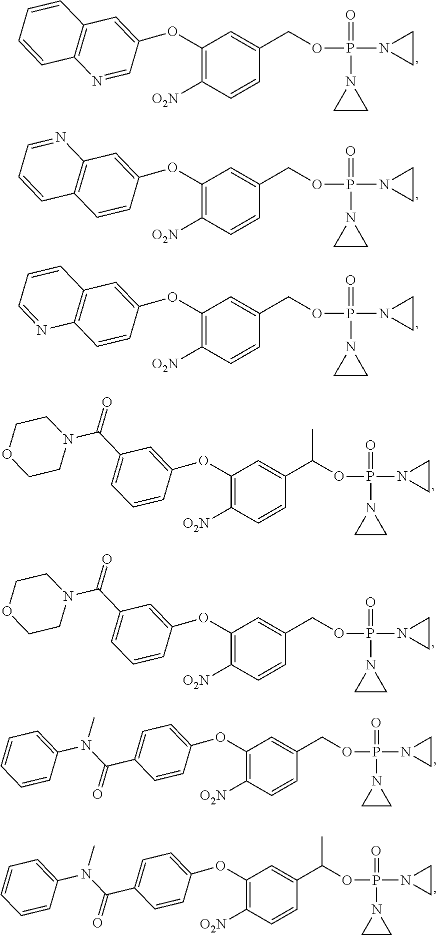 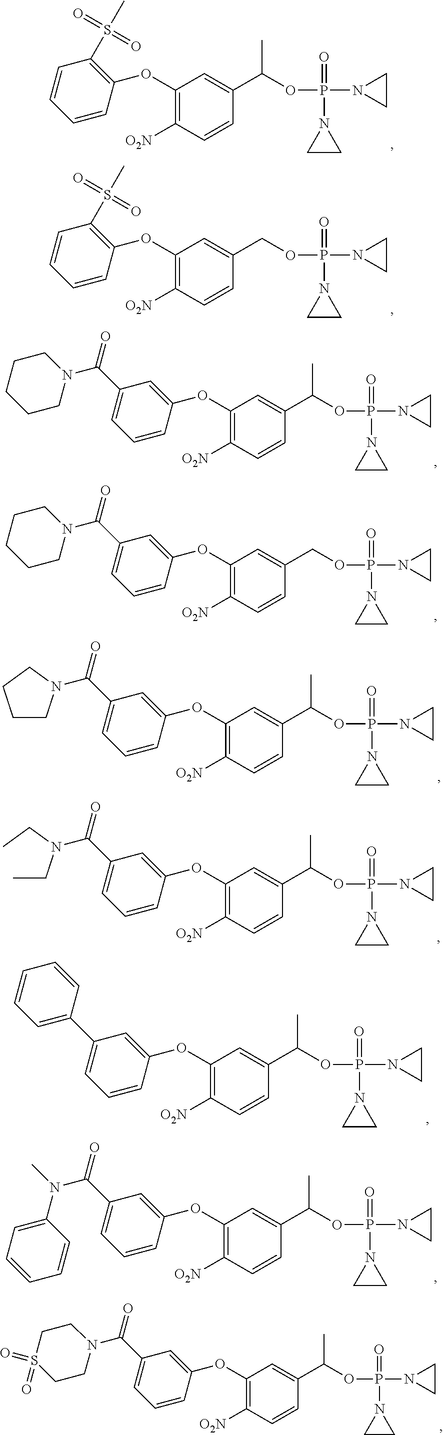 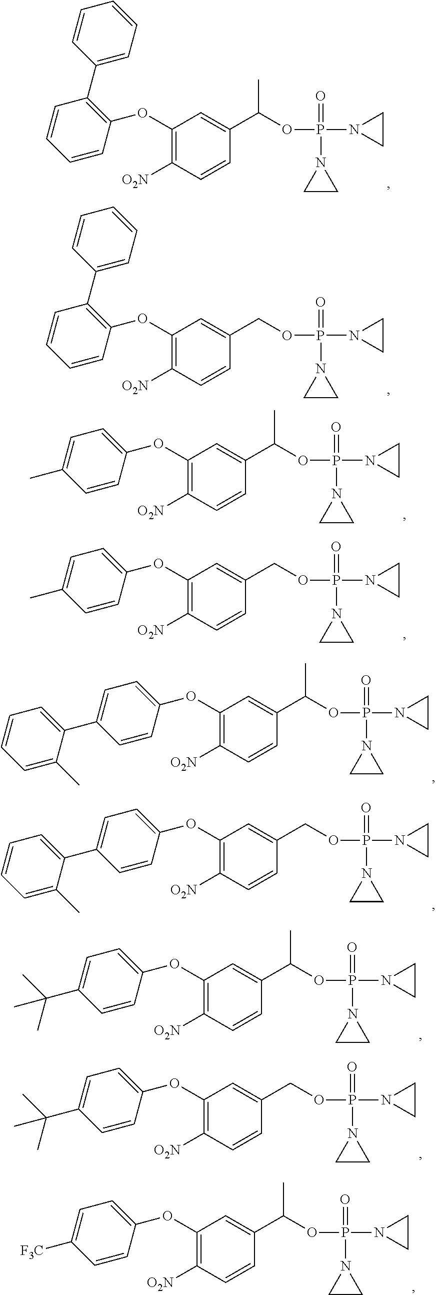 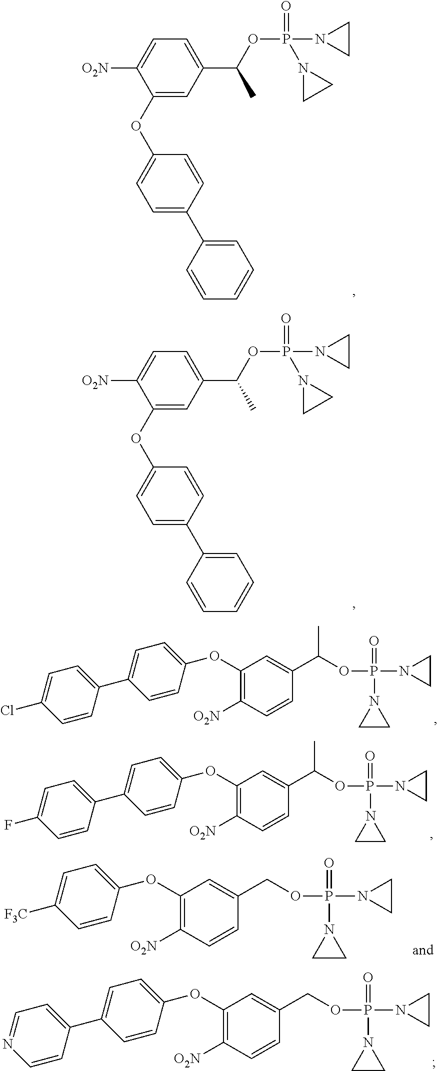
the compound of Formula (6) is selected from the group consisting of:

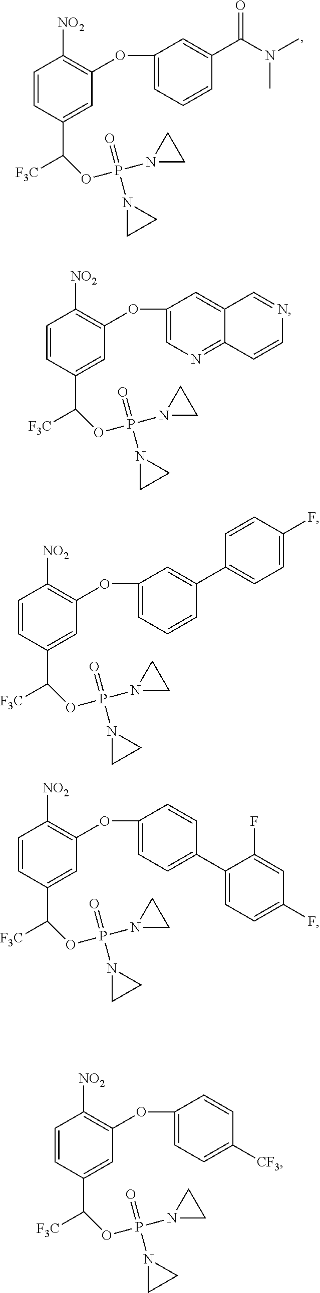 [SMILES: CN(C)C(=O)c1cccc(Oc2cc(C(OP(=O)(N3CC3)N3CC3)C(F)(F)F)ccc2[N+](=O)[O-])c1 ; O=[N+]([O-])c1ccc(C(OP(=O)(N2CC2)N2CC2)C(F)(F)F)cc1Oc1cnc2ccncc2c1 ; O=[N+]([O-])c1ccc(C(OP(=O)(N2CC2)N2CC2)C(F)(F)F)cc1Oc1cccc(-c2ccc(F)cc2)c1 ; O=[N+]([O-])c1ccc(C(OP(=O)(N2CC2)N2CC2)C(F)(F)F)cc1Oc1ccc(-c2ccc(F)cc2F)cc1 ; O=[N+]([O-])c1ccc(C(OP(=O)(N2CC2)N2CC2)C(F)(F)F)cc1Oc1ccc(C(F)(F)F)cc1]

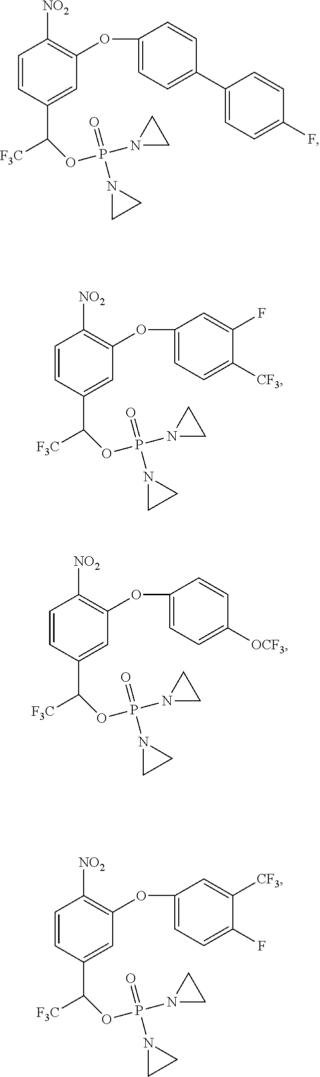 [SMILES: O=[N+]([O-])c1ccc(C(OP(=O)(N2CC2)N2CC2)C(F)(F)F)cc1Oc1ccc(-c2ccc(F)cc2)cc1 ; O=[N+]([O-])c1ccc(C(OP(=O)(N2CC2)N2CC2)C(F)(F)F)cc1Oc1ccc(C(F)(F)F)c(F)c1 ; O=[N+]([O-])c1ccc(C(OP(=O)(N2CC2)N2CC2)C(F)(F)F)cc1Oc1ccc(OC(F)(F)F)cc1 ; O=[N+]([O-])c1ccc(C(OP(=O)(N2CC2)N2CC2)C(F)(F)F)cc1Oc1ccc(F)c(C(F)(F)F)c1]

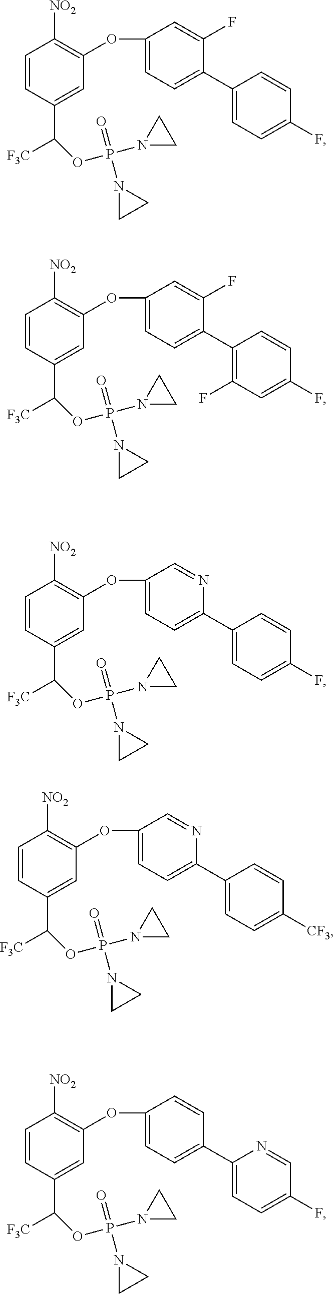 [SMILES: O=[N+]([O-])c1ccc(C(OP(=O)(N2CC2)N2CC2)C(F)(F)F)cc1Oc1ccc(-c2ccc(F)cc2)c(F)c1 ; O=[N+]([O-])c1ccc(C(OP(=O)(N2CC2)N2CC2)C(F)(F)F)cc1Oc1ccc(-c2ccc(F)cc2F)c(F)c1 ; O=[N+]([O-])c1ccc(C(OP(=O)(N2CC2)N2CC2)C(F)(F)F)cc1Oc1ccc(-c2ccc(F)cc2)nc1 ; O=[N+]([O-])c1ccc(C(OP(=O)(N2CC2)N2CC2)C(F)(F)F)cc1Oc1ccc(-c2ccc(C(F)(F)F)cc2)nc1 ; O=[N+]([O-])c1ccc(C(OP(=O)(N2CC2)N2CC2)C(F)(F)F)cc1Oc1ccc(-c2ccc(F)cn2)cc1]

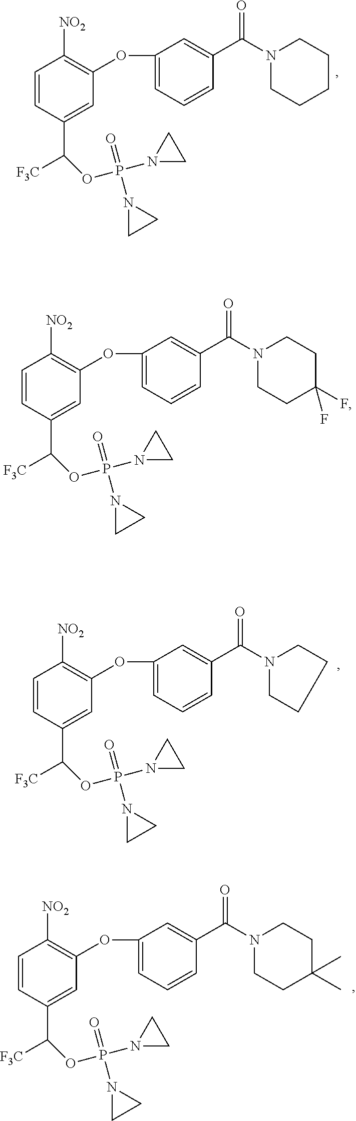 [SMILES: O=C(c1cccc(Oc2cc(C(OP(=O)(N3CC3)N3CC3)C(F)(F)F)ccc2[N+](=O)[O-])c1)N1CCCCC1 ; O=C(c1cccc(Oc2cc(C(OP(=O)(N3CC3)N3CC3)C(F)(F)F)ccc2[N+](=O)[O-])c1)N1CCC(F)(F)CC1 ; O=C(c1cccc(Oc2cc(C(OP(=O)(N3CC3)N3CC3)C(F)(F)F)ccc2[N+](=O)[O-])c1)N1CCCC1 ; CC1(C)CCN(C(=O)c2cccc(Oc3cc(C(OP(=O)(N4CC4)N4CC4)C(F)(F)F)ccc3[N+](=O)[O-])c2)CC1]

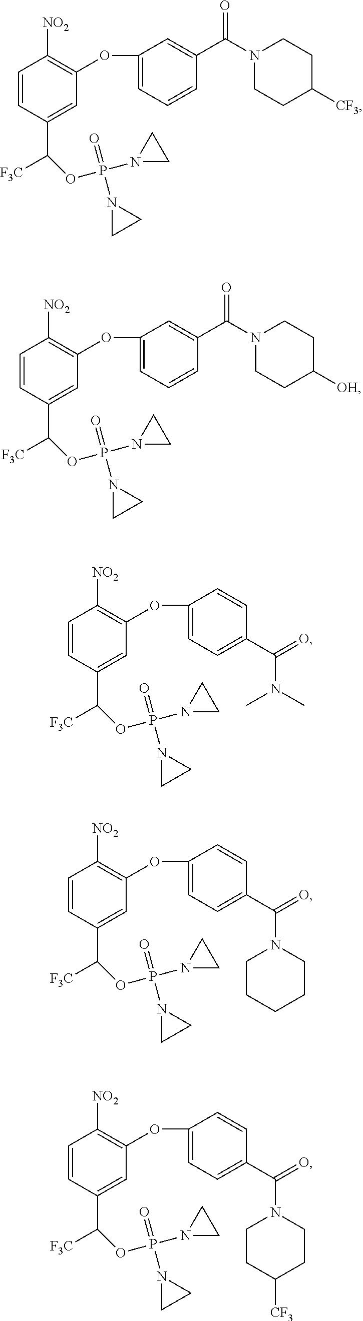 [SMILES: O=C(c1cccc(Oc2cc(C(OP(=O)(N3CC3)N3CC3)C(F)(F)F)ccc2[N+](=O)[O-])c1)N1CCC(C(F)(F)F)CC1 ; O=C(c1cccc(Oc2cc(C(OP(=O)(N3CC3)N3CC3)C(F)(F)F)ccc2[N+](=O)[O-])c1)N1CCC(O)CC1 ; CN(C)C(=O)c1ccc(Oc2cc(C(OP(=O)(N3CC3)N3CC3)C(F)(F)F)ccc2[N+](=O)[O-])cc1 ; O=C(c1ccc(Oc2cc(C(OP(=O)(N3CC3)N3CC3)C(F)(F)F)ccc2[N+](=O)[O-])cc1)N1CCCCC1 ; O=C(c1ccc(Oc2cc(C(OP(=O)(N3CC3)N3CC3)C(F)(F)F)ccc2[N+](=O)[O-])cc1)N1CCC(C(F)(F)F)CC1]

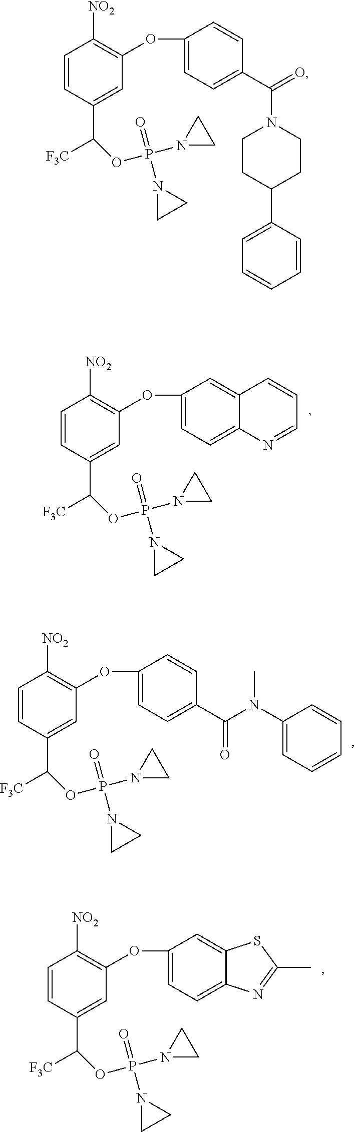 [SMILES: O=C(c1ccc(Oc2cc(C(OP(=O)(N3CC3)N3CC3)C(F)(F)F)ccc2[N+](=O)[O-])cc1)N1CCC(c2ccccc2)CC1 ; O=[N+]([O-])c1ccc(C(OP(=O)(N2CC2)N2CC2)C(F)(F)F)cc1Oc1ccc2ncccc2c1 ; CN(C(=O)c1ccc(Oc2cc(C(OP(=O)(N3CC3)N3CC3)C(F)(F)F)ccc2[N+](=O)[O-])cc1)c1ccccc1 ; Cc1nc2ccc(Oc3cc(C(OP(=O)(N4CC4)N4CC4)C(F)(F)F)ccc3[N+](=O)[O-])cc2s1]

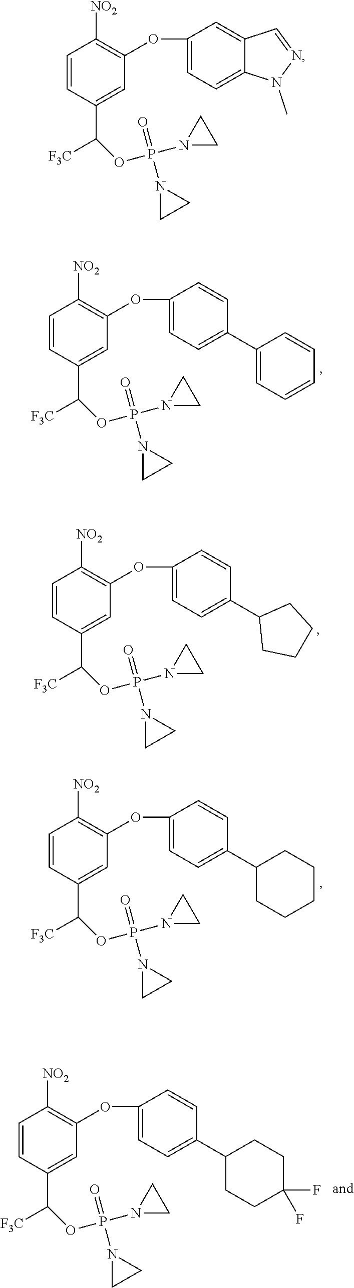 [SMILES: Cn1ncc2cc(Oc3cc(C(OP(=O)(N4CC4)N4CC4)C(F)(F)F)ccc3[N+](=O)[O-])ccc21 ; O=[N+]([O-])c1ccc(C(OP(=O)(N2CC2)N2CC2)C(F)(F)F)cc1Oc1ccc(-c2ccccc2)cc1 ; O=[N+]([O-])c1ccc(C(OP(=O)(N2CC2)N2CC2)C(F)(F)F)cc1Oc1ccc(C2CCCC2)cc1 ; O=[N+]([O-])c1ccc(C(OP(=O)(N2CC2)N2CC2)C(F)(F)F)cc1Oc1ccc(C2CCCCC2)cc1 ; O=[N+]([O-])c1ccc(C(OP(=O)(N2CC2)N2CC2)C(F)(F)F)cc1Oc1ccc(C2CCC(F)(F)CC2)cc1]

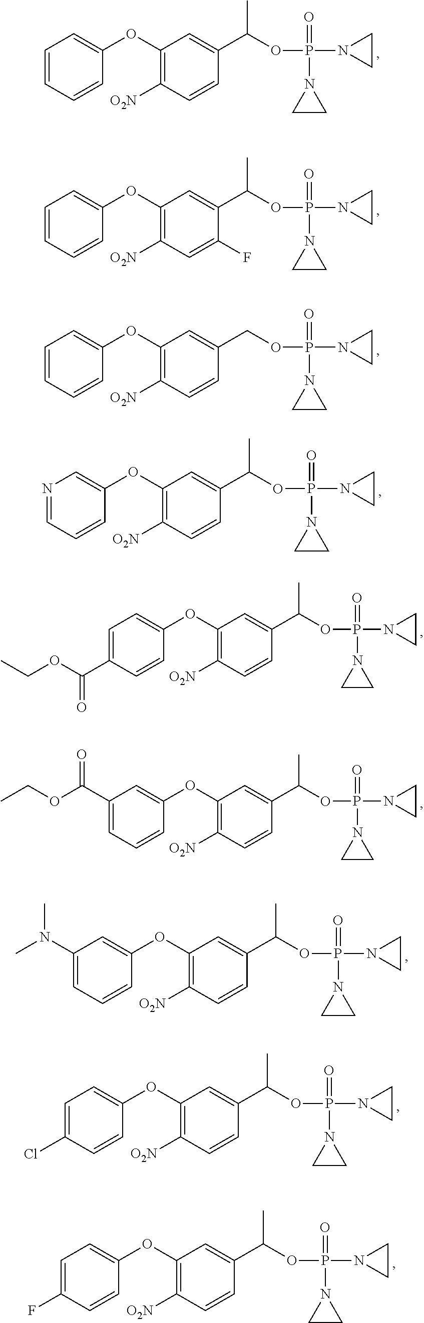

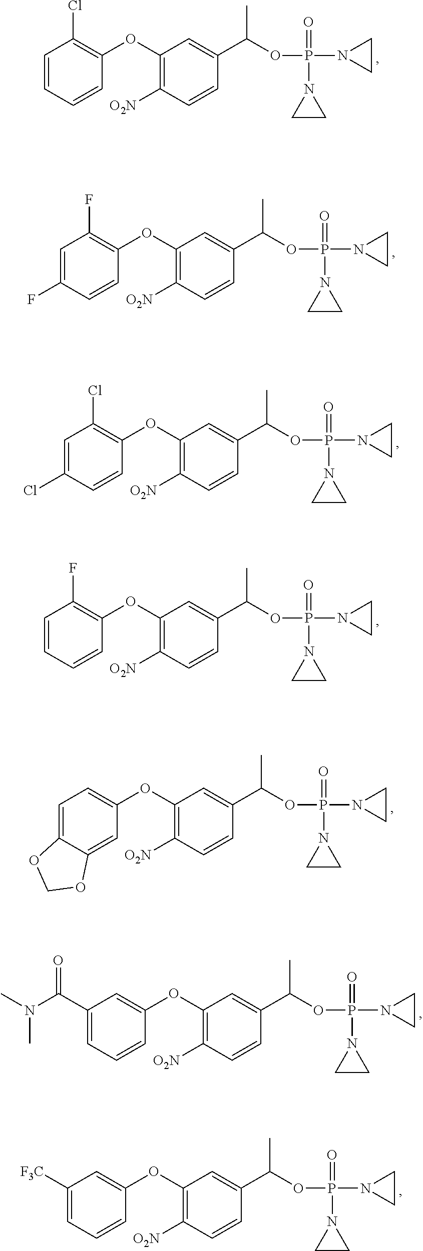

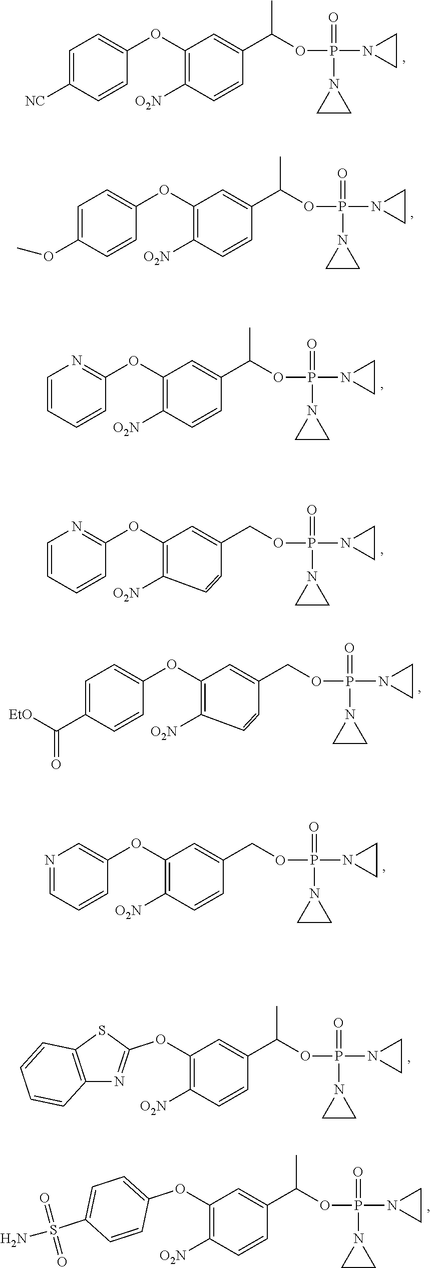

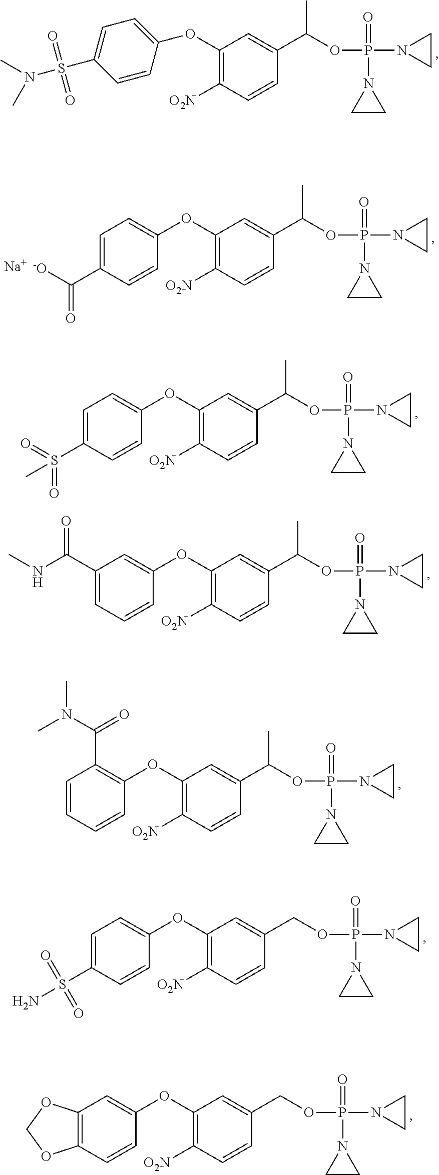

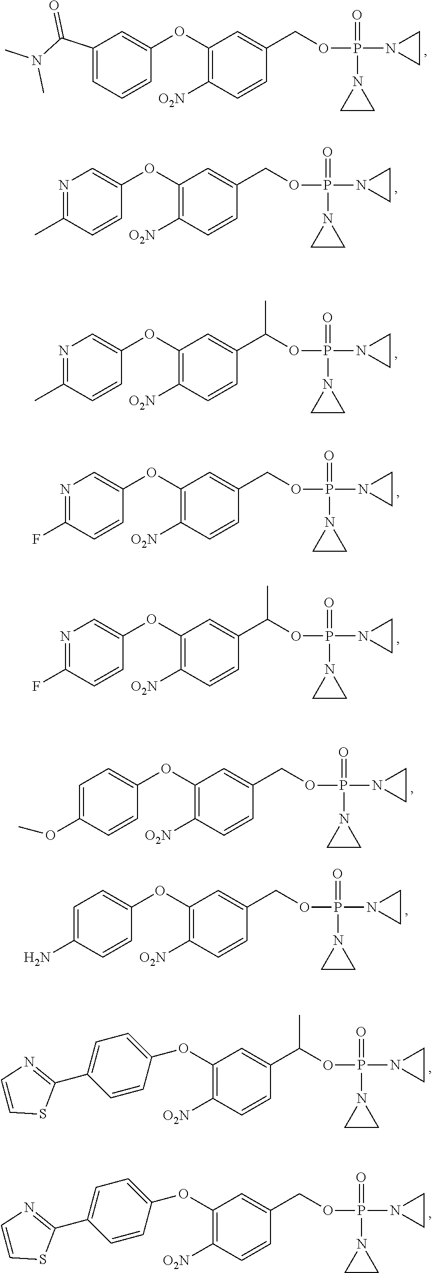

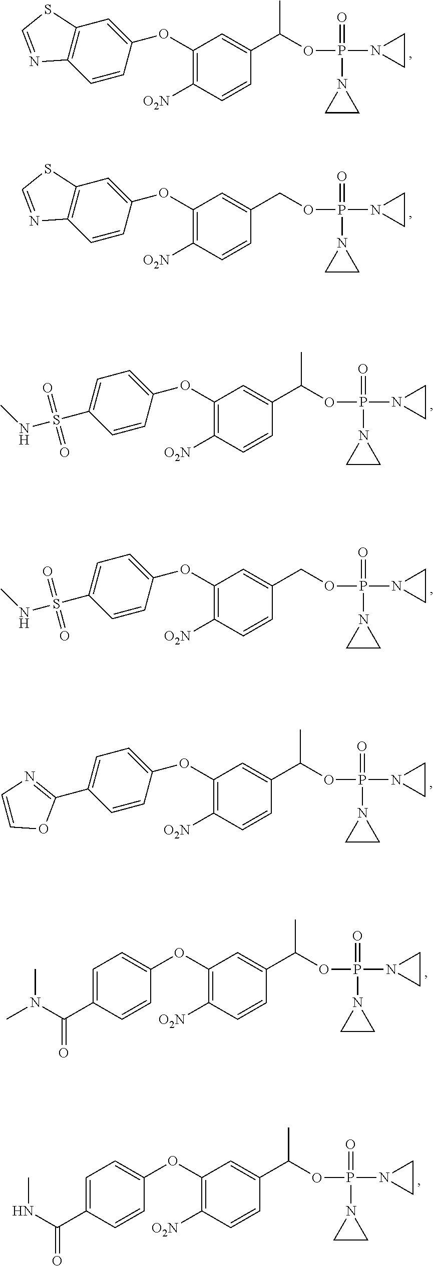

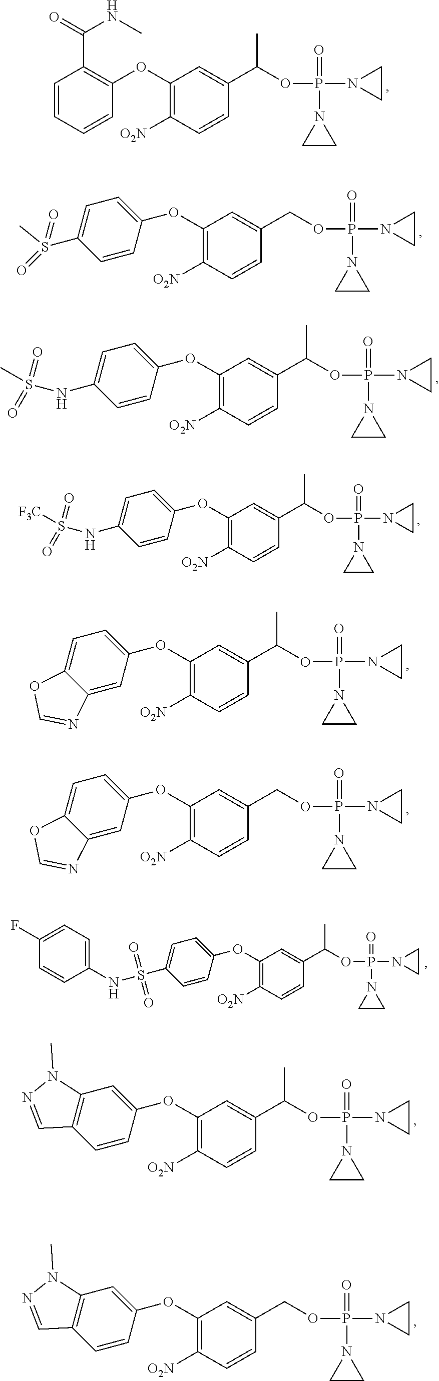

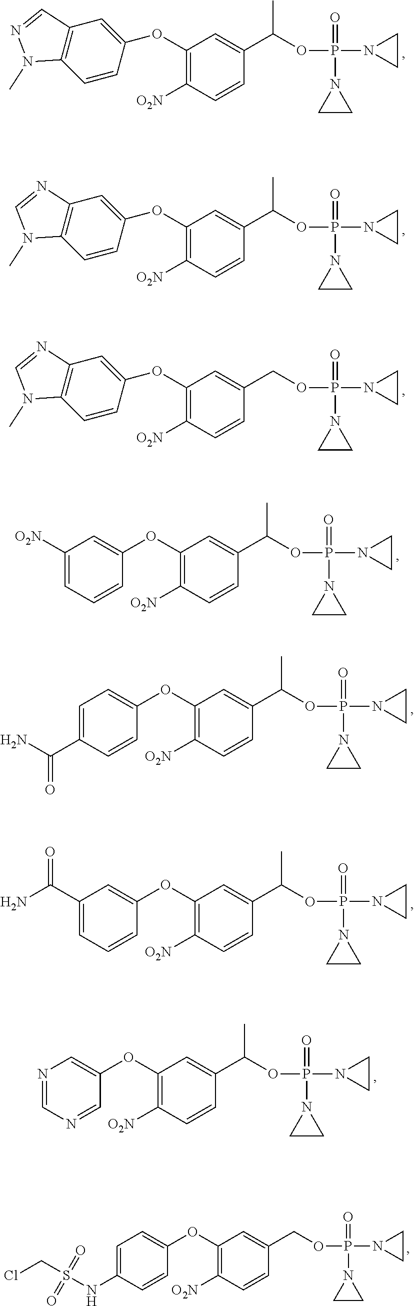

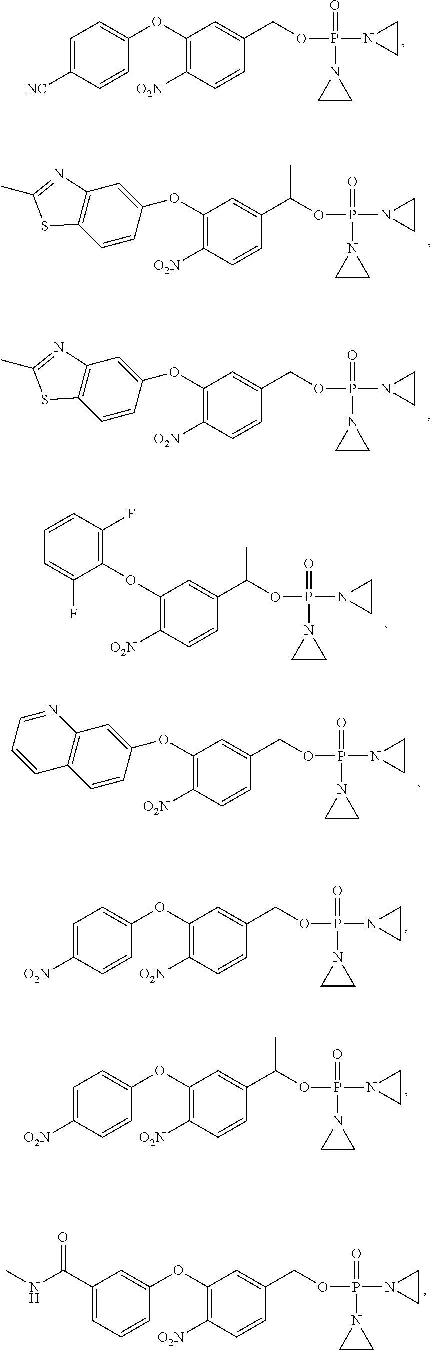

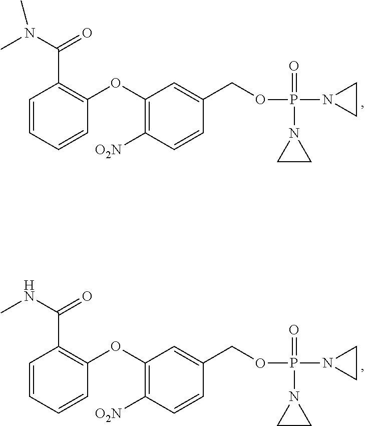 [SMILES: CN(C)C(=O)c1ccccc1Oc1cc(COP(=O)(N2CC2)N2CC2)ccc1[N+](=O)[O-] ; CNC(=O)c1ccccc1Oc1cc(COP(=O)(N2CC2)N2CC2)ccc1[N+](=O)[O-]]

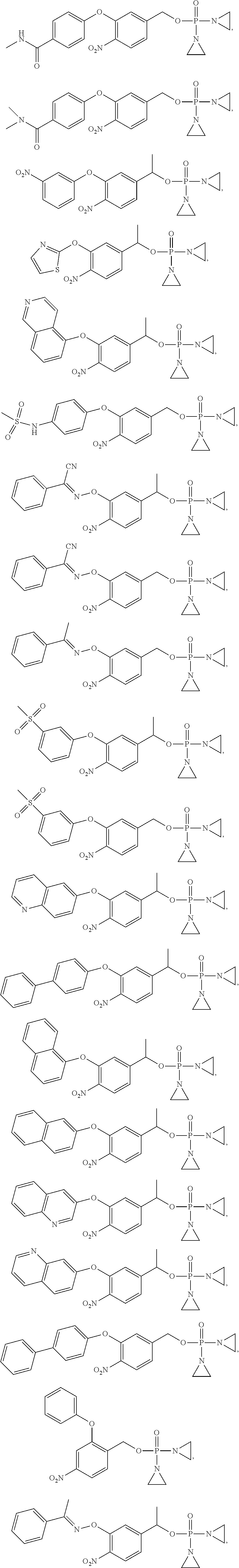

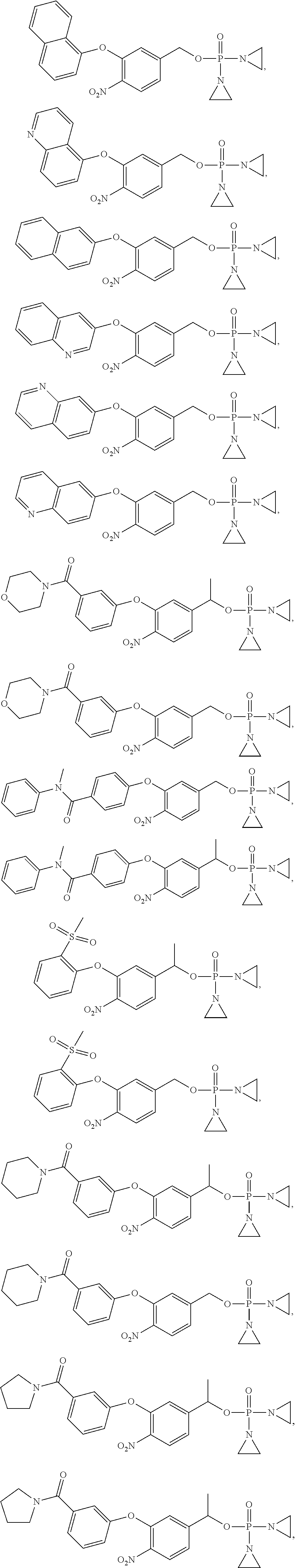

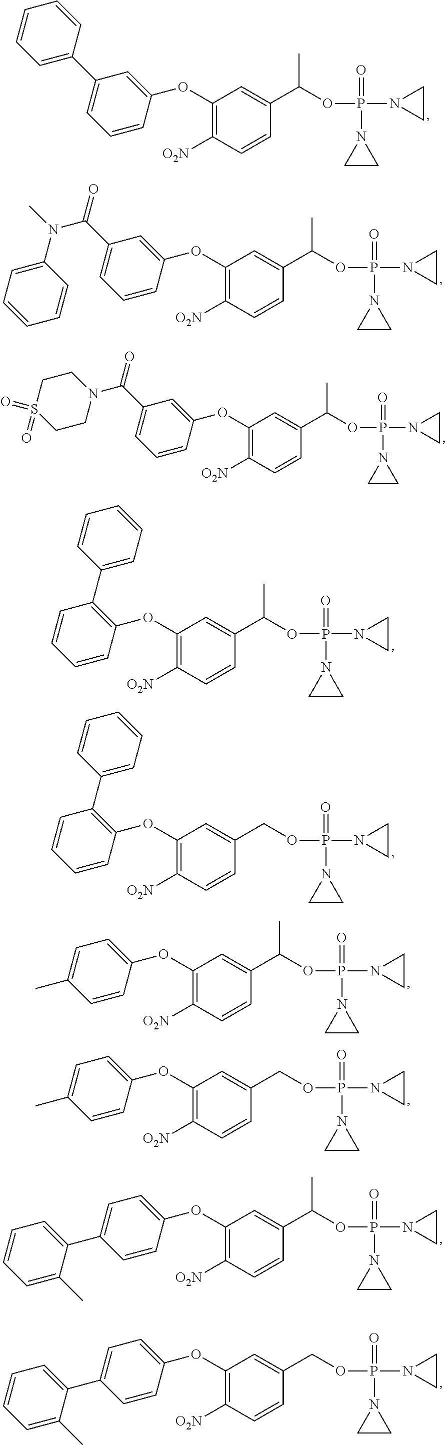

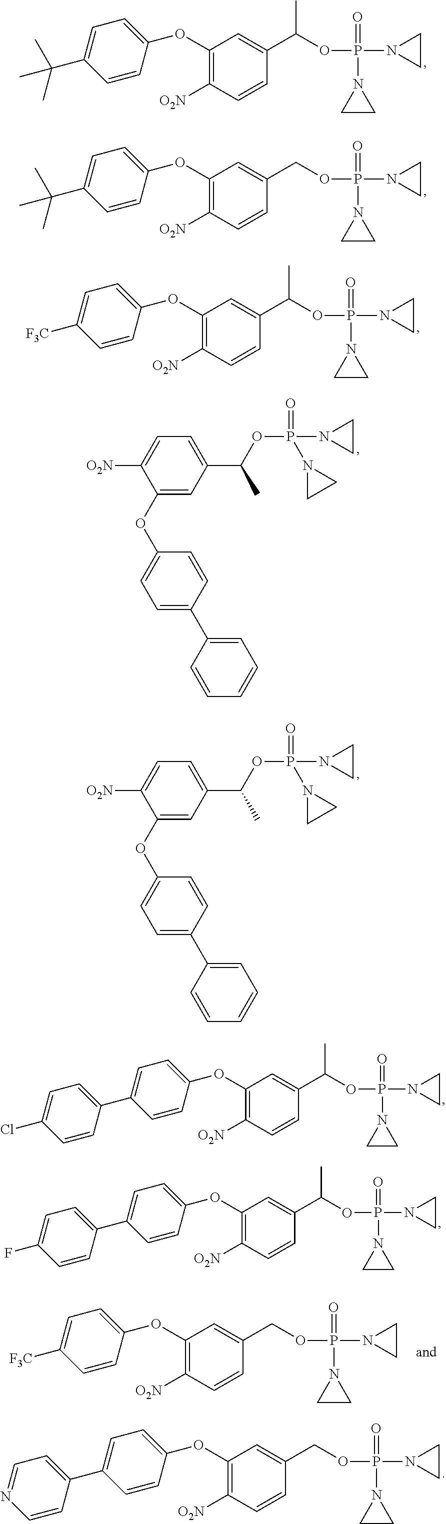

8. Pharmaceutical use of an AKR1C3-activated DNA alkylating agent prodrug compound, wherein the compound is used in the manufacture of a drug monotherapy or in combination with other therapeutic drugs for treating cancer and tumor patients having KRAS mutations.

9. The pharmaceutical use according to claim 8, wherein the compound is selected from structural Formulae 1/2/3/4/5/6 and salts, esters, solvates, isotopic isomers thereof: 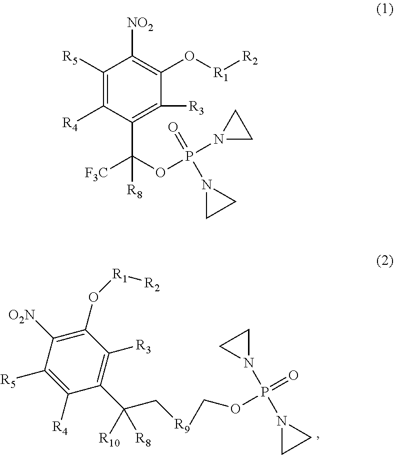
wherein the definitions of R₁, R₂, R₃, R₄, R₅, R₈, R₉, and R₁₀ are described in the claims of Patent Application PCT/CN2020/089692 with Publication No. WO2020228685A1; 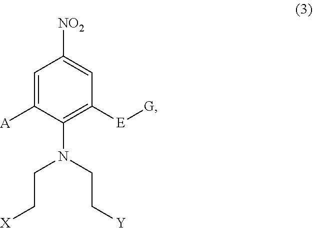
wherein the definitions of A, E, G, X, and Y are described in the claims of Patent Application PCT/NZ2019/050030 with Publication No. WO2019190331A1 (corresponding to Chinese Patent Application No. 2019800234236 with Publication No. CN111918864A); 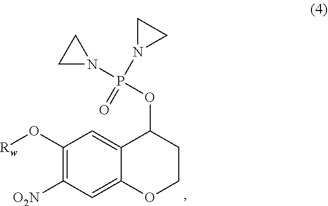
wherein the definition of R_{w} is described in the claims of Patent Application PCT/CN2020/120281 with Publication No. WO2021068952A1; 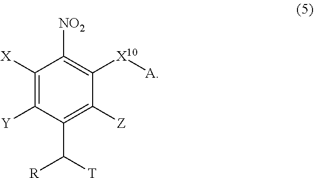
wherein the definitions of X, Y, Z, R, T, A, and X¹⁰ are described in the claims of Patent Application PCT/US2016/062114 with Publication No. WO2017087428A1 (corresponding to Chinese Patent Application No. 2016800200132 with Publication No. CN108136214A); 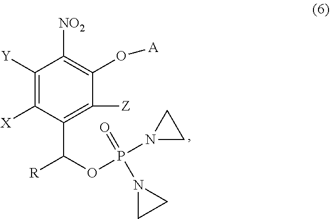
wherein:
A is a substituted or unsubstituted C₆-C₁₀ aryl, biaryl or substituted biaryl, 5-15 membered heteroaryl, or —N═CR¹R², wherein the substituents are selected from the group consisting of halogeno, —CN, —NO₂, —O—(CH₂)—O—, —CO₂H and salts thereof, —OR¹⁰⁰, —CO₂R¹⁰⁰, —CONR¹⁰¹R¹⁰², —NR¹⁰¹R¹⁰², —NR¹⁰⁰SO₂R¹⁰⁰, —SO₂R¹⁰⁰, —SO₂NR¹⁰¹R¹⁰², C₁-C₆ alkyl, and C₃-C₁₀ heterocyclyl;
wherein R¹⁰⁰, R¹⁰¹ and R¹⁰² are each independently hydrogen, C₁-C₈ alkyl, or C₆-C₁₂ aryl; or R¹⁰¹ and R¹⁰² together with the nitrogen atom to which they are attached form a 5-7 membered heterocycle;
wherein the alkyl group and the aryl group are each substituted by 1-3 halogeno groups or 1-3 C₁-C₆ alkyl groups;
R¹ and R² are each independently phenyl or methyl;
X, Y and Z are each independently hydrogen or halogeno; and
R is hydrogen or C₁-C₆ alkyl or halogen-substituted alkyl.

10. The pharmaceutical use according to claim 8, wherein the cancer is selected from the group consisting of ovarian cancer, breast cancer, pancreatic cancer, fallopian tube cancer, primary peritoneal cancer, gastric cancer, prostate cancer, liver cancer, colon cancer, rectal cancer, lung cancer, and bladder cancer.

11. The pharmaceutical use according to claim 8 or 9, wherein the KRAS mutation is selected from the group consisting of KRAS-G12D mutation, KRAS-G12V mutation and KRAS-G12C mutation;
preferably, the KRAS mutation is selected from the KRAS-G12D mutation.

12. The pharmaceutical use according to claim 11, wherein the TMB (Tumor Mutation Load (burden)) level of the mutation is medium.

13. The pharmaceutical use according to claim 8 or 9, wherein the other therapeutic drugs are selected from the group consisting of KRAS inhibitors and immunotherapy drugs, wherein the KRAS inhibitors are selected from the group consisting of sotorasib (AMG510), adagrasib (MRTX849), GDC6036, LY3499446, JNJ74699157 (ARS3248) and D-1553, and the immunotherapy drugs are selected from the group consisting of PD-1 monoclonal antibodies and PD-L1 monoclonal antibodies.
